(19) United States
(12) Patent Application Publication
(10) Pub. No.: US 2026/0207568 A1
(43) Pub. Date: Jul. 23, 2026

(54) COMPOSITIONS AND METHODS FOR IMPROVING NEUROLOGICAL DISEASES AND DISORDERS

(71) Applicant: CuraSen Therapeutics, Inc., San Carlos, CA (US)

(72) Inventors: Anthony P. Ford, San Carlos, CA (US); Gabriel Vargas, San Carlos, CA (US); Wei Chen, Saratoga, CA (US); Renee S. Martin, San Carlos, CA (US)

(21) Appl. No.: 19/139,176

(22) PCT Filed: Dec. 18, 2023
(86) PCT No.: PCT/US2023/084629

§ 371 Date: Jun. 13, 2025

Related U.S. Application Data
(60) Provisional application No. 63/434,386, filed on Dec. 21, 2022.
(60) Provisional application No. 63/434,765, filed on Dec. 22, 2022.

(51) Int. Cl. A61K 31/44; A61K 31/138; A61K 31/166; A61K 31/18; A61P 25/16; A61P 25/24; A61P 25/28

(52) U.S. Cl. CPC A61K 31/44; A61K 31/138; A61K 31/166; A61K 31/18; A61P 25/16; A61P 25/24; A61P 25/28

(57) ABSTRACT

In various aspects and embodiments provided are compositions and methods for identifying subjects in need of improving cognition and/or treating a neurodegenerative disease in a subject and treating such subject. More specifically, the disclosure in some embodiments includes administration of a β-AR agonist (such as a β-agent) and a peripherally acting β-blocker (PABRA) to a subject in need thereof.

### CROSS-REFERENCE TO RELATED APPLICATIONS

This application claims the benefit of priority under 37 C.F.R. § 119(e) of U.S. Provisional Application Nos. 63/434,386 filed Dec. 21, 2022 and 63/434,765 filed Dec. 22, 2022. The disclosures of the prior applications are considered part of and are herein incorporated by reference in the disclosure of this application in their entirety.

### FIELD OF THE DISCLOSURE

The present disclosure relates generally to compositions and methods for treating various conditions and indications including indications that are or are associated with cognition and/or neurodegenerative diseases or conditions in a subject.

### BACKGROUND OF THE INVENTION

United States Patent Application Publication Number 20130096126 discloses “a method for enhancing learning or memory of both in a mammal having impaired learning or memory or both from a neuro-degenerative disorder, which entails the step of administering at least one compound or a salt thereof which is a β₁-ARenergic receptor agonist, partial agonist or receptor ligand in an amount effective to improve the learning or memory or both of said mammal.”

United States Patent Application Publication Number 20140235726 discloses “a method of improving cognition in a subject with Down syndrome, which entails administering one or more β₂ adrenergic receptor agonists to the subject in an amount and with a frequency effective to improve cognition of the subject as measured by contextual learning tests.”

United States Patent Application Publication Number 20160184241 discloses “a method of improving cognition in a subject with Down syndrome, which entails intranasally administering one or more β₂-AR agonists or pharmaceutically-acceptable salts of either or both to the subject in an amount and with a frequency effective to improve cognition of the subject as measured contextual learning tests.”

PCT Application Publication Number WO2017115873 discloses “a combination of two or more compounds selected from the group consisting of compounds represented by the Compound No. 1-130, a preventive or therapeutic agent for Alzheimer's disease (AD)” and states “In an attempt to achieve the aforementioned object, the present inventors have screened an existing drug library consisting of 1280 kinds of pharmaceutical compounds approved by the Food and Drug Administration (FDA) in America by using nerve cells induced to differentiate from iPS cells derived from AD subjects, and extracted 129 kinds (including one kind of concomitant drug) of compounds that improve AP pathology in the nerve cells as candidate therapeutic drugs for AD.”

PCT Application Publication Number WO2006108424 states “[t]he invention furthermore relates to dermatological compositions without skin sensitization properties and which contain an enantiomerically pure enantiomer of a β₂ adrenoceptor agonist.

PCT Application Publication Number WO2018195473 provides “methods of treating a subject who has a synucleinopathy (e.g., Parkinson's disease) that include administering to a subject in need of such treatment therapeutically effective amounts of a β₂-adrenoreceptor agonist and at least one therapeutic agent.”

PCT Application WO2019/241736 (Ford) discloses “compositions and methods for improving cognition and/or treating a neurodegenerative disease in a subject“and that the methods many” . . . include identifying a subject in need of, or desiring improvement of, cognitive function and/or treatment of a neurodegenerative disease and administering to the subject a P agonist and optionally a peripherally acting P-blocker (PABRA).” Ford further discloses that “[e]xamples of selective peripherally acting b-blockers (PABRA) that may in certain embodiments be used in the methods disclosed herein include nadolol, atenolol, sotalol and labetalol.”

PCT Application WO/2018/195473 (Sherzer) discloses “[a]method of treating a subject who has a synucleinopathy, the method comprising: administering to a subject in need of such treatment therapeutically effective amounts of a β₂-adrenoreceptor agonist and at least one therapeutic agent selected from the group consisting of: a synucleinopathy therapeutic agent, a β2-adrenoreceptor antagonist and a health supplement, . . . to thereby treat Parkinson's disease in the subject . . . wherein the β2-adrenoreceptor antagonist is selected from the group consisting of carteolol, carvedilol, labetalol, nadolol, penbutolol, pindolol, sotalol, timolol, oxprenolol and butaxamine.”

PCT Application Publication Number WO2021127210 discloses methods that involve administering to a subject a β2-AR agonist and a peripherally acting P-blocker (PABRA), wherein the peripherally acting β-blocker (PABRA) is administered in a sub-therapeutic dose.

### SUMMARY OF THE INVENTION

In one aspect, a method for improving cognitive function and/or treating a neurodegenerative disease is provided wherein the method includes administering a therapeutically effective amount of a β-AR agonist (such as a β-agent) and a sub-therapeutic dose of a peripherally acting β-blocker (PABRA) to a subject. In one embodiment, a method for improving cognitive function and/or treating a neurodegenerative disease is provided wherein the method includes administering a therapeutically effective amount of a β-AR agonist (such as a β-agent) and a sub-therapeutic dose of a peripherally acting β-blocker (PABRA) to a subject. In one embodiment, a method for improving cognitive function and/or treating a neurodegenerative disease is provided wherein the method includes administering a therapeutically effective amount of a β-AR agonist (such as a β-agent) and a sub-therapeutic dose of a peripherally acting β-blocker (PABRA) to a subject.

In certain embodiments the subject has, or has been identified as having one or more conditions selected from the group consisting of major depressive disorder, treatment resistant depression (MDD/TRD), late age depression, anhedonia, post-traumatic stress disorder (PTSD), schizophrenia, cognitive/emotional impairment associated with schizophrenia, seasonal affective disorder (SAD). In certain embodiments the subject has, or has been identified as having major depressive disorder. In certain embodiments the subject has, or has been identified as having treatment resistant depression (MDD/TRD). In certain embodiments the subject has, or has been identified as having late age depression. In certain embodiments the subject has, or has been identified as having anhedonia. In certain embodiments the subject has, or has been identified as having post-traumatic stress disorder (PTSD). In certain embodiments the subject has, or has been identified as having schizophrenia. In certain embodiments the subject has, or has been identified as having cognitive/emotional impairment associated with schizophrenia. In certain embodiments the subject has, or has been identified as having seasonal affective disorder (SAD).

The term “β-agent” as used herein means a compound with a structure of Formula (I), Formula (I”), Formula (II), Formula (III), Formula (I′), Formula (II′), Formula (III′), Formula (IV′), Formula (V′), Formula (VI′), Formula (VII′), Formula (VIII′), Formula (IX′), Formula (X′), Formula (XI′), Formula (XII′), Formula (XIII′), Formula (XIV′), Formula (XV′), Formula (XVI′), Formula (XVII′), Formula (XVIII′), Formula (XIX′), Formula (XX′), Formula (XXI′), Formula (XXII′), Formula (XXIII′), Formula (XXIV′), or Formula (XXV′) as provided herein; or an optically pure stereoisomer, pharmaceutically acceptable salt, solvate, or prodrug thereof. In various embodiments, the β-agent is a compound provided in Table 1 herein. In some embodiments, the β-agent is Compound 03-5, or an optically pure stereoisomer, pharmaceutically acceptable salt, solvate, or prodrug thereof. In certain embodiments, a β-agent as disclosed herein is an agonist, partial agonist or antagonist of an adrenergic receptor; in some embodiments the β-agent is a β-AR agonist, in some embodiments the β-agent is a β1-adrenergic receptor agonist, β2-adrenertic receptor agonist or non-selective β1/β2-adrenergic receptor agonist; in some embodiments the β-agent is a β1-adrenergic receptor agonist; in some embodiments the β-agent is a β2-adrenergic receptor agonist; in some embodiments the β-agent is a non-selective β1/β2-adrenergic agonist.

In some embodiments a β-agent is a compound according to Formula (I) or an optically pure stereoisomer, pharmaceutically acceptable salt, solvate, prodrug thereof

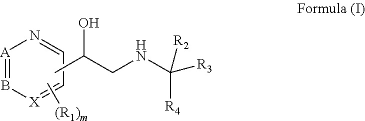

In some embodiments, each A, B, and X is independently a nitrogen or carbon. In some embodiments, each R₁ is independently selected from the group consisting of hydrogen, halogen, cyano, nitro, pentafluorosulfanyl, unsubstituted or substituted sulfonyl, substituted amino, unsubstituted or substituted alkyl, unsubstituted or substituted alkoxy, unsubstituted or substituted alkenyl, unsubstituted or substituted alkynyl, unsubstituted or substituted cycloalkyl, unsubstituted or substituted —(C═O)-alkyl, unsubstituted or substituted —(C═O)-cycloalkyl, unsubstituted or substituted —(C═O)-aryl, unsubstituted or substituted —(C═O)-heteroaryl, unsubstituted or substituted aryl, and unsubstituted or substituted heteroaryl. In some embodiments, m is an integer selected from 0 to 4.

In some embodiments, R₂, R₃, and R₄ are independently selected from the group consisting of H, halogen, hydroxyl, cyano, nitro, unsubstituted or substituted amino, unsubstituted or substituted alkyl, unsubstituted or substituted alkoxy, unsubstituted or substituted alkenyl, unsubstituted or substituted alkynyl, unsubstituted or substituted cycloalkyl, unsubstituted or substituted aryl, unsubstituted or substituted heteroaryl,

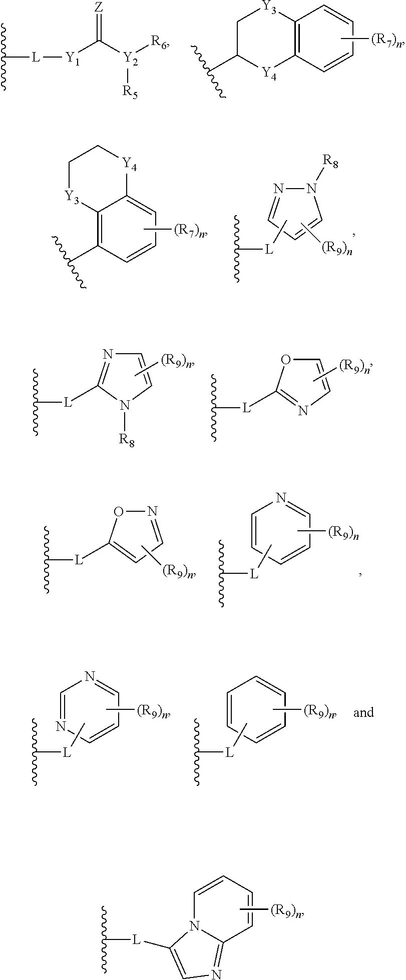

or R₂ and R₃ together with the carbon form an unsubstituted or substituted 3-7 membered cycloalkyl or heterocycle ring.

In some embodiments, L is a C1-C5 alkyl linker optionally substituted, each Y₁, Y₂, Y₃, and Y₄ is independently a covalent bond, a carbon, an oxygen, or a nitrogen, optionally substituted with hydrogen, unsubstituted or substituted alkyl, or unsubstituted or substituted cycloalkyl, and Z is O or S.

In some embodiments, R₅ and R₆ are independently selected from hydrogen, unsubstituted or substituted alkyl, or R₅ and R₆ are cyclically linked and together with Y₂ to form an optionally substituted cycloalkyl or heterocycle, each R₇ is independently selected from the group consisting of hydrogen, halogen, cyano, nitro, hydroxyl, unsubstituted or substituted amino, unsubstituted or substituted alkyl, unsubstituted or substituted alkoxy, unsubstituted or substituted alkenyl, unsubstituted or substituted alkynyl, unsubstituted or substituted cycloalkyl, unsubstituted or substituted aryl, and unsubstituted or substituted heteroaryl.

In some embodiments, n is an integer selected from 0 to 4, R₈ is selected from the group consisting of hydrogen, cyano, unsubstituted or substituted alkyl, and unsubstituted or substituted aryl, and R₉ is selected from the group consisting of hydrogen, halogen, cyano, unsubstituted or substituted alkyl, unsubstituted or substituted alkoxy, and unsubstituted or substituted amino.

Also disclosed herein is a β-agent that is a compound according to Formula (II) or an optically pure stereoisomer, pharmaceutically acceptable salt, solvate, or prodrug thereof

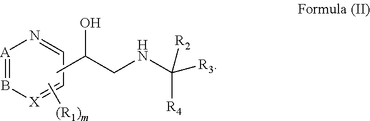

In some embodiments, each A, B, and X is independently a nitrogen or carbon. In some embodiments, each R₁ is independently selected from the group consisting of hydrogen, halogen, cyano, nitro, pentafluorosulfanyl, unsubstituted or substituted sulfonyl, substituted amino, unsubstituted or substituted alkyl, unsubstituted or substituted alkoxy, unsubstituted or substituted alkenyl, unsubstituted or substituted alkynyl, unsubstituted or substituted cycloalkyl, unsubstituted or substituted —(C═O)-alkyl, unsubstituted or substituted —(C═O)-cycloalkyl, unsubstituted or substituted —(C═O)-aryl, unsubstituted or substituted —(C═O)-heteroaryl, unsubstituted or substituted aryl, and unsubstituted or substituted heteroaryl. In some embodiments, m is an integer selected from 0 to 4.

In some embodiments, R₂, R₃, and R₄ are independently selected from the group consisting of H, halogen, hydroxyl, cyano, nitro, unsubstituted or substituted amino, unsubstituted or substituted alkyl, unsubstituted or substituted alkoxy, un-substituted or substituted alkenyl, unsubstituted or substituted alkynyl, unsubstituted or substituted cycloalkyl, unsubstituted or substituted aryl, unsubstituted or substituted heteroaryl,

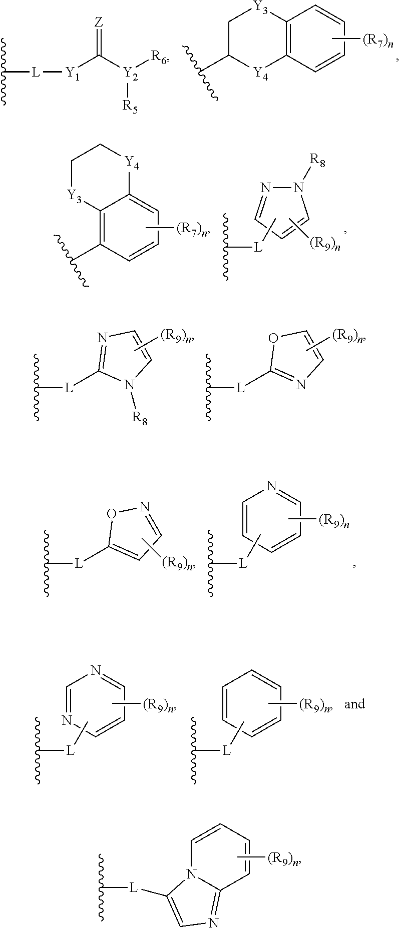

or R₂ and R₃ together with the carbon form an unsubstituted or substituted 3-7 membered cycloalkyl or heterocycle ring.

In some embodiments, L is a C1-C5 alkyl linker optionally substituted, each Y₁, Y₂, Y₃, and Y₄ is independently a covalent bond, a carbon, an oxygen, or a nitrogen, optionally substituted with hydrogen, unsubstituted or substituted alkyl, or unsubstituted or substituted cycloalkyl, and Z is O or S.

In some embodiments, R₅ and R₆ are independently selected from hydrogen, unsubstituted or substituted alkyl, or R₅ and R₆ are cyclically linked and together with Y₂ to form an optionally substituted cycloalkyl or heterocycle, each R₇ is independently selected from the group consisting of hydrogen, halogen, cyano, nitro, hydroxyl, unsubstituted or substituted amino, unsubstituted or substituted alkyl, unsubstituted or substituted alkoxy, unsubstituted or substituted alkenyl, unsubstituted or substituted alkynyl, unsubstituted or substituted cycloalkyl, unsubstituted or substituted aryl, and unsubstituted or substituted heteroaryl.

In some embodiments, n is an integer selected from 0 to 4, R₈ is selected from the group consisting of hydrogen, cyano, unsubstituted or substituted alkyl, and unsubstituted or substituted aryl, and R₉ is selected from the group consisting of hydrogen, halogen, cyano, unsubstituted or substituted alkyl, unsubstituted or substituted alkoxy, and unsubstituted or substituted amino.

In further embodiments, a β-agent is a compound according to Formula (III) or an optically pure stereoisomer, pharmaceutically acceptable salt, solvate, or prodrug thereof

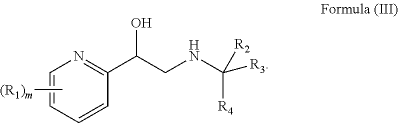

In some embodiments, each R₁ is independently selected from the group consisting of hydrogen, halogen, cyano, nitro, pentafluorosulfanyl, unsubstituted or substituted sulfonyl, substituted amino, unsubstituted or substituted alkyl, unsubstituted or substituted alkoxy, unsubstituted or substituted alkenyl, unsubstituted or substituted alkynyl, unsubstituted or substituted cycloalkyl, unsubstituted or substituted —(C═O)-alkyl, unsubstituted or substituted —(C═O)-cycloalkyl, unsubstituted or substituted —(C═O)-aryl, unsubstituted or substituted —(C═O)-heteroaryl, unsubstituted or substituted aryl, and unsubstituted or substituted heteroaryl. m is an integer selected from 0 to 4.

In some embodiments, R₂, R₃, and R₄ are independently selected from the group consisting of H, halogen, hydroxyl, cyano, nitro, unsubstituted or substituted amino, unsubstituted or substituted alkyl, unsubstituted or substituted alkoxy, unsubstituted or substituted alkenyl, unsubstituted or substituted alkynyl, unsubstituted or substituted cycloalkyl, unsubstituted or substituted aryl, unsubstituted or substituted heteroaryl,

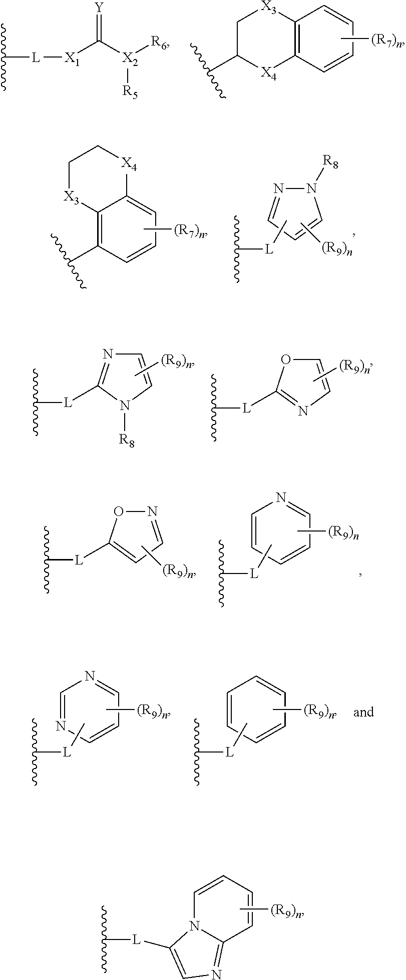

or R₂ and R₃ together with the carbon form an unsubstituted or substituted 3-7 membered cycloalkyl or heterocycle ring.

In some embodiments, L is a C1-C5 alkyl linker optionally substituted, each X₁, X₂, X₃, and X₄ is independently a covalent bond, a carbon, an oxygen, or a nitrogen, optionally substituted with hydrogen, unsubstituted or substituted alkyl, or unsubstituted or substituted cycloalkyl, and Y is O or S.

In some embodiments, R₅ and R₆ are independently selected from hydrogen, unsubstituted or substituted alkyl, or R₅ and R₆ are cyclically linked and together with Y₂ to form an optionally substituted cycloalkyl or heterocycle, each R₇ is independently selected from the group consisting of hydrogen, halogen, cyano, nitro, hydroxyl, unsubstituted or substituted amino, unsubstituted or substituted alkyl, unsubstituted or substituted alkoxy, unsubstituted or substituted alkenyl, unsubstituted or substituted alkynyl, unsubstituted or substituted cycloalkyl, unsubstituted or substituted aryl, and unsubstituted or substituted heteroaryl.

In some embodiments, n is an integer selected from 0 to 4, R₈ is selected from the group consisting of hydrogen, cyano, unsubstituted or substituted alkyl, and unsubstituted or substituted aryl, and R₉ is selected from the group consisting of hydrogen, halogen, cyano, unsubstituted or substituted alkyl, unsubstituted or substituted alkoxy, and unsubstituted or substituted amino.

Further disclosed herein is a compound according to Formula (I′):

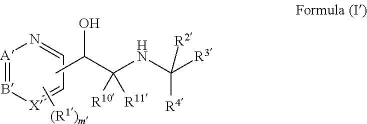

or a pharmaceutically acceptable salt thereof,
wherein:
A′, B′, and X′ are each independently nitrogen or carbon;
each R^{1′} is independently halogen, —R′, —CN, —NO₂, —SF₅, —OR^{x}, —NR^{x}₂, —NHR^{x}, —SO₂R′, —C(O)R′, —C(O)NR′₂;
each R′ is independently hydrogen or an optionally substituted group selected from: C₁₋₆ aliphatic, a 3-8 membered saturated or partially unsaturated monocyclic carbocyclic ring, phenyl, an 8-10 membered bicyclic partially unsaturated or aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic partially unsaturated or heteroaromatic ring having 1-5 heteroatoms independently selected from nitrogen, oxygen, or sulfur;
each R^{x} is independently an optionally substituted group selected from: C₁₋₆ aliphatic, a 3-8 membered saturated or partially unsaturated monocyclic carbocyclic ring, phenyl, an 8-10 membered bicyclic partially unsaturated or aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic partially unsaturated or heteroaromatic ring having 1-5 heteroatoms independently selected from nitrogen, oxygen, or sulfur;
m′ is an integer selected from 0 to 4;
R^{2′}, R^{3′}, and R^{4′} are each independently halogen, —R′, —CN, —NO₂, —OR′, —NR′₂,

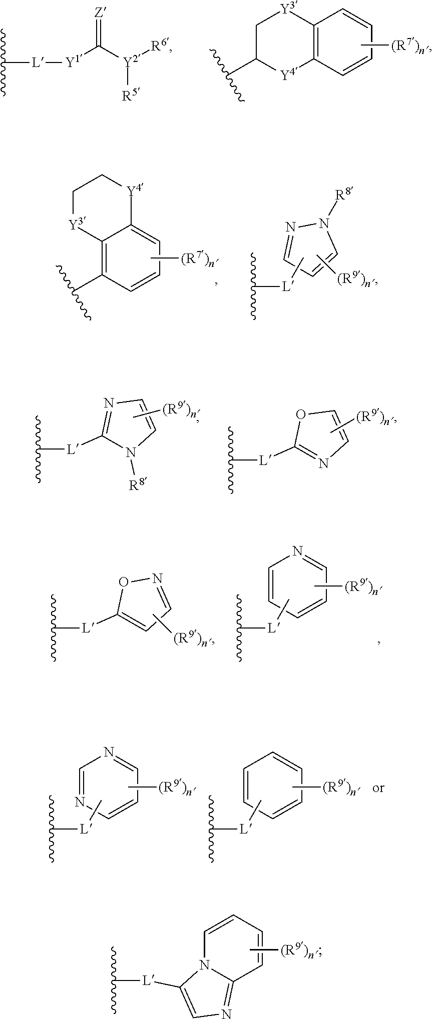

or
R^{2′} and R^{3′} together with the carbon form an optionally substituted 3-7 membered saturated carbocyclic ring; an optionally substituted 5-6 membered monocyclic heteroaryl ring having 1-4 heteroatoms independently selected from nitrogen, oxygen, and sulfur; an optionally substituted 3-7 membered saturated or a partially unsaturated monocyclic heterocyclic ring having 1-2 heteroatoms independently selected from nitrogen, oxygen, and sulfur;
L′ is optionally substituted C₁₋₅ alkylene;
Y^{1′}, Y^{2′}, Y^{3′}, and Y^{4′} are each independently a covalent bond, a carbon, an oxygen, or a nitrogen, optionally substituted with hydrogen, an optionally substituted C₁₋₆ alkyl, or an optionally substituted 3-7 membered saturated carbocyclic ring;
Z′ is O or S;
R^{5′} and R^{6′} are each independently hydrogen or optionally substituted alkyl, or
R^{5′} and R^{6′} are cyclically linked and, together with Y^{2′}, to form an optionally substituted 3-7 membered saturated carbocyclic ring; an optionally substituted 5-6 membered monocyclic heteroaryl ring having 1-4 heteroatoms independently selected from nitrogen, oxygen, and sulfur; an optionally substituted 3-7 membered saturated or partially unsaturated monocyclic heterocyclic ring having 1-2 heteroatoms independently selected from nitrogen, oxygen, and sulfur; or an optionally substituted 7-12 membered saturated or partially unsaturated bicyclic heterocyclic ring having 1-4 heteroatoms independently selected from nitrogen, oxygen, and sulfur;
each R^{7′} is independently —R′, halogen, —CN, —NO₂, —NR′₂, or —OR′;
n′ is an integer selected from 0 to 4;
R^{8′} is hydrogen, —CN, optionally substituted alkyl, or an optionally substituted aryl ring; and
each R^{9′} is independently hydrogen, halogen, —CN, —OR^{x}, —NR′₂, or optionally substituted alkyl; and
R^{10′} and R^{11′} are each independently hydrogen or optionally substituted C₁₋₂ aliphatic.

Further disclosed herein is a compound according to Formula (I″):

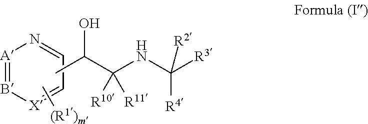

or a pharmaceutically acceptable salt thereof,
wherein:
A′, B′, and X′ are each independently nitrogen or carbon;
each R^{1′} is independently halogen, —R′, —CN, —NO₂, —SF₅, —OR^{x}, —NR^{x}₂, —NHR^{x}, —SO₂R′, —C(O)R′, —C(O)NR′₂, —NR′C(O)R′, —NR′CO₂R′, or —CO₂R′;
each R′ is independently hydrogen or an optionally substituted group selected from: C₁₋₆ aliphatic, a 3-8 membered saturated or partially unsaturated monocyclic carbocyclic ring, phenyl, an 8-10 membered bicyclic partially unsaturated or aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic partially unsaturated or heteroaromatic ring having 1-5 heteroatoms independently selected from nitrogen, oxygen, or sulfur;
each R^{x} is independently an optionally substituted group selected from: C₁₋₆ aliphatic, a 3-8 membered saturated or partially unsaturated monocyclic carbocyclic ring, phenyl, an 8-10 membered bicyclic partially unsaturated or aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic partially unsaturated or heteroaromatic ring having 1-5 heteroatoms independently selected from nitrogen, oxygen, or sulfur;
m′ is an integer selected from 0 to 4;
R^{2′}, R^{3′}, and R^{4′} are each independently halogen, —R′, —CN, —NO₂, —OR′, —NR′₂,

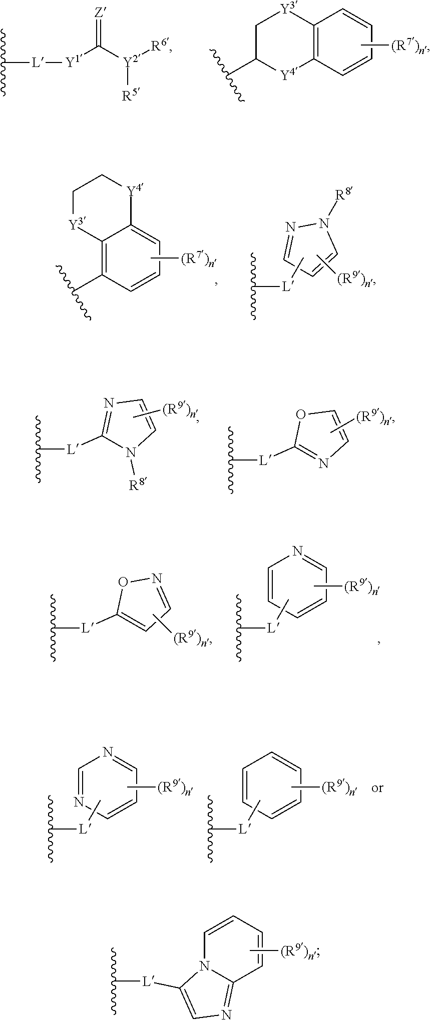

or
R^{2′} and R^{3′} together with the carbon form an optionally substituted 3-7 membered saturated carbocyclic ring; an optionally substituted 5-6 membered monocyclic heteroaryl ring having 1-4 heteroatoms independently selected from nitrogen, oxygen, and sulfur; an optionally substituted 3-7 membered saturated or a partially unsaturated monocyclic heterocyclic ring having 1-2 heteroatoms independently selected from nitrogen, oxygen, and sulfur;
L′ is optionally substituted C₁₋₅ alkylene;
Y¹, Y^{2′}, Y^{3′}, and Y^{4′} are each independently a covalent bond, a carbon, an oxygen, or a nitrogen, optionally substituted with hydrogen, an optionally substituted C₁₋₆ alkyl, or an optionally substituted 3-7 membered saturated carbocyclic ring;
Z′ is O or S;
R^{5′} and R^{6′} are each independently hydrogen or optionally substituted alkyl, or
R^{5′} and R^{6′} are cyclically linked and, together with Y^{2′}, to form an optionally substituted 3-7 membered saturated carbocyclic ring; an optionally substituted 5-6 membered monocyclic heteroaryl ring having 1-4 heteroatoms independently selected from nitrogen, oxygen, and sulfur; an optionally substituted 3-7 membered saturated or partially unsaturated monocyclic heterocyclic ring having 1-2 heteroatoms independently selected from nitrogen, oxygen, and sulfur; or an optionally substituted 7-12 membered saturated or partially unsaturated bicyclic heterocyclic ring having 1-4 heteroatoms independently selected from nitrogen, oxygen, and sulfur;
each R^{7′} is independently —R′, halogen, —CN, —NO₂, —NR′₂, or —OR′;
n′ is an integer selected from 0 to 4;
R^{8′} is hydrogen, —CN, optionally substituted alkyl, or an optionally substituted aryl ring; and
each R^{9′} is independently hydrogen, halogen, —CN, —OR^{x}, —NR′₂, or optionally substituted alkyl; and
R^{10′} and R^{11′} are each independently hydrogen or optionally substituted C₁₋₂ aliphatic.

In some embodiments a β-agent is a compound with the following structure:

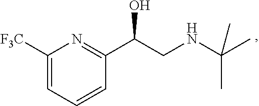 [SMILES: CC(C)(C)NC[C@H](O)c1cccc(C(F)(F)F)n1]

or a pharmaceutically acceptable salt thereof.

In some embodiments a β-agent is a compound with the following structure:

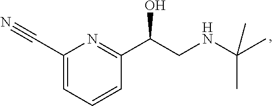 [SMILES: CC(C)(C)NC[C@H](O)c1cccc(C#N)n1]

or a pharmaceutically acceptable salt thereof.

In some embodiments a β-agent is a compound with the following structure:

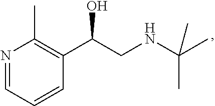

or a pharmaceutically acceptable salt thereof.

In one aspect, a method for improving cognitive function and/or treating a neurodegenerative disease is provided wherein the method includes administering a therapeutically effective amount Compound 03-5, or an optically pure stereoisomer, pharmaceutically acceptable salt, solvate, or prodrug thereof, and a sub-therapeutic dose of a peripherally acting β-blocker (PABRA) to a subject. In one embodiment, a method for improving cognitive function and/or treating a neurodegenerative disease is provided wherein the method includes administering a therapeutically effective amount of Compound 03-5 and a sub-therapeutic dose of a peripherally acting β-blocker (PABRA) to a subject. In one embodiment, a method for improving cognitive function and/or treating a neurodegenerative disease is provided wherein the method includes administering a therapeutically effective amount of Compound 03-5 and a sub-therapeutic dose of a peripherally acting β-blocker (PABRA) to a subject.

As used herein, the term “subject” can be used interchangeably with “subject” and refers to an individual that receives a composition or treatment as disclosed herein or is subjected to a method of the disclosure. In certain embodiments a subject or subject may have been diagnosed with a condition, disease or disorder and a composition or method of the disclosure is administered/applied with the intention of treating condition, disease or disorder. In some embodiments a subject or subject is any individual that receives a composition or method of the disclosure and has not necessarily been diagnosed with any particular condition, disease or disorder. In some embodiments, a subject or subject is any individual desiring an improvement in cognition or cognitive function. In various embodiments, a subject or subject may be a human or any other animal (canine, feline, etc.,).

In some embodiments of the methods and compositions provided herein, the purpose of the PABRA is not to directly treat a specific disease indication or condition, but rather to offset undesirable peripheral side effects of a β-AR agonist (such as a β-agent) (e.g., the PABRA may be administered to reduce, restrict, or counter any adverse effect(s) of the β-AR agonist (such as a β-agent), such as cardiac effects or performance-enhancing effects, thus, reducing the likelihood of abuse), and therefore in some embodiments, the PABRA dose may be lower than that generally used in previously approved therapeutic situations and indications where the PABRA is intended to directly treat a specific disease. As used herein, the term “sub-therapeutic dose” means a dose of an agent that is less than the minimum dose that is independently effective to treat a specific disease indication. In some embodiments, a sub-therapeutic dose is less than the lowest dose for which an agent is independently approved to treat any specific disease indication by a regulatory agency. In some embodiments, a sub-therapeutic dose is less than the lowest dose for which an agent is approved to treat any specific disease indication by the United States FDA. In some embodiments, a sub-therapeutic dose is less than the lowest dose for which an agent is approved to treat any specific disease indication by a regulatory agency (such as the US FDA). In certain embodiments, a subtherapeutic dose of a PABRA is sufficient to off-set or counter one or more undesirable side effects of a β-AR agonist (such as a β-agent), but the dose is less than what would generally be administered to independently treat a disease or disorder. For example, in some embodiments a sub-therapeutic dose may be 90% or less; or 85% or less; or 80% or less; or 75% or less; or 70% or less; or 65% or less; or 60% or less; or 55% or less; or 50% or less; or 45% or less; or 40% or less; or 35% or less; or 30% or less; or 25% or less; or 20% or less; or 15% or less; or 10% or less; or 5% or less; or 4% or less; or 3% or less; or 2.5% or less; or 2% or less; or 1.5% or less; or 1% or less; or 0.5% or less as compared to a dose that the agent is effective for, or approved for treating a specific disease indication. In certain embodiments, a sub-therapeutic dose for a PABRA may be about 90%; or about 85%; or about 80%; or about 75%; or about 70%; or 6 about 5%; or about 60%; or about 55%; or about 50%; or about 45%; or about 40%; or about 35%; or about 30%; or 25%; or about 20%; or about 15%; or about 10% or less; about 5%; or about 4%; or about 3%; or about 2.5%; or about 2%; or about 1.5% or less; or about 1%; or about 0.5% as compared to a dose that the agent is effective for, or approved for, treating a specific disease indication. For example, the PABRA nadolol at a dose of 40 mg once daily is approved in the United States for treatment of hypertension and angina pectoris, therefore a sub-therapeutic dose of nadolol in certain embodiments would be a dose that is less than 40 mg daily; for example a sub-therapeutic dose of nadolol may be 90% or less; or 85% or less; or 80% or less; or 75% or less; or 70% or less; or 65% or less; or 60% or less; or 55% or less; or 50% or less; or 45% or less; or 40% or less; or 35% or less; or 30% or less; or 25% or less; or 20% or less; or 15% or less; or 10% or less; or 5% or less; or 4% or less; or 3% or less; or 2.5% or less; or 2% or less; or 1.5% or less; or 1% or less; or 0.5% or less as compared to the 40 mg daily dose; or in some embodiments a sub-therapeutic dose of nadolol may be about 90%; or about 85%; or about 80%; or about 75%; or about 70%; or 6 about 5%; or about 60%; or about 55%; or about 50%; or about 45%; or about 40%; or about 35%; or about 30%; or 25%; or about 20%; or about 15%; or about 10% or less; about 5%; or about 4%; or about 3%; or about 2.5%; or about 2%; or about 1.5% or less; or about 1%; or about 0.5% of a 40 mg daily dose. In some embodiments, the peripherally acting β-blocker (PABRA) is nadolol and is administered in a total daily dose of about 0.01 to 15 mg, 0.1 to 15 mg, 0.1 to 10 mg, 0.1 to 1 mg, 0.1 to 0.5 mg, 0.2 to 0.3 mg, 0.23 to 0.27 mg; 0.1 to 5 mg, 1 to 15 mg, 1 to 10 mg, 1 to 5 mg, 5 to 10 mg, 10 mg or less, 7 mg or less, 5 mg or less, 1 mg or less, about 0.01 mg, about 0.05 mg; about 0.1 mg, about 0.2 mg, about 0.25 mg, about 0.3 mg, about 0.4 mg, about 0.5 mg, about 1 mg, about 2 mg, about 3 mg, about 4 mg, about 5 mg, about 6 mg, about 7 mg, about 8 mg, about 9 mg, or about 10 mg. In some embodiments the aforementioned doses of nadolol are weekly doses, or are twice-weekly doses. Another example of a PABRA that could be used in the methods described herein is Atenolol. Atenolol approved for various indications including hypertension, angina pectoris prophylaxis, angina pectoris, and myocardial infarction at doses ranging from 25-200 mg once daily. Accordingly, a sub-therapeutic dose of atenolol in certain embodiments would be a dose that is less than 25 mg daily; for example a sub-therapeutic dose of atenolol may be 90% or less; or 85% or less; or 80% or less; or 75% or less; or 70% or less; or 65% or less; or 60% or less; or 55% or less; or 50% or less; or 45% or less; or 40% or less; or 35% or less; or 30% or less; or 25% or less; or 20% or less; or 15% or less; or 10% or less; or 5% or less; or 4% or less; or 3% or less; or 2.5% or less; or 2% or less; or 1.5% or less; or 1% or less; or 0.5% or less as compared to a 25 mg daily dose; or in some embodiments a sub-therapeutic dose of atenolol may be about 90%; or about 85%; or about 80%; or about 75%; or about 70%; or 6 about 5%; or about 60%; or about 55%; or about 50%; or about 45%; or about 40%; or about 35%; or about 30%; or 25%; or about 20%; or about 15%; or about 10% or less; about 5%; or about 4%; or about 3%; or about 2.5%; or about 2%; or about 1.5% or less; or about 1%; or about 0.5% of a 25 mg daily dose. In some embodiments, the peripherally acting β-blocker (PABRA) is atenolol and is administered in a dose of about 0.01 to 15 mg, 0.1 to 15 mg, 0.1 to 10 mg, 0.1 to 1 mg, 0.1 to 0.5 mg, 0.2 to 0.3 mg, 0.23 to 0.27 mg; 0.1 to 5 mg, 1 to 15 mg, 1 to 10 mg, 1 to 5 mg, 5 to 10 mg, 10 mg or less, 7 mg or less, 5 mg or less, 1 mg or less, about 0.01 mg, about 0.05 mg; about 0.1 mg, about 0.2 mg, about 0.25 mg, about 0.3 mg, about 0.4 mg, about 0.5 mg, about 1 mg, about 2 mg, about 3 mg, about 4 mg, about 5 mg, about 6 mg, about 7 mg, about 8 mg, about 9 mg, or about 10 mg. In some embodiments the aforementioned doses of atenolol are weekly doses, or are twice-weekly doses.

In certain embodiments, a PABRA as used herein may have relatively limited CNS (blood-brain barrier) penetration and thus be preferentially active in the periphery.

In certain embodiments of the methods and compositions disclosed herein, the β-AR agonist (such as a β-agent) is administered in a dose that is therapeutically effective in improving cognition and/or treating a neurodegenerative disease in a subject. In some embodiments, the β-AR agonist (such as a β-agent) can be administered at a dose of from about 0.01 to 100 mg. In some embodiments, the β-AR agonist (such as a β-agent) can be administered at a dose of from about 30 to 160 μg. In some embodiments, the β-AR agonist (such as a β-agent) can be administered at a dose of from about 50 to 160 μg. For some embodiments, the β-AR agonist (such as a β-agent) can be administered at a dose of from about 1 to 300 μg, 5 to 200 μg, 10 to 180 μg, 10 to 40 μg, 20 to 50 μg, 40 to 80 μg, 50 to 100 μg, 100 to 200 μg, 30 to 160 μg, 50 to 160 μg, 80 to 160 μg, 100 to 160 μg, 120 to 160 μg, 140 to 160 μg, 150 to 170 μg, 30 to 140 μg, 50 to 140 μg, 80 to 140 μg, 100 to 140 μg, 120 to 140 μg, 30 to 120 μg, 50 to 120 μg, 80 to 120 μg, 100 to 120 μg, 30 to 100 μg, 50 to 100 μg, 80 to 100 μg, 30 to 80 μg, 50 to 80 μg, 30 to 50 μg, about 10 μg, about 20 μg, about 25 μg, about 30 μg, about 40 μg, about 50 μg, about 60 μg, about 70 μg, about 80 μg, about 90 μg, about 100 μg, about 110 μg, about 120 μg, about 125 μg, about 130 μg, about 140 μg, about 150 μg, or about 160 μg, about 170 μg, about 175 μg, about 180 μg, about 190 μg, about or 200 μg. In some embodiments, the β-AR agonist (such as a β-agent) can be administered in a dose from 150 μg to 1 mg; or from 200 μg to 500 μg, or about 250 μg, or about 300 μg, or about 400 μg, or about 500 μg. In some embodiments, the β-AR agonist (such as a β-agent) can be administered in a dose from 0.5-50 mg; or 1-25 mg; or 1-10 mg; or 10-20 mg; or 25-50 mg; or mg; or 2-8 mg; or about 1 mg; or about 2 mg; or about 3 mg; or about 4 mg, or about 5 mg; or about 6 mg; or about 7 mg; or about 8 mg; or about 10 mg; or abut 15 mg; or about 20 mg; or about 25 mg; or about 30 mg; or about 40 mg; or about 50 mg. In some embodiments of the aspects or embodiments provided herein the β-AR agonist (such as a β-agent) is administered in a dose that is from 0.5-20 mg; or 1-10 mg; or 2-8 mg; or about 1 mg; or about 2 mg; or about 3 mg; or about 4 mg; or about 5 mg; or about 6 mg; or about 7 mg; or about 8 mg; or about 10 mg. In some embodiments the aforementioned doses are daily doses, twice daily doses, weekly doses, or twice-weekly doses.

In some embodiments of any of the methods or compositions provided herein, the β-AR agonist (such as a β-agent) is administered in the morning. As used herein, the term “morning” means before 1 PM; or before noon; or before 11:30 AM; or before 11 AM; or before 10:30 AM; or before 10 AM; or before 9:30 AM; or before 9 AM; or before 8:30 AM; or before 8 AM; or within 30 mins from the time the subject awakes; or within 45 mins from time the subject awakes; or within 60 mins from the time the subject awakes; or within 90 mins from the time the subject awakes; or within 2 hours from the time the subject awakes; or within 2.5 hours from the time the subject awakes; or within 3 hours from the time the subject awakes; or within 3.5 hours from the time the subject awakes; or within 4 hours from the time the subject awakes; or within 5 hours from the time the subject awakes; or within 6 hours from the time the subject awakes; or or within 30 mins from the time the subject awakes; or within 45 mins from the time the subject awakes; or within 60 mins from the time the subject awakes; or within 90 mins from the time the subject awakes; or within 2 hours from the time the subject awakes; or within 2.5 hours from the time the subject awakes; or within 3 hours from the time the subject awakes; or within 3.5 hours from the time the subject awakes; or within 4 hours from the time the subject awakes; or within 5 hours from the time the subject awakes; or within 6 hours from the time the subject awakes; or before the subject eats after waking up; or at least 15 mins before the subject eats after waking up; or at least 30 mins before the subject eats after waking up; or at least 45 mins before the subject eats after waking up; or at least 1 hour before the subject eats after waking up.

In certain embodiments of the methods and compositions disclosed herein, the β-agent is Compound 03-5, or an optically pure stereoisomer, pharmaceutically acceptable salt, solvate, or prodrug thereof, and is administered in a dose that is therapeutically effective in improving cognition and/or treating a neurodegenerative disease in a subject. In some embodiments, Compound 03-5 can be administered at a dose of from about 0.01 to 100 mg. In some embodiments, Compound 03-5 can be administered at a dose of from about 30 to 160 μg. In some embodiments, the Compound 03-5 can be administered at a dose of from about 50 to 160 μg. For some embodiments, Compound 03-5 can be administered at a dose of from about 1 to 300 μg, 5 to 200 μg, 10 to 180 μg, 10 to 40 μg, 20 to 50 μg, 40 to 80 μg, 50 to 100 μg, 100 to 200 μg, 30 to 160 μg, 50 to 160 μg, 80 to 160 μg, 100 to 160 μg, 120 to 160 μg, 140 to 160 μg, 150 to 170 μg, 30 to 140 μg, 50 to 140 μg, 80 to 140 μg, 100 to 140 μg, 120 to 140 μg, 30 to 120 μg, 50 to 120 μg, 80 to 120 μg, 100 to 120 μg, 30 to 100 μg, 50 to 100 μg, 80 to 100 μg, 30 to 80 μg, 50 to 80 μg, 30 to 50 μg, about 10 μg, about 20 μg, about 25 μg, about 30 μg, about 40 μg, about 50 μg, about 60 μg, about 70 μg, about 80 μg, about 90 μg, about 100 μg, about 110 μg, about 120 μg, about 125 μg, about 130 μg, about 140 μg, about 150 μg, or about 160 μg, about 170 μg, about 175 μg, about 180 μg, about 190 μg, about or 200 μg. In some embodiments Compound 03-5 can be administered in a dose from 150 μg to 1 mg; or from 200 μg to 500 μg, or about 250 μg, or about 300 μg, or about 400 μg, or about 500 μg. In some embodiments, the Compound 03-05 can be administered in a dose from 0.5-50 mg; or 1-25 mg; or 3-20; or 1-20; or 1-10 mg; or 10-20 mg; or 25-50 mg; or mg; or 2-8 mg; or about 1 mg; or about 2 mg; or about 3 mg; or about 4 mg, or about 5 mg; or about 6 mg; or about 7 mg; or about 8 mg; or about 10 mg; or about 11; or about 12; or about 13; or about 14; or abut 15 mg; or about 20 mg; or about 25 mg; or about 30 mg; or about 40 mg; or about 50 mg. In some embodiments of the aspects or embodiments provided herein Compound 03-5 is administered in a dose that is from 0.5-20 mg; or 1-10 mg; or 2-8 mg; or about 1 mg; or about 2 mg; or about 3 mg; or about 4 mg; or about 5 mg; or about 6 mg; or about 7 mg; or about 8 mg; or about 10 mg. In some embodiments the aforementioned doses are daily doses, twice daily doses, weekly doses, or twice-weekly doses.

For some embodiments the doses of any agent provided herein can be a total daily dose. In some embodiments the total daily dose as provided herein is achieved by dosing once daily, in some embodiments the total daily dose is achieved by dosing twice daily, and in yet other embodiments the total daily dose is achieved by dosing more than two times daily. In certain embodiments, the doses of any agent provided herein can be a dose administered weekly or twice weekly. For some embodiments, the therapeutically effective amount of β-agent and the sub-therapeutic dose of the peripherally acting β-blocker (PABRA) are administered for a period of weeks or more; or three weeks or more; or five weeks or more; or ten weeks or more; or twenty weeks or more; or a year or more.

In one aspect, a method for improving cognitive function and/or treating a neurodegenerative disease is provided wherein the method includes administering a therapeutically effective amount of a β-AR agonist (such as a β-agent) and a peripherally acting β-blocker (PABRA) to a subject, wherein the peripherally acting β-blocker (PABRA) is administered in a dose of about 15 mg or less. In some embodiments, the peripherally acting β-blocker (PABRA; such as nadolol or atenolol) is administered in a dose of about 0.01 to 15 mg, 0.1 to 15 mg, 0.1 to 10 mg, 0.1 to 1 mg, 0.1 to 0.5 mg, 0.2 to 0.3 mg, 0.23 to 0.27 mg; 0.1 to 5 mg, 1 to 15 mg, 1 to 10 mg, 1 to 5 mg, 5 to 10 mg, 10 mg or less, 7 mg or less, 5 mg or less, 1 mg or less, about 0.01 mg, about 0.05 mg; about 0.1 mg, about 0.2 mg, about 0.25 mg, about 0.3 mg, about 0.4 mg, about 0.5 mg, about 1 mg, about 2 mg, about 3 mg, about 4 mg, about 5 mg, about 6 mg, about 7 mg, about 8 mg, about 9 mg, or about 10 mg. For some embodiments where it is not indicated differently, the above-mentioned doses are a total daily dose. For some, the above-mentioned doses are a total weekly dose. For some embodiments, the therapeutically effective amount of β-AR agonist (such as a β-agent) and the dose of the peripherally acting β-blocker (PABRA) are administered for a period of weeks or more.

In one aspect, a method for improving cognitive function and/or treating a neurodegenerative disease is provided wherein the method includes administering a therapeutically effective amount of Compound 03-5, or an optically pure stereoisomer, pharmaceutically acceptable salt, solvate, or prodrug thereof, and a peripherally acting p-blocker (PABRA) to a subject, wherein the peripherally acting β-blocker (PABRA) is administered in a dose of about 15 mg or less. In some embodiments, the peripherally acting p-blocker (PABRA; such as nadolol or atenolol) is administered in a dose of about 0.01 to 15 mg, 0.1 to 15 mg, 0.1 to 10 mg, 0.1 to 1 mg, 0.1 to 0.5 mg, 0.2 to 0.3 mg, 0.23 to 0.27 mg; 0.1 to 5 mg, 1 to 15 mg, 1 to 10 mg, 1 to 5 mg, 5 to 10 mg, 10 mg or less, 7 mg or less, 5 mg or less, 1 mg or less, about 0.01 mg, about 0.05 mg; about 0.1 mg, about 0.2 mg, about 0.25 mg, about 0.3 mg, about 0.4 mg, about 0.5 mg, about 1 mg, about 2 mg, about 3 mg, about 4 mg, about 5 mg, about 6 mg, about 7 mg, about 8 mg, about 9 mg, or about 10 mg. For some embodiments where it is not indicated differently, the above-mentioned doses are a total daily dose. For some, the above-mentioned doses are a total weekly dose. For some embodiments, the therapeutically effective amount of β-AR agonist (such as a β-agent) and the dose of the peripherally acting β-blocker (PABRA) are administered for a period of weeks or more.

The methods provided herein may further include subjecting the subject to brain imaging to determine regional metabolic activation and/or cerebral perfusion in cerebrocortical, forebrain, midbrain and brainstem areas and/or to identify whether said subject is in need of or desiring improvement of cognitive function and/or treatment of a neurodegenerative disease. In some embodiments, the brain imaging is fluorodeoxyglucose positron emission tomography (FDG-PET), used alone or in combination with other imaging approaches such as magnetic resonance imaging (MRI) and CT. In some embodiments, the brain imaging is, or can include, magnetic resonance imaging-arterial spin labeling (MRI-ASL), or magnetic resonance imaging-blood oxygenation level dependent computerized tomography (MRI-BOLD) or MRI-neuromelanin. In some embodiments the brain imaging may include MRI-ASL used to monitor cerebral blood flow, including, for example, cerebral blood flow to the hippocampus or thalamus. In some embodiments, of the aspects and embodiments disclosed herein, “improving cognition and/or treating a neurodegenerative disease” in a subject may include improving cognitive and executive function, improving inflammatory status in cerebral or cerebrospinal fluid (CSF) samples, attenuating proteinopathy burden (for example, based on imaging or CSF sampling; plasma or serum sampling is also contemplated) and/or improving regional cerebral metabolic status (reversing hypometabolism) or perfusion in the subject. In certain embodiments of the methods and compositions disclosed herein the β-AR agonist (such as a β-agent) is administered in a dose that is therapeutically effective in improving cognition and/or treating a neurodegenerative disease in a subject. As such, in certain embodiments, “identifying a subject in need of or desiring improvement of cognitive function and/or treatment of a neurodegenerative disease” may include identifying a subject in need of or desiring improvement of cognitive and executive function, improvement of inflammatory status in cerebral or CSF samples, attenuation of proteinopathy burden (for example, based on imaging or CSF, plasma or serum sampling) and/or improvement of regional cerebral metabolic/perfusion status (reversing hypometabolism or hypoperfusion). In another aspect, a method is provided wherein the method includes subjecting a subject to brain imaging to determine regional metabolic activation or perfusion in cerebrocortical, forebrain, midbrain and brainstem areas and/or to identify whether said subject is in need of or desiring improvement of cognitive function and/or treatment of a neurodegenerative disease, and administering to said subject a β-AR agonist (such as a β-agent) and a peripherally acting β-blocker (PABRA) to improve cognition and/or treat a neurodegenerative disease in said subject, wherein the peripherally acting β-blocker (PABRA) is administered in a dose of about 15 mg or less. In a similar aspect, a method is provided wherein the method includes subjecting a subject to brain imaging to determine regional metabolic or perfusion activation in cerebrocortical, forebrain, midbrain and brainstem areas and/or to identify whether said subject is in need of or desiring improvement of cognitive function and/or treatment of a neurodegenerative disease, and administering to said subject a β-AR agonist (such as a β-agent) and a peripherally acting β-blocker (PABRA) to improve cognition and/or treat a neurodegenerative disease in said subject, wherein the peripherally acting β-blocker (PABRA) is administered in a sub-therapeutic dose.

The method can further include subsequently re-subjecting said subject to brain imaging to determine any improvement in regional metabolic activation in cerebrocortical, forebrain, midbrain and brainstem areas, cognitive function and/or treatment of said neurodegenerative disease. In some embodiments, the brain imaging is FDG-PET, used alone or in combination with other imaging approaches such as MRI and CT. In some embodiments, the brain imaging is, or can include, MRI-ASL or MRI-BOLD.

In yet another aspect, a method is provided wherein the method includes subjecting a subject to brain imaging to determine regional metabolic activation in forebrain, midbrain and brainstem areas, and administering to said subject a β-AR agonist (such as a β-agent) and a peripherally acting β-blocker (PABRA), wherein the peripherally acting β-blocker (PABRA) is administered in a dose of about 15 mg or less. In a related aspect, a method is provided wherein the method includes subjecting a subject to brain imaging to determine regional metabolic activation in forebrain, midbrain and brainstem areas, and administering to said subject a β-AR agonist (such as a β-agent) and a peripherally acting β-blocker (PABRA), wherein the peripherally acting β-blocker (PABRA) is administered in a sub-therapeutic dose. The method can further include subsequently re-subjecting said subject to brain imaging to determine any improvement in regional metabolic or perfusion activation in cerebrocortical, limbic, forebrain, midbrain and brainstem areas, cognitive function. In some embodiments, the brain imaging is FDG-PET, used alone or in combination with other imaging approaches such as MRI and CT. In some embodiments, the brain imaging is, or can include, MRI-ASL or MRI-BOLD. In some embodiments the brain imaging may include MRI-ASL used to monitor cerebral blood flow, including, for example, cerebral blood flow to the hippocampus; and an improvement of cerebral blood flow (for example to the hippocampus) in the subsequent MRI-ASL is indicative of effective action of the of β-AR agonist (such as a β-agent) and/or improved cognition in the subject.

In some embodiments, a detectable label is provided, which can generate a spatial pattern of the brain imaging result. In some embodiments, 2-[¹⁸F]fluoro-2-deoxy-D-glucose (¹⁸FDG) can be used for FDG-PET, which can provide characteristic spatial patterns of brain metabolism and can help clinicians to make a reasonably accurate and early diagnosis for appropriate management or prognosis.

In some embodiments a detectable label on blood water molecules is produced by magnetic RF treatment of blood in the neck, which can generate a spatial pattern of the brains perfusion as an imaging result. In some such embodiments, MRI-ASL is used, which can provide characteristic spatial patterns of brain perfusion and can help clinicians to make a reasonably accurate and early diagnosis for appropriate management or prognosis.

In some aspects, a method for improving cognitive function and/or treating a neurodegenerative disease is provided wherein the method includes administering to said subject a β-AR agonist (such as a β-agent) and a peripherally acting β-blocker (PABRA) to improve cognition and/or treat a neurodegenerative disease in said subject, wherein the peripherally acting β-blocker (PABRA) is administered in a dose of about 15 mg or less. In some related aspects, a method for improving cognitive function and/or treating a neurodegenerative disease is provided wherein the method includes administering to said subject a β-AR agonist (such as a β-agent) and a peripherally acting β-blocker (PABRA) to improve cognition and/or treat a neurodegenerative disease in said subject, wherein the peripherally acting β-blocker (PABRA) is administered in a sub-therapeutic dose.

The method in some embodiments may further include subjecting a subject to brain imaging to determine regional metabolic activation in forebrain, midbrain and brainstem areas and/or to identify whether said subject is in need of or desiring improvement of cognitive function and/or treatment of a neurodegenerative disease. In some embodiments, the brain imaging is fluorodeoxyglucose positron emission tomography (FDG-PET), used alone or in combination with other imaging approaches such as magnetic resonance imaging (MRI) and CT. In some embodiments, the brain imaging is, or can include, MRI-ASL or MRI-BOLD or MRI-neuromelanin. In some embodiments of the aspects and embodiments disclosed herein, “improving cognition and/or treating a neurodegenerative disease” in a subject may include improving cognitive and executive function, improving inflammatory status in cerebral or cerebrospinal fluid (CSF) samples, attenuating proteinopathies burden (for example, based on imaging or CSF, plasma or serum sampling) and/or improving regional cerebral metabolic status (reversing hypometabolism) in the subject. Likewise, in certain embodiments, “identifying a subject in need of or desiring improvement of cognitive function and/or treatment of a neurodegenerative disease” may include identifying a subject in need of or desiring improvement of cognitive and executive function, improvement of inflammatory status in cerebral or CSF, plasma or serum samples, attenuation of proteinopathies burden (for example, based on imaging or CSF sampling) and/or improvement of regional cerebral metabolic status (reversing hypometabolism). In another aspect, a method is provided wherein the method includes subjecting a subject to brain imaging to determine regional metabolic activation in forebrain, midbrain and brainstem areas and/or to identify whether said subject is in need of or desiring improvement of cognitive function and/or treatment of a neurodegenerative disease, and administering to said subject a β-AR agonist (such as a β-agent) and a peripherally acting β-blocker (PABRA) to improve cognition and/or treat a neurodegenerative disease in said subject, wherein the peripherally acting β-blocker (PABRA) is administered in a dose of about 15 mg or less. In a related aspect, a method is provided wherein the method includes subjecting a subject to brain imaging to determine regional metabolic activation in forebrain, midbrain and brainstem areas and/or to identify whether said subject is in need of or desiring improvement of cognitive function and/or treatment of a neurodegenerative disease, and administering to said subject a β-AR agonist (such as a β-agent) and a peripherally acting β-blocker (PABRA) to improve cognition and/or treat a neurodegenerative disease in said subject, wherein the peripherally acting β-blocker (PABRA) is administered in a sub-therapeutic dose. For some embodiments, the peripherally acting β-blocker (PABRA) is administered to reduce, restrict, or counter any adverse effects of the β-AR agonist (such as a β-agent), e.g., performance-enhancing effects, and reduces the likelihood of abuse. In a similar aspect, a method is provided wherein the method includes subjecting a subject to brain imaging to determine regional metabolic activation in forebrain, midbrain and brainstem areas and/or to identify whether said subject is in need of or desiring improvement of cognitive function and/or treatment of a neurodegenerative disease, and administering to said subject Compound 03-5, or an optically pure stereoisomer, pharmaceutically acceptable salt, solvate, or prodrug thereof, and a peripherally acting β-blocker (PABRA) to improve cognition and/or treat a neurodegenerative disease in said subject, wherein the peripherally acting β-blocker (PABRA) is administered in a dose of about 15 mg or less. In another aspect, a method is provided wherein the method includes subjecting a subject to brain imaging to determine regional metabolic activation in forebrain, midbrain and brainstem areas and/or to identify whether said subject is in need of or desiring improvement of cognitive function and/or treatment of a neurodegenerative disease, and administering to said subject Compound 03-5, or an optically pure stereoisomer, pharmaceutically acceptable salt, solvate, or prodrug thereof, and a peripherally acting β-blocker (PABRA) to improve cognition and/or treat a neurodegenerative disease in said subject, wherein the peripherally acting β-blocker (PABRA) is administered in a sub-therapeutic dose. For some embodiments, the peripherally acting β-blocker (PABRA) is administered to reduce, restrict, or counter any adverse effects of the β-AR agonist (such as a β-agent), e.g., performance-enhancing effects, and reduces the likelihood of abuse.

The method can further include subsequently re-subjecting said subject to brain imaging to determine any improvement in regional metabolic or perfusion activation in cerebrocortical, forebrain, midbrain and brainstem areas, cognitive function and/or treatment of said neurodegenerative disease. In some embodiments, the brain imaging is FDG-PET, used alone or in combination with other imaging approaches such as MRI and CT. In some embodiments, the brain imaging is, or can include, MRI-ASL or MRI-BOLD. In yet another aspect, a method is provided wherein the method includes subjecting a subject to brain imaging determine regional metabolic activation in forebrain, midbrain and brainstem areas; administering to said subject a β-AR agonist (such as a β-agent) and a peripherally acting P-blocker (PABRA); and subsequently re-subjecting said subject to brain imaging to determine any improvement in regional metabolic activation in forebrain, midbrain and brainstem areas, cognitive function. In some embodiments, the brain imaging is FDG-PET, used alone or in combination with other imaging approaches such as MRI and CT. In some embodiments, the brain imaging is, or can include, MRI-ASL or MRI-BOLD. In some embodiments, the subject does not have Alzheimer's disease. In some embodiments, the subject does not have Down Syndrome. In some embodiments, the subject does not have Parkinson's disease. In some embodiments, the subject does not have dementia with Lewy bodies.

In some embodiments of any of the aspects and embodiments herein, the β-AR agonist (such as a β-agent) can be administered at a dose of from about 0.01 to 100 mg. In some embodiments, the β-AR agonist (such as a β-agent) can be administered at a dose of from about 30 to 160 μg. In some embodiments, the β-AR agonist (such as a β-agent) can be administered at a dose of from about 50 to 160 μg. For some embodiments, the β-AR agonist (such as a β-agent) can be administered at a dose of from about 1 to 300 μg, 5 to 200 μg, 10 to 180 μg, 10 to 40 μg, 20 to 50 μg, 40 to 80 μg, 50 to 100 μg, 100 to 200 μg, 30 to 160 μg, 50 to 160 μg, 80 to 160 μg, 100 to 160 μg, 120 to 160 μg, 140 to 160 μg, 150 to 170 μg, 30 to 140 μg, 50 to 140 μg, 80 to 140 μg, 100 to 140 μg, 120 to 140 μg, 30 to 120 μg, 50 to 120 μg, 80 to 120 μg, 100 to 120 μg, 30 to 100 μg, 50 to 100 μg, 80 to 100 μg, 30 to 80 μg, 50 to 80 μg, 30 to 50 μg, about 10 μg, about 20 μg, about 25 μg, about 30 μg, about 40 μg, about 50 μg, about 60 μg, about 70 μg, about 80 μg, about 90 μg, about 100 μg, about 110 μg, about 120 μg, about 125 μg, about 130 μg, about 140 μg, about 150 μg, or about 160 μg, about 170 μg, about 175 μg, about 180 μg, about 190 μg, about or 200 μg. In some embodiments, the β-AR agonist (such as a β-agent) can be administered in a dose from 150 μg to 1 mg; or from 200 μg to 500 μg, or about 250 μg, or about 300 μg, or about 400 μg, or about 500 μg. In some embodiments, the P-AR agonist (such as a β-agent) can be administered in a dose from 0.5-50 mg; or 1-25 mg; or 1-10 mg; or 10-20 mg; or 25-50 mg; or mg; or 2-8 mg; or about 0.25 mg; or about 0.5 mg; or about 0.75 mg; or about 1 mg; or about 2 mg; or about 3 mg; or about 4 mg, or about 5 mg; or about 6 mg; or about 7 mg; or about 8 mg; or about 10 mg; or about 11 mg, or about 12 mg, or about 13 mg, or about 14 mg, or about 15 mg; or about 20 mg; or about 25 mg; or about 30 mg; or about 40 mg; or about 50 mg. In some embodiments of the aspects or embodiments provided herein the β-AR agonist (such as a β-agent) is administered in a dose that is from 0.5-20 mg; or 1-10 mg; or 2-8 mg; or about 1 mg; or about 2 mg; or about 3 mg; or about 4 mg; or about 5 mg; or about 6 mg; or about 7 mg; or about 8 mg; or about 10 mg; or about 11 mg; or about 12 mg; or about 13 mg; or about 15 mg. In some embodiments the aforementioned doses are daily doses, twice daily doses, weekly doses, or twice-weekly doses. For some embodiments, the dose of β-AR agonist (such as a β-agent) and the peripherally acting β-blocker (PABRA) are administered or weekly for a period of weeks or more.

For some embodiments, nadolol is a mixture of diastereomers, e.g., 2, 3, 4 or more. For some embodiments, the nadolol administered is a specific enantiomerically pure isomer.

In some embodiments, the brain imaging is fluorodeoxyglucose positron emission tomography (FDG-PET), used alone or in combination with other imaging approaches such as magnetic resonance imaging (MRI) and CT. In some embodiments, the brain imaging is, or can include, MRI-ASL or MRI-BOLD. In some embodiments of the aspects and embodiments disclosed herein, “improving cognition and/or treating a neurodegenerative disease” in a subject may include improving cognitive and executive function, improving inflammatory status in cerebral or cerebrospinal fluid (CSF) samples, attenuating proteinopathies burden (for example, based on imaging or CSF sampling) and/or improving regional cerebral metabolic status (reversing hypometabolism) in the subject. Likewise, in certain embodiments, “identifying a subject in need of or desiring improvement of cognitive function and/or treatment of a neurodegenerative disease” may include identifying a subject in need of or desiring improvement of cognitive and executive function, improvement of inflammatory status in cerebral or CSF samples, attenuation of proteinopathies burden (for example, based on imaging or CSF sampling) and/or improvement of regional cerebral metabolic status (reversing hypometabolism).

In some embodiments, the brain imaging is fluorodeoxyglucose positron emission tomography (FDG-PET), used alone or in combination with other imaging approaches such as magnetic resonance imaging (MRI) and CT. In some embodiments, the brain imaging is, or can include, MRI-ASL or MRI-BOLD. In some embodiments of the aspects and embodiments disclosed herein, “improving cognition and/or treating a neurodegenerative disease” in a subject may include improving cognitive and executive function, improving inflammatory status in cerebral or cerebrospinal fluid (CSF) samples, attenuating proteinopathies burden (for example, based on imaging or CSF sampling) and/or improving regional cerebral metabolic status (reversing hypometabolism) in the subject. Likewise, in certain embodiments, “identifying a subject in need of or desiring improvement of cognitive function and/or treatment of a neurodegenerative disease” may include identifying a subject in need of or desiring improvement of cognitive and executive function, improvement of inflammatory status in cerebral or CSF samples, attenuation of proteinopathies burden (for example, based on imaging or CSF sampling) and/or improvement of regional cerebral metabolic status (reversing hypometabolism). In another aspect, a method is provided wherein the method includes subjecting a subject to brain imaging to determine regional metabolic activation in forebrain, midbrain and brainstem areas and/or to identify whether said subject is in need of or desiring improvement of cognitive function and/or treatment of a neurodegenerative disease, and administering to said subject clenbuterol or tulobuterol and nadolol to improve cognition and/or treat a neurodegenerative disease in said subject, wherein nadolol is administered in a dose of about 15 mg or less. In a related aspect, a method is provided wherein the method includes subjecting a subject to brain imaging to determine regional metabolic activation in forebrain, midbrain and brainstem areas and/or to identify whether said subject is in need of or desiring improvement of cognitive function and/or treatment of a neurodegenerative disease, and administering to said subject clenbuterol or tulobuterol and nadolol to improve cognition and/or treat a neurodegenerative disease in said subject, wherein nadolol is administered in a sub-therapeutic dose.

The method can further include subsequently re-subjecting said subject to brain imaging to determine any improvement in regional metabolic activation in forebrain, midbrain and brainstem areas, cognitive function and/or treatment of said neurodegenerative disease. In some embodiments, the brain imaging is FDG-PET, used alone or in combination with other imaging approaches such as MRI and CT. In some embodiments, the brain imaging is, or can include, MRI-ASL or MRI-BOLD. In yet another aspect, a method is provided wherein the method includes subjecting a subject to brain imaging determine regional metabolic activation in forebrain, midbrain and brainstem areas; administering to said subject clenbuterol or tulobuterol and nadolol to improve cognition and/or treat a neurodegenerative disease in said subject, wherein nadolol is administered in a dose of about 15 mg or less; and subsequently re-subjecting said subject to brain imaging to determine any improvement in regional metabolic activation in forebrain, midbrain and brainstem areas, cognitive function. In some embodiments, the brain imaging is FDG-PET, used alone or in combination with other imaging approaches such as MRI and CT. In some embodiments, the brain imaging is, or can include, MRI-ASL or MRI-BOLD.

In some aspects, a method is provided which includes treating a subject identified as having diminished cognitive function and/or being in need of or desiring improvement of cognitive function and/or treatment of a neurodegenerative disease by administering the subject a pharmaceutical composition including a β-agent, β₁-AR agonist, a β₂-AR agonist, a peripherally acting β-blocker (PABRA), or any combination thereof. In some embodiments, the method further includes assessing effectiveness of the treatment. In some embodiments, the treatment is assessed by subjecting the subject to a test to assess improved cognitive function or amelioration of the neurodegenerative disease. In some embodiments, the method further includes adjusting administration of the pharmaceutical composition by adjusting dosage of the pharmaceutical composition and/or timing of administration of the pharmaceutical composition.

In some embodiments of any of the aspects or embodiments provided herein, the methods or compositions include a β-agent and a PABRA. In some embodiments of any of the aspects or embodiments provided herein, the methods or compositions include a β-agent and a PABRA.

As used herein, the term “β agonist” or “β-AR agonist” are used interchangeably to mean an agent that acts as an agonist of a β-adrenergic receptor (β-AR). A β agonist may be a β₁ agonist, a β₁ agonist, or a non-selective β agonist. In some embodiments a β-AR agonist is a β-agent.

As used herein, the term “β₁ agonist” is used to mean β₁-adrenergic receptor agonist or β₁-AR agonist. In certain embodiments the term β₁ agonist is understood to include compounds that are primarily β₁ agonists, but which may also exhibit some peripheral agonism for other adrenergic receptors, such as β₂-adrenergic receptors. In this application, the terms “β₁-adrenergic receptor agonist”, “β₁-AR agonist”, “β₁ AR agonist” and “β₁ agonist” may be used interchangeably. In certain embodiments, the term β₁-AR agonist expressly includes both selective and partial agonists, as well as biased and non-biased agonists. Examples of β₁ adrenergic agonists include, for example, xamoterol, noradrenalin, isoprenaline, dopamine and dobutamine and the pharmaceutically-acceptable salts of any of the above. Partial agonists and ligands of the β₁-AR are known. Further, using the methodology of Kolb et al., but for β₁-AR instead, one skilled in the art could determine new ligands by structure-based discovery. See Proc. Natl. Acad. Sci. USA 2009, 106, 6843-648.

As used herein, the term “β₂ agonist” is used to mean β₂-adrenergic receptor agonist or β₂-AR agonist. In certain embodiments, the term β₂ agonist is understood to include compounds that are primarily β₂ agonists, but which may also exhibit some peripheral agonism for other adrenergic receptors, such as β₁-adrenergic receptors. In this application the terms “β₂-adrenergic receptor agonist”, “β₂-AR agonist”, “β₂AR agonist” and “β₂ agonist” may be used interchangeably. In some embodiments the term β₂-AR agonist expressly includes both selective and partial agonists. β₂ agonists that may be used in accordance with various aspects and embodiments of the present disclosure may be short-acting, long-acting or ultra long-acting. Examples of short-acting β₂ agonists that may be used are salbutamol, levosalbutamol, terbutaline, pirbuterol, procaterol, metaproterenol, bitolterol mesylate, oritodrine, isoprenaline, salmefamol, fenoterol, terbutaline, albuterol, and isoetharine. Examples of long-acting β₂ agonists that may be used are salmeterol, bambuterol, formoterol and clenbuterol. Examples of ultra long-acting β₂ agonists include indacaterol, vilanterol and olodaterol. Other examples of β₂ agonists include tulobuterol, mabuterol, and ritodrine.

As used herein, the term “peripherally acting β-blocker (PABRA)” means a β adrenergic receptor antagonist or simply a β₁-, β₂- or non-selective β-blocker. Examples of selective peripherally acting β-blockers (PABRA) that may in certain embodiments be used in the methods disclosed herein include nadolol, atenolol, sotalol and labetalol. In certain embodiments a β-blocker that can be used in the methods herein is one or more selected from the group consisting of acebutolol, betaxolol, bisoprolol, celiprolol, esmolol, metaprolol and nevivolol; in other embodiments the methods do not use acebutolol, betaxolol, bisoprolol, celiprolol, esmolol, metaprolol or nevivolol as a β-blocker. Peripherally acting β-blocker (PABRA) can be used to reduce, restrict, or counter any adverse effects of the β-agent, β₁-AR agonist and/or β₂-AR agonist, e.g., performance enhancing effects, and therefore reduces any risk of abuse. For example, nadolol can be used to reduce, restrict, or counter any peripheral P agonist effects of a β-agent.

The term “about” as used herein means in quantitative terms plus or minus 10%. For example, “about 3%” would encompass 2.7-3.3% and “about 10%” would encompass 9-11%. Moreover, where “about” is used herein in conjunction with a quantitative term it is understood that in addition to the value plus or minus 10%, the exact value of the quantitative term is also contemplated and described. For example, the term “about 3%” expressly contemplates, describes and includes exactly 3%.

In certain embodiments a peripherally acting β-blocker (PABRA) is administered to the subject prior to administration of a β-AR agonist (such as a β-agent). In other embodiments, a peripherally acting β-blocker (PABRA) is administered to the subject concurrently with the administration of a β-AR agonist (such as a β-agent). In other embodiments, a peripherally acting β-blocker (PABRA) is co-administered to the subject in a single dosing formulation, in a single tablet and/or in a single capsule.

In certain embodiments of the compositions and methods provided herein, one or more peripherally acting β-blocker (PABRA) are administered prior to or concurrently with a β-AR agonist (such as a β-agent) in order to inhibit or preclude agonism of peripheral β₁ and/or β₂ adrenergic receptors by the β-AR agonist (such as a β-agent). In various embodiments it is preferred to block peripheral β₁ and/or β₂ adrenergic receptors in accordance with the compositions and methods of the present disclosure in order to preclude, or at least minimize, any adverse effects, e.g., peripheral cardiac effects, on humans being treated.

In certain embodiments, it may be desirable for a PABRA to be administered prior to a β-AR agonist (such as a β-agent) such as to occupy peripheral β-ARs before the β-AR agonist has access to the receptors. Accordingly, in some embodiments, a PABRA and a β-AR agonist (such as a β-agent) are administered once daily (for example once daily in the morning) at a dose specified herein, the PABRA is administered prior to (i.e., 15 mins to 6 hours; or 15 mins to 3 hours; or 1 to 6 hours; or 1 to 5 hours; or 1 to 4 hours; or 1-3 hours; or 1.5 to 2.5 hours; or 2-3 hours; or 2-4 hours; or 2-5 hours; or 1.5 to 3 hours; or 1.5 to 3.5 hours; or 1.5 to 4 hours; or about 30 mins, or about 1 hour; or about 1.5 hours; or about 2 hours; or about 2.5 hours; or about 3 hours; or about 3.5 hours; or about 4 hours; or about 5 hours; 12 hours; one day) the β-AR agonist. In some embodiments a PABRA and a β-AR agonist (such as a β-agent) are administered once daily (for example once daily in the morning) at a dose specified herein, wherein for the first day only the PABRA (without the β-AR agonist) at a dose such as specified herein is administered; and for each day after the first day both the PABRA and the β-AR agonist (such as a β-agent; and at a dose such as specified herein) are administered concurrently (for example, in a single, or joint, formulation such as described herein). In some embodiments a PABRA and a β-AR agonist (such as a β-agent) are administered once daily (for example once daily in the morning) at a dose specified herein, wherein for the first and second day only the PABRA (without the β-AR agonist) at a dose such as specified herein is administered; and for each day after the second day both the PABRA and the β-AR agonist (such as a β-agent; and at a dose such as specified herein) are administered concurrently (for example, in a single, or joint, formulation such as described herein).

In certain embodiments of the methods provided herein, the β-AR agonist (such as a β-agent) is administered orally, intravenously, intramuscularly, transdermally, by inhalation or intranasally. In certain embodiments of the methods provided herein, the β-AR agonist (such as a β-agent) is administered orally.

In certain embodiments of the methods provided herein, the peripherally acting β-blocker (PABRA) is administered orally, intravenously, intramuscularly, by inhalation or intranasally. In certain embodiments of the methods provided herein, the peripherally acting β-blocker (PABRA) is administered orally.

In certain embodiments of the methods provided herein, the β-AR agonist (such as a β-agent) and the peripherally acting β-blocker (PABRA) are administered to the subject in a single formulation. In some embodiments, the single formulation is in the form of a tablet. For some embodiments both agents (β-AR agonist (such as a β-agent) and PABRA) are present in a tablet. For some embodiments, the tablet includes 30 to 160 μg of β-AR agonist (such as a β-agent), and/or 0.1 mg to 10 mg of β-AR agonist (such as a β-agent), and from about 0.1 to 15 mg of the peripherally acting β-blocker (PABRA). In some embodiments, the tablet includes the peripherally acting β-blocker (PABRA) in a sub-therapeutic dose. In some embodiments, the tablet includes the peripherally acting β-blocker (PABRA) in an amount that is 0.01 to 15 mg, 0.1 to 15 mg, 0.1 to 10 mg, 0.1 to 1 mg, 0.1 to 0.5 mg, 0.2 to 0.3 mg, 0.23 to 0.27 mg; 0.1 to 5 mg, 1 to 15 mg, 1 to 10 mg, 1 to 5 mg, 5 to 10 mg, 10 mg or less, 7 mg or less, 5 mg or less, 1 mg or less, about 0.01 mg, about 0.05 mg; about 0.1 mg, about 0.2 mg, about 0.25 mg, about 0.3 mg, about 0.4 mg, about 0.5 mg, about 1 mg, about 2 mg, about 3 mg, about 4 mg, about 5 mg, about 6 mg, about 7 mg, about 8 mg, about 9 mg, or about 10 mg. In some embodiments, the tablet includes the peripherally acting β-blocker (PABRA, such as nadolol or atenolol) in an amount that results in a dose of about 90% or less; or 85% or less; or 80% or less; or 75% or less; or 70% or less; or 65% or less; or 60% or less; or 55% or less; or 50% or less; or 45% or less; or 40% or less; or 35% or less; or 30% or less; or 25% or less; or 20% or less; or 15% or less; or 10% or less; or 5% or less; or 4% or less; or 3% or less; or 2.5% or less; or 2% or less; or 1.5% or less; or 1% or less; or 0.5% or less as compared to the 5 mg twice daily (or 10 mg total daily) dose; or in some embodiments a sub-therapeutic dose of a PABRA in the tablet may be about 90%; or about 85%; or about 80%; or about 75%; or about 70%; or 6 about 5%; or about 60%; or about 55%; or about 50%; or about 45%; or about 40%; or about 35%; or about 30%; or 25%; or about 20%; or about 15%; or about 10% or less; about 5%; or about 4%; or about 3%; or about 2.5%; or about 2%; or about 1.5% or less; or about 1%; or about 0.5% as compared to a dose that the agent is effective for, or approved for treating a specific disease indication. For some embodiments the tablet having the aforementioned doses is administered daily. For some embodiments the tablet having the aforementioned doses is administered weekly. In some embodiments, the tablet includes the peripherally acting β-blocker (PABRA) in an amount from about 5 to 10 mg. In some embodiments, the β-AR agonist (such as a β-agent) is present in the tablet from about 0.01 to 100 mg. For some embodiments, the β-AR agonist (such as a β-agent) is present in the tablet from about 30 to 160 μg, 50 to 160 μg, 80 to 160 μg, 100 to 160 μg, 120 to 160 μg, 140 to 160 μg, 30 to 140 μg, 50 to 140 μg, 80 to 140 μg, 100 to 140 μg, 120 to 140 μg, 30 to 120 μg, 50 to 120 μg, 80 to 120 μg, 100 to 120 μg, 30 to 100 μg, 50 to 100 μg, 80 to 100 μg, 30 to 80 μg, 50 to 80 μg, 30 to 50 μg, 30 μg, 40 μg, 50 μg, 60 μg, 70 μg, 80 μg, 90 μg, 100 μg, 110 μg, 120 μg, 130 μg, 140 μg, 150 μg, or 160 μg. For some embodiments, the β-AR agonist (such as a β-agent) is present in the tablet from 0.5-50 mg; or 1-25 mg; or 1-10 mg; or 10-20 mg; or 25-50 mg; or mg; or 2-8 mg; or about 0.25 mg; or about 0.5 mg; or about 0.75 mg; or about 1 mg; or about 2 mg; or about 3 mg; or about 4 mg, or about 5 mg; or about 6 mg; or about 7 mg; or about 8 mg; or about 10 mg; or about 11 mg, or about 12 mg, or about 13 mg, or about 14 mg, or about 15 mg; or about 20 mg; or about 25 mg; or about 30 mg; or about 40 mg; or about 50 mg. For some embodiments, the above-mentioned doses are a total daily dose. For some embodiments, the above-mentioned doses are a weekly dose. For some embodiments, the dose of β-AR agonist (such as a β-agent) and the peripherally acting β-blocker (PABRA) in a tablet are administered for a period of weeks or more.

In certain embodiments of the methods provided herein, the β-AR agonist (such as a β-agent) and the peripherally acting β-blocker (PABRA) are administered to the subject in a joint formulation. For some embodiments, joint formulation includes from about 30 to 160 μg of the β-AR agonist (such as a β-agent) and 15 mg or less of the peripherally acting β-blocker (PABRA). For some embodiments, joint formulation includes from about 0.5 to 20 mg of the β-AR agonist (such as a β-agent), and 15 mg or less of the peripherally acting β-blocker (PABRA). In some embodiments, the joint formulation includes the peripherally acting β-blocker (PABRA) in an amount from about 0.1 to 15 mg, 0.1 to 10 mg, 0.1 to 1 mg, 0.1 to 5 mg, 1 to 15 mg, 1 to 10 mg, 1 to 5 mg, 10 mg or less, 7 mg or less, 5 mg or less, 1 mg or less, 0.1 mg, 0.5 mg, 1 mg, 2 mg, 3 mg, 4 mg, 5 mg, 6 mg, 7 mg, 8 mg, 9 mg, or 10 mg. In some embodiments, the joint formulation includes the peripherally acting β-blocker (PABRA) in an amount from about 5 to 10 mg. In some embodiments, the β-AR agonist (such as a β-agent) is present in the joint formulation from about 0.01 to 100 mg. For some embodiments, the β-AR agonist (such as a β-agent) is present in the joint formulation from about 30 to 160 μg, 50 to 160 μg, 80 to 160 μg, 100 to 160 μg, 120 to 160 μg, 140 to 160 μg, 30 to 140 μg, 50 to 140 μg, 80 to 140 μg, 100 to 140 μg, 120 to 140 μg, 30 to 120 μg, 50 to 120 μg, 80 to 120 μg, 100 to 120 μg, 30 to 100 μg, 50 to 100 μg, 80 to 100 μg, 30 to 80 μg, 50 to 80 μg, 30 to 50 μg, 30 μg, 40 μg, 50 μg, 60 μg, 70 μg, 80 μg, 90 μg, 100 μg, 110 μg, 120 μg, 130 μg, 140 μg, 150 μg, or 160 μg. In some embodiments, the β-AR agonist (such as a β-agent) is present in the joint formulation from about 0.5-50 mg; or 1-25 mg; or 1-10 mg; or 10-20 mg; or 25-50 mg; or mg; or 2-8 mg; or about 1 mg; or about 2 mg; or about 3 mg; or about 4 mg, or about 5 mg; or about 6 mg; or about 7 mg; or about 8 mg; or about 10 mg; or about 0.25 mg; or about 0.5 mg; or about 0.75 mg; or about 15 mg; or about 20 mg; or about 25 mg; or about 30 mg; or about 40 mg; or about 50 mg. For some embodiments, the above-mentioned doses are a total daily dose. For some embodiments the doses of the joint formulations are administered weekly and the dose is total weekly dose. For some embodiments, the dose of β-AR agonist (such as a β-agent) and the peripherally acting β-blocker (PABRA) are administered daily or weekly for a period of weeks or more.

For some embodiments of the methods and compositions provided herein, both Compound 03-5, or an optically pure stereoisomer, pharmaceutically acceptable salt, solvate, or prodrug thereof, and nadolol are administered to the subject orally. For some embodiments, of the methods provided herein, Compound 03-5 and nadolol are administered to the subject orally and both agents are present in a tablet. For some embodiments, the tablet includes from about 0.01 to 100 mg of Compound 03-5 and from about 0.1 to 15 mg of nadolol. In some embodiments, the tablet includes nadolol in an amount from about 5 to 10 mg. In some embodiments, the tablet includes nadolol in an amount from about 0.1 to 15 mg, 0.1 to 10 mg, 0.1 to 1 mg, 0.1 to 5 mg, 1 to 15 mg, 1 to 10 mg, 1 to 5 mg, 10 mg or less, 7 mg or less, 5 mg or less, 1 mg or less, 0.1 mg, 0.5 mg, 1 mg, 2 mg, 3 mg, 4 mg, 5 mg, 6 mg, 7 mg, 8 mg, 9 mg, or 10 mg. For some embodiments, nadolol is a mixture of four diastereomers. For some embodiments, the nadolol administered is a specific enantiomerically pure isomer.

For some embodiments, of the methods provided herein, Compound 03-5 and nadolol are administered to the subject orally and both agents are present in a capsule. For some embodiments, the capsule includes from about 0.01 to 100 mg of Compound 03-5 and from about 0.1 to 15 mg of nadolol. In some embodiments, the capsule includes nadolol in an amount from about 5 to 10 mg. In some embodiments, the capsule includes nadolol in an amount from about 0.1 to 15 mg, 0.1 to 10 mg, 0.1 to 1 mg, 0.1 to 5 mg, 1 to 15 mg, 1 to 10 mg, 1 to 5 mg, 10 mg or less, 7 mg or less, 5 mg or less, 1 mg or less, 0.1 mg, 0.5 mg, 1 mg, 2 mg, 3 mg, 4 mg, 5 mg, 6 mg, 7 mg, 8 mg, 9 mg, or 10 mg. For some embodiments, nadolol is a mixture of four diastereomers. For some embodiments, the nadolol administered is a specific enantiomerically pure isomer.

In some embodiments, Compound 03-5, or an optically pure stereoisomer, pharmaceutically acceptable salt, solvate, or prodrug thereof, is present in a tablet from about 0.01 to 100 mg. For some embodiments, Compound 03-5 is present in the tablet from about 30 to 160 μg, 50 to 160 μg, 80 to 160 μg, 100 to 160 μg, 120 to 160 μg, 140 to 160 μg, 30 to 140 μg, 50 to 140 μg, 80 to 140 μg, 100 to 140 μg, 120 to 140 μg, 30 to 120 μg, 50 to 120 μg, 80 to 120 μg, 100 to 120 μg, 30 to 100 μg, 50 to 100 μg, 80 to 100 μg, 30 to 80 μg, 50 to 80 μg, 30 to 50 μg, 30 μg, 40 μg, 50 μg, 60 μg, 70 μg, 80 μg, 90 μg, 100 μg, 110 μg, 120 μg, 130 μg, 140 μg, 150 μg, or 160 μg. For some embodiments, Compound 03-5 is present in the tablet from about 0.5-50 mg; or 1-25 mg; or 1-10 mg; or 10-20 mg; or 25-50 mg; or mg; or 2-8 mg; or about 0.25 mg; or about 0.5 mg; or about 0.75 mg; or about 1 mg; or about 2 mg; or about 3 mg; or about 4 mg, or about 5 mg; or about 6 mg; or about 7 mg; or about 8 mg; or about 10 mg; or about 0.25 mg; or about 0.5 mg; or about 0.75 mg; or about 15 mg; or about 20 mg; or about 25 mg; or about 30 mg; or about 40 mg; or about 50 mg. For some embodiments, the tablet would be a total daily dose and is expected to be administered daily for a period of weeks or more. For some embodiments, the tablet would be a total weekly dose and is expected to be administered weekly for a period of weeks or more.

For some embodiments, nadolol can reduce, restrict, or counter any adverse effects of compound 03-5, e.g., potential performance enhancing effects, which reduce the likelihood of abuse.

Clenbuterol, and certain other P-agonists, have hypertrophic and lipolytic properties side effect that have resulted in illicit abuse by athletes and individuals desiring muscle building, athletic performance-enhancing, and/or weight loss. These side effects and propensity for abuse have created hurdles for regulatory approval (such as FDA approval) and create a certain level of a public health risk. However, the hypertrophic and lipolytic actions are caused in large part by activation of peripheral P receptors; accordingly the hypertrophic and lipolytic side effects and propensity for abuse can be reduced, mitigated or eliminated by co-administering a PABRA such as disclosed herein in combination with a β-AR agonist (such as a β-agent). In particular if the β-AR agonist (such as a β-agent) and PABRA are made and sold only in single formulations having both agents such as described herein, then it will be very difficult or impossible for those seeking illicit use or abuse to separate the agents to make a product that would be effective for muscle building, athletic performance-enhancing, or weight loss illicit use. Accordingly, in some aspects and embodiments, provided are compositions and methods that involve a single formulation (such as, for example an oral tablet or a oral capsule) having a β-AR agonist (such as a β-agent) and PABRA, that is effective for improving cognition (a CNS action) but that have a reduced risk of illicit use/abuse as compared to a formulation having only a β-AR agonist (such as a β-agent) without a PABRA. In many embodiments a sub-therapeutic dose of the PABRA is sufficient to counteract the side effects of the β-AR agonist (such as a β-agent), accordingly, a single formulation (such as, for example an oral tablet) as described herein having a β-AR agonist (such as a β-agent) and PABRA may have a therapeutically active dose of the β-AR agonist (such as a β-agent) and a sub-therapeutic dose of the PABRA.

In some embodiments of the aspects and embodiments provided herein, the subject is identified as having a neurodegenerative disease that is one or more selected from the group consisting of MCI (mild cognitive impairment), aMCI (amnestic MCI), Vascular Dementia, Mixed Dementia, FTD (fronto-temporal dementia; Pick's disease), HD (Huntington disease), Rett Syndrome, PSP (progressive supranuclear palsy), CBD (corticobasal degeneration), SCA (spinocerebellar ataxia), MSA (Multiple system atrophy), SDS (Shy-Drager syndrome), olivopontocerebellar atrophy, TBI (traumatic brain injury), CTE (chronic traumatic encephalopathy), stroke, WKS (Wernicke-Korsakoff syndrome; alcoholic dementia & thiamine deficiency), normal pressure hydrocephalus, hypersomnia/narcolepsy, ASD (autistic spectrum disorders), FXS (fragile X syndrome), TSC (tuberous sclerosis complex), prion-related diseases (CJD etc.), depressive disorders, ALS (amyotrophic lateral sclerosis) and PSP (progressive supranuclear palsy), and Gabriel's mood related disorders: Adjustment Disorder With Depressed Mood, Persistent Depressive Disorder (Dysthymia), Premenstrual Dysphoric Disorder, Depressive Disorder Due to Another Medical Condition, DLB (dementia with Lewy bodies), PD (Parkinson's disease), PDD (PD dementia), ADHD (attention deficit hyperactivity disorder), Alzheimer's disease (AD), early AD, and Down Syndrome (DS). In some embodiments the of the subject is identified as having a neurodegenerative disease that is one or more selected from the group consisting of MCI, aMCI, Vascular Dementia, Mixed Dementia, FTD (fronto-temporal dementia; Pick's disease), HD (Huntington disease), Rett Syndrome, PSP (progressive supranuclear palsy), CBD (corticobasal degeneration), SCA (spinocerebellar ataxia), MSA (Multiple system atrophy), SDS (Shy-Drager syndrome), olivopontocerebellar atrophy, TBI (traumatic brain injury), CTE (chronic traumatic encephalopathy), stroke, WKS (Wernicke-Korsakoff syndrome; alcoholic dementia & thiamine deficiency), normal pressure hydrocephalus, hypersomnia/narcolepsy, ASD (autistic spectrum disorders), FXS (fragile X syndrome), TSC (tuberous sclerosis complex), prion-related diseases (CJD etc.), depressive disorders, DLB (dementia with Lewy bodies), PD (Parkinson's disease), PDD (PD dementia), and ADHD (attention deficit hyperactivity disorder). In some embodiments the subject does not have Alzheimer's disease (AD). In some embodiments the subject does not have Down Syndrome. In some embodiments the subject does not have Parkinson's disease. In some embodiments the subject does not have dementia with Lewy bodies.

In some embodiments, the subject is subjected to a cognition test or model after said administration. In some embodiments, the subject is subjected to a cognition test or model after said administration wherein the cognition test or model is a memory test; a diagnostic indicator of mental status, brain function, mental condition; a contextual learning test and/or brain imaging. In some embodiments, the subject is subjected to a cognition test or model before said administration. In some embodiments, the subject is subjected to a cognition test or model before said administration wherein the cognition test or model is a memory test; a diagnostic indicator of mental status, brain function, mental condition; a contextual learning test and/or brain imaging. In some embodiments the subject is subjected to a cognition test or model such as a memory test; a diagnostic indicator of mental status, brain function, mental condition; a contextual learning test and/or brain imaging before said administration and the cognition test or model is used to identify a subject in need of or desiring improvement of cognitive function and/or treatment of a neurodegenerative disease in accordance with the methods and compositions provided herein. In some embodiments, the subject is subjected to a cognition test or model before and after said administration. In some embodiments, the subject is subjected to a cognition test or model before and after said administration wherein the cognition test or model is a memory test; a diagnostic indicator of mental status, brain function, mental condition; a contextual learning test and/or brain imaging.

In certain embodiments, the subject demonstrates improved cognition following said administration. In some embodiments, the subject demonstrates improved cognition as demonstrated by an improvement in a cognition test or model; a memory test; a diagnostic indicator of mental status, brain function, mental condition; a contextual learning test; brain imaging or the like in the subject.

“Improving cognition,” “improved cognition” or “improvement in cognition” means an improvement in an individual's cognitive capacity, or memory, or the like. In certain embodiments, the methods described herein result in an improvement cognition, for example as demonstrated by an improvement in a cognition test, a memory test, brain imaging and/or a contextual learning test in the subject. In some embodiments, the methods described herein result in an improvement in a contextual learning test in the subject wherein said contextual learning test is a spatial contextual learning test or Arizona Cognitive Test Battery (ACTB).

In some embodiments, the subject is a mammal. In some embodiments the subject is a human. In some embodiments, the subject is a child human. In some embodiments the subject is an adult human. Child, as used herein, means a human from about 5 to 20 years of age. Adult, as used herein, means a human from about 21 years of age and older.

### BRIEF DESCRIPTION OF THE DRAWINGS

The accompanying drawings, which are incorporated in and constitute a part of this specification, exemplify various embodiments of the present invention and, together with the description, serve to explain and illustrate principles of the present disclosure. The drawings are intended only to illustrate major features of the exemplary embodiments in a diagrammatic manner.

FIG. 1 shows a graph of cerebral blood flow in subjects after being administered a single dose of clenbuterol and/or nadolol relative to their baseline.

FIG. 2 shows a graph of cerebral blood flow in subjects after being administered a single dose of clenbuterol and/or nadolol relative to their baseline.

FIG. 3 shows a graph of cerebral blood flow in subjects after being administered a single dose of clenbuterol and subjects after being administered a single dose of pindolol relative to their baseline.

FIG. 4 shows a graph of cerebral blood flow in subjects after being administered a single dose of clenbuterol in varying amounts relative to their baseline.

FIG. 5 shows a graph of cerebral blood flow in subjects after being administered a single dose of clenbuterol in varying amounts and subjects after being administered a single dose of clenbuterol and nadolol relative to their baseline.

FIG. 6 shows that after dosing with a single dose of 160 μg of clenbuterol there is a global increase in cerebral perfusion. The legend on the right shows the different regions of interest (ROIs). The data are plotted as change from baseline in cerebral blood flow in different regions of the brain.

FIG. 7 shows a perfusion MRI-ASL image of the hippocampus as the region of interest (ROI). Six healthy subjects aged 44-52 were treated with a single dose of 80 μg clenbuterol. The Baseline vs. post-dose paired t-tests results: p=0.019. The color scale is shown in the middle and indicates cerebral blood flow with low values in red and high values in yellow.

FIG. 8 shows that in a cohort 5 of the study, “estimated doses” of clenbuterol were based on dose equivalents calculated from PK modeling of exposures at 24 hours (estimated dose of 50 μg) and 48 hours (estimated dose of 30 μg) after a single dose of 80 μg clenbuterol administered to subjects on Day 1.

FIG. 9 shows improved adaptive tracking in response to clenbuterol.

FIG. 10 shows effects of clenbuterol and a β₂-AR antagonist/β₁-AR partial agonist on the visual verbal learning test (VVLT).

FIGS. 11A-11B shows the effects of Compound 03-5 monotherapy (6 mg) on heart rate (FIG. 11A) and administered 2 hours after nadolol (1-40 mg) (FIG. 11B). Heart rate was measured by triplicate ECG recordings. Data are presented as means changes from time-matched measures recorded on the day before the first dose of study drug (Day −1).

FIG. 12 shows low dose nadolol Inhibits Peripheral effects of Compound 03-5 on blood potassium levels Dose-dependent increases in hypokalemia receiving Compound 03-5 monotherapy (0.3 mg-6 mg) in Cohorts A1, A2, A3, A4 and A5, and attenuation of hypokalemia in subjects dosed with nadolol (1-40 mg) in Cohorts D1 and D2. Data are presented as individual observations for all available subjects.

FIG. 13 is a chart showing low CNS uptake of nadolol. Time-matched concentrations for Compound 03-5 and nadolol in plasma and cerebrospinal fluid (CSF) from subjects in Cohort D1 were determined in samples collected after administration of nadolol and Compound 03-5 on Day 2 (N=3-4 subjects) and Day 6 (N=4 subjects). Data are presented as mean±SEM observations from N=3-4 subjects per dose level.

FIG. 14 shows improvements in cognition with Compound 03-5 in the presence of nadolol. CANTAB data from healthy adults 55-75 years of age (N=4) who received once-daily 3 mg nadolol and Compound 03-5 (1, 3, and 10 mg) on Day 1, Day 2 and Day 3. Data show all available data in Cohort D2 (N=4 on Apr. 22, 2021) for 2 of the tests in the CANTAB battery. Data are presented as mean changes from the within-day pre-dose assessment.

FIG. 15 shows a schema for the CLIN-011 Study.

FIGS. 16A-16B illustrates Clenbuterol+Nadolol Increases the Number of Words Recalled in PDRBD Subjects (A) Immediately After Presenting the Words, and (B) 45 Minutes After Presenting the Words.

FIG. 17 illustrates accuracy for Detecting Happiness in Participants with PDRBD.

FIG. 18 illustrates reaction Time for Accuracy for Detecting Happiness in Participants with MCI.

FIG. 19 illustrates effects of Clenbuterol+Nadolol on Vital Signs (Pulse Rate, and Blood Pressure) are modest Compared with Placebo.

FIG. 20 illustrates effects of Clenbuterol+Nadolol on QTcF are Not Different from Placebo.

FIGS. 21A-21D illustrates representative ASL Images from 1 Healthy Volunteer (A) at baseline, (B) following a Single Dose of 160 μg Clenbuterol, (C) Following 5 mg Nadolol, and (D) following Re-administration of a Single Dose of 160 μg Clenbuterol in the presence of Nadolol.

FIGS. 22A-22B shows effects of Single Doses of 20-160 μg Clenbuterol on Cerebral Blood Flow in the Brain and Regions of Interest in Healthy Volunteers Without (A) and With Nadolol (B).

FIGS. 23A-23B shows effects of Single Doses of 80 μg Clenbuterol on Cerebral Blood Flow (A) and Heart Rate (B) in Subjects with MCI or PD With (N=4) and Without Nadolol (N=4).

FIG. 24 shows a summary of TEAEs reported in more than 1 subject, and plasma concentrations of Glucose and Potassium.

### DETAILED DESCRIPTION OF THE INVENTION

In certain aspects and embodiments of the present disclosure, compositions and methods result in an improved cognition, raised cerebral metabolic activity and/or improved inflammatory control in a subject. In some embodiments, the methods described herein result in an improvement cognition, for example as demonstrated by an improvement in a cognition test or model; a memory test; a diagnostic indicator of mental status, brain function, mental condition; a contextual learning test; or the like in the subject. Such cognitive tests, diagnostics and models are well known in the art. In various aspects and embodiments, any of many accepted contextual learning tests for animals or humans can be used to assess baseline cognitive function and/or to measure or quantify improved cognitive function. In some embodiments, the compositions and methods described herein may result in an improvement one or more tests, diagnostics and models as follows. Likewise for the raised cerebral metabolic activity and improved inflammatory control—these in certain embodiments may be imaged via FDG-PET and via sampling of cerebrospinal fluid (CSF) allowing measures of inflammatory cytokines and markers of glial cell activation. In some embodiments, magnetic resonance imaging-arterial spin labeling (MRI-ASL) can be used for neuroimaging. In some embodiments, magnetic resonance imaging-blood oxygenation level dependent computerized tomography (MRI-BOLD) can be used for neuroimaging. In various embodiments, FDG-PET may be used alone or in combination with CT and/or MRI including MRI-ASL and/or MRI-BOLD. For example, FDG-PET and MRI-BOLD may be used, or FDG-PET and MRI-ASL may be used. Alternatively, FDG-PET, MRI-BOLD and MRI-ASL may be used. Alternatively, MRI, including MRI-BOLD and MRI-ASL, may be used alone or in combination, and optionally with CT.

### β-Agents

Alkyl groups refer to univalent groups derived from alkanes by removal of a hydrogen atom from any carbon atom, which include straight chain and branched chain with from 1 to 12 carbon atoms, and typically from 1 to about 10 carbons or in some embodiments, from 1 to about 6 carbon atoms, or in other embodiments having 1, 2, 3 or 4 carbon atoms. Examples of straight chain alkyl groups include, but are not limited to, methyl, ethyl, n-propyl, n-butyl, n-pentyl, and n-hexyl groups. Examples of branched chain alkyl groups include, but are not limited to isopropyl, isobutyl, sec-butyl and tert-butyl groups. Alkyl groups may be substituted or unsubstituted. Representative substituted alkyl groups may be mono-substituted or substituted more than once, such as, but not limited to, mono-, di-, or tri-substituted. As used herein, the term alkyl, unless otherwise stated, refers to both cyclic and noncyclic groups.

The terms “cyclic alkyl” or “cycloalkyl” refer to univalent groups derived from cycloalkanes by removal of a hydrogen atom from a ring carbon atom. Cycloalkyl groups are saturated or partially saturated non-aromatic structures with a single ring or multiple rings including isolated, fused, bridged, and spiro ring systems, having 3 to 14 carbon atoms, or in some embodiments, from 3 to 12, or 3 to 10, or 3 to 8, or 3, 4, 5, 6 or 7 carbon atoms. Cycloalkyl groups may be substituted or unsubstituted. Representative substituted cycloalkyl groups may be mono-substituted or substituted more than once, such as, but not limited to, mono-, di-, or tri-substituted. Examples of monocyclic cycloalkyl groups include, but are not limited to cyclopropyl, cyclobutyl, cyclopentyl, and cyclohexyl groups. Examples of multi-cyclic ring systems include, but are not limited to, bicycle[4.4.0]decane, bicycle[2.2.1]heptane, spiro[2.2]pentane, and the like. (Cycloalkyl)oxy refers to —O-cycloalkyl. (Cycloalkyl)thio refers to —S-cycloalkyl. This term also encompasses oxidized forms of sulfur, such as —S(O)-cycloalkyl, or —S(O)₂-cycloalkyl.

Alkenyl groups refer to straight and branched chain and cycloalkyl groups as defined above, with one or more double bonds between two carbon atoms. Alkenyl groups may have 2 to about 12 carbon atoms, or in some embodiment from 1 to about 10 carbons or in other embodiments, from 1 to about 6 carbon atoms, or 1, 2, 3 or 4 carbon atoms in other embodiments. Alkenyl groups may be substituted or unsubstituted. Representative substituted alkenyl groups may be mono-substituted or substituted more than once, such as, but not limited to, mono-, di-, or tri-substituted. Examples of alkenyl groups include, but are not limited to, vinyl, allyl, —CH═CH(CH₃), —CH═C(CH₃)₂, —C(CH₃)=CH₂, cyclopentenyl, cyclohexenyl, butadienyl, pentadienyl, and hexadienyl, among others.

Alkynyl groups refer to straight and branched chain and cycloalkyl groups as defined above, with one or more triple bonds between two carbon atoms. Alkynyl groups may have 2 to about 12 carbon atoms, or in some embodiment from 1 to about 10 carbons or in other embodiments, from 1 to about 6 carbon atoms, or 1, 2, 3 or 4 carbon atoms in other embodiments. Alkynyl groups may be substituted or unsubstituted. Representative substituted alkynyl groups may be mono-substituted or substituted more than once, such as, but not limited to, mono-, di-, or tri-substituted. Exemplary alkynyl groups include, but are not limited to, ethynyl, propargyl, and —C≡C(CH₃), among others.

Aryl groups are cyclic aromatic hydrocarbons that include single and multiple ring compounds, including multiple ring compounds that contain separate and/or fused aryl groups. Aryl groups may contain from 6 to about 18 ring carbons, or in some embodiments from 6 to 14 ring carbons or even 6 to 10 ring carbons in other embodiments. Aryl group also includes heteroaryl groups, which are aromatic ring compounds containing 5 or more ring members, one or more ring carbon atoms of which are replaced with heteroatom such as, but not limited to, N, O, and S. Aryl groups may be substituted or unsubstituted. Representative substituted aryl groups may be mono-substituted or substituted more than once, such as, but not limited to, mono-, di-, or tri-substituted. Aryl groups include, but are not limited to, phenyl, biphenylenyl, triphenylenyl, naphthyl, anthryl, and pyrenyl groups. Aryloxy refers to —O-aryl. Arylthio refers to —S-aryl, wherein aryl is as defined herein. This term also encompasses oxidized forms of sulfur, such as —S(O)-aryl, or —S(O)₂-aryl. Heteroaryloxy refers to —O-heteroaryl. Heteroarylthio refers to —S-heteroaryl. This term also encompasses oxidized forms of sulfur, such as —S(O)-heteroaryl, or —S(O)₂-heteroaryl.

Suitable heterocyclyl groups include cyclic groups with atoms of at least two different elements as members of its rings, of which one or more is a heteroatom such as, but not limited to, N, O, or S. Heterocyclyl groups may include 3 to about 20 ring members, or 3 to 18 in some embodiments, or about 3 to 15, 3 to 12, 3 to 10, or 3 to 6 ring members. The ring systems in heterocyclyl groups may be unsaturated, partially saturated, and/or saturated. Heterocyclyl groups may be substituted or unsubstituted. Representative substituted heterocyclyl groups may be mono-substituted or substituted more than once, such as, but not limited to, mono-, di-, or tri-substituted. Exemplary heterocyclyl groups include, but are not limited to, pyrrolidinyl, tetrahydrofuryl, dihydrofuryl, tetrahydrothienyl, tetrahydrothiopyranyl, Piperidyl, morpholinyl, thiomorpholinyl, thioxanyl, Piperazinyl, azetidinyl, aziridinyl, imidazolidinyl, pyrazolidinyl, thiazolidinyl, tetrahydrothiophenyl, tetrahydrofuranyl, dioxolyl, furanyl, thiophenyl, pyrrolyl, imidazolyl, pyrazolyl, pyrazolinyl, triazolyl, tetrazolyl, oxazolyl, isoxazolyl, thiazolyl, thiazolinyl, oxetanyl, thietanyl, homopiperidyl, oxepanyl, thiepanyl, oxazepinyl, diazepinyl, thiazepinyl, 1,2,3,6-tetrahydropyridyl, indolinyl, 2H-pyranyl, 4H-pyranyl, dioxolanyl, dioxanyl, purinyl, quinolizinyl, cinnolinyl, phthalazinyl, pteridinyl, and benzothiazolyl groups. Heterocyclyloxy refers to —O-heterocycyl. Heterocyclylthio refers to —S-heterocycyl. This term also encompasses oxidized forms of sulfur, such as —S(O)-heterocyclyl, or —S(O)₂-heterocyclyl.

Polycyclic or polycyclyl groups refer to two or more rings in which two or more carbons are common to the two adjoining rings, wherein the rings are “fused rings”; if the rings are joined by one common carbon atom, these are “spiro” ring systems. Rings that are joined through non-adjacent atoms are “bridged” rings. Polycyclic groups may be substituted or unsubstituted. Representative polycyclic groups may be substituted one or more times.

Halogen groups include F, Cl, Br, and I; nitro group refers to —NO₂; cyano group refers to —CN; isocyano group refers to —N≡C; epoxy groups encompass structures in which an oxygen atom is directly attached to two adjacent or non-adjacent carbon atoms of a carbon chain or ring system, which is essentially a cyclic ether structure. An epoxide is a cyclic ether with a three-atom ring.

An alkoxy group is a substituted or unsubstituted alkyl group, as defined above, singular bonded to oxygen. Alkoxy groups may be substituted or unsubstituted. Representative substituted alkoxy groups may be substituted one or more times. Exemplary alkoxy groups include, but are not limited to, methoxy, ethoxy, propoxy, butoxy, pentoxy, hexoxy, isopropoxy, sec-butoxy, tert-butoxy, cyclopropyloxy, cyclobutyloxy, cyclopentyloxy, and cyclohexyloxy groups.

As described herein, β-agent compounds of the present disclosure may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this disclosure are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR^{∘}; SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}; —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —S(O)(NR^{∘})R^{∘}; —S(O)₂N=C(NR^{∘}₂)₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; —SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{●}; -(haloR^{●}); —(CH₂)₀₋₂OH; —(CH₂)₀₋₂OR^{●}; —(CH₂)₀₋₂CH(OR^{●})₂; —O(haloR^{●}); —CN; —N₃; —(CH₂)₀₋₂C(O)R^{●}; —(CH₂)₀₋₂C(O)OH; —(CH₂)₀₋₂C(O)OR^{●}; —(CH₂)₀₋₂SR^{●}; —(CH₂)₀₋₂SH; —(CH₂)₀₋₂NH₂; —(CH₂)₀₋₂NHR^{●}; —(CH₂)₀₋₂NR^{●}₂; —NO₂, —SiR^{●}₃; —OSiR^{●}₃; —C(O)SR^{●}; —(C₁₋₄ straight or branched alkylene)C(O)OR^{●}; or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O; ═S; =NNR*₂; =NNHC(O)R*; =NNHC(O)OR*; ═NNHS(O)₂R*; ═NR*; ═NOR*; —O(C(R*₂))₂₋₃O—; or —S(C(R*₂))₂₋₃S—; wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{●}; -(haloR^{●}); —OH, —OR^{●}; —O(haloR^{●}); —CN; —C(O)OH; —C(O)OR^{●}; —NH₂; —NHR^{●}; —NR′₂; or —NO₂; wherein each R^{●} is unsubstituted or where preceded by “halo” is substituted only with one or more halogens, and is independently C₁₋₄ aliphatic, —CH₂Ph; —O(CH₂)₀₋₁Ph; or a 5-6-membered saturated; partially unsaturated; or aryl ring having 0-4 heteroatoms independently selected from nitrogen, oxygen, or sulfur.

Suitable substituents on a substitutable nitrogen of an “optionally substituted” group include —R^{†}; —NR^{†}₂; —C(O)R^{†}; —C(O)OR^{†}; —C(O)C(O)R^{†}; —C(O)CH₂C(O)R^{†}; —S(O)₂R^{†}; —S(O)₂NR^{†}₂; —C(S)NR^{†}₂; —C(NH)NR^{†}₂; or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of Rt, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of Rt are independently halogen, —R^{●}; -(haloR^{●}); —OH; —OR*; —O(haloR^{●}); —CN; —C(O)OH; —C(O)OR^{●}; —NH₂; —NHR^{●}; —NR^{●}₂; or —NO₂; wherein each R^{●} is unsubstituted or where preceded by “halo” is substituted only with one or more halogens, and is independently C₁₋₄ aliphatic, —CH₂Ph; —O(CH₂)₀₋₁Ph; or a 5-6-membered saturated; partially unsaturated; or aryl ring having 0-4 heteroatoms independently selected from nitrogen, oxygen, or sulfur.

Thiol refers to —SH. Thiocarbonyl refers to (=S). Sulfonyl refers to —SO₂-alkyl, —SO₂-substituted alkyl, —SO₂-cycloalkyl, —SO₂-substituted cycloalkyl, —SO₂-aryl, —SO₂-substituted aryl, —SO₂-heteroaryl, —SO₂-substituted heteroaryl, —SO₂-heterocyclyl, and —SO₂-substituted heterocyclyl. Sulfonylamino refers to —NR^{a}SO₂alkyl, —NR^{a}SO₂-substituted alkyl, —NR^{a}SO₂cycloalkyl, —NR^{a}SO₂substituted cycloalkyl, —NR^{a}SO₂aryl, —NR^{a}SO₂substituted aryl, —NR^{a}SO₂heteroaryl, —NR^{a}SO₂ substituted heteroaryl, —NR^{a}SO₂heterocyclyl, —NR^{a}SO₂ substituted heterocyclyl, wherein each R^{a} independently is as defined herein.

Carboxyl refers to —COOH or salts thereof. Carboxyester refers to —C(O)O-alkyl, —C(O)O— substituted alkyl, —C(O)O-aryl, —C(O)O-substituted aryl, —C(O)P-cycloalkyl, —C(O)O— substituted cycloalkyl, —C(O)O-heteroaryl, —C(O)O-substituted heteroaryl, —C(O)O— heterocyclyl, and —C(O)O-substituted heterocyclyl. (Carboxyester)amino refers to —NR^{a}—C(O)O-alkyl, —NR^{a}—C(O)O-substituted alkyl, —NR^{a}—C(O)O-aryl, —NR^{a}—C(O)O-substituted aryl, —NR^{a}—C(O)β-cycloalkyl, —NR^{a}—C(O)O-substituted cycloalkyl, —NR^{a}—C(O)O-heteroaryl, —NR^{a}—C(O)O-substituted heteroaryl, —NR^{a}—C(O)O-heterocyclyl, and —NR^{a}—C(O)O-substituted heterocyclyl, wherein R^{a} is as recited herein. (Carboxyester)oxy refers to —O—C(O)O-alkyl, —O—C(O)O— substituted alkyl, —O—C(O)O-aryl, —O—C(O)O-substituted aryl, —O—C(O)P-cycloalkyl, —O—C(O)O-substituted cycloalkyl, —O—C(O)O-heteroaryl, —O—C(O)O-substituted heteroaryl, —O—C(O)O-heterocyclyl, and —O—C(O)O-substituted heterocyclyl. Oxo refers to (═O).

The terms “amine” and “amino” refer to derivatives of ammonia, wherein one of more hydrogen atoms have been replaced by a substituent which include, but are not limited to alkyl, alkenyl, aryl, and heterocyclyl groups. In some embodiments, substituted amino can include —NH—CO—R. Carbamate groups refers to —O(C═O)NR₁R₂, where R₁ and R₂ are independently hydrogen, aliphatic groups, aryl groups, or heterocyclyl groups.

Aminocarbonyl refers to —C(O)N(R^{b})₂, wherein each R^{b} independently is selected from hydrogen, alkyl, substituted alkyl, aryl, substituted aryl, cycloalkyl, substituted cycloalkyl, heteroaryl, substituted heteroaryl, heterocyclyl, substituted heterocyclyl. Also, each R^{b} may optionally be joined together with the nitrogen bound thereto to form a heterocyclyl or substituted heterocyclyl group, provided that both R^{b} are not both hydrogen. Aminocarbonylalkyl refers to -alkylC(O)N(R^{b})₂, wherein each R^{b} independently is selected from hydrogen, alkyl, substituted alkyl, aryl, substituted aryl, cycloalkyl, substituted cycloalkyl, heteroaryl, substituted heteroaryl, heterocyclyl, substituted heterocyclyl. Also, each R^{b} may optionally be joined together with the nitrogen bound thereto to form a heterocyclyl or substituted heterocyclyl group, provided that both R^{b} are not both hydrogen. Aminocarbonylamino refers to —NR^{a}C(O)N(R^{b})₂, wherein R^{a} and each R^{b} are as defined herein. Aminodicarbonylamino refers to —NR^{a}C(O)C(O)N(R^{b})₂, wherein R^{a} and each R^{b} are as defined herein. Aminocarbonyloxy refers to —O—C(O)N(R^{b})₂, wherein each R^{b} independently is as defined herein. Aminosulfonyl refers to —SO₂N(R^{b})₂, wherein each R^{b} independently is as defined herein.

Imino refers to —N═R^{c} wherein R^{c} may be selected from hydrogen, aminocarbonylalkyloxy, substituted aminocarbonylalkyloxy, aminocarbonylalkylamino, and substituted aminocarbonylalkylamino.

Additionally, unless otherwise stated, structures depicted herein are also meant to include compounds that differ only in the presence of one or more isotopically enriched atoms. For example, compounds having the present structures including the replacement of hydrogen by deuterium (e.g., D or H²) or tritium (e.g., T or H³), or the replacement of a carbon by a ¹³C- or ¹⁴C-enriched carbon are included and are within the scope of this invention. Such compounds are useful, for example, as analytical tools, as probes in biological assays, or as therapeutic agents in accordance with the present invention.

Pharmaceutically acceptable salts of compounds described herein include conventional nontoxic salts or quaternary ammonium salts of a compound, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like. In other cases, described compounds may contain one or more acidic functional groups and, thus, are capable of forming pharmaceutically acceptable salts with pharmaceutically acceptable bases. These salts can likewise be prepared in situ in the administration vehicle or the dosage form manufacturing process, or by separately reacting the purified compound in its free acid form with a suitable base, such as the hydroxide, carbonate or bicarbonate of a pharmaceutically acceptable metal cation, with ammonia, or with a pharmaceutically acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, Piperazine and the like.

“Prodrug” refers to a derivative of an active agent that requires a transformation within the body to release the active agent. In certain embodiments, the transformation is an enzymatic transformation. Prodrugs are frequently, although not necessarily, pharmacologically inactive until converted to the active agent. “Promoiety” refers to a form of protecting group that, when used to mask a functional group within an active agent, converts the active agent into a prodrug. In some cases, the promoiety will be attached to the drug via bond(s) that are cleaved by enzymatic or non-enzymatic means in vivo. Any convenient prodrug forms of the subject compounds can be prepared, e.g., according to the strategies and methods described by Rautio et al. (“Prodrugs: design and clinical applications”, Nature Reviews Drug Discovery 7, 255-270 (February 2008)).

Disclosed herein is a β-agent compound according to Formula (I) or an optically pure stereoisomer, pharmaceutically acceptable salt, solvate, or prodrug thereof

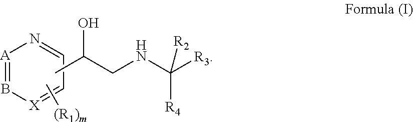

Each A, B, and X can be independently a nitrogen or carbon. Each R₁ can be independently hydrogen, halogen, cyano, nitro, pentafluorosulfanyl, unsubstituted or substituted sulfonyl, substituted amino, unsubstituted or substituted alkyl, unsubstituted or substituted alkoxy, unsubstituted or substituted alkenyl, unsubstituted or substituted alkynyl, unsubstituted or substituted cycloalkyl, unsubstituted or substituted —(C═O)-alkyl, unsubstituted or substituted —(C═O)-cycloalkyl, unsubstituted or substituted —(C═O)-aryl, unsubstituted or substituted —(C═O)-heteroaryl, unsubstituted or substituted aryl, or unsubstituted or substituted heteroaryl. m can be an integer selected from 0 to 4.

R₂, R₃, and R₄ can be independently H, halogen, hydroxyl, cyano, nitro, unsubstituted or substituted amino, unsubstituted or substituted alkyl, unsubstituted or substituted alkoxy, unsubstituted or substituted alkenyl, unsubstituted or substituted alkynyl, unsubstituted or substituted cycloalkyl, unsubstituted or substituted aryl, unsubstituted or substituted heteroaryl,

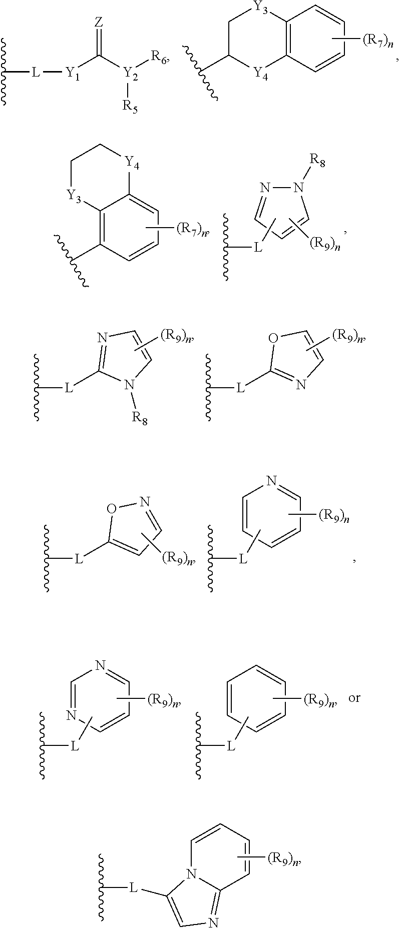

or R₂ and R₃ together with the carbon can form an unsubstituted or substituted 3-7 membered cycloalkyl or heterocycle ring.

L can be a C₁-C₅ alkyl linker optionally substituted, each Y₁, Y₂, Y₃, and Y₄ can be independently a covalent bond, a carbon, an oxygen, or a nitrogen, optionally substituted with hydrogen, unsubstituted or substituted alkyl, or unsubstituted or substituted cycloalkyl, and Z can be O or S.

R₅ and R₆ can be independently hydrogen, unsubstituted or substituted alkyl, or R₅ and R₆ are cyclically linked and together with Y₂ to form an optionally substituted cycloalkyl or heterocycle, each R₇ is independently selected from the group consisting of hydrogen, halogen, cyano, nitro, hydroxyl, unsubstituted or substituted amino, unsubstituted or substituted alkyl, unsubstituted or substituted alkoxy, unsubstituted or substituted alkenyl, unsubstituted or substituted alkynyl, unsubstituted or substituted cycloalkyl, unsubstituted or substituted aryl, or unsubstituted or substituted heteroaryl.

n can be an integer selected from 0 to 4, R₈ can be hydrogen, cyano, unsubstituted or substituted alkyl, and unsubstituted or substituted aryl, and R₉ is selected from the group consisting of hydrogen, halogen, cyano, unsubstituted or substituted alkyl, unsubstituted or substituted alkoxy, or unsubstituted or substituted amino.

Also disclosed herein is a β-agent compound according to Formula (II) or an optically pure stereoisomer, pharmaceutically acceptable salt, solvate, or prodrug thereof

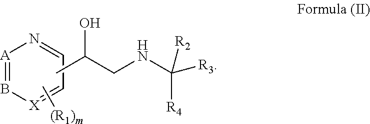

Each A, B, and X can be independently a nitrogen or carbon. Each R₁ can be hydrogen, halogen, cyano, nitro, pentafluorosulfanyl, unsubstituted or substituted sulfonyl, substituted amino, unsubstituted or substituted alkyl, unsubstituted or substituted alkoxy, unsubstituted or substituted alkenyl, unsubstituted or substituted alkynyl, unsubstituted or substituted cycloalkyl, unsubstituted or substituted —(C═O)-alkyl, unsubstituted or substituted —(C═O)-cycloalkyl, unsubstituted or substituted —(C═O)-aryl, unsubstituted or substituted —(C═O)-heteroaryl, unsubstituted or substituted aryl, or unsubstituted or substituted heteroaryl. m can be an integer selected from 0 to 4.

R₂, R₃, and R₄ can be independently H, halogen, hydroxyl, cyano, nitro, unsubstituted or substituted amino, unsubstituted or substituted alkyl, unsubstituted or substituted alkoxy, unsubstituted or substituted alkenyl, unsubstituted or substituted alkynyl, unsubstituted or substituted cycloalkyl, unsubstituted or substituted aryl, unsubstituted or substituted heteroaryl,

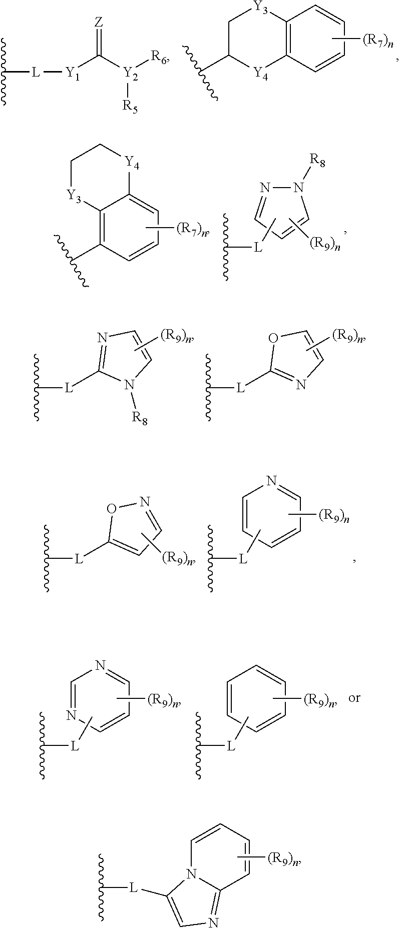

or R₂ and R₃ together with the carbon can form an unsubstituted or substituted 3-7 membered cycloalkyl or heterocycle ring.

L can be a C₁-C₅ alkyl linker optionally substituted, each Y₁, Y₂, Y₃, and Y₄ can be independently a covalent bond, a carbon, an oxygen, or a nitrogen, optionally substituted with hydrogen, unsubstituted or substituted alkyl, or unsubstituted or substituted cycloalkyl, and Z can be O or S.

R₅ and R₆ can be independently hydrogen, unsubstituted or substituted alkyl, or R₅ and R₆ can be cyclically linked and together with Y₂ to form an optionally substituted cycloalkyl or heterocycle, each R₇ can be hydrogen, halogen, cyano, nitro, hydroxyl, unsubstituted or substituted amino, unsubstituted or substituted alkyl, unsubstituted or substituted alkoxy, unsubstituted or substituted alkenyl, unsubstituted or substituted alkynyl, unsubstituted or substituted cycloalkyl, unsubstituted or substituted aryl, or unsubstituted or substituted heteroaryl.

n can be an integer selected from 0 to 4, R₈ can be hydrogen, cyano, unsubstituted or substituted alkyl, and unsubstituted or substituted aryl, and R₉ is selected from the group consisting of hydrogen, halogen, cyano, unsubstituted or substituted alkyl, unsubstituted or substituted alkoxy, or unsubstituted or substituted amino.

Further disclosed herein is a compound according to Formula (III) or an optically pure stereoisomer, pharmaceutically acceptable salt, solvate, or prodrug thereof

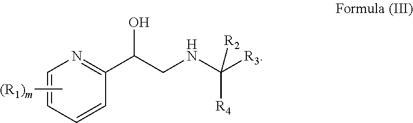

Each R₁ can be independently hydrogen, halogen, cyano, nitro, pentafluorosulfanyl, unsubstituted or substituted sulfonyl, substituted amino, unsubstituted or substituted alkyl, unsubstituted or substituted alkoxy, unsubstituted or substituted alkenyl, unsubstituted or substituted alkynyl, unsubstituted or substituted cycloalkyl, unsubstituted or substituted —(C═O)-alkyl, unsubstituted or substituted —(C═O)-cycloalkyl, unsubstituted or substituted —(C═O)-aryl, unsubstituted or substituted —(C═O)-heteroaryl, unsubstituted or substituted aryl, or unsubstituted or substituted heteroaryl. m can be an integer selected from 0 to 4.

R₂, R₃, and R₄ can be independently H, halogen, hydroxyl, cyano, nitro, unsubstituted or substituted amino, unsubstituted or substituted alkyl, unsubstituted or substituted alkoxy, unsubstituted or substituted alkenyl, unsubstituted or substituted alkynyl, unsubstituted or substituted cycloalkyl, unsubstituted or substituted aryl, unsubstituted or substituted heteroaryl,

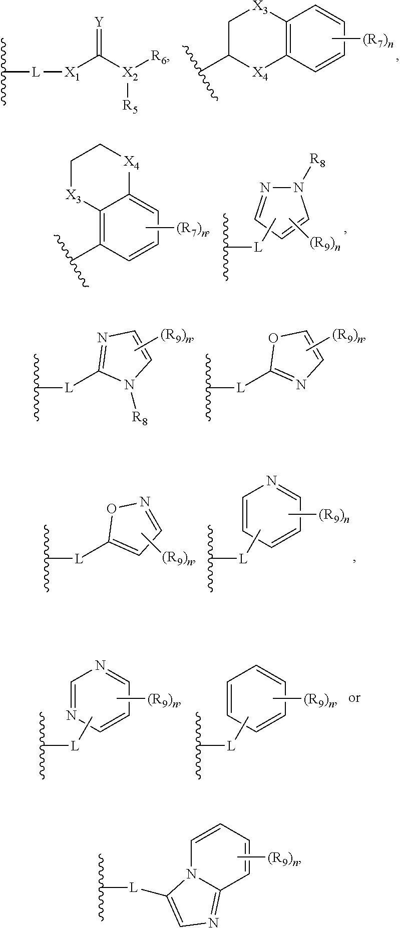

or R₂ and R₃ together with the carbon can form an unsubstituted or substituted 3-7 membered cycloalkyl or heterocycle ring.

L can be a C1-C5 alkyl linker optionally substituted, each X₁, X₂, X₃, and X₄ can be independently a covalent bond, a carbon, an oxygen, or a nitrogen, optionally substituted with hydrogen, unsubstituted or substituted alkyl, or unsubstituted or substituted cycloalkyl, and Y can be O or S.

R₅ and R₆ can be independently hydrogen, unsubstituted or substituted alkyl, or R₅ and R₆ can be cyclically linked and together with Y₂ to form an optionally substituted cycloalkyl or heterocycle, each R₇ can be independently hydrogen, halogen, cyano, nitro, hydroxyl, unsubstituted or substituted amino, unsubstituted or substituted alkyl, unsubstituted or substituted alkoxy, unsubstituted or substituted alkenyl, unsubstituted or substituted alkynyl, unsubstituted or substituted cycloalkyl, unsubstituted or substituted aryl, or unsubstituted or substituted heteroaryl.

n can be an integer selected from 0 to 4, R₈ can be hydrogen, cyano, unsubstituted or substituted alkyl, and unsubstituted or substituted aryl, and R₉ is selected from the group consisting of hydrogen, halogen, cyano, unsubstituted or substituted alkyl, unsubstituted or substituted alkoxy, or unsubstituted or substituted amino.

Further disclosed herein is a β-agent compound according to Formula (I′):

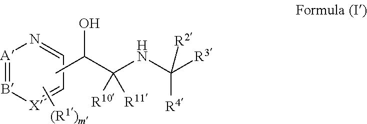

or a pharmaceutically acceptable salt thereof,
wherein:
A′, B′, and X′ are each independently nitrogen or carbon;
each R^{1′} is independently halogen, —R′, —CN, —NO₂, —SF₅, —OR^{x}, —NR^{x}₂, —NHR^{x}, —SO₂R′, —C(O)R′, —C(O)NR′₂;
each R′ is independently hydrogen or an optionally substituted group selected from: C₁₋₆ aliphatic, a 3-8 membered saturated or partially unsaturated monocyclic carbocyclic ring, phenyl, an 8-10 membered bicyclic partially unsaturated or aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic partially unsaturated or heteroaromatic ring having 1-5 heteroatoms independently selected from nitrogen, oxygen, or sulfur;
each R^{X} is independently an optionally substituted group selected from: C₁₋₆ aliphatic, a 3-8 membered saturated or partially unsaturated monocyclic carbocyclic ring, phenyl, an 8-10 membered bicyclic partially unsaturated or aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic partially unsaturated or heteroaromatic ring having 1-5 heteroatoms independently selected from nitrogen, oxygen, or sulfur;
m′ is an integer selected from 0 to 4;
R^{2′}, R^{3′}, and R^{4′} are each independently halogen, —R′, —CN, —NO₂, —OR′, —NR′₂,

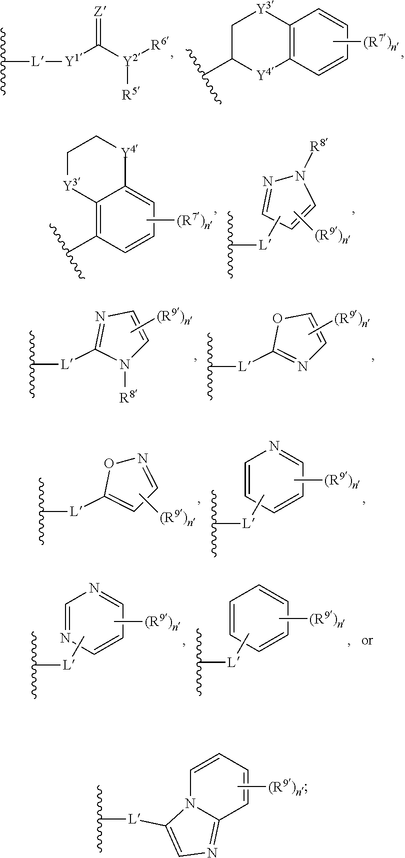

or
R^{2′} and R^{3′} together with the carbon form an optionally substituted 3-7 membered saturated carbocyclic ring; an optionally substituted 5-6 membered monocyclic heteroaryl ring having 1-4 heteroatoms independently selected from nitrogen, oxygen, and sulfur; an optionally substituted 3-7 membered saturated or a partially unsaturated monocyclic heterocyclic ring having 1-2 heteroatoms independently selected from nitrogen, oxygen, and sulfur;
L′ is optionally substituted C₁₋₅ alkylene;
Y^{1′}, Y^{2′}, Y^{3′}, and Y^{4′} are each independently a covalent bond, a carbon, an oxygen, or a nitrogen, optionally substituted with hydrogen, an optionally substituted C₁₋₆ alkyl, or an optionally substituted 3-7 membered saturated carbocyclic ring;
Z′ is O or S;
R^{5′} and R^{6′} are each independently hydrogen or optionally substituted alkyl, or
R^{5′} and R^{6′} are cyclically linked and, together with Y^{2′}, to form an optionally substituted 3-7 membered saturated carbocyclic ring; an optionally substituted 5-6 membered monocyclic heteroaryl ring having 1-4 heteroatoms independently selected from nitrogen, oxygen, and sulfur; an optionally substituted 3-7 membered saturated or partially unsaturated monocyclic heterocyclic ring having 1-2 heteroatoms independently selected from nitrogen, oxygen, and sulfur; or an optionally substituted 7-12 membered saturated or partially unsaturated bicyclic heterocyclic ring having 1-4 heteroatoms independently selected from nitrogen, oxygen, and sulfur;
each R^{7′} is independently —R′, halogen, —CN, —NO₂, —NR′₂, or —OR′;
n′ is an integer selected from 0 to 4;
R^{8′} is hydrogen, —CN, optionally substituted alkyl, or an optionally substituted aryl ring;
each R^{9′} is independently hydrogen, halogen, —CN, —OR^{x}, —NR′₂, or optionally substituted alkyl; and
R^{10′} and R^{11′} are each independently hydrogen or optionally substituted C₁₋₂ aliphatic.

Further disclosed herein is a β-agent compound according to Formula (I″):

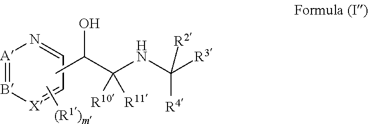

or a pharmaceutically acceptable salt thereof,
wherein:
A′, B′, and X′ are each independently nitrogen or carbon;
each R^{1′} is independently halogen, —R′, —CN, —NO₂, —SF₅, —OR^{x}, —NR^{x}₂, —NHR^{x}, —SO₂R′, —C(O)R′, —C(O)NR′₂, —NR′C(O)R′, —NR′CO₂R′, or —CO₂R′;
each R′ is independently hydrogen or an optionally substituted group selected from: C₁₋₆ aliphatic, a 3-8 membered saturated or partially unsaturated monocyclic carbocyclic ring, phenyl, an 8-10 membered bicyclic partially unsaturated or aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic partially unsaturated or heteroaromatic ring having 1-5 heteroatoms independently selected from nitrogen, oxygen, or sulfur;
each R^{x} is independently an optionally substituted group selected from: C₁₋₆ aliphatic, a 3-8 membered saturated or partially unsaturated monocyclic carbocyclic ring, phenyl, an 8-10 membered bicyclic partially unsaturated or aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic partially unsaturated or heteroaromatic ring having 1-5 heteroatoms independently selected from nitrogen, oxygen, or sulfur;
m′ is an integer selected from 0 to 4;
R^{2′}, R³, and R^{4′} are each independently halogen, —R′, —CN, —NO₂, —OR′, —NR′₂,

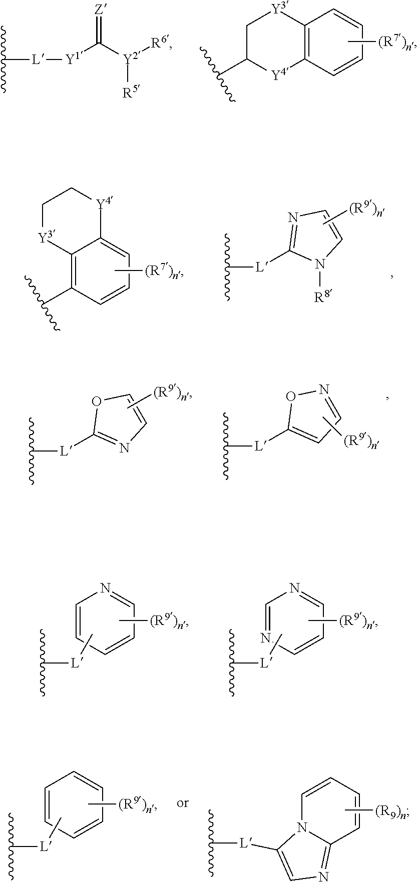

or
R^{2′} and R^{3′} together with the carbon form an optionally substituted 3-7 membered saturated carbocyclic ring; an optionally substituted 5-6 membered monocyclic heteroaryl ring having 1-4 heteroatoms independently selected from nitrogen, oxygen, and sulfur; an optionally substituted 3-7 membered saturated or a partially unsaturated monocyclic heterocyclic ring having 1-2 heteroatoms independently selected from nitrogen, oxygen, and sulfur;
L′ is optionally substituted C₁₋₅ alkylene;
Y^{1′}, Y^{2′}, Y^{3′}, and Y^{4′} are each independently a covalent bond, a carbon, an oxygen, or a nitrogen, optionally substituted with hydrogen, an optionally substituted C₁₋₆ alkyl, or an optionally substituted 3-7 membered saturated carbocyclic ring;
Z′ is O or S;
R^{5′} and R^{6′} are each independently hydrogen or optionally substituted alkyl, or
R^{5′} and R^{6′} are cyclically linked and, together with Y^{2′}, to form an optionally substituted 3-7 membered saturated carbocyclic ring; an optionally substituted 5-6 membered monocyclic heteroaryl ring having 1-4 heteroatoms independently selected from nitrogen, oxygen, and sulfur; an optionally substituted 3-7 membered saturated or partially unsaturated monocyclic heterocyclic ring having 1-2 heteroatoms independently selected from nitrogen, oxygen, and sulfur; or an optionally substituted 7-12 membered saturated or partially unsaturated bicyclic heterocyclic ring having 1-4 heteroatoms independently selected from nitrogen, oxygen, and sulfur;
each R^{7′} is independently —R′, halogen, —CN, —NO₂, —NR′₂, or —OR′;
n′ is an integer selected from 0 to 4;
R^{8′} is hydrogen, —CN, optionally substituted alkyl, or an optionally substituted aryl ring;
each R^{9′} is independently hydrogen, halogen, —CN, —OR^{x}, —NR′₂, or optionally substituted alkyl; and
R^{10′} and R^{11′} are each independently hydrogen or optionally substituted C₁₋₂ aliphatic.

As defined above and described herein, A′ is nitrogen or carbon. In some embodiments A′ is nitrogen. In some embodiments A′ is carbon.

In some embodiments A′ is selected from those depicted in Table 1, below.

As defined above and described herein, B′ is nitrogen or carbon. In some embodiments B′ is nitrogen. In some embodiments B′ is carbon.

In some embodiments B′ is selected from those depicted in Table 1, below.

As defined above and described herein, X′ is nitrogen or carbon. In some embodiments X′ is nitrogen. In some embodiments X′ is carbon.

In some embodiments X′ is selected from those depicted in Table 1, below.

As defined above, each R^{1′} is independently halogen, —R′, —CN, —NO₂, —SF₅, —OR^{x}, —NR^{x}₂, —NHR^{x}, —SO₂R′, —C(O)R′, —C(O)NR′₂, —NR′C(O)R′, —NR′CO₂R′, or —CO₂R′.

In some embodiments, R^{1′} is hydrogen. In some embodiments, R^{1′} is halogen. In some embodiments, R^{1′} is —R′. In some embodiments, R^{1′} is cyano. In some embodiments, R^{1′} is —NO₂. In some embodiments, R^{1′} is —SF₅. In some embodiments, R^{1′} is —OR^{x}. In some embodiments, R^{1′} is —NR^{x}₂. In some embodiments, R^{1′} is —NHR^{x}. In some embodiments, R^{1′} is —SO₂R′. In some embodiments, R^{1′} is —C(O)R′. In some embodiments, R^{1′} is —C(O)NR′₂. In some embodiments, R^{1′} is —NR′C(O)R′. In some embodiments, R^{1′} is —NR′CO₂R′. In some embodiments, R^{1′} is —CO₂R′.

In some embodiments, R^{1′} is —Br. In some embodiments, R^{1′} is —Cl. In some embodiments, R^{1′} is —F.

In some embodiments, R^{1′} is —CH₃. In some embodiments, R^{1′} is —CH₂CH₃. In some embodiments, R^{1′} is —CH(CH₃)₂.

In some embodiments, R^{1′} is —CF₃. In some embodiments, R^{1′} is —CF₂H. In some embodiments, R^{1′} is —CFH₂. In some embodiments, R^{1′} is —CF₂CH₃. In some embodiments, R^{1′} is —CH₂CF₃. In some embodiments, R^{1′} is —C≡CCH. In some embodiments, R^{1′} is vinyl. In some embodiments, R^{1′} is —C≡CCF₃. In some embodiments, R^{1′} is —CO₂H.

In some embodiments, R^{1′} is —CN.

In some embodiments, R^{1′} is —OCH₃. In some embodiments, R^{1′} is —OCH₂CH₃. In some embodiments, R^{1′} is —OCH(CH₃)₂. In some embodiments, R^{1′} is —OCF₃. In some embodiments, R^{1′} is —NHCH₃. In some embodiments, R^{1′} is —NHCD₃. In some embodiments, R^{1′} is —N(CD₃)CO₂tBu. In some embodiments, R^{1′} is —NHCH₂CH₃. In some embodiments, R^{1′} is —NHCH₂(CH₃)₂. In some embodiments, R^{1′} is —NHCH₂CF₃. In some embodiments, R^{1′} is —NHPh. In some embodiments, R^{1′} is —NHAc. In some embodiments, R_{1′} is —N(CH₃)₂. In some embodiments, R^{1′} is

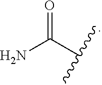 [SMILES: *C(N)=O]

In some embodiments, R^{1′} is

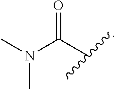 [SMILES: *C(=O)N(C)C]

In some embodiments, R^{1′} is

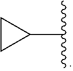 [SMILES: *C1CC1]

In some embodiments, R^{1′} is

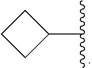 [SMILES: *C1CCC1]

In some embodiments, R^{1′} is

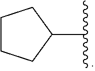 [SMILES: *C1CCCC1]

In some embodiments, R^{1′} is

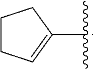 [SMILES: *C1=CCCC1]

In some embodiments, R^{1′} is

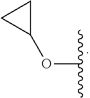 [SMILES: *OC1CC1]

In some embodiments, R^{1′} is

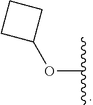 [SMILES: *OC1CCC1]

In some embodiments, R^{1′} is

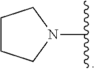 [SMILES: *N1CCCC1]

In some embodiments, R^{1′} is

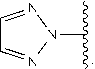 [SMILES: *n1nccn1]

In some embodiments, R^{1′} is

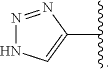 [SMILES: *c1c[nH]nn1]

In some embodiments, R^{1′} is

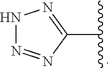 [SMILES: *c1nn[nH]n1]

In some embodiments, R^{1′} is

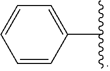 [SMILES: *c1ccccc1]

In some embodiments, R^{1′} is

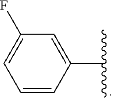 [SMILES: *c1cccc(F)c1]

In some embodiments, R^{1′} is

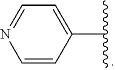 [SMILES: *c1ccncc1]

In some embodiments, R^{1′} is

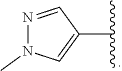 [SMILES: *c1cnn(C)c1]

In some embodiments, R^{1′} is

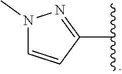 [SMILES: *c1ccn(C)n1]

In some embodiments, R^{1′} is

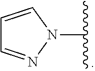 [SMILES: *n1cccn1]

In some embodiments, R^{1′} is

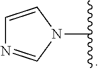 [SMILES: *n1ccnc1]

In some embodiments, R^{1′} is

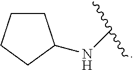 [SMILES: *NC1CCCC1]

In some embodiments, R^{1′} is

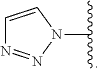 [SMILES: *n1ccnn1]

In some embodiments, R^{1′} is

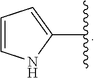 [SMILES: *c1ccc[nH]1]

In some embodiments, R^{1′} is

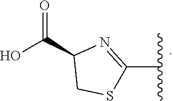 [SMILES: *C1=N[C@H](C(=O)O)CS1]

In some embodiments, R^{1′} is

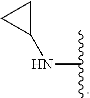 [SMILES: *NC1CC1]

In some embodiments, R^{1′} is

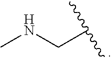 [SMILES: *CNC]

In some embodiments, R^{1′} is

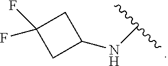 [SMILES: *NC1CC(F)(F)C1]

In some embodiments, R^{1′} is

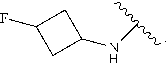 [SMILES: *NC1CC(F)C1]

In some embodiments, R^{1′} is

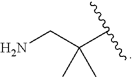 [SMILES: *C(C)(C)CN]

In some embodiments, R^{1′} is selected from those depicted in Table 1, below.

As defined above, each R′ is independently hydrogen or an optionally substituted group selected from C₁₋₆ aliphatic, a 3-8 membered saturated or partially unsaturated monocyclic carbocyclic ring, phenyl, an 8-10 membered bicyclic partially unsaturated or aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic partially unsaturated or heteroaromatic ring having 1-5 heteroatoms independently selected from nitrogen, oxygen, or sulfur.

In some embodiments, R′ is hydrogen.

In some embodiments, R′ is an optionally substituted C₁₋₆ aliphatic. For instance, in some embodiments, R′ is —CF₃, —CF₂H, or —CFH₂.

In some embodiments, R′ is an optionally substituted 3-8 membered saturated monocyclic carbocyclic ring.

In some embodiments, R′ is an optionally substituted 3-8 membered partially unsaturated monocyclic carbocyclic ring.

In some embodiments, R′ is an optionally substituted phenyl.

In some embodiments, R′ is an optionally substituted 8-10 membered bicyclic partially unsaturated carbocyclic ring.

In some embodiments, R′ is an optionally substituted 8-10 membered bicyclic aromatic carbocyclic ring.

In some embodiments, R′ is an optionally substituted 4-8 membered saturated monocyclic heterocyclic ring having 1-2 heteroatoms independently selected from nitrogen, oxygen, or sulfur.

In some embodiments, R′ is an optionally substituted 4-8 membered partially unsaturated monocyclic heterocyclic ring having 1-2 heteroatoms independently selected from nitrogen, oxygen, or sulfur.

In some embodiments, R′ is an optionally substituted 5-6 membered monocyclic heteroaromatic ring having 1-4 heteroatoms independently selected from nitrogen, oxygen, or sulfur.

In some embodiments, R′ is an optionally substituted 8-10 membered bicyclic partially unsaturated ring having 1-5 heteroatoms independently selected from nitrogen, oxygen, or sulfur.

In some embodiments, R′ is an optionally substituted 8-10 membered bicyclic heteroaromatic ring having 1-5 heteroatoms independently selected from nitrogen, oxygen, or sulfur.

In some embodiments, R′ is selected from those depicted in Table 1, below.

As defined above, each R^{x} is independently an optionally substituted group selected from C₁₋₆ aliphatic, a 3-8 membered saturated or partially unsaturated monocyclic carbocyclic ring, phenyl, an 8-10 membered bicyclic partially unsaturated or aromatic carbocyclic ring, a 4-8 membered saturated or partially unsaturated monocyclic heterocyclic ring having 1-2 heteroatoms independently selected from nitrogen, oxygen, or sulfur, a 5-6 membered monocyclic heteroaromatic ring having 1-4 heteroatoms independently selected from nitrogen, oxygen, or sulfur, or an 8-10 membered bicyclic partially unsaturated or heteroaromatic ring having 1-5 heteroatoms independently selected from nitrogen, oxygen, or sulfur.

In some embodiments, R^{x} is an optionally substituted C₁₋₆ aliphatic. For instance, in some embodiments, R^{x} is —CF₃, —CF₂H, or —CFH₂. In some embodiments, R^{x} is C₁₋₆ alkyl.

As defined above, m′ is an integer selected from 0 to 4.

In some embodiments, m′ is 0. In some embodiments, m′ is 1. In some embodiments, m′ is 2. In some embodiments, m′ is 3. In some embodiments, m′ is 4.

As defined above, R^{2′}, R^{3′}, and R^{4′} are each independently halogen, —R′, —CN, —OH, —OR′, —NR′₂, —NHR′, —NH₂,

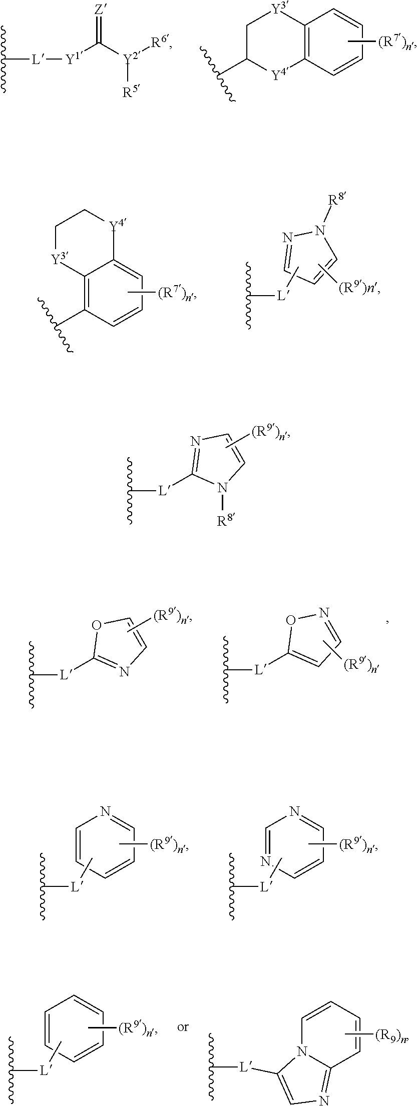

or R^{2′} and R^{3′} together with the carbon form an optionally substituted 3-7 membered saturated carbocyclic ring; an optionally substituted 5-6 membered monocyclic heteroaryl ring having 1-4 heteroatoms independently selected from nitrogen, oxygen, and sulfur; an optionally substituted 3-7 membered saturated or a partially unsaturated monocyclic heterocyclic ring having 1-2 heteroatoms independently selected from nitrogen, oxygen, and sulfur;

In some embodiments, R^{2′} is hydrogen. In some embodiments, R^{2′} is halogen. In some embodiments, R^{2′} is —R′. In some embodiments, R^{2′} is —CN. In some embodiments, R^{2′} is —NO₂. In some embodiments, R^{2′} is —OH. In some embodiments, R^{2′} is —OR′. In some embodiments, R^{2′} is —NR′₂. In some embodiments, R^{2′} is —NHR′. In some embodiments, R^{2′} is —NH₂.

In some embodiments, R^{2′} is

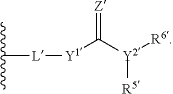

In some embodiments, R^{2′} is

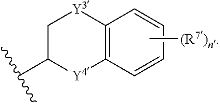

In some embodiments, R^{2′} is

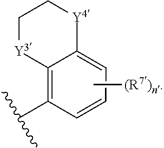

In some embodiments, R^{2′} is

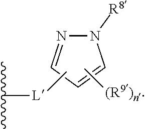

In some embodiments, R^{2′} is

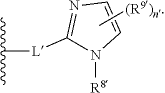

In some embodiments, R^{2′} is

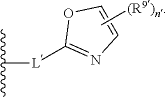

In some embodiments, R^{2′} is

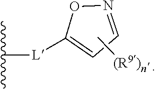

In some embodiments, R^{2′} is

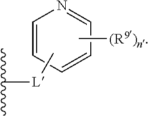

In some embodiments, R^{2′} is

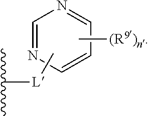

In some embodiments, R^{2′} is

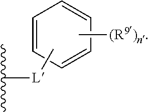

In some embodiments, R^{2′} is

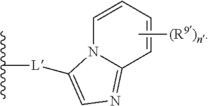

In some embodiments, R^{2′} is hydrogen. In some embodiments, R^{2′} is deuterium. In some embodiments, R^{2′} is —CH₃. In some embodiments, R^{2′} is —CD₃. In some embodiments, R² is

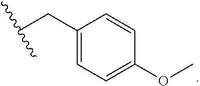 [SMILES: *Cc1ccc(OC)cc1]

In some embodiments, R^{3′} is hydrogen. In some embodiments, R^{3′} is halogen. In some embodiments, R^{3′} is —R′. In some embodiments, R^{3′} is —CN. In some embodiments, R^{3′} is —NO₂. In some embodiments, R^{3′} is —OH. In some embodiments, R^{3′} is —OR′. In some embodiments, R^{3′} is —NR′₂. In some embodiments, R^{3′} is —NHR′. In some embodiments, R^{3′} is —NH₂.

In some embodiments, R^{3′} is

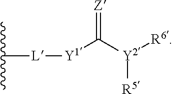

In some embodiments, R^{3′} is

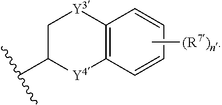

In some embodiments, R^{3′} is

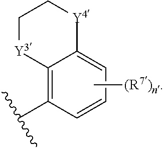

In some embodiments, R^{3′} is

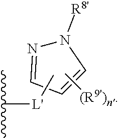

In some embodiments, R^{3′} is

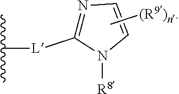

In some embodiments, R^{3′} is

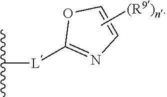

In some embodiments, R^{3′} is

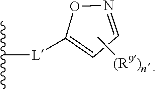

In some embodiments, R^{3′} is

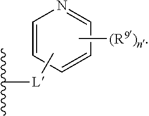

In some embodiments, R^{3′} is

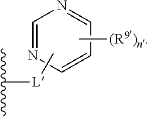

In some embodiments, R^{3′} is

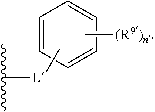

In some embodiments, R^{3′} is

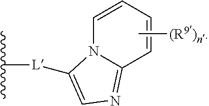

In some embodiments, R^{3′} is hydrogen. In some embodiments, R^{3′} is deuterium. In some embodiments, R^{3′} is —CH₃. In some embodiments, R^{3′} is —CD₃. In some embodiments, R^{3′} is

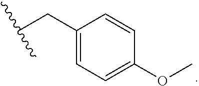 [SMILES: *Cc1ccc(OC)cc1]

In some embodiments, R^{2′} and R^{3′} together with the carbon form an optionally substituted 3-7 membered saturated carbocyclic ring; an optionally substituted 5-6 membered monocyclic heteroaryl ring having 1-4 heteroatoms independently selected from nitrogen, oxygen, and sulfur; an optionally substituted 3-7 membered saturated or a partially unsaturated monocyclic heterocyclic ring having 1-2 heteroatoms independently selected from nitrogen, oxygen, and sulfur.

In some embodiments, R^{2′} and R^{3′} together with the carbon form an optionally substituted 3-7 membered saturated carbocyclic ring.

In some embodiments, R^{2′} and R^{3′} together with the carbon form an optionally substituted 5-6 membered monocyclic heteroaryl ring having 1-4 heteroatoms independently selected from nitrogen, oxygen, and sulfur.

In some embodiments, R^{2′} and R^{3′} together with the carbon form an optionally substituted 3-7 membered saturated or a partially unsaturated monocyclic heterocyclic ring having 1-2 heteroatoms independently selected from nitrogen, oxygen, and sulfur.

In some embodiments, R^{2′} and R^{3′} together with the carbon form

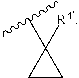

In some embodiments, R^{2′} and R^{3′} together with the carbon form

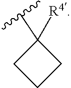

In some embodiments, R^{2′} and R^{3′} together with the carbon form

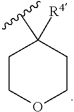

In some embodiments, R^{2′} and R^{3′} together with the carbon form

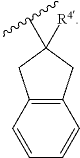

In some embodiments, R^{4′} is hydrogen. In some embodiments, R^{4′} is halogen. In some embodiments, R^{4′} is —R′. In some embodiments, R^{4′} is —CN. In some embodiments, R^{4′} is —NO₂. In some embodiments, R^{4′} is —OH. In some embodiments, R^{4′} is —OR′. In some embodiments, R^{4′} is —NR′₂. In some embodiments, R^{4′} is —NHR′. In some embodiments, R^{4′} is —NH₂. In some embodiments, R^{4′} is —CF₃.

In some embodiments, R^{4′} is

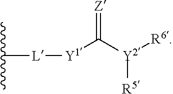

In some embodiments, R^{4′} is

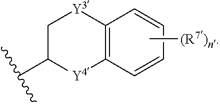

In some embodiments, R⁴ is

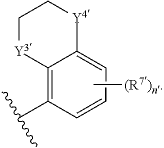

In some embodiments, R^{4′} is

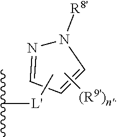

In some embodiments, R^{4′} is

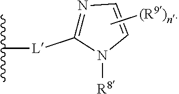

In some embodiments, R^{4′} is

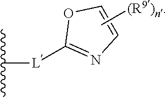

In some embodiments, R^{4′} is

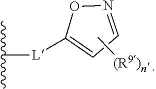

In some embodiments, R^{4′} is

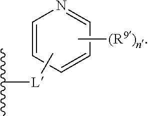

In some embodiments, R^{4′} is

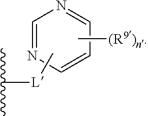

In some embodiments, R^{4′} is

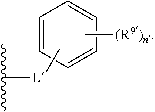

In some embodiments, R^{4′} is

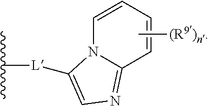

In some embodiments, R^{4′} is hydrogen. In some embodiments, R^{4′} is deuterium. In some embodiments, R^{4′} is —CH₃. In some embodiments, R^{4′} is —CD₃. some embodiments, R^{4′} is

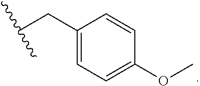 [SMILES: *Cc1ccc(OC)cc1]

In some embodiments, R^{2′}, R^{3′}, and R^{4′}, are each selected from those depicted in Table 1, below.

As defined above, L′ is optionally substituted C₁₋₅ alkylene.

In some embodiments, L′ is —CH₂—.

In some embodiments, L′ is selected from those depicted in Table 1, below.

As defined above, Y^{1′}, Y^{2′}, Y^{3′}, and Y^{4′} are each independently a covalent bond, a carbon, an oxygen; or a nitrogen, optionally substituted with hydrogen, an optionally substituted C₁₋₆ alkyl, or an optionally substituted 3-7 membered saturated carbocyclic ring.

In some embodiments, Y^{1′} is a covalent bond. In some embodiments, Y^{1′} is a carbon. In some embodiments, Y^{1′} is an oxygen. In some embodiments, Y^{1′} is a nitrogen, optionally substituted with hydrogen, an optionally substituted C₁₋₆ alkyl, or an optionally substituted 3-7 membered saturated carbocyclic ring.

In some embodiments, Y^{2′} is a covalent bond. In some embodiments, Y^{2′} is a carbon. In some embodiments, Y^{2′} is an oxygen. In some embodiments, Y^{2′} is a nitrogen, optionally substituted with hydrogen, an optionally substituted C₁₋₆ alkyl, or an optionally substituted 3-7 membered saturated carbocyclic ring.

In some embodiments, Y^{3′} is a covalent bond. In some embodiments, Y^{3′} is a carbon. In some embodiments, Y^{3′} is an oxygen. In some embodiments, Y^{3′} is a nitrogen, optionally substituted with hydrogen, an optionally substituted C₁₋₆ alkyl, or an optionally substituted 3-7 membered saturated carbocyclic ring.

In some embodiments, Y^{3′} is a covalent bond. In some embodiments, Y^{3′} is a carbon.

In some embodiments, Y^{4′} is a covalent bond. In some embodiments, Y^{4′} is a carbon. In some embodiments, Y^{4′} is an oxygen. In some embodiments, Y^{4′} is a nitrogen, optionally substituted with hydrogen, an optionally substituted C₁₋₆ alkyl, or an optionally substituted 3-7 membered saturated carbocyclic ring.

In some embodiments, Y^{4′} is a covalent bond. In some embodiments, Y^{4′} is a carbon.

In some embodiments, Y^{1′}, Y^{2′}, Y^{3′}, and Y^{4′} are each selected from those depicted in Table 1, below.

As defined above, Z′ is O or S.

In some embodiments, Z′ is O. In some embodiments, Z′ is S.

In some embodiments, Z′ is selected from those depicted in Table 1, below.

As defined above, R^{5′} and R^{6′} are each independently hydrogen or optionally substituted alkyl, or R^{5′} and R^{6′} are cyclically linked and, together with Y^{2′}, to form an optionally substituted 3-7 membered saturated carbocyclic ring; an optionally substituted 5-6 membered monocyclic heteroaryl ring having 1-4 heteroatoms independently selected from nitrogen, oxygen, and sulfur; an optionally substituted 3-7 membered saturated or partially unsaturated monocyclic heterocyclic ring having 1-2 heteroatoms independently selected from nitrogen, oxygen, and sulfur; or an optionally substituted 7-12 membered saturated or partially unsaturated bicyclic heterocyclic ring having 1-4 heteroatoms independently selected from nitrogen, oxygen, and sulfur.

In some embodiments, R^{5′} is hydrogen. In some embodiments, R^{5′} is an optionally substituted C₁₋₆ alkyl.

In some embodiments, R^{6′} is hydrogen. In some embodiments, R^{6′} is an optionally substituted C₁₋₆ alkyl.

In some embodiments, R^{5′} and R^{6′} are cyclically linked and together with Y^{2′} form an optionally substituted 3-7 membered saturated carbocyclic ring.

In some embodiments, R^{5′} and R^{6′} are cyclically linked and together with Y^{2′} form an optionally substituted 5-6 membered monocyclic heteroaryl ring having 1-4 heteroatoms independently selected from nitrogen, oxygen, and sulfur.

In some embodiments, R^{5′} and R^{6′} are cyclically linked and together with Y^{2′} form an optionally substituted 3-7 membered saturated or partially unsaturated monocyclic heterocyclic ring having 1-2 heteroatoms independently selected from nitrogen, oxygen, and sulfur.

In some embodiments, R^{5′} and R^{6′} are cyclically linked and together with Y^{2′} form an optionally substituted 7-12 membered saturated or partially unsaturated bicyclic heterocyclic ring having 1-4 heteroatoms independently selected from nitrogen, oxygen, and sulfur.

In some embodiments, R^{5′} and R^{6′} are each selected from those depicted in Table 1, below.

As defined above, each R^{7′} is independently —R′, halogen, —CN, —NO₂, —OH, —NR′₂, —NHR′, —NH₂, or —OR′.

In some embodiments, R^{7′} is hydrogen. In some embodiments, R^{7′} is halogen. In some embodiments, R^{7′} is —CN. In some embodiments, R^{7′} is —NO₂. In some embodiments, R^{7′} is —OH. In some embodiments, R^{7′} is —NR′₂. In some embodiments, R^{7′} is —NHR′. In some embodiments, R^{7′} is —NH₂. In some embodiments, R^{7′} is —OR′.

In some embodiments, each R^{7′} is independently selected from those depicted in Table 1, below.

As defined above, n′ is an integer selected from 0 to 4.

In some embodiments, n′ is 0. In some embodiments, n′ is 1. In some embodiments, n′ is 2. In some embodiments, n′ is 3. In some embodiments, n′ is 4.

As defined above, R^{8′} is hydrogen, —CN, optionally substituted alkyl, or an optionally substituted aryl ring.

In some embodiments, R^{8′} is hydrogen. In some embodiments, R^{8′} is —CN. In some embodiments, R^{8′} is an optionally substituted C₁₋₆ alkyl. In some embodiments, R^{8′} is an optionally substituted aryl ring.

In some embodiments, R^{8′} is selected from those depicted in Table 1, below.

As defined above, each R^{9′} is independently hydrogen, halogen, —CN, —OR′, —NR′₂, or optionally substituted alkyl.

In some embodiments, R^{9′} is hydrogen. In some embodiments, R^{9′} is halogen. In some embodiments, R^{9′} is —CN. In some embodiments, R^{9′} is —OR′. In some embodiments, R^{9′} is —NR′₂. In some embodiments, R^{9′} is —NHR′. In some embodiments, R^{9′} is —NH₂. In some embodiments, R^{9′} is an optionally substituted C₁₋₆ alkyl.

In some embodiments, R^{9′} is selected from those depicted in Table 1, below.

As defined above, R^{10′} and R^{11′} are each independently hydrogen or optionally substituted C₁₋₂ aliphatic. In some embodiments, R^{10′} and R^{11′} are each independently hydrogen, methyl, or ethyl.

In some embodiments, R^{10′} is hydrogen. In some embodiment, R^{10′} is an optionally substituted C₁ aliphatic. In some embodiment, R^{10′} is methyl. In some embodiment, R^{10′} is an optionally substituted C₂ aliphatic. In some embodiment, R^{10′} is ethyl.

In some embodiments, R^{10′} is selected from those depicted in Table 1, below.

In some embodiments, R^{11′} is hydrogen. In some embodiment, R^{11′} is an optionally substituted C₁ aliphatic. In some embodiment, R^{11′} is methyl. In some embodiment, R^{11′} is an optionally substituted C₂ aliphatic. In some embodiment, R^{11′} is ethyl.

In some embodiments, R^{11′} is selected from those depicted in Table 1, below.

Further disclosed herein is a β-agent compound according to Formula (II′):

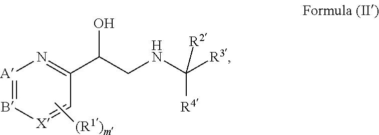

or a pharmaceutically acceptable salt thereof,
wherein each of A′, B′, X′, R^{1′}, R^{2′}, R^{3′}, R^{4′}, and m′ is as defined above and as described in embodiments provided herein, both singly and in combination.

Further disclosed herein is a β-agent compound according to Formula (III′):

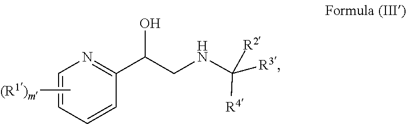

or a pharmaceutically acceptable salt thereof,
wherein each of R^{1′}, R^{2′}, R^{3′}, R^{4′}, and m′ is as defined above and as described in embodiments provided herein, both singly and in combination.

Further disclosed herein is a β-agent compound according to Formula (IV′):

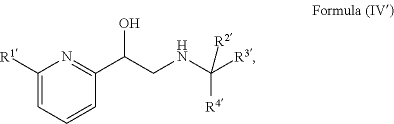

or a pharmaceutically acceptable salt thereof,
wherein each of R^{1′}, R^{2′}, R^{3′}, and R^{4′} is as defined above and as described in embodiments provided herein, both singly and in combination. In some such embodiments, R^{1′} is —CF₃. In some such embodiments, R^{1′} is —CF₂H. In some such embodiments, R^{1′} is —OCF₃. In some such embodiments, R^{1′} is —CN. In some such embodiments, R^{1′} is —C(O)NR′₂. In some such embodiments, R^{1′} is a cyclopropyl group. In some such embodiments, R^{1′} is a tetrazole. In some such embodiments, R^{1′} is phenyl. In some such embodiments, R^{1′} is —Br. In some such embodiments, R^{1′} is —CH₃.

Further disclosed herein is a β-agent compound according to Formula (V′):

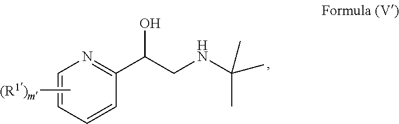

or a pharmaceutically acceptable salt thereof,
wherein each of R^{1′} and m′ is as defined above and as described in embodiments provided herein, both singly and in combination.

Further disclosed herein is a β-agent compound according to Formula (VI′):

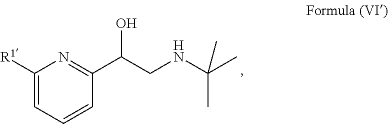

or a pharmaceutically acceptable salt thereof,
wherein R^{1′} is as defined above and as described in embodiments provided herein, both singly and in combination.

Further disclosed herein is a β-agent compound according to Formula (VII′):

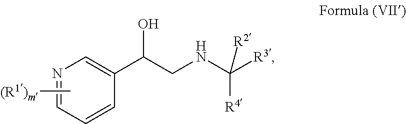

or a pharmaceutically acceptable salt thereof,
wherein each of R¹, R^{2′}, R^{3′}, R^{4′}, and m′ is as defined above and as described in embodiments provided herein, both singly and in combination.

Further disclosed herein is a β-agent compound according to Formula (VIII′):

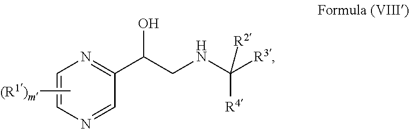

or a pharmaceutically acceptable salt thereof,
wherein each of R^{1′}, R^{2′}, R^{3′}, R^{4′}, and m′ is as defined above and as described in embodiments provided herein, both singly and in combination.

Further disclosed herein is a β-agent compound according to Formula (IX′):

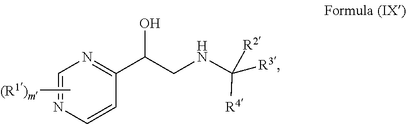

or a pharmaceutically acceptable salt thereof,
wherein each of R^{1′}, R^{2′}, R^{3′}, R^{4′}, and m′ is as defined above and as described in embodiments provided herein, both singly and in combination.

Further disclosed herein is a β-agent compound according to Formula (X′):

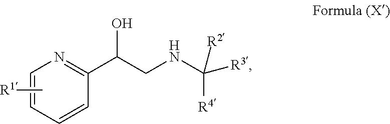

or a pharmaceutically acceptable salt thereof,
wherein
R^{1′} is halogen, —R^{x}, —CN, —NO₂, —SF₅, —OR′, —SO₂R′, or —C(O)R¹;
R^{2′}, R^{3′}, and R^{4′} are each independently halogen, —R′, —CN, —NO₂, —OR′, or —NR′₂, or
R^{2′} and R^{3′} together with the carbon form an optionally substituted 3-7 membered cycloalkyl or heterocycle ring; and
R′ and R^{x} are as defined above and as described in embodiments provided herein, both singly and in combination. In some such embodiments, R^{1′} is —CF₃. In some such embodiments, R^{1′} is —CF₂H. In some such embodiments, R^{1′} is —OCF₃. In some such embodiments, R^{1′} is —CN. In some such embodiments, R^{1′} is —C(O)NR′₂. In some such embodiments, R^{1′} is a cyclopropyl group. In some such embodiments, R^{1′} is a tetrazole. In some such embodiments, R^{1′} is phenyl. In some such embodiments, R^{1′} is —Br. In some such embodiments, R^{1′} is —CH₃.

Further disclosed herein is a β-agent compound according to Formula (XI′):

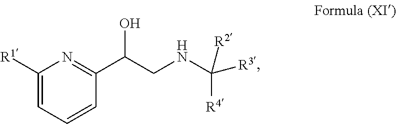

or a pharmaceutically acceptable salt thereof,
wherein:
R^{1′} is halogen, —R′, —CN, —NO₂, —SF₅, —OR′, —SO₂R′, or —C(O)R¹;
R^{2′}, R^{3′}, and R^{4′} are each independently halogen, —R′, —CN, —NO₂, —OR′, or —NR′₂, or
R^{2′} and R^{3′} together with the carbon form an optionally substituted 3-7 membered cycloalkyl or heterocycle ring; and
R′ and R^{x} are as defined above and as described in embodiments provided herein, both singly and in combination.

Further disclosed herein is a β-agent compound according to Formula (XII′):

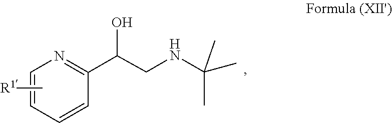

or a pharmaceutically acceptable salt thereof,
wherein:
R^{1′} is halogen, —R′, —CN, —NO₂, —SF₅, —OR′, —SO₂R′, or —C(O)R′; and
R′ and R^{X} are as defined above and as described in embodiments provided herein, both singly and in combination.

Further disclosed herein is a compound according to Formula (XIII′):

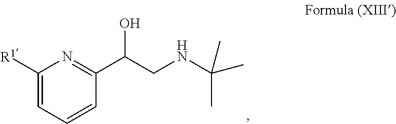

or a pharmaceutically acceptable salt thereof,
wherein:
R^{1′} is halogen, —R′, —CN, —NO₂, —SF₅, —OR′, —SO₂R′, or —C(O)R′; and
R′ and R^{X} are as defined above and as described in embodiments provided herein, both singly and in combination.

Further disclosed herein is a β-agent compound according to Formula (XIV′):

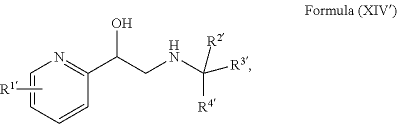

or a pharmaceutically acceptable salt thereof,
wherein
R^{1′} is halogen, —R′, —CN, or —NO₂;
R^{2′}, R^{3′}, and R^{4′} are each independently halogen, —R′, —CN, —NO₂, —OR′, or —NR′₂, or
R^{2′} and R^{3′} together with the carbon form an optionally substituted 3-7 membered cycloalkyl or heterocycle ring; and
R′ is as defined above and as described in embodiments provided herein, both singly and in combination.

Further disclosed herein is a compound according to Formula (XV′):

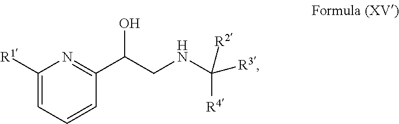

or a pharmaceutically acceptable salt thereof,
wherein:
R^{1′} is halogen, —R′, —CN, or —NO₂;
R^{2′}, R^{3′}, and R^{4′} are each independently halogen, —R′, —CN, —NO₂, —OR′, or —NR′₂, or
R^{2′} and R^{3′} together with the carbon form an optionally substituted 3-7 membered cycloalkyl or heterocycle ring; and
R′ is as defined above and as described in embodiments provided herein, both singly and in combination.

Further disclosed herein is a compound according to Formula (XVI′):

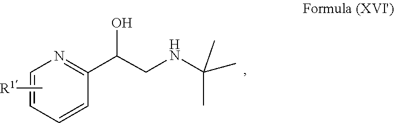

or a pharmaceutically acceptable salt thereof,
wherein:
R^{1′} is halogen, —R′, —CN, or —NO₂; and
R′ is as defined above and as described in embodiments provided herein, both singly and in combination.

Further disclosed herein is a compound according to Formula (XVII′):

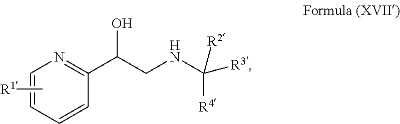

or a pharmaceutically acceptable salt thereof,
wherein
R^{1′} is halogen, —R′, —CN, or —NO₂;
each R′ is an optionally substituted C₁₋₆ aliphatic; and
R^{2′}, R^{3′}, and R^{4′} are each independently halogen, —R′, —CN, —NO₂, —OR′, or —NR′₂, or
R^{2′} and R^{3′} together with the carbon form an optionally substituted 3-7 membered cycloalkyl or heterocycle ring.

Further disclosed herein is a compound according to Formula (XVIII′):

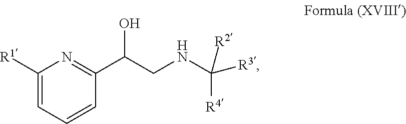

or a pharmaceutically acceptable salt thereof,
wherein:
R^{1′} is halogen, —R′, —CN, or —NO₂;
each R′ is an optionally substituted C₁₋₆ aliphatic; and
R^{2′}, R^{3′}, and R^{4′} are each independently halogen, —R′, —CN, —NO₂, —OR′, or —NR′₂, or
R^{2′} and R^{3′} together with the carbon form an optionally substituted 3-7 membered cycloalkyl or heterocycle ring.

Further disclosed herein is a compound according to Formula (XIX′):

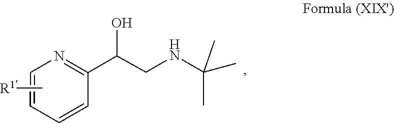

or a pharmaceutically acceptable salt thereof,
wherein:
R^{1′} is halogen, —R′, —CN, or —NO₂; and
R′ is optionally substituted C₁₋₆ aliphatic.

Further disclosed herein is a compound according to Formula (XX′):

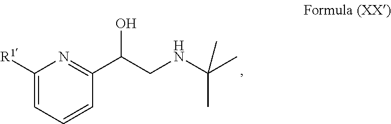

or a pharmaceutically acceptable salt thereof,
wherein:
R^{1′} is halogen, —R′, —CN, or —NO₂; and
R′ is an optionally substituted C₁₋₆ aliphatic.

Further disclosed herein is a compound according to Formula (XXI′):

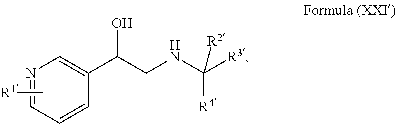

or a pharmaceutically acceptable salt thereof,
wherein
R^{1′} is halogen, —R′, —CN, or —NO₂;
each R′ is an optionally substituted C₁₋₆ aliphatic; and
R^{2′}, R^{3′}, and R^{4′} are each independently halogen, —R′, —CN, —NO₂, —OR′, or —NR′₂, or
R^{2′} and R^{3′} together with the carbon form an optionally substituted 3-7 membered cycloalkyl or heterocycle ring.

Further disclosed herein is a compound according to Formula (XXII′):

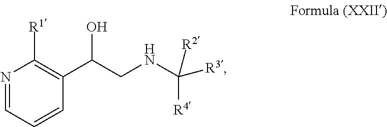

or a pharmaceutically acceptable salt thereof,
wherein:
R^{1′} is halogen, —R′, —CN, or —NO₂;
each R′ is an optionally substituted C₁₋₆ aliphatic; and
R^{2′}, R^{3′}, and R^{4′} are each independently halogen, —R′, —CN, —NO₂, —OR′, or —NR′₂, or
R^{2′} and R^{3′} together with the carbon form an optionally substituted 3-7 membered cycloalkyl or heterocycle ring.

Further disclosed herein is a compound according to Formula (XXIII′):

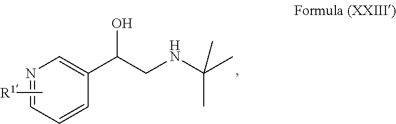

or a pharmaceutically acceptable salt thereof,
wherein:
R^{1′} is halogen, —R′, —CN, or —NO₂; and
R′ is optionally substituted C₁₋₆ aliphatic.

Further disclosed herein is a compound according to Formula (XXIV′):

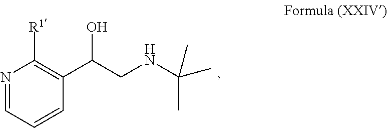

or a pharmaceutically acceptable salt thereof,
wherein:
R^{1′} is halogen, —R′, —CN, or —NO₂; and
R′ is an optionally substituted C₁₋₆ aliphatic.

Further disclosed herein is a compound according to Formula (XXV′):

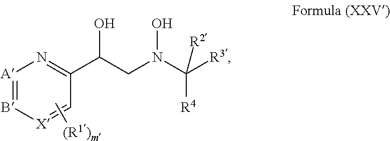

or a pharmaceutically acceptable salt thereof,
wherein each of A′, B′, X′, R^{1′}, R^{2′}, R^{3′}, R^{4′}, and m′ is as defined above and as described in embodiments provided herein, both singly and in combination.

TABLE 1
below illustrates exemplary β-agent compounds of the disclosure.

| Compound | | MS (m/z) |
|---|---|---|
| No. | Chemical Structure | (M + 1) |
| 03-1 | 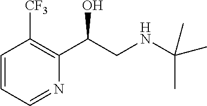 [SMILES: CC(C)(C)NC[C@H](O)c1ncccc1C(F)(F)F] | 263.2 |
| 03-2 | 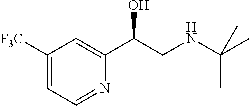 [SMILES: CC(C)(C)NC[C@H](O)c1cc(C(F)(F)F)ccn1] | 263.2 |
| 03-3 | 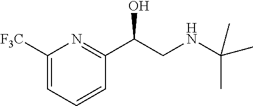 [SMILES: CC(C)(C)NC[C@H](O)c1cccc(C(F)(F)F)n1] | 263.2 |
| 03-4 | 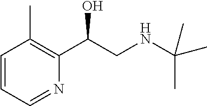 | 209.2 |
| 03-5 | 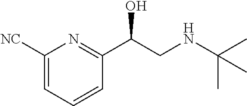 [SMILES: CC(C)(C)NC[C@H](O)c1cccc(C#N)n1] | 220.24 |
| 03-6 | 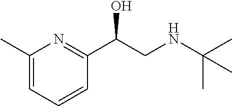 | 209.32 |
| 03-7 | 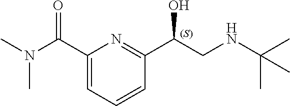 | 266.1 |
| 03-8 | 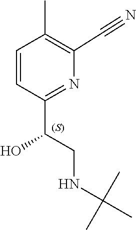 | 234.23 |
| 03-9 | 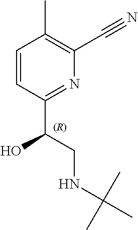 | 234.23 |
| 03-10 | 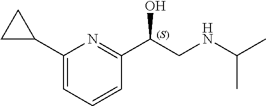 | 221.1 |
| 03-11 | 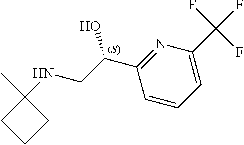 | 275.17 |
| 03-12 | 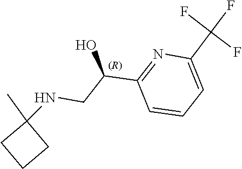 | 275.17 |
| 03-13 | 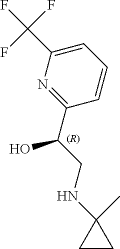 | 261.14 |
| 03-14 | 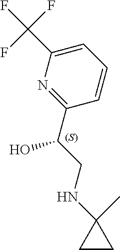 | 261.14 |
| 03-15 | 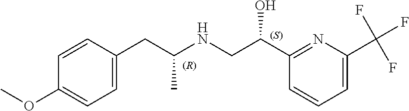 | 355.32 |
| 03-16 | 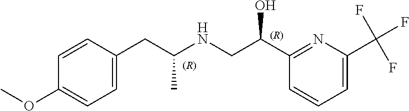 | 355.32 |
| 03-17 | 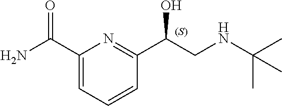 | 238.1 |
| 03-18 | 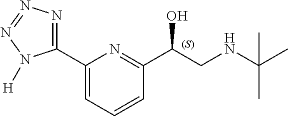 | 263.2 |
| 03-19 | 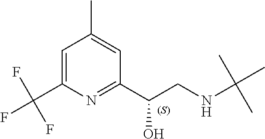 | 277.1 |
| 03-20 | 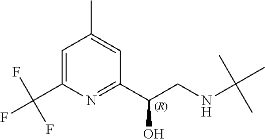 | 277.1 |
| 03-21 | 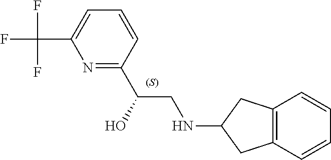 | 323.27 |
| 03-22 | 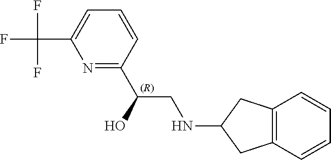 | 323.27 |
| 03-23 | 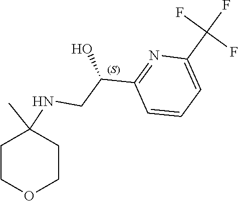 | 305.25 |
| 03-24 | 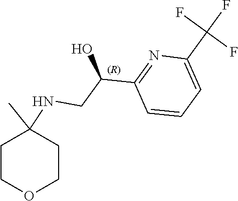 | 305.25 |
| 03-25 | 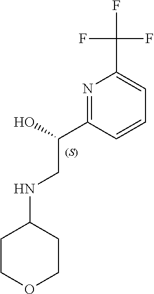 | 291.18 |
| 03-26 | 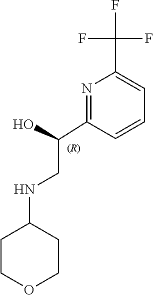 | 291.18 |
| 03-28 | 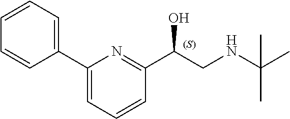 | 271.1 |
| 03-29 | 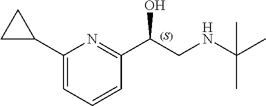 | 253.2 |
| 03-30 | 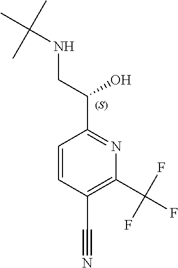 | 288.25 |
| 03-31 | 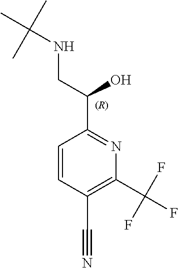 | 288.25 |
| 03-32 | 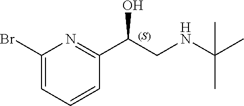 | 273.1 |
| 03-33 | 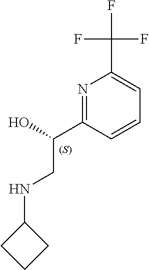 | 261.24 |
| 03-34 | 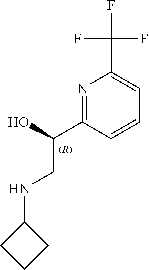 | 261.24 |
| 03-35 | 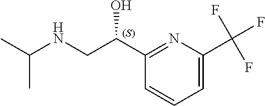 | 249.17 |
| 03-36 | 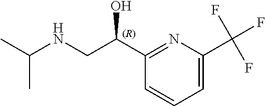 | 249.17 |
| 03-37 | 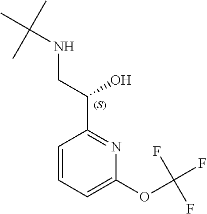 | 279.17 |
| 03-38 | 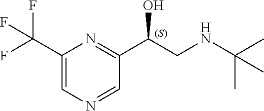 | 264.1 |
| 03-43 | 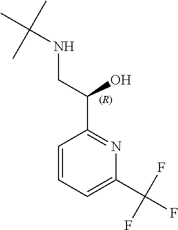 | 263.2 |
| 03-44 | 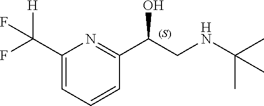 | 245.1 |
| 03-45 | 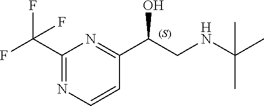 | 264.1 |
| 03-46 | 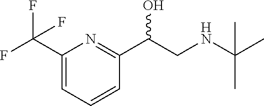 [SMILES: CC(C)(C)NCC(O)c1cccc(C(F)(F)F)n1] | 263.1 |
| 03-47 | 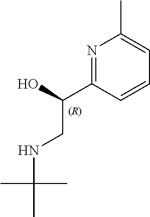 | 209.32 |
| 03-48 | 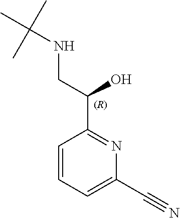 | 220.25 |
| 03-49 | 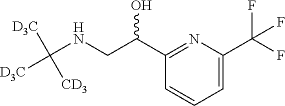 [SMILES: [2H]C([2H])([2H])C(NCC(O)c1cccc(C(F)(F)F)n1)(C([2H])([2H])[2H])C([2H])([2H])[2H]] | 272.1 |
| 03-50 | 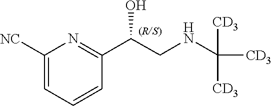 [SMILES: [2H]C([2H])([2H])C(NC[C@@H](O)c1cccc(C#N)n1)(C([2H])([2H])[2H])C([2H])([2H])[2H]] | 229.2 |
| 03-51 | 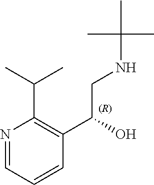 | 237.2 |
| 03-52 | 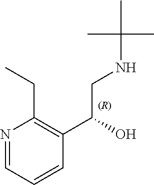 | 223.2 |
| 03-53 | 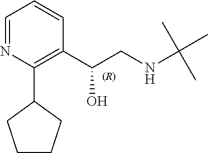 | 263.2 |
| 03-54 | 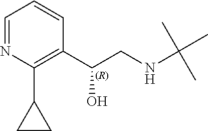 | 235.3 |
| 03-55 | 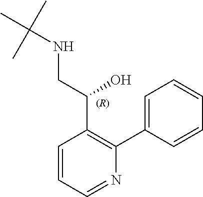 | 271.4 |
| 03-56 | 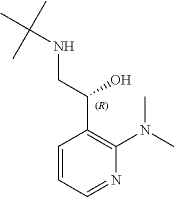 | 238.2 |
| 03-57 | 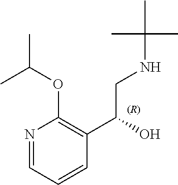 | 253.2 |
| 03-58 | 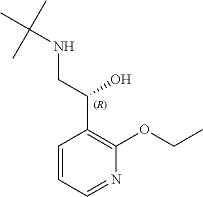 | 239.2 |
| 03-59 | 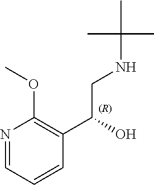 | 225.2 |
| 03-60 | 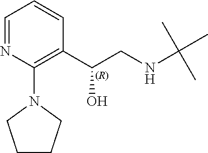 | 264.2 |
| 03-61 | 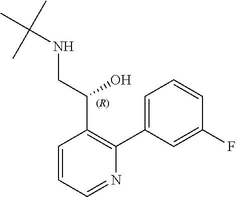 | 289.3 |
| 03-62 | 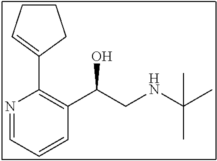 [SMILES: CC(C)(C)NC[C@H](O)c1cccnc1C1=CCCC1] | 261.4 |
| 03-63 | 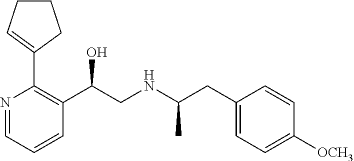 [SMILES: COc1ccc(C[C@@H](C)NC[C@H](O)c2cccnc2C2=CCCC2)cc1] | 353.5 |
| 03-64 | 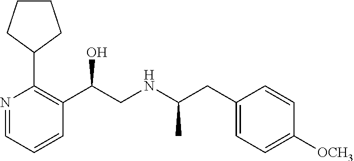 [SMILES: COc1ccc(C[C@@H](C)NC[C@H](O)c2cccnc2C2CCCC2)cc1] | 355.5 |
| 03-65 | 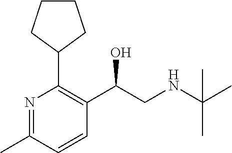 [SMILES: Cc1ccc([C@@H](O)CNC(C)(C)C)c(C2CCCC2)n1] | 277.4 |
| 03-66 | 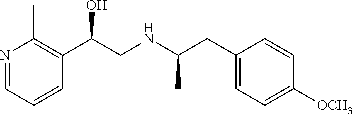 [SMILES: COc1ccc(C[C@@H](C)NC[C@H](O)c2cccnc2C)cc1] | 301.3 |
| 03-67 | 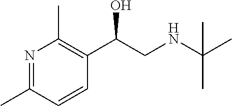 [SMILES: Cc1ccc([C@@H](O)CNC(C)(C)C)c(C)n1] | 223.3 |
| 03-68 | 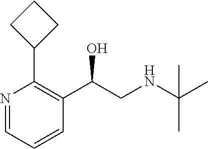 [SMILES: CC(C)(C)NC[C@H](O)c1cccnc1C1CCC1] | 249.3 |
| 03-70 | 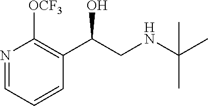 [SMILES: CC(C)(C)NC[C@H](O)c1cccnc1OC(F)(F)F] | 279.3 |
| 03-71 | 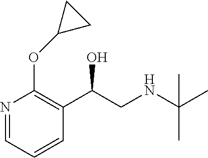 [SMILES: CC(C)(C)NC[C@H](O)c1cccnc1OC1CC1] | 251.3 |
| 03-72 | 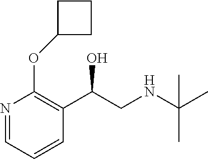 [SMILES: CC(C)(C)NC[C@H](O)c1cccnc1OC1CCC1] | 265.3 |
| 03-73 | 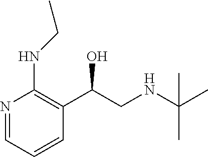 [SMILES: CCNc1ncccc1[C@@H](O)CNC(C)(C)C] | 238.4 |
| 03-74 | 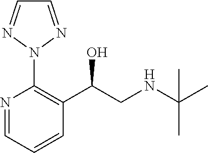 [SMILES: CC(C)(C)NC[C@H](O)c1cccnc1-n1nccn1] | 262.2 |
| 03-75 | 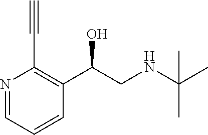 [SMILES: C#Cc1ncccc1[C@@H](O)CNC(C)(C)C] | 219.3 |
| 03-76 | 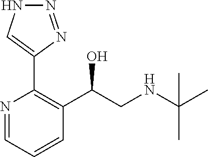 [SMILES: CC(C)(C)NC[C@H](O)c1cccnc1-c1c[nH]nn1] | 262.3 |
| 03-77 | 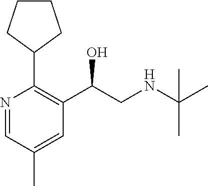 [SMILES: Cc1cnc(C2CCCC2)c([C@@H](O)CNC(C)(C)C)c1] | 277.4 |
| 03-78 | 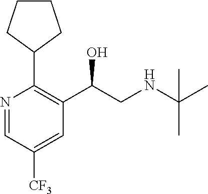 [SMILES: CC(C)(C)NC[C@H](O)c1cc(C(F)(F)F)cnc1C1CCCC1] | 331.3 |
| 03-79 | 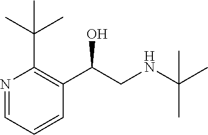 [SMILES: CC(C)(C)NC[C@H](O)c1cccnc1C(C)(C)C] | 251.4 |
| 03-80 | 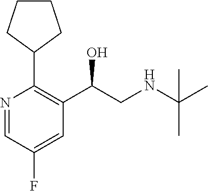 [SMILES: CC(C)(C)NC[C@H](O)c1cc(F)cnc1C1CCCC1] | 281.4 |
| 03-81 | 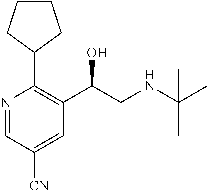 [SMILES: CC(C)(C)NC[C@H](O)c1cc(C#N)cnc1C1CCCC1] | |
| 03-82 | 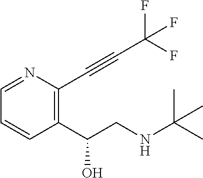 [SMILES: CC(C)(C)NC[C@H](O)c1cccnc1C#CC(F)(F)F] | |
| 03-83 | 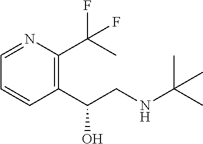 [SMILES: CC(C)(C)NC[C@H](O)c1cccnc1C(C)(F)F] | 259.3 |
| 03-84 | 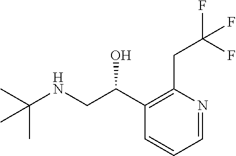 [SMILES: CC(C)(C)NC[C@H](O)c1cccnc1CC(F)(F)F] | 277.3 |
| 03-85 | 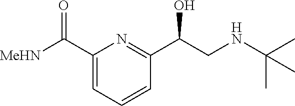 [SMILES: CNC(=O)c1cccc([C@@H](O)CNC(C)(C)C)n1] | 252.2 |
| 03-86 | 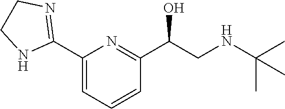 [SMILES: CC(C)(C)NC[C@H](O)c1cccc(C2=NCCN2)n1] | 263.1 |
| 03-87 | 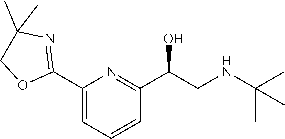 [SMILES: CC1(C)COC(c2cccc([C@@H](O)CNC(C)(C)C)n2)=N1] | |
| 03-88 | 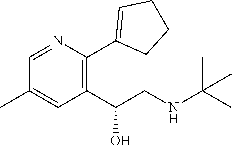 [SMILES: Cc1cnc(C2=CCCC2)c([C@@H](O)CNC(C)(C)C)c1] | 275.3 |
| 03-89 | 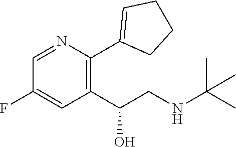 [SMILES: CC(C)(C)NC[C@H](O)c1cc(F)cnc1C1=CCCC1] | 279.4 |
| 03-90 | 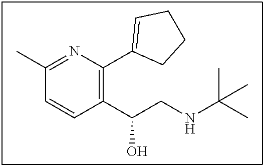 [SMILES: Cc1ccc([C@@H](O)CNC(C)(C)C)c(C2=CCCC2)n1] | 275.4 |
| 03-91 | 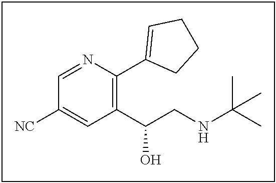 [SMILES: CC(C)(C)NC[C@H](O)c1cc(C#N)cnc1C1=CCCC1] | |
| 03-92 | 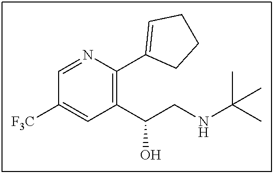 [SMILES: CC(C)(C)NC[C@H](O)c1cc(C(F)(F)F)cnc1C1=CCCC1] | 329.4 |
| 03-93 | 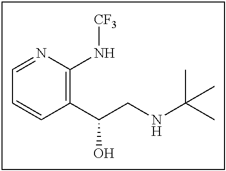 [SMILES: CC(C)(C)NC[C@H](O)c1cccnc1NC(F)(F)F] | |
| 03-94 | 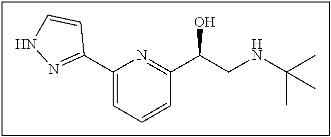 [SMILES: CC(C)(C)NC[C@H](O)c1cccc(-c2cc[nH]n2)n1] | |
| 03-95 | 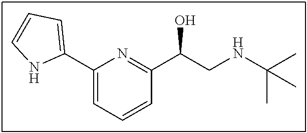 [SMILES: CC(C)(C)NC[C@H](O)c1cccc(-c2ccc[nH]2)n1] | 260.2 |
| 03-96 | 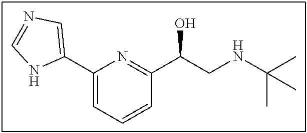 [SMILES: CC(C)(C)NC[C@H](O)c1cccc(-c2cnc[nH]2)n1] | |
| 03-97 | 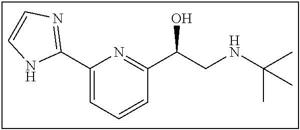 [SMILES: CC(C)(C)NC[C@H](O)c1cccc(-c2ncc[nH]2)n1] | |
| 03-98 | 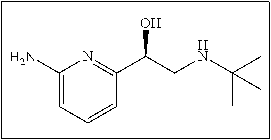 | 210.1 |
| 03-99 | 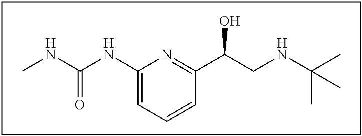 [SMILES: CNC(=O)Nc1cccc([C@@H](O)CNC(C)(C)C)n1] | |
| 03-100 | 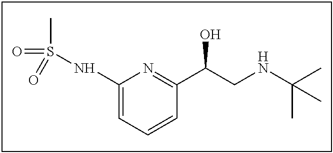 [SMILES: CC(C)(C)NC[C@H](O)c1cccc(NS(C)(=O)=O)n1] | |
| 03-101 | 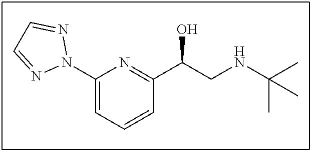 [SMILES: CC(C)(C)NC[C@H](O)c1cccc(-n2nccn2)n1] | 262.2 |
| 03-102 | 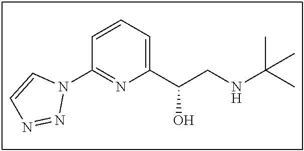 [SMILES: CC(C)(C)NC[C@H](O)c1cccc(-n2ccnn2)n1] | 262.2 |
| 03-103 | 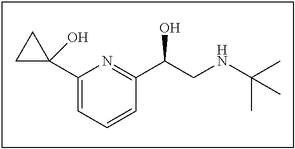 [SMILES: CC(C)(C)NC[C@H](O)c1cccc(C2(O)CC2)n1] | |
| 03-104 | 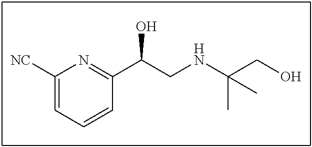 [SMILES: CC(C)(CO)NC[C@H](O)c1cccc(C#N)n1] | 236.2 |
| 03-105 | 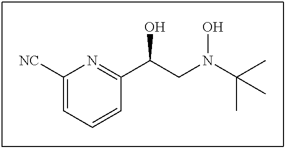 [SMILES: CC(C)(C)N(O)C[C@H](O)c1cccc(C#N)n1] | |
| 03-106 | 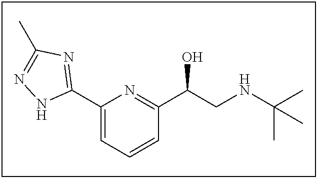 [SMILES: Cc1n[nH]c(-c2cccc([C@@H](O)CNC(C)(C)C)n2)n1] | |
| 03-107 | 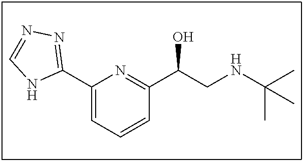 [SMILES: CC(C)(C)NC[C@H](O)c1cccc(-c2nnc[nH]2)n1] | |
| 03-108 | 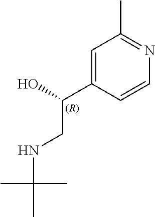 | 209.2 |
| 03-109 | 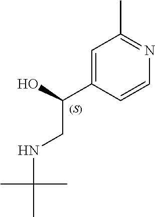 | 209.2 |
| 03-110 | 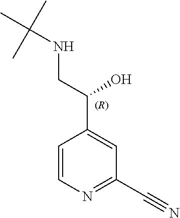 | 220.2 |
| 03-111 | 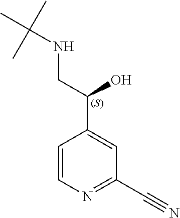 | 220.2 |
| 03-112 | 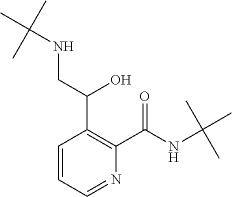 [SMILES: CC(C)(C)NCC(O)c1cccnc1C(=O)NC(C)(C)C] | 294.2 |
| 03-113 | 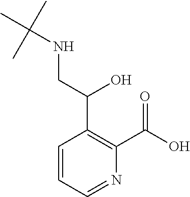 [SMILES: CC(C)(C)NCC(O)c1cccnc1C(=O)O] | 239.23 |
| 03-114 | 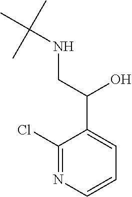 | 229.1 |
| 03-115 | 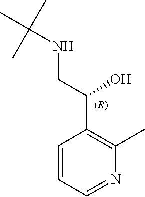 | 209.2 |
| 03-116 | 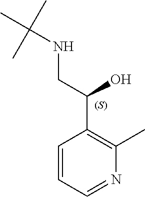 | 209.2 |
| 03-117 | 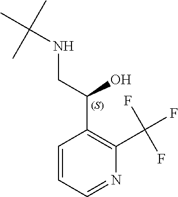 | 263.2 |
| 03-118 | 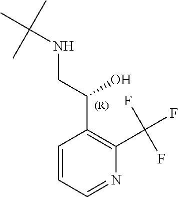 | 263.2 |
| 03-119 | 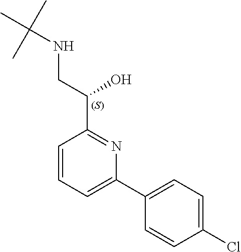 | 305.37 |
| 03-120 | 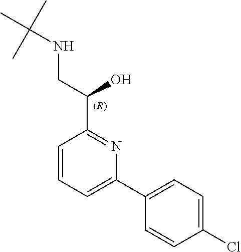 | 305.37 |
| 03-121 | 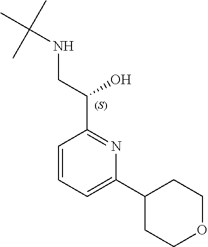 | 279.3 |
| 03-122 | 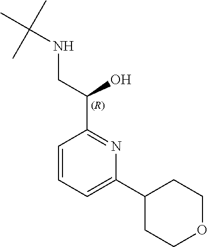 | 279.3 |
| 03-123 | 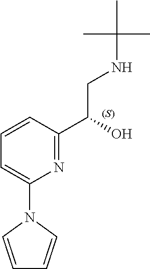 | 260.27 |
| 03-124 | 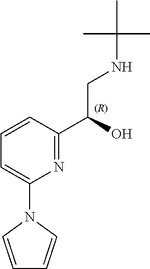 | 260.23 |
| 03-125 | 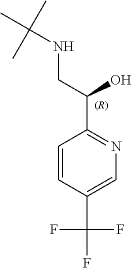 | 263.1 |
| 03-126 | 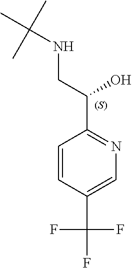 | 263.1 |
| 03-127 | 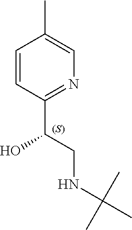 | 209.1 |
| 03-128 | 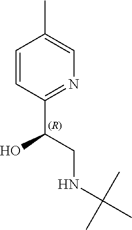 | 209.1 |
| 03-129 | 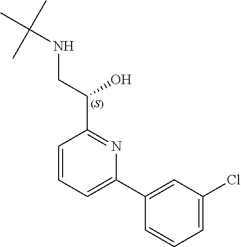 | 305.32 |
| 03-130 | 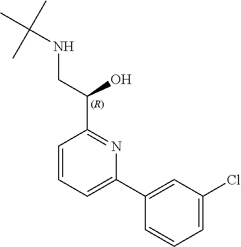 | 305.35 |
| 03-131 | 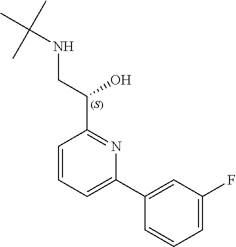 | 289.35 |
| 03-132 | 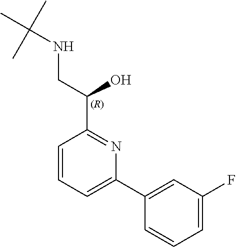 | 289.32 |
| 03-133 | 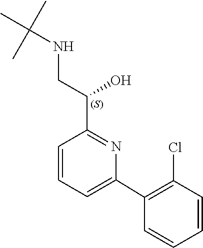 | 305.28 |
| 03-134 | 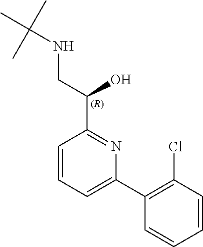 | 305.28 |
| 03-135 | 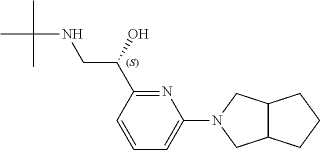 | 304.4 |
| 03-136 | 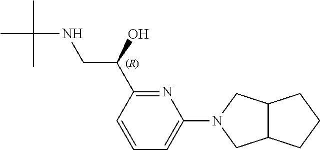 | 304.4 |
| 03-137 | 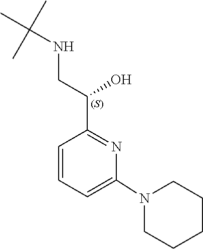 | 278.41 |
| 03-138 | 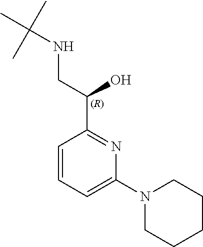 | 278.41 |
| 03-139 | 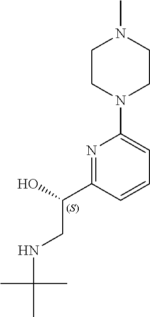 | 293.42 |
| 03-140 | 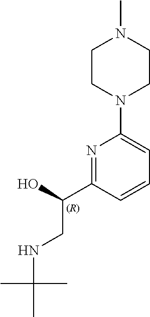 | 293.42 |
| 03-141 | 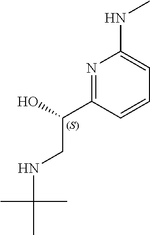 | 224.25 |
| 03-142 | 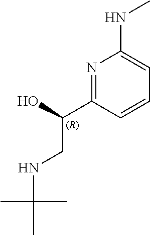 | 224.25 |
| 03-143 | 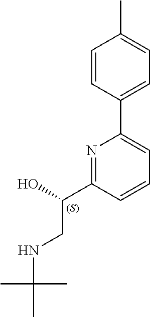 | 285.22 |
| 03-144 | 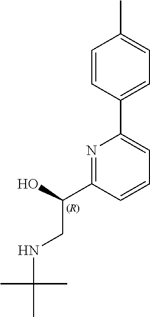 | 285.22 |
| 03-145 | 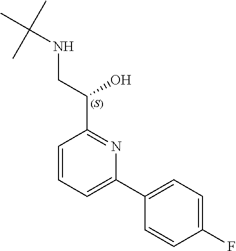 | 289.38 |
| 03-146 | 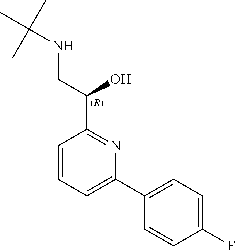 | 289.38 |
| 03-147 | 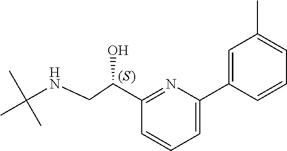 | 285.35 |
| 03-148 | 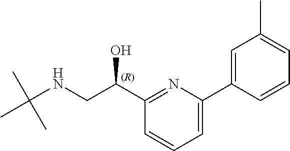 | 285.35 |
| 03-149 | 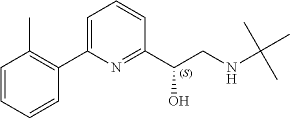 | 285.33 |
| 03-150 | 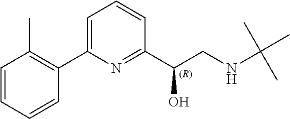 | 285.33 |
| 03-151 | 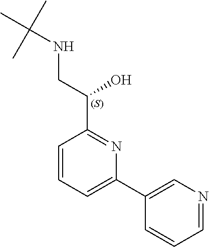 | 272.33 |
| 03-152 | 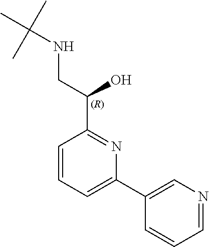 | 272.33 |
| 03-153 | 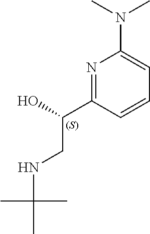 | 238.1 |
| 03-154 | 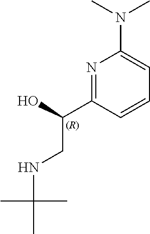 | 238.1 |
| 03-155 | 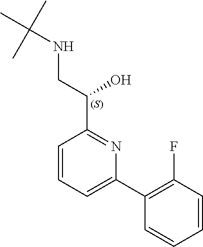 | 289.29 |
| 03-156 | 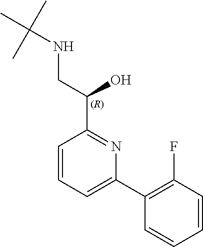 | 289.29 |
| 03-157 | 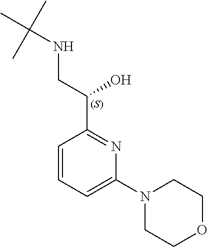 | 280.32 |
| 03-158 | 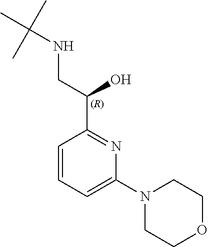 | 280.32 |
| 03-159 | 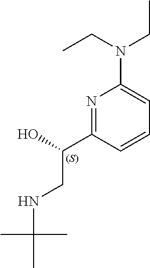 | 266.32 |
| 03-160 | 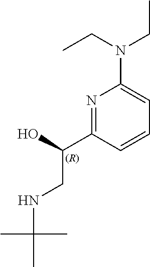 | 266.32 |
| 03-161 | 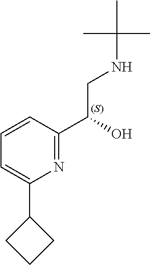 | 249.18 |
| 03-162 | 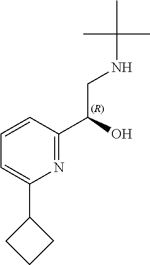 | 249.18 |
| 03-163 | 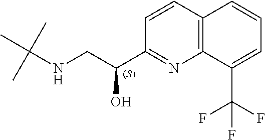 | 313.26 |
| 03-164 | 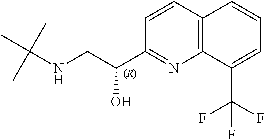 | 313.26 |
| 03-165 | 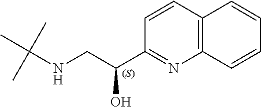 | 245.1 |
| 03-166 | 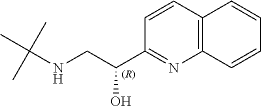 | 245.1 |
| 03-167 | 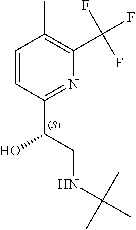 | 277.1 |
| 03-168 | 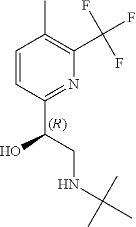 | 277.1 |
| 03-169 | 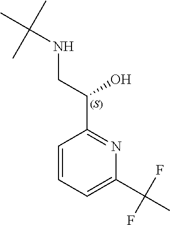 | 259.13 |
| 03-170 | 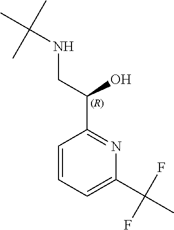 | 259.13 |
| 03-171 | 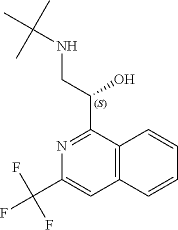 | 313.28 |
| 03-172 | 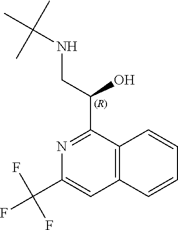 | 313.28 |
| 03-173 | 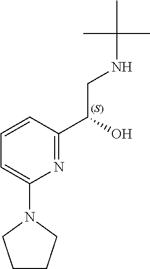 | 264.3 |
| 03-174 | 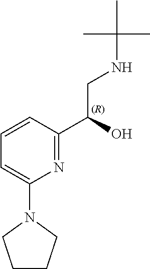 | 264.3 |
| 03-175 | 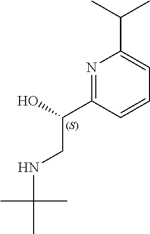 | 237.28 |
| 03-176 | 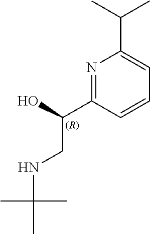 | 237.28 |
| 03-177 | 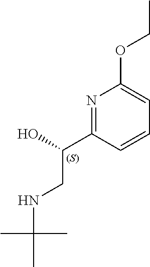 | 239.26 |
| 03-178 | 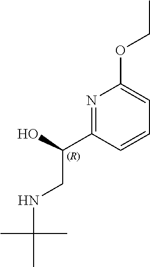 | 239.26 |
| 03-179 | 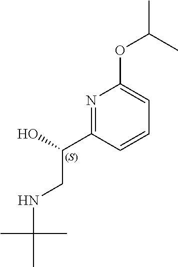 | 253.2 |
| 03-180 | 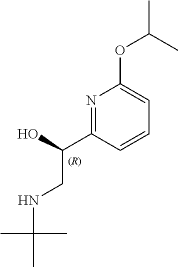 | 253.2 |
| 03-181 | 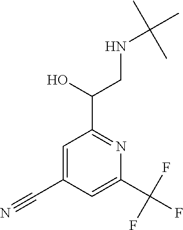 [SMILES: CC(C)(C)NCC(O)c1cc(C#N)cc(C(F)(F)F)n1] | 288.23 |
| 03-182 | 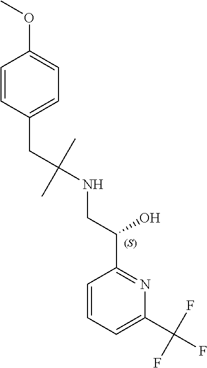 | 369.26 |
| 03-183 | 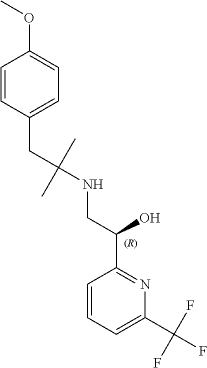 | 369.26 |
| 03-184 | 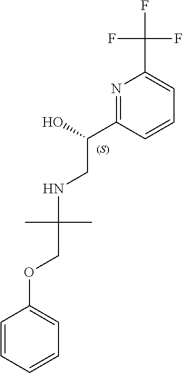 | 355.32 |
| 03-185 | 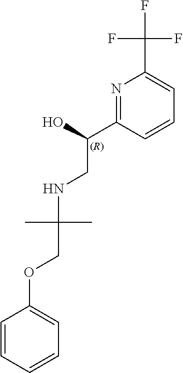 | 355.32 |
| 03-186 | 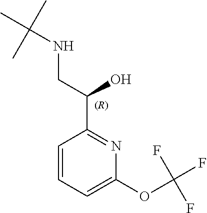 | 279.17 |
| 03-187 | 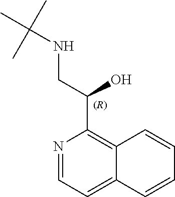 | 245.1 |
| 03-188 | 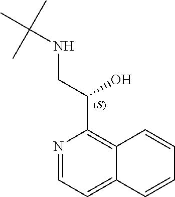 | 245.1 |
| 03-189 | 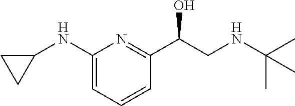 [SMILES: CC(C)(C)NC[C@H](O)c1cccc(NC2CC2)n1] | 250.2 |
| 03-190 | 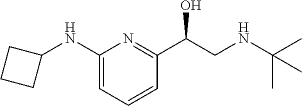 [SMILES: CC(C)(C)NC[C@H](O)c1cccc(NC2CCC2)n1] | 264.3 |
| 03-191 | 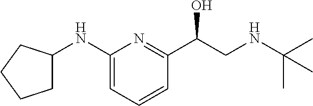 [SMILES: CC(C)(C)NC[C@H](O)c1cccc(NC2CCCC2)n1] | 278.4 |
| 03-192 | 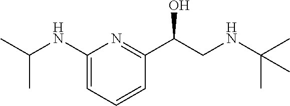 [SMILES: CC(C)Nc1cccc([C@@H](O)CNC(C)(C)C)n1] | 252.3 |
| 03-193 | 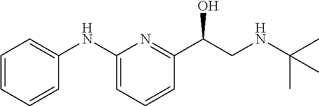 [SMILES: CC(C)(C)NC[C@H](O)c1cccc(Nc2ccccc2)n1] | 286.3 |
| 03-194 | 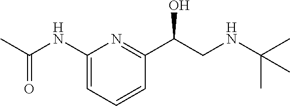 [SMILES: CC(=O)Nc1cccc([C@@H](O)CNC(C)(C)C)n1] | 252.3 |
| 03-195 | 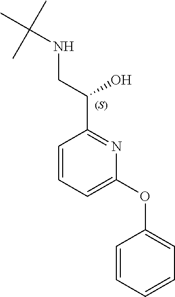 | 287.2 |
| 03-196 | 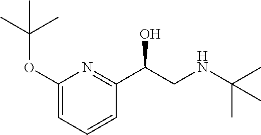 [SMILES: CC(C)(C)NC[C@H](O)c1cccc(OC(C)(C)C)n1] | 267.2 |
| 03-197 | 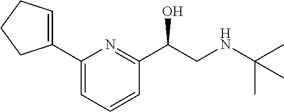 [SMILES: CC(C)(C)NC[C@H](O)c1cccc(C2=CCCC2)n1] | 261.3 |
| 03-198 | 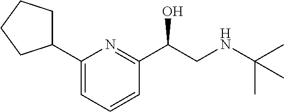 [SMILES: CC(C)(C)NC[C@H](O)c1cccc(C2CCCC2)n1] | 263.3 |
| 03-199 | 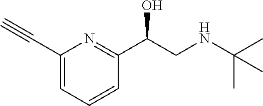 [SMILES: C#Cc1cccc([C@@H](O)CNC(C)(C)C)n1] | 219.2 |
| 03-200 | 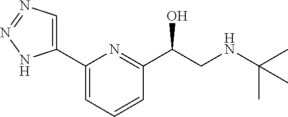 [SMILES: CC(C)(C)NC[C@H](O)c1cccc(-c2cnn[nH]2)n1] | 262.2 |
| 03-201 | 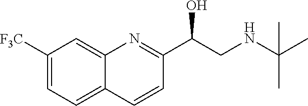 [SMILES: CC(C)(C)NC[C@H](O)c1ccc2ccc(C(F)(F)F)cc2n1] | |
| 03-202 | 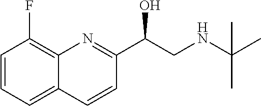 [SMILES: CC(C)(C)NC[C@H](O)c1ccc2cccc(F)c2n1] | |
| 03-203 | 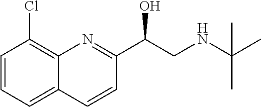 [SMILES: CC(C)(C)NC[C@H](O)c1ccc2cccc(Cl)c2n1] | |
| 03-204 | 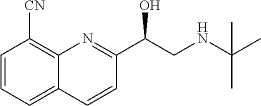 [SMILES: CC(C)(C)NC[C@H](O)c1ccc2cccc(C#N)c2n1] | |
| 03-205 | 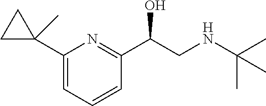 [SMILES: CC(C)(C)NC[C@H](O)c1cccc(C2(C)CC2)n1] | |
| 03-206 | 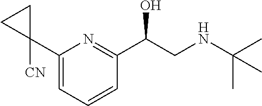 [SMILES: CC(C)(C)NC[C@H](O)c1cccc(C2(C#N)CC2)n1] | 260.4 |
| 03-207 | 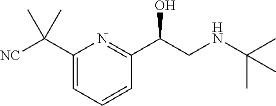 [SMILES: CC(C)(C)NC[C@H](O)c1cccc(C(C)(C)C#N)n1] | 262.3 |
| 03-208 | 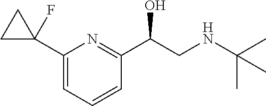 [SMILES: CC(C)(C)NC[C@H](O)c1cccc(C2(F)CC2)n1] | |
| 03-209 | 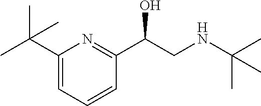 [SMILES: CC(C)(C)NC[C@H](O)c1cccc(C(C)(C)C)n1] | 251.2 |
| 03-210 | 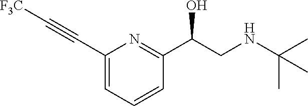 [SMILES: CC(C)(C)NC[C@H](O)c1cccc(C#CC(F)(F)F)n1] | |
| 03-211 | 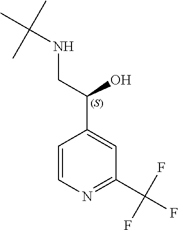 | 263.2 |
| 03-212 | 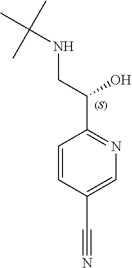 | 220.2 |
| 03-213 | 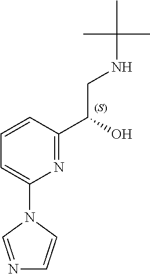 | 261.3 |
| 03-214 | 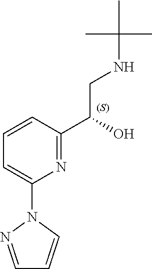 | 261.2 |
| 03-215 | 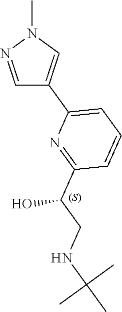 | 275.3 |
| 03-216 | 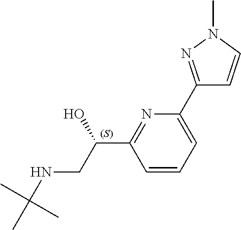 | 275.3 |
| 03-217 | 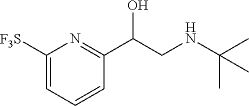 | |
| 03-218 | 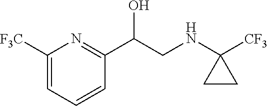 [SMILES: OC(CNC1(C(F)(F)F)CC1)c1cccc(C(F)(F)F)n1] | 315.2 |
| 03-219 | 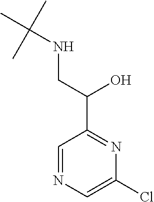 | 230.4 |
| 03-220 | 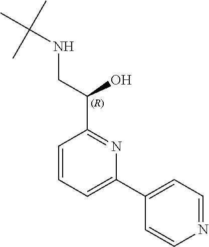 | 272.5 |
| 03-221 | 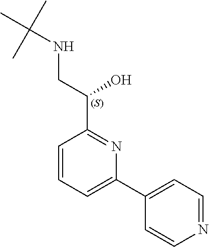 | 272.5 |
| 03-222 | 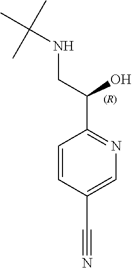 | 220.2 |
| 03-224 | 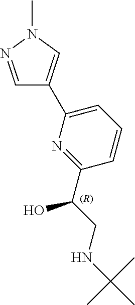 | 275.3 |
| 03-225 | 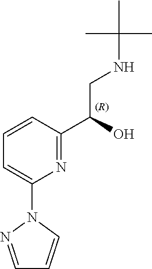 | 261.2 |
| 03-226 | 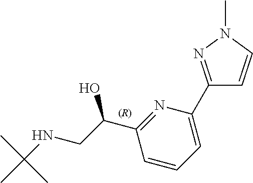 | 275.3 |
| 03-227 | 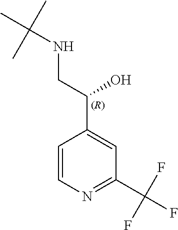 | 263.2 |
| 03-228 | 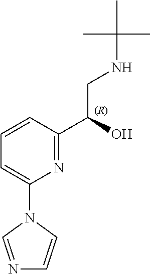 | 261.3 |
| 03-229 | 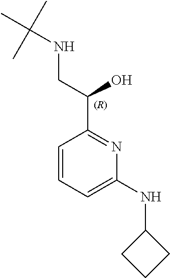 | 264.3 |
| 03-230 | 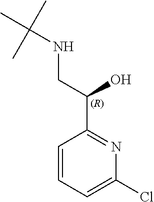 | 229.5 |
| 03-231 | 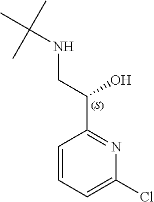 | 229.5 |
| 03-232 | 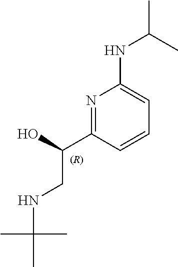 | 252.3 |
| 03-233 | 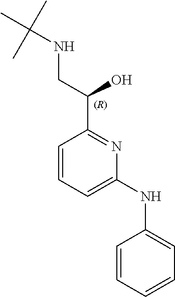 | 286.3 |
| 03-234 | 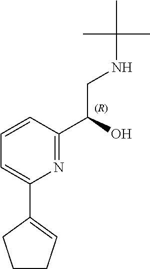 | 261.3 |
| 03-235 | 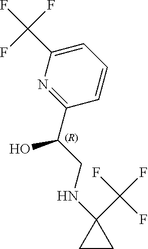 | 315.2 |
| 03-236 | 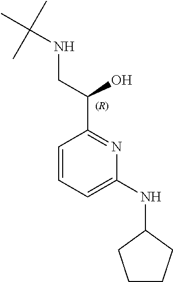 | 278.4 |
| 03-237 | 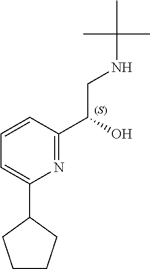 | 263.2 |
| 03-238 | 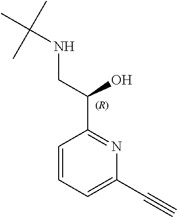 | 219.2 |
| 03-239 | 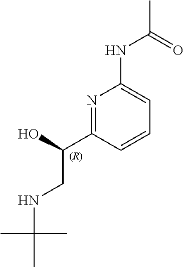 | 252.3 |
| 03-240 | 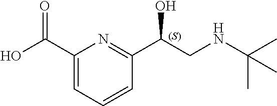 | 239.1 |
| 03-241 | 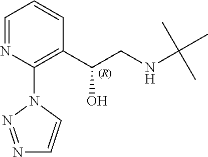 | 262.3 |
| 03-242 | 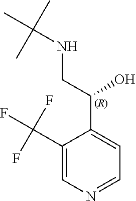 | 263.2 |
| 03-243 | 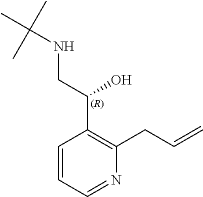 | 235.3 |
| 03-244 | 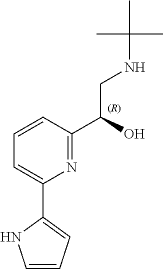 | 260.2 |
| 03-245 | 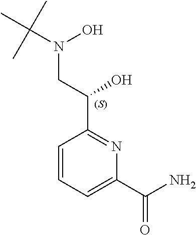 | 254.1 |
| 03-246 | 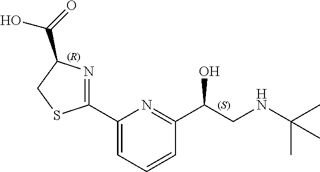 | 324.1 |
| 03-247 | 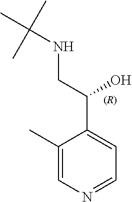 | 209.1 |
| 03-248 | 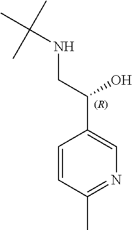 | 209.1 |
| 03-249 | 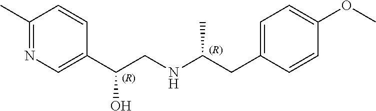 | 301.4 |
| 03-250 | 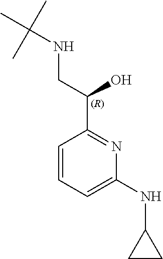 | 250.2 |
| 03-252 | 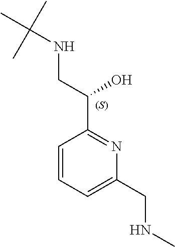 | 238.3 |
| 03-253 | 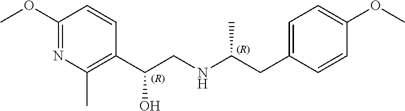 | 331.3 |
| 03-254 | 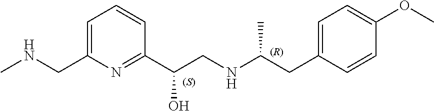 | 330.4 |
| 03-257 | 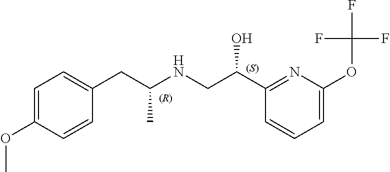 | 371.3 |
| 03-258 | 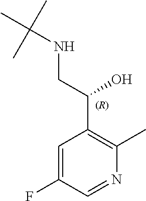 | 227.3 |
| 03-259 | 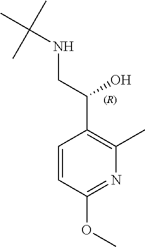 | 239.2 |
| 03-260 | 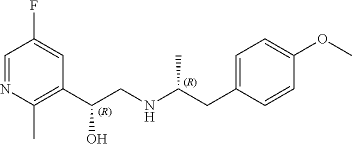 | 319.4 |
| 03-261 | 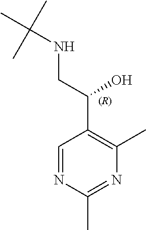 | 224.3 |
| 03-262 | 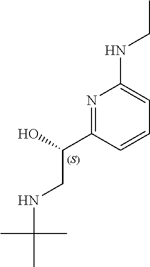 | 238.3 |
| 03-263 | 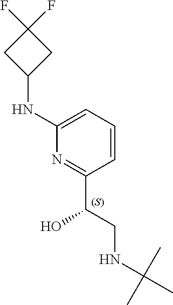 | 300.3 |
| 03-264 | 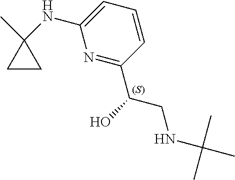 | 264.3 |
| 03-265 | 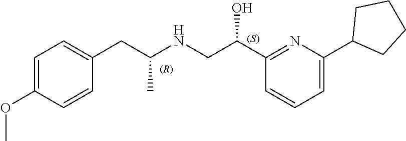 | 355.5 |
| 03-266 | 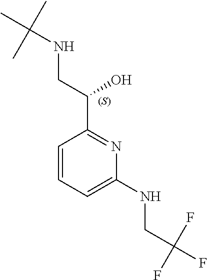 | 292.3 |
| 03-267 | 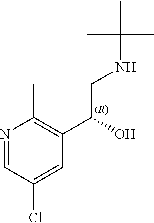 | 243.6 |
| 03-268 | 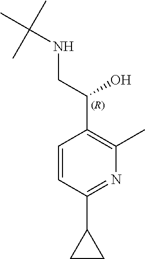 | 249.3 |
| 03-269 | 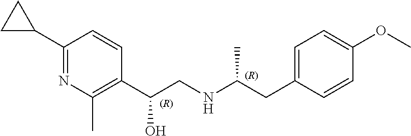 | 341.5 |
| 03-270 | 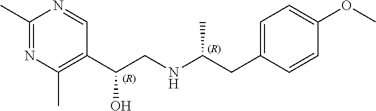 | 316.4 |
| 03-271 | 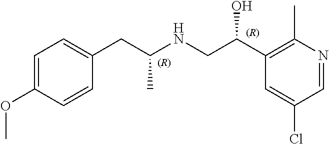 | 335.8 |
| 03-272 | 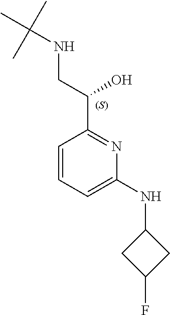 | 282.2 |
| 03-273 | 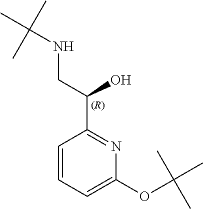 | 267.2 |
| 03-274 | 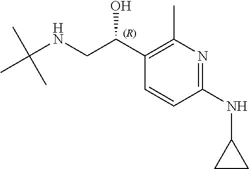 | 264.3 |
| 03-275 | 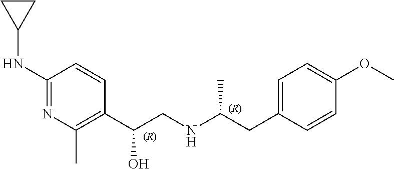 | 356.4 |
| 03-276 | 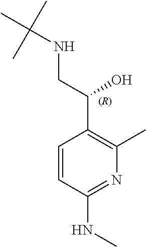 | 238.3 |
| 03-277 | 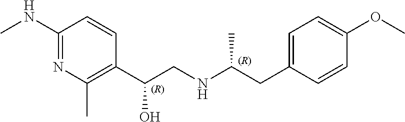 | 330.4 |
| 03-278 | 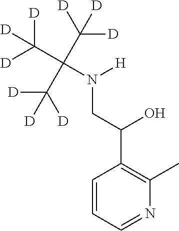 [SMILES: [2H]C([2H])([2H])C(NCC(O)c1cccnc1C)(C([2H])([2H])[2H])C([2H])([2H])[2H]] | 218.3 |
| 03-279 | 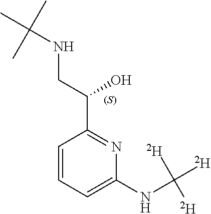 | 227.2 |
| 03-280 | 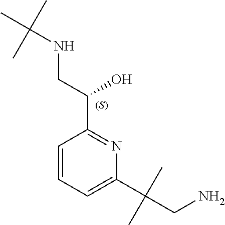 | 266.3 |
| 03-281 | 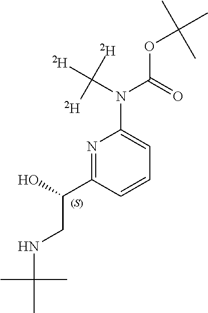 | 327.3 |

In some embodiments, a β-agent is an optically pure stereoisomer, pharmaceutically acceptable salt, solvate, or prodrug of a compound of Table 1.

### Human Models/Tests

There are many contextual learning tests used that are acknowledged and/or accepted in the art that in various embodiments may be used in conjunction with the compositions and methods disclosed herein to assess baseline cognitive function and/or to measure or quantify improved cognitive function in human subjects. For example, the contextual learning test used may be based upon single task learning, multiple task learning or spatial contextual memory. Contextual learning test evaluations based upon spatial contextual memory may be advantageous in assessing, for example, how well an individual is able to navigate a shopping mall, his or her neighborhood or a city transit or subway system as well as assessing any improvements in the ability to execute these tasks resulting from the treatment methods described herein. Other tests to evaluate effects include observations of mood, impulsivity, ADHD and the like. Such models include but are not limited to HADS, Starkstein Apathy Scale, Connors Adult ADHD Scale, or FERT.

An example of a simple spatial contextual learning test is contextual cuing, where humans learn to use repeated spatial configurations to facilitate a target search. A higher order spatial contextual learning test is serial learning, where humans learn to use subtle sequence regularities to respond more quickly and accurately to a series of events. See, for example, J. H. Howard Jr., et al., Neuropsychology, Vol. 18(1), January 2004, 124-134.

In some embodiments, cognition may be evaluated using the Mini-Mental State Examination (MMSE) and/or the Montreal Cognitive Assessment (MOCA).

Arizona Cognitive Test Battery (ACTB). A testing protocol that may be used in various embodiments is the Arizona Cognitive Test Battery (ACTB). See Edgin, J., et al. J. Neurodevelop. Disord. (2010) 2: 149-164. The ACTB has been developed specifically to assess the cognitive phenotype in DS, and includes various tests with various task demands and links with brain function. In more detail, tests are included for: 1) benchmarks, such as KBIT II verbal subscale and KBIT II non-verbal subscale IQ tests, 2) hippocampal function, 3) prefrontal function, 4) cerebellar function, 5) Finger sequencing tasks, 6) NEPSY visuomotor precision and 7) simple reaction time. Other tests include DSST, verbal fluency, ADAS-cog and the like.

In some embodiments, cognition may be evaluated using the Cambridge Neuropsychological Test Automated Battery (CANTAB) assessment (see, for example, Sahakian, et al., (1988). Brain. 111 (3): 695-718). Cognitive domains, such as attention, visuospatial working memory, episodic memory, speed of process and executive function can be assessed using the CANTAB Battery Test, which includes but is not limited to the following:
Reaction Time (RTI),
Paired Associates Learning (PAL),
Verbal Recognition Memory (VRM) Immediate Free Recall,
Rapid Visual Information Processing (RVP),
Spatial Working Memory (SWM),
Adaptive Tracking, and
VRM Delayed Free recall and Forced-Choice Recognition.

A correlation of domain/test, test description and certain primary abilities assessed in accordance with the ACTB is provided below:

| | | Primary Ability |
|---|---|---|
| Domain/Test | Description | Assessed |
| 1) Benchmark | Points to pictures based on word or | Verbal comprehension |
| KBIT-II verbal subscale | phrase | Problem solving |
| KBIT-II nonverbal subscale | Semantic or visuo-spatial | |
| | pattern completion | |
| 2) CANTAB spatial span | Touching boxes in order of | Immediate memory for |
| | changing color on screen | spatial-temporal sequence |
| 3) Prefrontal Modified dots | Press button below a cat, shifts to | Inhibitory control |
| task | new rule, press across screen for a | working memory |
| | frog, etc. | |
| 4) CANTAB IED | Forced-choice discrimination task | Set-shifting |
| | with change in relevant dimension | |
| 5) Hippocampal CANTAB | Recall for hidden abstract patterns | Spatial associative |
| paired associates | | memory |
| 6) Virtual computer- | Navigation of a virtual arena(via | Spatial memory |
| generated arena | joystick) to find a hidden target | |
| 7) Cerebellar | Sequences generated by tapping a | Motor sequencing |
| Finger-sequencing task | number of fingers (1, 2, 3, 4) to a | |
| | lever in succession | |
| 8) NEPSY visuo-motor | Follows two tracks with a pen | Visuo-motor tracking, |
| precision | | hand-eye coord. |
| 9) CANTAB simple reaction | Participants press button in | Motor response time and |
| time | response to a box presented on a | attention |
| | screen | |

The above battery of tests in some embodiments may all be performed in order to assess all major cognitive processes balanced by the practical need for testing under time constraints. The cognitive tests herein may in certain embodiments be used in subjects receiving treatment herein to monitor the subject's cognitive status and progression.

In some embodiments, the battery of tests may be conducted with a test group of individuals, and a control group individuals to demonstrate the effectiveness of various aspects and embodiments of the compositions and methods described herein. The test group may be treated with any of the treatment regimens described herein, and the control group is treated with placebo, such as a dextrose 5% saline solution by intranasal administration.

An improvement in cognitive function as defined herein as being at least a 10%, and preferably at least a 20% score improvement, on at least one, and preferably two or more, of the tests listed in the ATCB, for example. Anyone of the domain/tests listed for the ATCB above may be included in assessing whether an improvement occurred. Testing may be conducted after treatment or during treatment to ascertain whether modifications in dosage or frequency of treatment is warranted.

Brain Imaging. Generally, any non-invasive procedure many be used to both establish a baseline of brain pathology (existent or non-existent) from which baseline a treatment protocol is established. However, magnetic resonance imaging (MRI) may in some embodiments be preferred for neuroimaging examination because it allows for accurate measurement of the 3-dimensional (3D) volume of brain structures, especially the hippocampus and related regions. Such techniques are well known as described in U.S. Pat. No. 6,490,472, which patent is incorporated herein in the entirety.

Moreover, non-invasive optical imaging systems may also be used for monitoring neurological pathological events. See, for example, U.S. patent publication 2011/0286932, which is incorporated herein in the entirety. The technique described therein entails administration of a fluorescent marker to a human for staining AP peptides, imaging the retina of the DS human with an optical imaging system, and examining the images for stained AP peptides in order to determine whether onset of brain pathology (such as AD brain pathology) has occurred.

In certain embodiments, fluorodeoxyglucose positron emission tomography (FDG-PET) may be used for neuroimaging to determine cognitive function and/or identify a neurodegenerative disease in accordance with the compositions and methods described herein. The use of FDG-PET for monitoring cognitive function and/or diagnosing cognitive impairments or neurodegenerative diseases, and/or identifying subjects in need of or desiring a treatment to improve cognitive function is described in, for example Brown et al., RadioGraphics, (2014) 34:684-701, and Shivamurthy et al., AJR, (2015) 204:W76-W85; both hereby incorporated by reference in their entirety. In various embodiments, FDG-PET may be used alone or in combination with CT and/or MRI including MRI-ASL and/or MRI-BOLD. For example, FDG-PET and MRI-BOLD may be used, or FDG-PET and MRI-ASL may be used. Alternatively, FDG-PET, MRI-BOLD and MRI-ASL may be used. Alternatively, MRI, including MRI-BOLD and MRI-ASL, may be used alone or in combination, and optionally with CT.

### Alzheimer's Disease

AD brain pathology refers to the accumulation of highly degradation-resistant amyloid fibers that cause lesions in areas of the brain proximate thereto. Accumulation of these amyloid fibers to neurotoxic levels leads to destruction of nerve fibers, which, in turn, leads to the observed behavior associated with Alzheimer's dementia. Observed behavioral symptoms, which become progressively more severe with progression of the disease, often include loss of vocabulary, incorrect word substitutions (paraphasias), loss of reading and writing skills, increased risk of falling, wandering, loss of speech, apathy and even loss of muscle mass.

### Down Syndrome

Creation of several trisomic mouse models has greatly facilitated progress in the understanding the neurobiological basis of cognitive dysfunction in DS. Among the mouse models, the Ts65Dn mouse is best characterized. It has an extra copy of approximately 140 mouse genes on chromosome 16, orthologous to those on human chromosome 21 (HSA21). Almost all genes in HSA21 with potential role in nervous system abnormalities are also found in Ts65Dn mice. Similar to DS, alterations in the structure and function of the hippocampus and failure in the induction of long-term potentiation (LTP) have been extensively reported in Ts65Dn mice. Ts65Dn mice are the most widely used in DS research, and are considered to be an art-accepted model for investigations regarding DS in humans. Olson, L. E., et al., Dev. Dyn. 2004 July; 230(3):581-9.

DS is characterized by degeneration and dysfunction of multiple neuronal populations in the central nervous system (CNS). Among them, the hippocampal formation, i.e. the primary site for processing contextual learning shows significant abnormalities in DS. As a result, failure in contextual learning is a common finding in people with DS. To uncover the neurobiological basis of failed contextual learning in DS, the integrity of subcortical regions extensively projecting to the hippocampal formation have been examined. Through extensive innervation, these subcortical regions impose strong modulatory influence on hippocampal neurons. Among these subcortical regions, LC is of particular importance. LC neurons in the brainstem are the sole supplier of massive norepinephrine (NE)-ergic terminals for the hippocampus and play a significant role in wakefulness, attention, and navigational memory. Significant age-related degeneration of NE-ergic neurons of LC in Ts65Dn mice was found. Interestingly, the loss of LC terminals in Ts65Dn mice leads to further deterioration of cognitive dysfunction in these mice. Similarly, LC neurons undergo extensive age-dependent degeneration in DS. The critical role of NE-ergic system dysfunction in cognitive dysfunction in Ts65Dn has been supported by the fact that increasing brain NE levels with L-threo-3, 4-dihydroxyphenylserine (L-DOPS), i.e. a NE prodrug, restored contextual learning in Ts65Dn mice. Although L-DOPS is in phase III clinical trial for the treatment of primary autonomic failure associated with Parkinson's disease, it is yet to be approved by the FDA and its long-term effects particularly in children have yet to be explored.

With respect to the agents described herein, the terms “modulate” and “modulation” refers to the upregulation (i.e., activation or stimulation) or downregulation (i.e., inhibition or suppression) of a response. A “modulator” is an agent, compound, or molecule that modulates, and may be, for example, an agonist, antagonist, activator, stimulator, suppressor, or inhibitor. The terms “inhibit”, “reduce”, remove as used herein refer to any inhibition, reduction, decrease, suppression, downregulation, or prevention in expression, activity or symptom and include partial or complete inhibition of activity or symptom. Partial inhibition can imply a level of expression, activity or symptom that is, for example, less than 95%, less than 90%, less than 85%, less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, or less than 5% of the uninhibited expression, activity or symptom. The terms “eliminate” or “eradicate” indicate a complete reduction of activity or symptom.

As used herein, the term “a disorder” or “a disease” refers to any derangement or abnormality of function; a morbid physical or mental state. See Dorland's Illustrated Medical Dictionary, (W.B. Saunders Co. 27th ed. 1988).

As used herein, the term “treating” or “treatment” of any disease or disorder refers in one embodiment, to ameliorating the disease or disorder (i.e., slowing or arresting or reducing the development of the disease or at least one of the clinical symptoms thereof). In another embodiment “treating” or “treatment” refers to alleviating or ameliorating at least one physical parameter including those which may not be discernible by the subject. In yet another embodiment, “treating” or “treatment” refers to modulating the disease or disorder, either physically, (e.g., stabilization of a discernible symptom), physiologically, (e.g., stabilization of a physical parameter), or both. In yet another embodiment, “treating” or “treatment” refers to preventing or delaying the onset or development or progression of the disease or disorder.

In some embodiments, optically pure (S)-β agonist is used to the extent the β₂ agonist has a stereocenter, which is substantially free of (R)-β agonist. In some embodiments, optically pure (R)-β agonist is used, which is substantially free of (S)-β agonist. The term “pure”, as used herein, refers to substances that have been separated from at least some or most of the components with which they are associated in nature or when originally generated or with which they were associated prior to purification. In general, such purification involves action of the hand of man. Pure agents may be partially purified, substantially purified, or pure. Such agents may be, for example, at least 50%, 60%, 70%, 75%, 80%, 85%, 90%, 95%, 96%, 97%, 98%, 99%, or more than 99% pure. In some embodiments, a nucleic acid, polypeptide, or small molecule is purified such that it constitutes at least 75%, 80%, 85%, 90%, 95%, 96%, 97%, 98%, 99%, or more, of the total nucleic acid, polypeptide, or small molecule material, respectively, present in a preparation. In some embodiments, an organic substance, e.g., a nucleic acid, polypeptide, or small molecule, is purified such that it constitutes at least 75%, 80%, 85%, 90%, 95%, 96%, 97%, 98%, 99%, or more, of the total organic material present in a preparation. Purity may be based on, e.g., dry weight, size of peaks on a chromatography tracing (GC, HPLC, etc.), molecular abundance, electrophoretic methods, intensity of bands on a gel, spectroscopic data (e.g., NMR), elemental analysis, high throughput sequencing, mass spectrometry, or any art-accepted quantification method. In some embodiments, water, buffer substances, ions, and/or small molecules (e.g., synthetic precursors such as nucleotides or amino acids), can optionally be present in a purified preparation. A purified agent may be prepared by separating it from other substances (e.g., other cellular materials), or by producing it in such a manner to achieve a desired degree of purity.

In some embodiments, contemplated methods may include for example, administering prodrugs of the compounds described herein, or a pharmaceutical composition thereof. The term “prodrug” refers to compounds that are transformed in vivo to yield a disclosed compound or a pharmaceutically acceptable salt, hydrate or solvate of the compound. The transformation may occur by various mechanisms (such as by esterase, amidase, phosphatase, oxidative and or reductive metabolism) in various locations (such as in the intestinal lumen or upon transit of the intestine, blood or liver). Prodrugs are well known in the art (for example, see Rautio, Kumpulainen, et al., Nature Reviews Drug Discovery 2008, 7, 255). In some embodiments, the prodrug structures are constructed according to the disclosure in U.S. Pat. No. 9,849,134, which is incorporated by reference herein in the entirety.

For example, if a compound of the disclosure or a pharmaceutically acceptable salt, hydrate or solvate of the compound contains a carboxylic acid functional group, a prodrug can comprise an ester formed by the replacement of the hydrogen atom of the acid group with a group such as (C₁₋₈)alkyl, (C₂₋₁₂)alkylcarbonyloxymethyl, 1-(alkylcarbonyloxy)ethyl having from 4 to 9 carbon atoms, 1-methyl-1-(alkylcarbonyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N—(C₁₋₂)alkylamino-(C₂₋₃)alkyl (such as P-dimethylaminoethyl), carbamoyl-(C₁₋₂)alkyl, N,N-di(C₁₋₂)alkylcarbamoyl-(C₁₋₂)alkyl and Piperidino-, pyrrolidino- or morpholino(C₂₋₃)alkyl.

Similarly, if a compound of the disclosure contains an alcohol functional group, a prodrug can be formed by the replacement of the hydrogen atom of the alcohol group with a group such as (C₁₋₆)alkylcarbonyloxymethyl, 1-((C₁₋₆)alkylcarbonyloxy)ethyl, 1-methyl-1-((C₁₋₆)alkylcarbonyloxy)ethyl (C₁₋₆)alkoxycarbonyloxy)methyl, N—(C₁₋₆)alkoxycarbonylaminomethyl, succinoyl, (C₁₋₆)alkylcarbonyl, α-amino(C₁₋₄)alkylcarbonyl, arylalkylcarbonyl and α-aminoalkylcarbonyl, or α-aminoalkylcarbonyl α-aminoalkylcarbonyl, where each α-aminoalkylcarbonyl group is independently selected from the naturally occurring L-amino acids, P(O)(OH)₂, —P(O)(O(C₁₋₆)alkyl)₂ or glycosyl (the radical resulting from the removal of a hydroxyl group of the hemiacetal form of a carbohydrate).

If a compound of the disclosure incorporates an amine functional group, a prodrug can be formed, for example, by creation of an amide or carbamate, an N-alkylcarbonyloxyalkyl derivative, an (oxodioxolenyl)methyl derivative, an N-Mannich base, imine or enamine. In addition, a secondary amine can be metabolically cleaved to generate a bioactive primary amine, or a tertiary amine can metabolically cleaved to generate a bioactive primary or secondary amine. For examples, see Simplicio, et al., Molecules 2008, 13, 519 and references therein.

“Therapeutically effective amount” as used herein means the amount of a compound or composition (such as described herein) that causes at least one desirable change in a cell, population of cells, tissue, individual, subject or the like. In some embodiments a therapeutically effective amount as used herein means the amount of a compound or composition (such as described herein) that prevents or provides a clinically significant change in a disease or condition (e.g., reduce by at least about 30 percent, at least about 50 percent, or at least about 90 percent) or in one or more features of a disease or condition described herein. In some embodiments, the term “therapeutically effective amount” means an amount of a compound or composition as described herein effective or sufficient to improve cognition and/or treat a neurodegenerative disease in a subject. The term “frequency” as related thereto means the number of times a treatment is administered to a subject in order to obtain the result of improved cognition and/or treating a neurodegenerative disease in a subject.

### Diagnostics and Assessment of Treatment

In various aspects, the methods of the disclosure include diagnosing or otherwise identifying whether a subject is in need of or desiring improvement of cognitive function and/or treatment of a neurodegenerative disease. As discussed herein, this may be performed in a variety of ways as discussed herein and generally known in the art. For example, a subject diagnosis may be made by brain imaging. In various embodiments, FDG-PET may be used alone or in combination with CT and/or MRI including MRI-ASL and/or MRI-BOLD. For example, FDG-PET and MRI-BOLD may be used, or FDG-PET and MRI-ASL may be used. Alternatively, FDG-PET, MRI-BOLD and MRI-ASL may be used. Alternatively, MRI, including MRI-BOLD and MRI-ASL, may be used alone or in combination, and optionally with CT.

Along with identifying suitable subjects for treatment, diagnosis allows further determinations to be made regarding various aspects of the type and mode of treatment to be administered. For example, depending on the diagnosis, determinations may be made regarding the pharmaceutical active to be administered, the dosage of such actives as well as the timing schedule of administration.

A diagnostic method utilized with the methods of the disclosure may make use of a detectable label to diagnose or otherwise identify a subject that is in need of or desiring improvement of cognitive function and/or treatment of a neurodegenerative disease. The term “label” (also referred to as “detectable label”) refers to any moiety that facilitates detection and, optionally, quantification, of an entity that comprises it or to which it is attached. The label can be conjugated to or otherwise attached to a variety of entities, biological or otherwise. In general, a label may be detectable by, e.g., spectroscopic, photochemical, biochemical, immunochemical, electrical, optical, chemical or other means. In some embodiments a detectable label produces an optically detectable signal (e.g., emission and/or absorption of light), which can be detected e.g., visually or using suitable instrumentation such as a light microscope, a spectrophotometer, a fluorescence microscope, a fluorescent sample reader, a fluorescence activated cell sorter, a camera, or any device containing a photodetector. Labels that may be used in various embodiments include, e.g., organic materials (including organic small molecule fluorophores (sometimes termed “dyes”), quenchers (e.g., dark quenchers), polymers, fluorescent proteins); enzymes; inorganic materials such as metal chelates, metal particles, colloidal metal, metal and semiconductor nanocrystals (e.g., quantum dots); compounds that exhibit luminescence upon enzyme-catalyzed oxidation such as naturally occurring or synthetic luciferins (e.g., firefly luciferin or coelenterazine and structurally related compounds); haptens (e.g., biotin, dinitrophenyl, digoxigenin); radioactive atoms (e.g., radioisotopes such as ³H, ¹⁴C, ³²P, ³³P, ³⁵S, ¹²⁵I), stable isotopes (e.g., ¹³C, ²H); magnetic or paramagnetic molecules or particles, and the like. Fluorescent dyes include, e.g., acridine dyes; BODIPY, coumarins, cyanine dyes, napthalenes (e.g., dansyl chloride, dansyl amide), xanthene dyes (e.g., fluorescein, rhodamines), and derivatives of any of the foregoing. Examples of fluorescent dyes include Cy3, Cy3.5, Cy5, Cy5.5, Cy7, Alexa® Fluor dyes, DyLight® Fluor dyes, FITC, TAMRA, Oregon Green dyes, Texas Red, to name but a few. Fluorescent proteins include green fluorescent protein (GFP), blue, sapphire, yellow, red, orange, and cyan fluorescent proteins and fluorescent variants such as enhanced GFP (eGFP), mFruits such as mCherry, mTomato, mStrawberry; R-Phycoerythrin, and the like. Enzymes useful as labels include, e.g., enzymes that act on a substrate to produce a colored, fluorescent, or luminescent substance. Examples include luciferases, β-galactosidase, horseradish peroxidase, and alkaline phosphatase. Luciferases include those from various insects (e.g., fireflies, beetles) and marine organisms (e.g., cnidaria such as Renilla (e.g., Renilla reniformis, copepods such as Gaussia (e.g., Gaussia princeps) or Metridia (e.g., Metridia longa, Metridia pacifica), and modified versions of the naturally occurring proteins. A wide variety of systems for labeling and/or detecting labels or labeled entities are known in the art. Numerous detectable labels and methods for their use, detection, modification, and/or incorporation into or conjugation (e.g., covalent or noncovalent attachment) to biomolecules such as nucleic acids or proteins, and the like, are described in lain Johnson, I., and Spence, M. T. Z. (Eds.), The Molecular Probes® Handbook--A Guide to Fluorescent Probes and Labeling Technologies. 11th edition (Life Technologies/Invitrogen Corp.) available online on the Life Technologies website at invitrogen.com/site/us/en/home/References/Molecular-Probes-The-Handbook.html and Hermanson, G T., Bioconjugate Techniques, 2^{nd} ed., Academic Press (2008). Many labels are available as derivatives that are attached to or incorporate a reactive functional group so that the label can be conveniently conjugated to a biomolecule or other entity of interest that comprises an appropriate second functional group (which second functional group may either occur naturally in the biomolecule or may be introduced during or after synthesis). For example, an active ester (e.g., a succinimidyl ester), carboxylate, isothiocyanate, or hydrazine group can be reacted with an amino group; a carbodiimide can be reacted with a carboxyl group; a maleimide, iodoacetamide, or alkyl bromide (e.g., methyl bromide) can be reacted with a thiol (sulfhydryl); an alkyne can be reacted with an azide (via a click chemistry reaction such as a copper-catalyzed or copper-free azide-alkyne cycloaddition). Thus, for example, an N-hydroxysuccimide (NHS)-functionalized derivative of a fluorophore or hapten (such as biotin) can be reacted with a primary amine such as that present in a lysine side chain in a protein or in an aminoallyl-modified nucleotide incorporated into a nucleic acid during synthesis. A label may be directly attached to an entity or may be attached to an entity via a spacer or linking group, e.g., an alkyl, alkylene, aminoallyl, aminoalkynyl, or oligoethylene glycol spacer or linking group, which may have a length of, e.g., between 1 and 4, 4-8, 8-12, 12-20 atoms, or more in various embodiments. A label or labeled entity may be directly detectable or indirectly detectable in various embodiments. A label or labeling moiety may be directly detectable (i.e., it does not require any further reaction or reagent to be detectable, e.g., a fluorophore is directly detectable) or it may be indirectly detectable (e.g., it is rendered detectable through reaction or binding with another entity that is detectable, e.g., a hapten is detectable by immunostaining after reaction with an appropriate antibody comprising a reporter such as a fluorophore or enzyme; an enzyme acts on a substrate to generate a directly detectable signal). A label may be used for a variety of purposes in addition to or instead of detecting a label or labeled entity. For example, a label can be used to isolate or purify a substance comprising the label or having the label attached thereto.

The term “labeled” is used herein to indicate that an entity (e.g., a molecule, such as a biological or small molecule, organic compound, probe, cell, tissue, and the like) comprises or is physically associated with (e.g., via a covalent bond or noncovalent association) a label, such that the entity can be detected. In some embodiments a detectable label is selected such that it generates a signal that can be measured and whose intensity is related to (e.g., proportional to) the amount of the label. In some embodiments two or more different labels or labeled entities are used or present in a composition. In some embodiments the labels may be selected to be distinguishable from each other. For example, they may absorb or emit light of different wavelengths. In some embodiments the labels may be selected to interact with each other. For example, a first label may be a donor molecule that transfers energy to a second label, which serves as an acceptor molecule through nonradiative dipole—coupling as in resonance energy transfer (RET), e.g., Forster resonance energy transfer (FRET, also commonly called fluorescence resonance energy transfer).

Nuclear imaging is one of the most important tools of diagnostic medicine wherein an estimated 12-14 million nuclear medicine procedures are performed each year in the United States alone. Diagnostic nuclear imaging is therefore crucial for studies which determine the cause of a medical problem based on organ function, in contrast to radiographic studies, which determine the presence of disease based on static structural appearance.

Diagnostic radiopharmaceuticals and radiotracers are often designed or selected capable of selective binding to specific receptors by means of a binding moiety, such as an antibody, a specific inhibitor or other target-specific ligand. These targeted markers can therefore concentrate more rapidly in areas of interest, such as inflamed tissues, tumors, malfunctioning organs or an organ undergoing heightened expression of certain proteins. Thus, a blood circulating radiopharmaceutical is Picked up by a specific organ or pathological tissue to a different extent than by other or non-pathological tissue. For example, a highly vascularized tissue (e.g., of a growing tumor) may concentrate more of a radiopharmaceutical while an ischemic tissue may concentrate less of the radiopharmaceutical than the surrounding tissues. Nuclear imaging relies on these general phenomena of varied distribution of radiopharmaceutical according to different tissue as well as different pathologies. As a result, specific tissue types (e.g., tumor tissues) may be distinguished from other tissues in radioactive-emission imaging.

Radiopharmaceuticals, which may be used in the process of differential diagnosis of pathologies may be conjugated to targeting (recognition binding) moieties and include a wide range of radioisotopes as mentioned below. Such radiopharmaceuticals therefore include recognition moieties such as, for example, monoclonal antibodies (which bind to a highly specific pre-determined target), fibrinogen (which is converted into fibrin during blood clotting), glucose and other chemical moieties and agents. Commonly used diagnostic conjugated radiopharmaceuticals include, for example, 2-[¹⁸F]fluoro-2-deoxy-D-glucose (¹⁸FDG), ¹¹¹In-Pentetreotide ([¹¹¹In-DTPA-D-Phe¹]-octreotide), L-3-[¹²³I]-Iodo-α-methyl-tyrosine (IMT), O-(2-[¹⁸F]fluoroethyl)-L-tyrosine (L-[¹⁸F]FET), ¹¹¹In-Capromab Pendetide (CYT-356, Prostascint) and ¹¹¹In-Satumomab Pendetide (Oncoscint).

Two basic techniques are widely used for nuclear imaging: positron emission tomography (PET) and single photon emission computed tomography (SPECT). PET detects photons generated through positron-electron annihilation of positrons from a diagnostic radiopharmaceutical tracer placed in the subject, e.g., subject, to be imaged, and analyzes the photon energy and trajectory to generate tomographic images of the subject. SPECT generates images by computer analysis of photon emission events from a diagnostic radiopharmaceutical tracer having gamma emitting isotopes. Both PET and SPECT require the detection and analysis of single photon events, which are characterized by low signal to noise ratio and scarcity relative to the background radiation. Other constraints on the PET and SPECT image qualities include the sensitivity, temporal and spatial resolution, dynamic range, response time and counting rate characteristics of the data acquisition probe devices, e.g., photomultipliers and the like.

Radioisotopes that emit both high energy y and/or low energy y, P and/or positron radiation and which can be used per se or as a part of a compound as radiopharmaceuticals, include, without limitation, technetium-99m (^{99m}Tc), gallium-67 (⁶⁷Ga), thallium-201 (²⁰¹Tl), 111indium-(¹¹¹In), iodine-123 (¹²³I), iodine-125 (¹²⁵I), iodine-131 (¹³¹I), xenon-133 (¹³³Xe), and fluorine-18 (¹⁸F). All these isotopes, except ^{99m}Tc, ¹³¹I and ¹³³Xe, are produced in particle accelerators.

Non-limiting examples of commonly used radiotracers include ^{99m}Tc-Arcitumomab (CEA-Scan™) which is a monoclonal antibody for imaging colorectal tissues afflicted with colorectal cancer, ^{99m}Tc-sestamibi (Cardiolite™) and ^{99m}Tc-tetrofosmin (Myoview™) for imaging the heart of a subject for myocardial perfusion, ¹¹¹In-Capromab pendetide (ProstaScint™) which is a monoclonal antibody for imaging prostate tissues afflicted with prostate cancer, ^{99m}Tc-Fanolesomab (NeutroSpec™) which is a monoclonal antibody for imaging inflamed and infectious tissues and ⁹⁰Y/111In-Zevalin (Ibritumomab Tiuxetan) which is a monoclonal antibody directed against the CD20 antigen, whereby this antigen is found on the surface of normal and malignant B lymphocytes.

Any diagnostic radiopharmaceutical can be utilized in the kit of the present embodiments. Exemplary radiopharmaceuticals that can be utilized in this context of the present invention include, without limitation, ³H-water, ³H-inulin, ¹¹C-carbonmonoxide, ¹³N-ammonia, ¹⁴C-inulin, ¹⁵O—H₂O, ¹⁵O—O₂, ¹⁸F-fluorodeoxyglucose, ¹⁸F-sodium fluoride, ⁵¹Cr-erythrocytes (RBC), ⁵⁷Co-vitamin B12 (cyanocobalamin), ⁵⁸Co-vitamin B12 (cyanocobalamin), ⁵⁹Fe-citrate, ⁶⁰Co-vitamin B12 (cyanocobalamin), ⁶⁷Ga-citrate, ⁶⁸Ga-citrate, ⁷⁵Se-selenomethionine, ^{81m}Kr-krypton for inhalation, oral administration or injections, ⁸²Rb, ¹⁵Sr-nitrate, ⁹⁰Y/¹¹¹In-ibritumomab tiuxetan (⁹⁰Y/¹¹¹In-Zevalin), ^{99m}Tc-albumin microspheres, ^{99m} Tc-disofenin, lidofenin and mebrofenin, ⁹⁹mTc-DMSA, ^{99m}Tc-DTPA (injection), ^{99m}Tc-DTPA (aerosol), ^{99m}Tc-ECD (ethylene cystate dimer), ^{99m}Tc-exametazime (HMPAO), ^{99m}Tc-glucoheptonate, ⁹⁹mTc-HEDP, ⁹⁹mTc-HMDP, ⁹⁹mTc-HSA, ⁹⁹mTc-MAA, ⁹⁹mTc-MAG.sub.3, ⁹⁹mTc-MDP, ⁹⁹mTc-tetrofosmin (Myoview), ^{99m}Tc-sestamibi (Cardiolite), ^{99m}Tc-oral administrations, ^{99m}Tc-pertechnetate, ^{99m}Tc-pyrophosphate, ^{99m}Tc-RBC in vitro and in vivo labeling, ^{99m}Tc-sulfur colloid, ^{99m}Tc-teboroxime, ^{99m}Tc-white blood cells, ¹¹¹In-ibritumomab tiuxetan (¹¹¹In-Zevalin), ¹¹¹In-DTPA, ¹¹¹In-platelets, ¹¹¹In-RBC, ¹¹¹In-white blood cells, ¹²³I-hippuran, ¹²³I-IMP, ¹²³I-mIBG, ¹²³I-sodium iodide, ¹²⁴I-sodium iodide, ¹²⁵I-fibrinogen, ¹²⁵I-IMP, ¹²⁵I-mIBG, ¹²⁵I-sodium iodide, ¹²⁶I-sodium iodide, ¹³⁰I-sodium iodide, ¹³¹I-hippuran, ¹³¹I-HSA, ¹³¹I-MAA, ¹³¹I-mIBG, ¹³¹I-Rose Bengal, ¹³¹I-sodium iodide, ¹²⁷Xe-inhalation and injection, ¹³³Xe-inhalation and injection, ¹⁹⁷Hg-chlormerodrin, ¹⁹⁸Au-colloid and ²⁰¹Tl-chloride.

The diagnostic methods described herein may also but utilized to assess the effectiveness of a particular therapeutic regimen. For example, a subject that has been identified as being in need of or desiring improvement of cognitive function and/or treatment of a neurodegenerative disease and which is being treated, may be diagnosed or otherwise assessed to determine the effectiveness of the treatment regime. While the diagnosis or assessment may be performed by any method known in the art, cognitive testing or brain imaging may be used to determine improvement of cognitive function or amelioration of a disease. In embodiments, cognitive testing or brain imaging may be used alone or in combination. In embodiments where brain imaging is utilized, FDG-PET may be used alone or in combination with CT and/or MRI including MRI-ASL and/or MRI-BOLD. For example, FDG-PET and MRI-BOLD may be used, or FDG-PET and MRI-ASL may be used. Alternatively, FDG-PET, MRI-BOLD and MRI-ASL may be used. Alternatively, MRI, including MRI-BOLD and MRI-ASL, may be used alone or in combination, and optionally with CT.

The assessment of treatment efficacy may be utilized to alter the treatment regime of a subject. For example, the assessment may be utilized to alter dosing, timing of administration, and/or the actives of the pharmaceutical composition. In embodiments, the dosage of a particular pharmaceutical agent being administered to the subject may be lowered by combining administration with a different agent. In this manner, treatment may be optimized by altering the pharmaceutical composition to include different combinations of β-agent, β₁-AR agonist, β₂-AR agonist, and peripherally acting β-blocker (PABRA). Dosing may also be altered depending on the timing of administration. For example, a shorter duration between each administration of the pharmaceutical composition may require a lower dose of active agent, while a longer duration between each administration of the pharmaceutical composition may require a higher dose of active agent, either of which may improve the treatment regime as determined by diagnosis or assessment of the subject.

In one embodiment, a subject may be assessed a single time during the course of treatment to optimize the treatment regime. Alternatively, the subject may be assessed multiple times over the course of treatment to continually optimize the treatment regime as directed by a medical professional.

### Dosage, Administration and Pharmaceutical Formulation

The term “pharmaceutically-accepted salts” means acid addition salts that are commonly used in human or veterinary medicine and are deemed safe for use. Examples for the present disclosure include, but are not limited to, salts obtained from the following acids: acetic, ascorbic, benzenesulfonic, benzoic, camphosulfonic, citric, ethanesulfonic, edisylic, fumaric, gentisic, gluconic, glucoronic, glutamic, hippuric, hydrobromic, isethionic, lactic, nitric, phosphoric, succinic, sulfuric and tartaric, for example. Any hydrated forms of such salts are also included in this definition. Thus, for example, both fumarate and hemifumarate salts are specifically contemplated as well as any hydrates thereof. For example, fumarate dihydrate may be specifically mentioned.

The pharmaceutical preparation in some embodiments may be in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form. Preferably, the unit dosage form is a tablet. The composition can, if desired, also contain other compatible therapeutic agents. Preferred pharmaceutical preparations can deliver the compounds of the disclosure in a sustained release formulation.

For a binding agent, composition, or compound according to the present disclosure, the dosage form may optionally be a liquid dosage form. Solutions can be prepared in water suitably mixed with a surfactant such as hydroxypropylcellulose or an emulsifier such as polysorbate. Dispersions can also be prepared in glycerol, liquid polyethylene glycols, DMSO and mixtures thereof with or without alcohol, and in oils. Under ordinary conditions of storage and use, these preparations contain a preservative to prevent the growth of microorganisms. Conventional procedures and ingredients for the selection and preparation of suitable formulations are described, for example, in Remington's Pharmaceutical Sciences (2003-20th edition) and in The United States Pharmacopeia: The National Formulary (USP 24 NF19) published in 1999. Formulations optionally contain excipients including, but not limited to, a buffering agents, an anti-oxidant, a stabilizer, a carrier, a diluent, and an agent for pH adjustment. The pharmaceutical forms suitable for injectable use include sterile aqueous solutions or dispersion and sterile powders for the extemporaneous preparation of sterile injectable solutions or dispersions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl, or benzyl alcohol; alkyl parabens such as methyl or propyl paraben; catechol; resorcinol; cyclohexanol; 3-pentanol; and m-cresol); low molecular weight (less than about 10 residues) polypeptides; proteins such as serum, albumin, gelatin, or immunoglobulins; hydrophilic polymers such as polyvinylpyrrolidone; amino acids such as glycine, glutamine, asparagine, histidine, arginine or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN, PLURONICS or polyethylene glycol (PEG).

In various embodiments, the dose of an agent may be determined by the human subject's body weight. For example, an absolute dose of an agent of about 30 to 160 μg for a pediatric human subject of about 0 to about 5 kg (e.g. about 0, or about 1, or about 2, or about 3, or about 4, or about 5 kg); or about 30 to 160 μg for a pediatric human subject of about 6 to about 8 kg (e.g. about 6, or about 7, or about 8 kg), or about 30 to 160 μg for a pediatric human subject of about 9 to about 13 kg (e.g. 9, or about 10, or about 11, or about 12, or about 13 kg); or about 30 to 160 μg for a pediatric human subject of about 14 to about 20 kg (e.g. about 14, or about 16, or about 18, or about 20 kg), or about 30 to 160 μg for a pediatric human subject of about 21 to about 30 kg (e.g. about 21, or about 23, or about 25, or about 27, or about 30 kg), or about 30 to 160 μg for a pediatric human subject of about 31 to about 33 kg (e.g. about 31, or about 32, or about 33 kg), or about 30 to 160 μg for an adult human subject of about 34 to about 50 kg (e.g. about 34, or about 36, or about 38, or about 40, or about 42, or about 44, or about 46, or about 48, or about 50 kg), or 30 to 160 μg for an adult human subject of about 51 to about 75 kg (e.g. about 51, or about 55, or about 60, or about 65, or about 70, or about 75 kg), or about 30 to 160 μg for an adult human subject of greater than about 114 kg (e.g. about 114, or about 120, or about 130, or about 140, or about 150 kg).

In certain embodiments, an agent in accordance with the methods provided herein is administered orally, subcutaneously (s.c.), intravenously (i.v.), intramuscularly (i.m.), intranasally or topically. Administration of an agent described herein can, independently, be one to four times daily; or one or two times weekly; or one to four times per month; or one to six times per year or once every two, three, four or five years. Administration can be for the duration of one day or one month, two months, three months, six months, one year, two years, three years, and may even be for the life of the human subject. The dosage may be administered as a single dose or divided into multiple doses. In some embodiments, an agent is administered about 1 to about 3 times (e.g. 1, or 2 or 3 times).

The compounds of this disclosure may be prepared or isolated in general by synthetic and/or semi-synthetic methods known to those skilled in the art for analogous compounds and by methods described in detail in the Examples, herein. In one embodiment, the compounds selected from those compounds set forth in Table 1 were prepared by the methods illustrated in Scheme A.

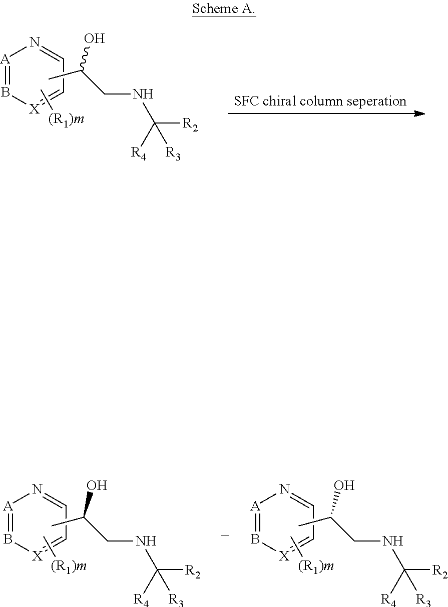

In one embodiment, the compounds selected from those compounds set forth in Table 1 were prepared by the methods illustrated in Scheme B.

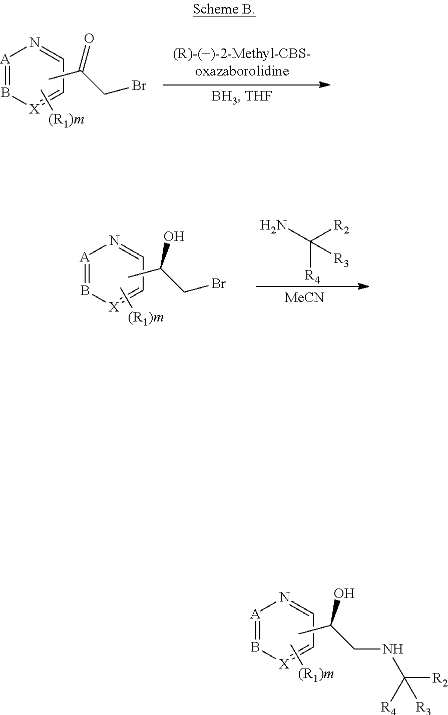

In one embodiment, the compounds selected from those compounds set forth in Table 1 were prepared by the methods illustrated in Scheme C.

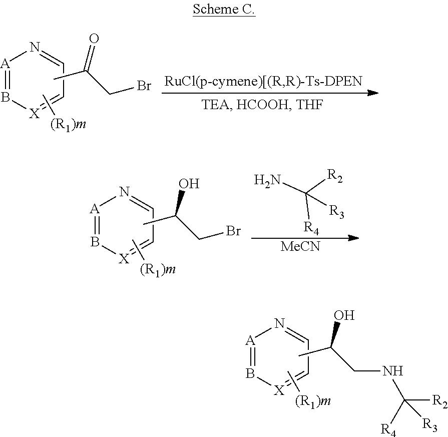

In one embodiment, the compounds selected from those compounds set forth in Table 1 were prepared by the methods illustrated in Scheme D.

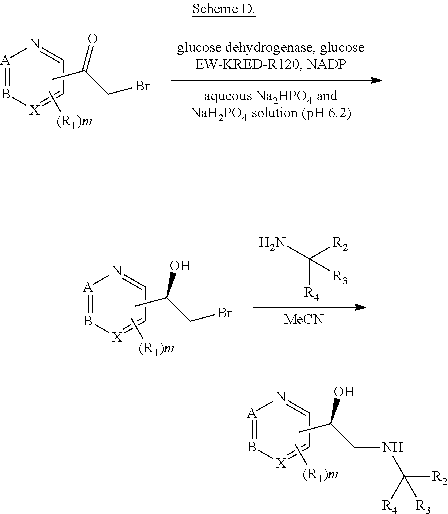

In one embodiment, the compounds selected from those compounds set forth in Table 1 were prepared by the methods illustrated in Scheme E.

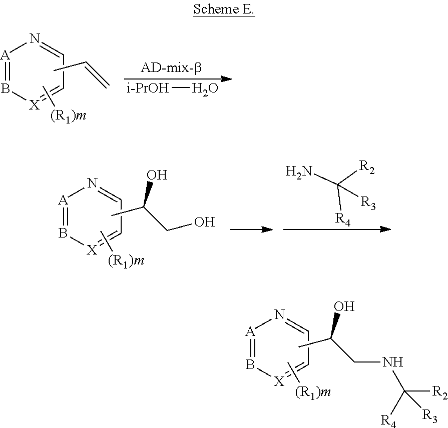

In one embodiment, the compounds selected from those compounds set forth in Table 1 were prepared by the methods illustrated in Scheme F.

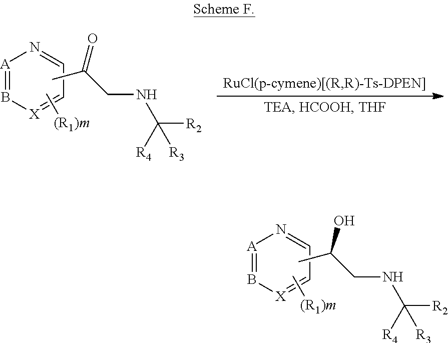

In one embodiment, the compounds selected from those compounds set forth in Table 1 were prepared by the methods illustrated in Scheme G.

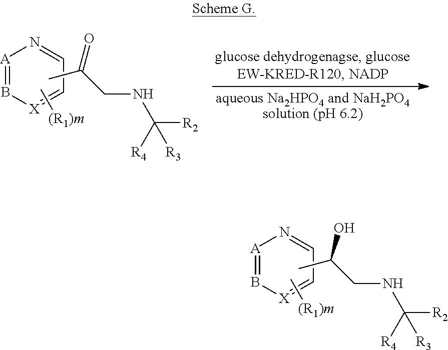

### EXAMPLES

The present disclosure will be further described in the following examples, which do not limit the scope of the present disclosure.

### Example 1: Treatment of Human Subjects with Clenbuterol

Subjects are screened using FDG-PET brain imaging. The identified as diagnosed with one or more of MCI, aMCI, Vascular Dementia, Mixed Dementia, FTD (fronto-temporal dementia; Pick's disease), HD (Huntington disease), Rett Syndrome, PSP (progressive supranuclear palsy), CBD (corticobasal degeneration), SCA (spinocerebellar ataxia), MSA (Multiple system atrophy), SDS (Shy-Drager syndrome), olivopontocerebellar atrophy, TBI (traumatic brain injury), CTE (chronic traumatic encephalopathy), stroke, WKS (Wernicke-Korsakoff syndrome; alcoholic dementia & thiamine deficiency), normal pressure hydrocephalus, hypersomnia/narcolepsy, ASD (autistic spectrum disorders), FXS (fragile X syndrome), TSC (tuberous sclerosis complex), prion-related diseases (CJD etc.), depressive disorders, DLB (dementia with Lewy bodies), PD (Parkinson's disease), PDD (PD dementia), or ADHD (attention deficit hyperactivity disorder).

A single dose of clenbuterol was provided to the subjects ranging in an amount from 30 to 160 μg. A single dose of nadolol was also administered in some subjects in an amount of 5 mg to counter any adverse effects of the clenbuterol. The subject are tracked over the course of 3 days after the single dose of clenbuterol and/or nadolol. The subjects demonstrated robust global increase in cerebral blood flow from the baseline following treatment with clenbuterol and/or nadolol.

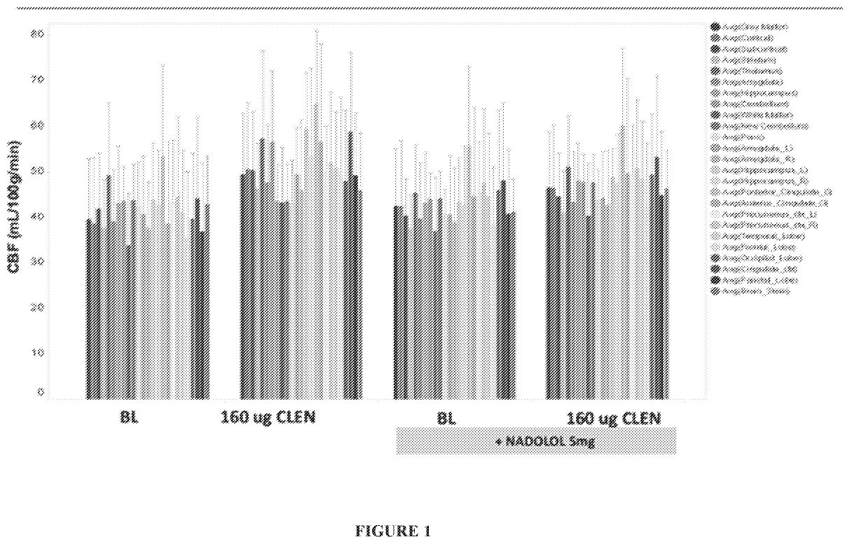

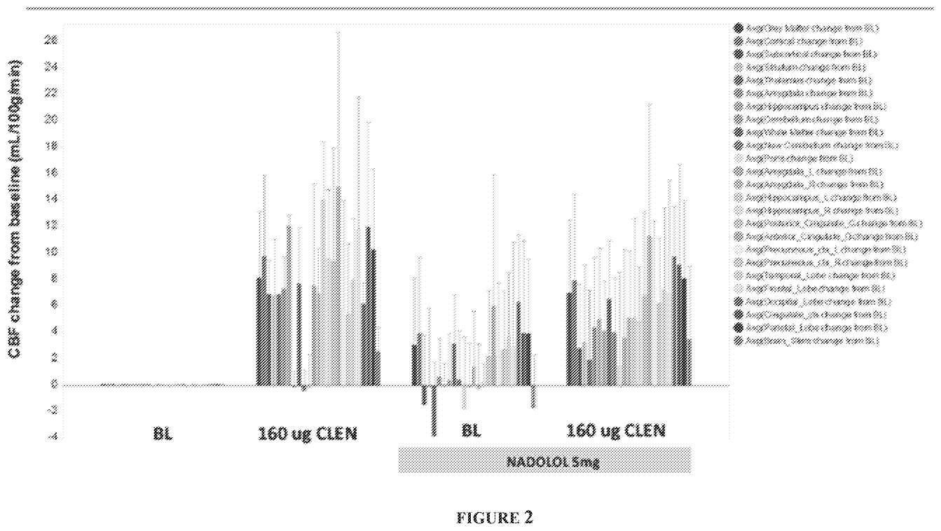

As shown in FIG. 1, a first group of subjects was administered a single dose of clenbuterol in an amount of 160 μg and a second group of subjects was administered a single dose of clenbuterol in an amount of 160 μg and nadolol in an amount of 5 mg. Relative to their baseline prior to the single dose of treatment, clenbuterol produces a robust global increase in cerebral blood flow (CBF) relative to the baseline in these subjects. The second group of subjects also demonstrated a robust global increase in cerebral blood flow (CBF) relative to the baseline in these subjects, in which nadolol was also administered with clenbuterol to counter any adverse effects of clenbuterol.

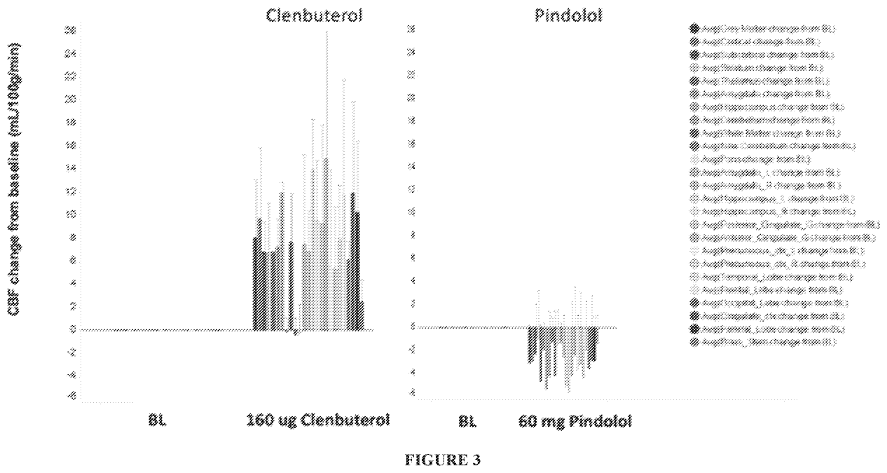

As shown in FIG. 3, a first group of subjects was administered a single dose of clenbuterol in an amount of 160 μg and a second group of subjects was administered a single dose of pindolol in an amount of 60 mg. Treatment with clenbuterol showed a positive increase in cerebral blood flow relative to the base line. Treatment with pindolol showed a decrease in cerebral blood flow relative to the base line.

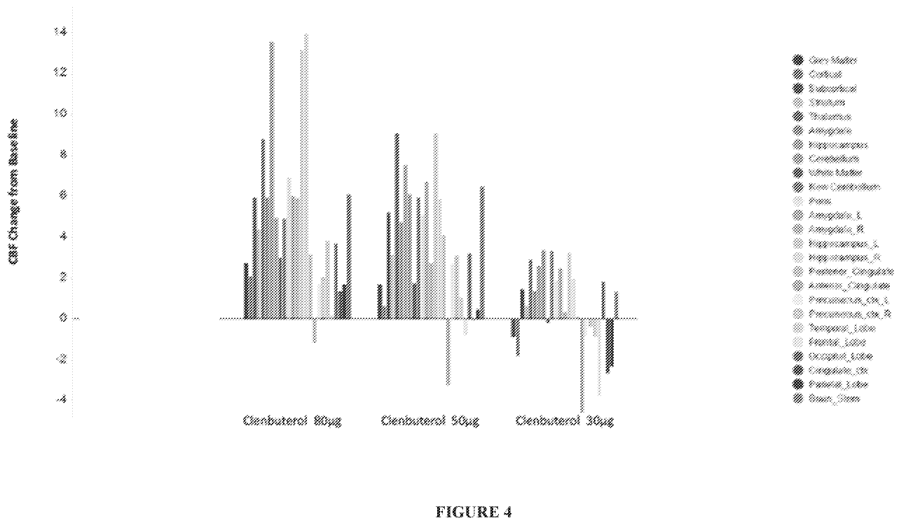

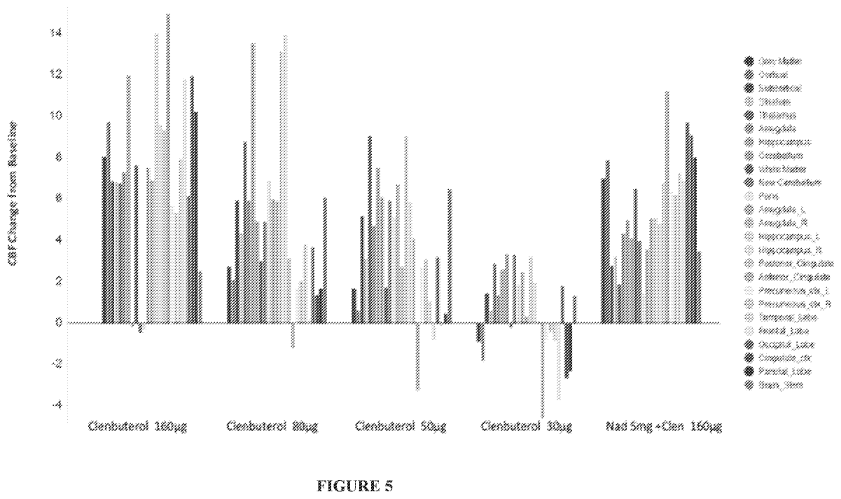

As shown in FIGS. 4 and 5, a groups of subjects were administered a single dose of varying amounts of clenbuterol ranging from 30 to 160 μg, and another group of subjects was administered a single dose of clenbuterol in an amount of 160 μg and nadolol in an amount of 5 mg to counter any adverse effects of clenbuterol. The subjects were tracked over the course of 3 days. Relative to their baseline prior to the single dose of treatment, clenbuterol in an amount ranging from 30 to 160 μg produces a robust global increase in cerebral blood flow (CBF) relative to the baseline in these subjects. The subjects administered a single dose of clenbuterol in an amount of 160 μg and nadolol in an amount of 5 mg also showed a robust global increase in cerebral blood flow (CBF) relative to the baseline.

In some embodiments, cognitive tests and/or FDG-PET imaging can be used. In some embodiments, magnetic resonance imaging-arterial spin labeling (MRI-ASL) can be used for neuroimaging. In some embodiments, magnetic resonance imaging-blood oxygenation level dependent computerized tomography (MRI-BOLD) can be used for neuroimaging.

### Example 2: Synthesis of Compound 03-5 and 03-48

The below Scheme illustrates the synthesis of compound 03-5 and 03-48.

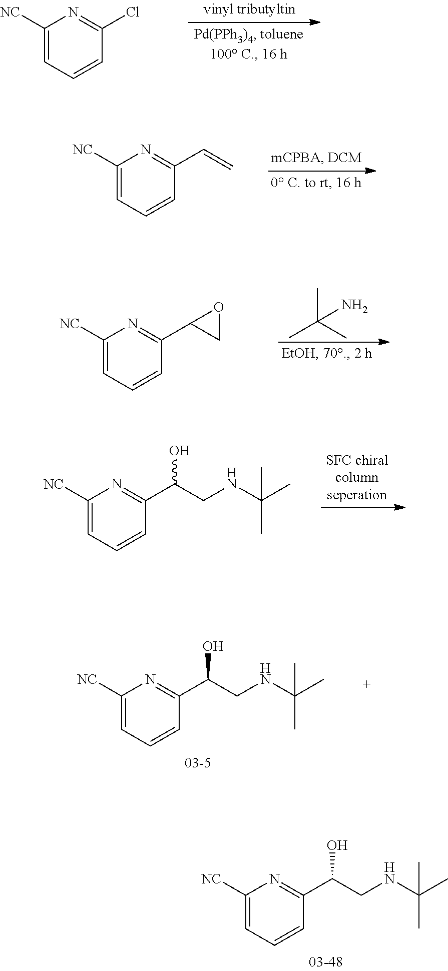

### Step 1. Synthesis of 2-cyano-6-vinylpyridine

To a stirred mixture of 2-chloro-6-cyanopyridine (8.0 g, 69.3 mmol), 1-vinyltri-n-butyltin (21.97 g, 69.29 mmol, 20.34 mL), and Pd(PPh₃)₄ (3.34 g, 3.61 mmol) in anhydrous toluene (150 mL) was bubbled with N₂ for 5 min, before heating to 80° C. overnight. After cooling, the reaction mixture was poured into an aqueous solution of KF (40 g in 200 mL) and stirred for 30 min. The mixture was then filtered through celite and solid was washed with EtOAc (2×50 mL). The aqueous phase of the filtrate was separated and extracted with EtOAc (2×250 mL). The combined organic phases were washed with brine, dried over Na₂SO₄, filtered and concentrated under reduced pressure. The residue was purified by flash chromatography eluting with Hexanes/EtOAc (silica, 95/5 to 90/10) to provide 2-cyano-6-vinylpyridine as a pale yellow liquid (6.5 g, 86%). MS (m/z): 131.1 (M+H)⁺.

### Step 2: Synthesis of 6-(oxiran-2-yl)picolinonitrile

To a stirred solution of 2-cyano-6-vinylpyridine (6.5 g, 49.94 mmol) in DCM (300 mL) was added mCPBA (61.56 g, 249.72 mmol) at 0° C. slowly portion wise over a period of 30 min and stirred at RT for 24 h. After completion of reaction, reaction mixture was cooled to 5° C. and added aqueous saturated NaHCO₃ solution and extracted with DCM (200 mL×2). Organic layers were combined, dried over Na₂SO₄, filtered, and concentrated under reduced pressure. The residue was purified by flash chromatography eluting with Hexanes/EtOAc (silica, 90/10 to 80/20) to provide 6-(oxiran-2-yl)picolinonitrile as a colorless liquid (3.85 g, 52%). MS (m/z): 147.1 (M+H)⁺.

### Step 3: Synthesis of (S)-6-(2-(tert-butylamino)-1-hydroxyethyl)picolinonitrile and (R)-6-(2-(tert-butylamino)-1-hydroxyethyl)picolinonitrile

To a stirred solution of 6-(oxiran-2-yl) picolinonitrile (3.5 g, 18.2 mmol) in ethanol (25 mL) was added tert-butylamine (6.66 g, 91.0 mmol). The reaction mixture was stirred at 80° C. for 3 h in a sealed tube, while monitoring reaction by TLC and LCMS. After completion of reaction, solvent was evaporated to get a residue, which was purified by reverse phase chromatography to provide desired products as a racemic mixture. A racemic mixture was separated by SFC (Chiralpak AS-H (30*250) mm, 5μ column, using CO₂: 80% Co-solvent: 20% (0.2% isopropylamine in IPA as eluent) to provide compound 03-5 (S)-6-(2-(tert-butylamino)-1-hydroxyethyl)picolinonitrile (1.05 g, 26.3%) and compound 03-48 (R)-6-(2-(tert-butylamino)-1-hydroxyethyl)picolinonitrile (0.98 g, 24.5%) as white solids. Compound 03-5: ¹HNMR 400 MHz, DMSO-d6: δ 8.03 (t, J=8.00 Hz, 1H), 7.90 (dd, J=0.80 Hz, 7.60 Hz, 1H), 7.82 (d, J=8.00 Hz, 1H), 5.63 (s, 1H), 4.60 (q, J=4.40 Hz, 1H), 2.86-2.80 (m, 1H), 2.67-2.49 (m, 1H), 1.44-1.40 (m, 1H), 0.98 (s, 9H). Compound 03-48: ¹HNMR 400 MHz, DMSO-d6: δ 8.03 (t, J=7.60 Hz, 1H), 7.90 (d, J=6.80 Hz, 1H), 7.82 (d, J=8.00 Hz, 1H), 5.62 (s, 1H), 4.60 (s, 1H), 2.81-2.82 (m, 1H), 2.62-2.64 (m, 1H), 1.44 (s, 1H), 0.98 (s, 9H).

### Example 3: Cerebral Perfusion

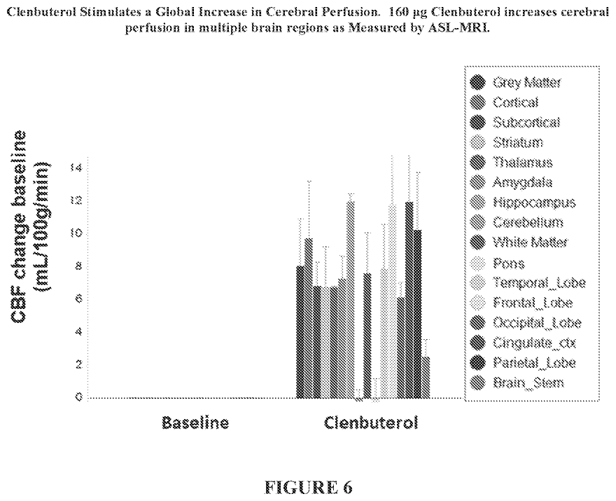

Several recent studies have demonstrated the clinical relevance of cerebral perfusion (De Vis 2018, Staffaroni 2019). These studies demonstrate that cerebral perfusion declines with age, is correlated with the progression of AD, and is strongly correlated with cognitive performance such that subjects with higher cerebral perfusion tend to perform better in cognitive tests. Additionally, a study in AD subjects demonstrated that the clinical effect of donepezil could be predicted by the perfusion increase seen after a single dose of the drug such that the subjects who had an increase in perfusion after acute administration were the same subjects who had a cognitive improvement after 6 months of treatment with the drug (Tepmongkol 2019). In a clinical study, healthy subjects were administered doses of clenbuterol ranging from 20 to 160 μg and ASL MRI was conducted prior to and after dosing with an objective to ascertain whether this neuroimaging method enables the detection of a clinically relevant CNS signal. The neuroimaging data from the study using ASL MRI demonstrated a clinically relevant signal, an increase in cerebral perfusion after a single dose of clenbuterol. specifically, 160 μg of clenbuterol causes a robust global increase in cerebral perfusion and in particular in areas such as the hippocampus, thalamus, and cortex, all of which are very relevant in the pathogenesis of neurodegenerative disorders (see FIG. 6)

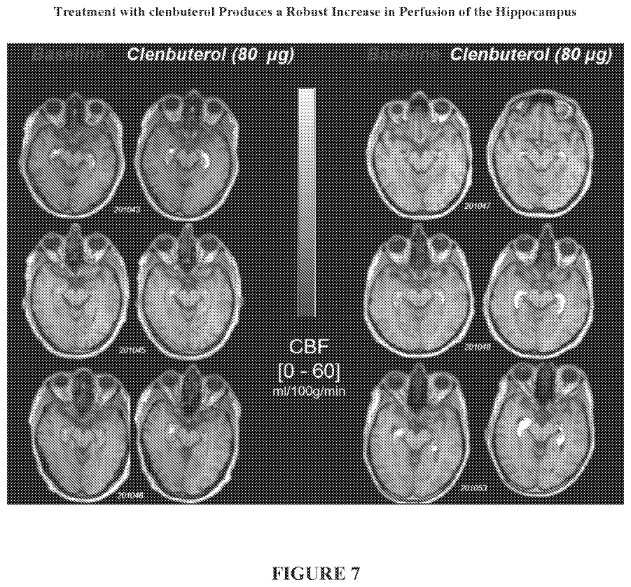

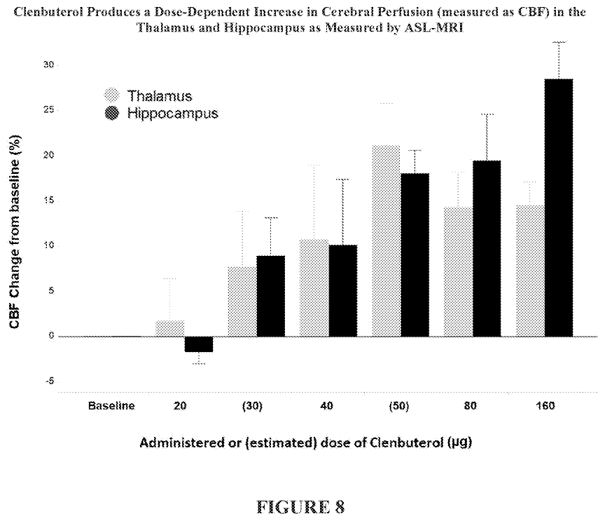

In a region of interest (ROI) analysis focusing on the hippocampus, which is well understood to be affected in neurodegenerative disorders, a single dose of 80 μg of clenbuterol causes a robust increase in perfusion (see FIG. 7). In this cohort of 6 healthy subjects treated with a single dose of 80 μg of clenbuterol every subject had an increase in hippocampal perfusion, which on average was 25%. The neuroimaging data from the study using ASL MRI demonstrated that doses of 80 and 160 μg of clenbuterol stimulate a robust, global increase in perfusion. In particular, areas of the brain thought to be relevant to the neuropathology of neurodegenerative disorders demonstrate significant improvements in perfusion in the range of 25% (FIGS. 6 and 7). An ROI analysis of the hippocampus in 6 healthy subjects aged 44 to 52 demonstrates a robust increase in this area of the brain for each subject FIG. 7. Taken together with other cohorts in which ASL MRI was conducted, a clear dose response relationship is seen between dose of clenbuterol and cerebral perfusion (FIG. 8). Doses below 30 μg do not produce significant cerebral perfusion increases as measured by CBF and a dose of 40 μg produces a minimal increase while doses of 80 and 160 μg produce global increases in cerebral perfusion, with particularly robust increases of 20% to 25% in areas of the brain relevant to neurodegenerative disorders such as the hippocampus and the thalamus (FIG. 8, Bartsch 2015, Leh 2016). Our hypothesis is that by improving cerebral perfusion, particularly in areas of the brain that are relevant for symptoms that are commonly found in neurodegenerative diseases such as PD and AD, the administration of a β-AR agonist (such as a β-agent) will have a positive effect on clinically relevant symptoms such as memory and cognition. In particular for cognition, preliminary data from the study suggest that a single dose of 160 μg of clenbuterol improves cognition in healthy subjects as measured by adaptive tracking and word recall.

### Example 4: Treatment of Human Subjects with a β-Agent and PABRA

The procedures described in Examples 1 and 3 are repeated, replacing clenbuterol or other β₂-AR agonist with a β-agent (such as, for example Compound 03-5). The starting dose of the β-agent is determined by preclinical study and the clinical dose is optimized.

### Example 5: Clinical Effectiveness

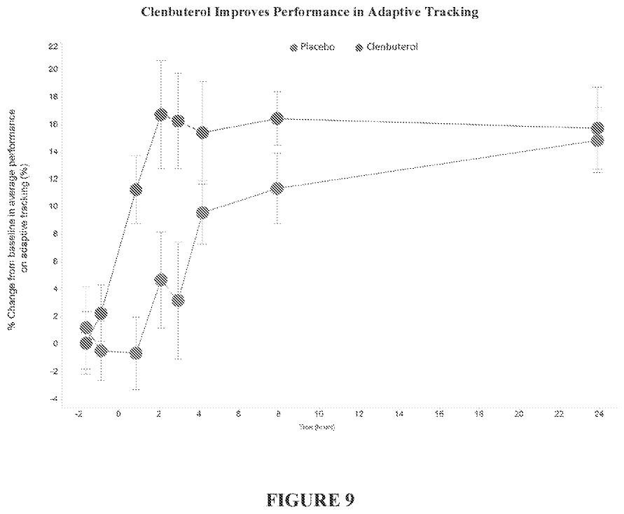

Adaptive tracking measures visuomotor coordination and vigilance. In this test, the subject uses a joystick to move a small dot so that it stays within a continuously moving circle on a computer screen (Boland 1984). During the test, the speed of the circle is adjusted in response to the subject's ability to keep the dot in the circle, ensuring that the test is adapted to the individual subject. Results suggest that after a single dose of 160 μg clenbuterol performance in adaptive tracking improves as measured by the percent time that the subject is able to keep the small dot within the moving circle (see FIG. 9). The improvement shown by subjects is in the same range as that seen with subjects treated with the acetylcholinesterase inhibitor donepezil, which is in clinical use for the treatment of mild to moderate AD (Groeneveld 2016).

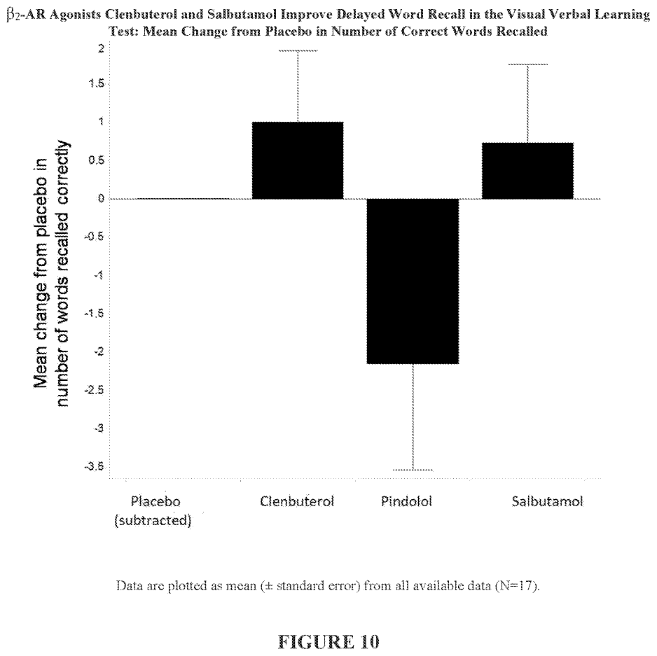

The visual verbal learning test (VVLT) is a test for learning and memory (de Haas 2009). Subjects are presented 30 words on a screen, one at a time, for 1 second with a 1-second interval between words over a total of 1 minute. This is repeated in 3 trials. After each trial, subjects are asked to recall as many words as they can. After the third trial, there is a delay of 2.5 hours and subjects are then tested once for delayed recall. Clenbuterol improved performance in VVLT in both the immediate recall (Trial 1, not shown) and the delayed recall (see FIG. 10). The effect for clenbuterol is an improvement in approximately 1.5 to 2 correctly recalled words, which is clinically meaningful. Since this was a crossover study, everyone who completed Part A was dosed with the 3 agents plus placebo. The β₂-AR agonists tested in this study, clenbuterol and salbutamol, had positive effects on the VVLT. In contrast, the β₂-AR antagonist/β₁-AR partial agonist pindolol had a detrimental effect on this learning and memory test.

### Example 4: Treatment of Human Subjects with Compound 03-5 and Nadolol

Healthy volunteers were be enrolled into 2 cohorts in a study to undertake within-subject dose titration of Compound 03-5 and/or nadolol in order to explore doses/dose combinations that mitigate peripheral effects of Compound 03-5, e.g. on heart rate, while preserving possible central effects of Compound 03-5 on cerebral perfusion and pupillary light reflex.

### A. Cohort D1.

Cohort D1 enrolled 8 healthy subjects and undertook an evaluation of the effects of treatment on ECG following repeat dosing of Compound 03-5 in the presence of escalating doses of nadolol according to following schematic.
[symbol] (Compound 03-5 dosing day(s). A single dose of nadolol (up to 40 mg) will be pre-administered or co-administered with Compound 03-5 on one or more of the dosing days. The dose of Compound 03-5 and/or nadolol may vary over the 7 dosing days. For example, the dose of Compound 03-5 maybe 3 mg on Day 1 through Day 6, and increase at the direction of the DLRM to 6 mg on Day 7, while the dose of nadolol may increase daily from N mg on Day 2 through 5 mg on both Day 6 and Day 7.
Part D will be initiated after DLRM review of safety and PK (where available) data through at least Day 4 from the first cohort in Part B. Enrollment into Part D will commence after DLRM review.
‡ One CSF sample will be collected from each subject in this cohort for determination of concentrations of Compound 03-5 and nadolol (if relevant): at approximately 2 hours after dosing on Day 2 from 4 subjects, and at approximately 2 hours after dosing on Day 6 from the remaining 4 subjects,

Eight healthy subjects aged 18-50 years were enrolled into Cohort D1 and received escalating doses of Compound 03-5 (3-12 mg) administered once daily 2 hours follow pre-administration of nadolol (1-40 mg). On all dosing days, study drug(s) were administered orally after an overnight fast of at least 8 hours.

Routine measures of ECG, vital signs, safety labs, physical exams and plasma sample collections for analysis of drug pharmacokinetics (PK) were undertaken according to the Schedule of Events (Table 1). A single sample of cerebrospinal fluid (CSF) was collected from 7 of the 8 subjects enrolled in Cohort D1 for determination of concentrations of Compound 03-5 and nadolol on either Day 2 (N=3) or Day 6 (N=4).

TABLE 2
Schedule of Events for Cohort D1

| | Screening | | | | | | | | | | | EOS |
|---|---|---|---|---|---|---|---|---|---|---|---|---|
| | Day −28 to −2 | Day −1 | Day 1 | Day 2 | Day 3 | Day 4 | Day 5 | Day 6 | Day 7 | Day 8 | Day 9 | Day 15¹² |
| Outpatient visit | X | | | | | | | | | | | X |
| In-patient admission¹ | | X | | | | | | | | | | |
| In-patient stay | | | X | X | X | X | X | X | X | X | | |
| Discharge² | | | | | | | | | | | X | |
| Informed consent³ | X | | | | | | | | | | | |
| Evaluate Inclusion/ | X | X | | | | | | | | | | |
| Exclusion criteria | | | | | | | | | | | | |
| Medical history | X | | | | | | | | | | | |
| Urine tests for drugs, | X | X | | | | | | | | | | |
| cotinine | | | | | | | | | | | | |
| Alcohol breath test | X | X | | | | | | | | | | |
| Hepatitis and HIV | X | | | | | | | | | | | |
| serologies | | | | | | | | | | | | |
| Evaluation of SARS- | X | X | | | | | | | | | | |
| CoV-2 infection⁴ | | | | | | | | | | | | |
| Height, weight | X | | | | | | | | | | | |
| Serum pregnancy or | X | | | | | | | | | | | |
| FSH test⁵ | | | | | | | | | | | | |
| Urine pregnancy | | X | | | | | | | | | | X |
| tests⁵ | | | | | | | | | | | | |
| Compound 03-5 | | | X | X | X | X | X | X | X | | | |
| administration¹³ | | | | | | | | | | | | |
| nadolol | | | X | X | X | X | X | X | X | | | |
| administration¹³ | | | | | | | | | | | | |
| Vital signs⁶ | X | X | X | X | X | X | X | X | X | X | X | X |
| Physical exam, | X | | | | | | | | | | | X |
| complete | | | | | | | | | | | | |
| Physical exam, | | X | X¹¹ | | X¹¹ | | | | | | X | |
| abbreviated | | | | | | | | | | | | |
| Safety labs⁷ | X | X | X | X | X | X | X | X | X | | X | X |
| ECG⁸ | X | X | X | X | X | X | X | X | X | X | X | X |
| Assessment of AEs | | X | X | X | X | X | X | X | X | X | X | X |
| Concomitant | X | X | X | X | X | X | X | X | X | X | X | X |
| medications | | | | | | | | | | | | |
| Plasma PK sample | | | X | X | X | X | X | X | X | X | X | |
| collection⁹ | | | | | | | | | | | | |
| CSF PK sample | | | | X | | | | X | | | | |
| collection¹⁰ | | | | | | | | | | | | |

AE = adverse event;
ECG = electrocardiogram;
HIV = human immunodeficiency virus,
PK = pharmacokinetics;
EOS = End of Study
¹Subjects were admitted on Day −1.
²Subjects were discharged on the afternoon of Day 9.
³Informed consent was obtained before any study-related procedures are performed.
⁴SARS-CoV-2 evaluation included testing from throat or nasal swab for current infection at Screening, and/or evaluation of possible ongoing infection based on body temperature (>37.3° C.), blood oxygen (<90%) and Investigator judgement at the start of confinement.
⁵For females of childbearing potential, a serum β-hCG pregnancy test was performed at screening, and urine dipstick test was performed on Day −1 and at EOS. For postmenopausal women, an FSH test will be performed at Screening.
⁶Vital signs including temperature, respirations, triplicate blood pressure, and triplicate heart rate measurements (done 3 times separated by approximately 1 minute in supine position) were assessed after the subject had been at rest in the supine position for at least 5 minutes on Day −1 and at the times identified below.
At Screening and on Day −1
On Day 1 through Day 7: twice within 1 hour prior to dosing nadolol, twice within 1 hour prior to the daily dose of Compound 03-5, and after the first daily dose of Compound 03-5 at hour 0.25 ± 0.1, 0.5 ± 0.25, 1 ± 0.25, 2 ± 0.25, 4 ± 0.5, 8 ± 1 and 12 ± 1.
On Day 8 at 24 ± 1 hours after the last dose of Compound 03-5
On Day 9 at 48 ± 2 hours after the last dose of Compound 03-5
Day 15 (EOS) at any time during the site visit
⁷Safety labs, including hematology, clinical chemistries, urinalysis, and cardiac troponin, were evaluated at 2-6 hours after administration of Compound 03-5 Days 1, 2, 4, and 7, and at any time of day on all other scheduled assessment days.
⁸Triplicate ECGs were obtained at the times listed below. Unless otherwise stated, subjects will be required to rest in a supine position for at least 5 minutes prior to the recording of ECG.
At screening;
on Day −1 at the same times of day as planned for Day 1 (±0.5 hours);
On Day 1 through Day 7: twice within 1 hour prior to dosing nadolol, twice within 1 hour prior to the daily dose of Compound 03-5, and after the daily dose of Compound 03-5 at hour 0.25 ± 0.1, 0.5 ± 0.25, 1 ± 0.25, 2 ± 0.25, 3 ± 0.5, 4 ± 0.5, 6 ± 0.5, 8 ± 1, 10 ± 1, 12 ± 1, and 14 ± 1;
On Day 8: 24 ± 1 and 36 ± 1 hours after the last dose of Compound 03-5 on Day 7;
On Day 9: 48 ± 2 hours after the last dose of Compound 03-5;
Day 15 (EOS) at any time during the site visit.
⁹Plasma PK samples were collected for analysis of Compound 03-5 and nadolol concentrations at the following times:
On Days 1 through 7 within 1 hour prior to first dose of nadolol (if administered), within 0.5 hour prior to first dose of Compound 03-5 and after the first dose of Compound 03-5 at hour 0.25 ± 0.1, 0.5 ± 0.1, 1 ± 0.25, 2 ± 0.5 (to be collected within 30 minutes of the CSF sample), 4 ± 0.5, 6 ± 1, and 12 ± 1;
On Day 8: at 24 (±1) and 36 (±1) hours after the last dose of Compound 03-5;
And on Day 9 at 48 (±2) hours after the last dose of Compound 03-5.
¹⁰A single CSF sample was collected from each subject enrolled in this cohort for determination of concentrations of Compound 03-5 and nadolol.
Attempted from 4 subjects, but successfully collected in only 3 subjects at 2 ± 0.5 hours after dosing on Day 2.
from the remaining 4 subjects at approximately 2 ± 0.5 after dosing on Day 6.
A time-matched plasma PK sample was collected within ±0.5 hours of the CSF collection.
¹¹An abbreviated physical exam was conducted at the time of the predicted Cₘₐₓ at 2 hours after dosing (or within a 4-hour window thereafter) on Day 1.
¹²End of Study (EOS) was conducted on Day 15 ± 3 days.
¹³nadolol (1-40 mg) was pre-administered 2 hour before Compound 03-5 (3-12 mg) on each of the 7 dosing days according to the dose escalation plan provided in the Study Schematic.

### A. Cohort D2.

In Cohort D2, 8 healthy volunteers aged 55-75 years were admitted on Day −1 for a 5-day confinement period at the research facility. During this confinement, subjects received escalating doses of open label Compound 03-5 (1-10 mg) once-daily from Day 1 through Day 3 2 hours after pre-administration of open-label nadolol (3 mg) one each for the 3 dosing days according to the the following schematic. Subjects remained in-residency until all study procedures are completed on Day 4.
[symbol] Compound 03-5 dose administration. Compound 03-5 was administered as escalating doses on Day 1 (1 mg), Day 2 (3 mg) and Day 3 (10 mg). CANTAB was conducted twice in all subjects on the day prior to start of dosing (Day −1) to familiarize subjects with the platform, and prior to dosing and approximately 3 hours after Compound 03-5 administration on Days 1, 2, and 3.

TABLE 3
Schedule of Events for Cohort D2

| | Screening | | | | | | EOS |
|---|---|---|---|---|---|---|---|
| | Day −28 to −2 | Day −1 | Day 1 | Day 2 | Day 3 | Day 4 | Day 15¹⁵ |
| Outpatient visit | X | | | | | | X |
| In-patient admission¹ | | X | | | | | |
| In-patient stay | | | X | X | X | | |
| Discharge² | | | | | | X | |
| Informed consent³ | X | | | | | | |
| Evaluate Inclusion/Exclusion | X | X | | | | | |
| criteria | | | | | | | |
| Medical history | X | | | | | | |
| Urine tests for drugs, cotinine | X | X | | | | | |
| Alcohol breath test | X | X | | | | | |
| Hepatitis and HIV serologies | X | | | | | | |
| Evaluation of SARS-CoV-2 infection⁴ | X | X | | | | | |
| Height, weight | X | | | | | | |
| Serum pregnancy or FSH test⁵ | X | | | | | | |
| Urine pregnancy test⁵ | | X | | | | | X |
| Study drug administration | | | X | X | X | | |
| (Compound 03-5 dose escalation | | | | | | | |
| with nadolol)⁶ | | | | | | | |
| CANTAB⁷ | | X | X | X | X | | |
| Pupillometry⁹ | | | X | X | X | | |
| Vital signs¹⁰ | X | X | X | X | X | X | X |
| Physical exam, complete | X | | | | | | X |
| Physical exam, abbreviated¹¹ | | X | X | X | X | X | |
| Safety labs¹² | X | X | X | X | X | X | X |
| ECG¹³ | X | X | X | X | X | X | X |
| Assessment of AEs | | X | X | X | X | X | X |
| Concomitant medications | X | X | X | X | X | X | X |
| Plasma PK sample collection¹⁴ | | | X | X | X | X | |

AE = adverse event;
ECG = electrocardiogram;
HIV = human immunodeficiency virus,
PK = pharmacokinetics;
EOS = End of Study
¹Subjects were admitted on Day −1.
²Subjects were discharged on the afternoon of Day 4.
³Informed consent was obtained before any study-related procedures are performed.
⁴SARS-CoV-2 evaluation included testing from throat or nasal swab for current infection at Screening, and/or evaluation of possible ongoing infection based on body temperature (>37.3° C.), blood oxygen (<90%) and Investigator judgement at the start of confinement.
⁵For females of childbearing potential, a serum β-hCG pregnancy test was performed at screening, and urine dipstick test was performed on Day −1 and at EOS. For postmenopausal women, an FSH test will be performed at Screening.
⁶nadolol (3 mg) was pre-administered 2 hour before Compound 03-5. Compound 03-5 was administered once daily as escalating doses of 1, 3, 10 mg on Day 1, Day 2 and Day 3.
⁷CANTAB was administered twice on Day −1, once to familiarize the subject with the tests and equipment, and a second time at least 3 hours later (as a pre-dose measure). On Days 1, 2, and 3, CANTAB was administered within 2 hours prior to administration of nadolol, and repeated starting 3 ± 1 hours after dosing of Compound 03-5.
⁹Pupillary light reflex was measured twice for each eye at the following times at sites where operationally feasible:
On Day 1: prior to dosing and at 2 ± 0.5, 4 ± 2 and 6 ± 1 hours after last administration of Compound 03-5 on Day 1
On Day 2: at 2 ± 0.5, 4 ± 2 and 6 ± 1 hours after last administration of Compound 03-5 on Day 2
On Day 3: at 2 ± 0.5, 4 ± 2 and 6 ± 1 hours after last administration of Compound 03-5 on Day 3
¹⁰Vital signs including temperature, respirations, triplicate blood pressure, and triplicate heart rate measurements (done 3 times separated by approximately 1 minute in supine position) were assessed after the subject had been at rest in the supine position for at least 5 minutes on Day −1 and at the times identified below.
At Screening and on Day −1
On Day 1, Day 2 and Day 3: once within 2 hours prior to dosing nadolol, once within 1 hour prior to the daily dose of Compound 03-5, and after the daily dose of Compound 03-5 at hour 1 ± 0.25, 2 ± 0.25, 3 ± 0.5, 4 ± 0.5, 5 ± 1, 6 ± 1, 7 ± 1, 8 ± 1, 9 ± 1, and 10 ± 1.
On Day 4: at 24 ± 1 hour after the last administration of Compound 03-5 on Day 3.
Day 15 (EOS) at any time during the site visit
¹¹An abbreviated physical exam was conducted at 2 hours after the first administration of Compound 03-5 or within a 4-hour window thereafter on Days 1, 2, and 3.
¹²Safety labs, including standard panels for hematology, clinical chemistries, urinalysis, and cardiac troponin were evaluated between 2 - to 6 hours after administration of Compound 03-5 on Days 1, 2, and 3, and at any time of day on Day 15 (EOS).
¹³Triplicate ECGs obtained at the times listed below. Subjects were in a supine position for at least 5 minutes prior to the recording of ECG.
At screening;
on Day −1 at the same times of day as planned for Day 1 (±0.5 hours).
On Day 1, Day 2 and Day 3: once within 2 hours prior dosing nadolol, once within 2 hour priors to the daily dose of Compound 03-5, and after the daily dose of Compound 03-5 at hour 0.25 ± 0.2, 0.5 ± 0.25, 1 ± 0.25, 2 ± 0.25, 3 ± 0.5, 4 ± 0.5, 5 ± 1, 6 ± 1, 7 ± 1, 8 ± 1, 9 ± 1, and 10 ± 1;
On Day 4: at 24 ± 1 hour and 30 ± 1 after the last administration of Compound 03-5 on Day 3;
Day 15 (EOS) at any time during the site visit.
¹⁴Plasma PK samples were collected for analysis of Compound 03-5 and nadolol at the following times:
On Days 1, 2, and 3 within 1 hour prior to dosing of nadolol, within 0.5 hour prior to dosing of Compound 03-5 and at the following times after the first daily dose of Compound 03-5 1 ± 0.25, 2 ± 0.25, 4 ± 0.5, and 6 ± 1.
On Day 4: at 24 ± 1 and 30 ± 1 hour after the last administration of Compound 03-5 on Day 3.
¹⁵End of Study (EOS) visit was conducted on Day 15 ± 5 days. .

### Results.

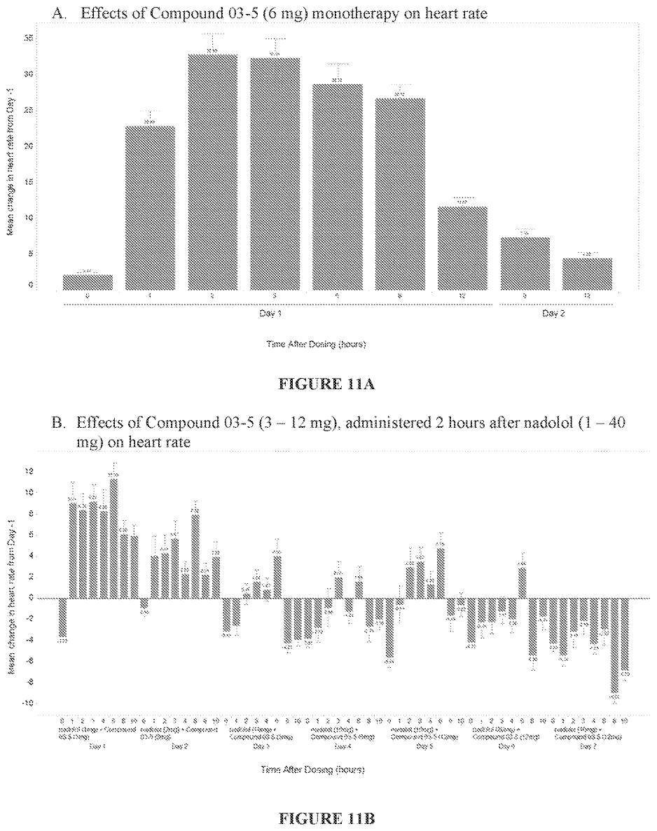

In earlier cohorts, maximum mean increases in heart rate up to approximately 30 beats per minute were observed following monotherapy with 6 mg Compound 03-5. FIGS. 11A-11B. This effect was substantially attenuated to a mean maximum increase of 7 beats per minute by pre-administration of 1 mg nadolol. Concomitant attenuation of the effects of Compound 03-5 on blood glucose and potassium, tachycardia, tremor and palpitations was also noted. These effects of Compound 03-5 are similar to the widely reported responses reported with marketed β₂-AR agonists such as salbutamol.

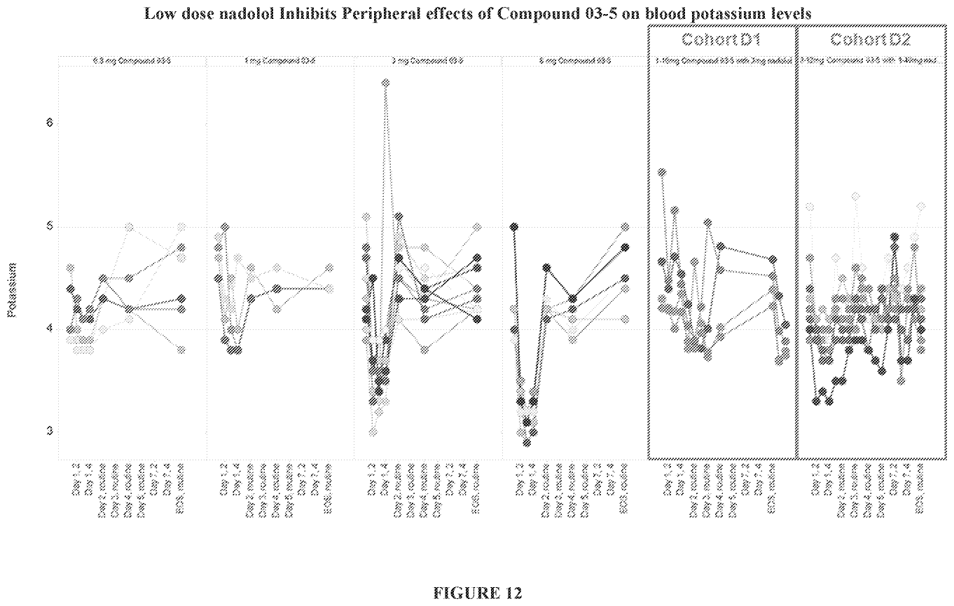

In Cohort D1, as with other cohorts, pre-administration of 1 mg nadolol substantially attenuated the effects of Compound 03-5, administered orally at doses up to 6 mg, to increase heart rate and other peripheral effects of Compound 03-5 including hyper glycemia and hypokalemia. FIGS. 11A-11B and FIG. 12.

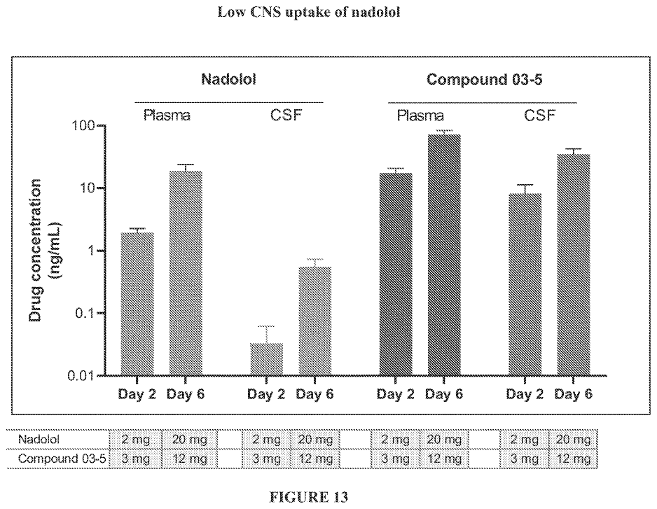

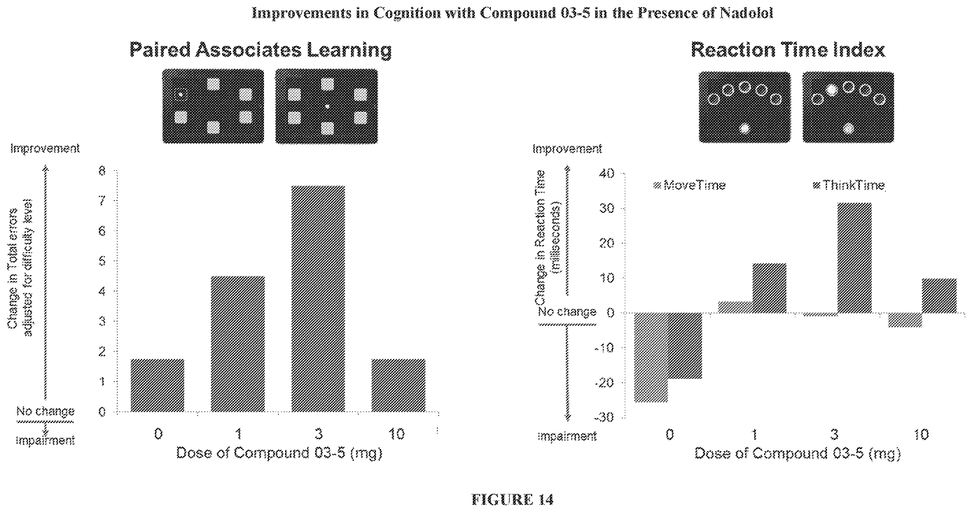

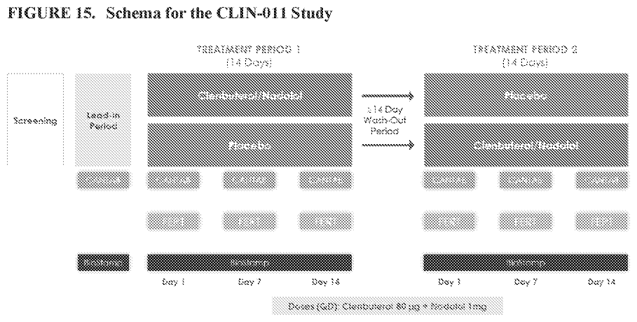

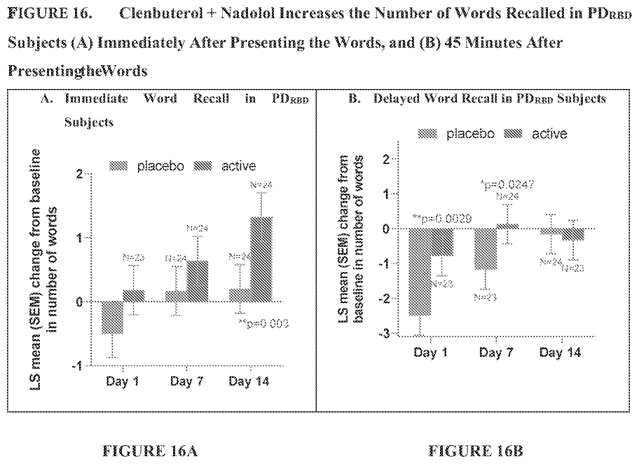

Drug concentrations in cerebrospinal fluid (CSF) of subjects enrolled in Cohort D1 were measured after they received Compound 03-5 (3 mg [N=3] or 12 mg [N=4]), and nadolol (2 mg [N=3] or 20 mg ([N=4]). The estimated concentrations of nadolol in CSF demonstrate that it is substantially peripherally restricted with approximately 2-3% of time-matched plasma concentration detected. In contrast, the concentrations of Compound 03-5 in CSF were approximately 50% of the time-matched plasma concentration. FIG. 13.

### Conclusion.

Nadolol is approved for treatment of xx at doses in humans from 40 mg up to 320 mg/day.

In earlier cohorts, maximum mean increases in heart rate up to approximately 20 beats per minute were observed following monotherapy with 6 mg Compound 03-5. This effect was substantially attenuated to a mean maximum increase of 7 beats per minute by pre-administration of 1 mg nadolol.

In Cohort D1, pre-administered nadolol (1 mg up to 40 mg) blocked the peripheral effects of Compound 03-5 (3-12 mg) on heart rate, glucose, potassium, and other clinical observations associated with β₂-AR agonists such as tachycardia, tremor and palpitations. Emerging preliminary data on CSF concentrations of nadolol in Cohort D1 of this study demonstrate that nadolol is a β-AR antagonist with very low CNS penetration.

In Cohort D2, pre-administered nadolol (3 mg) similarly blocked the peripheral effects of Compound 03-5 (1-10 mg) on heart rate, etc. However, improvements in performance in the CANTAB cognition battery were observed and are presumed mediated by the selective stimulation of β₂-ARs in the brain by Compound 03-5.

The effects of low dose nadolol (3 mg) to control untoward peripheral effects of the selective b2-AR agonist, without inhibiting the pro-cognitive central effects, coupled with the evidence of low CNS penetration of nadolol observed in Cohort D1, support the use of low dose nadolol for specific and selective control of peripheral effects of β₂-AR agonists intended for treatment of CNS diseases.

### Example 5. Phase II, Randomized, Placebo-Controlled, Double-Blind, Crossover Study of the Pharmacodynamic Effects of Clenbuterol (CST-103) Co-Administered with Nadolol (CST-107) on the Central Nervous System in Subjects with Neurodegenerative Disorders

### Study Objectives

Study objectives included:
(a) Identification a central nervous system (CNS) signal in one of the planned pharmacodynamic measurements after multiple oral doses of CST-103 in the presence of CST 107 in subjects with neurodegenerative disorders:
1. Parkinson's disease (PD) with REM sleep behavior disorder (RBD) and depressive symptoms (PD_{RBD})
2. Mild cognitive impairment (MCI) with depressive symptoms

These pharmacodynamic assessments compared the effect of CST-103 co-administered with CST-107 with that of placebo on measures of cognition using e.g. the Cambridge Neuropsychological Test Automated Battery (CANTAB), and social cognition using the Facial Expression Recognition Task (FERT) and evaluation of the safety and tolerability of 80 μg CST-103 (clenbuterol) when administered with 1 mg CST-107 (nadolol) for 14 weeks compared with placebo.

### Methodology

This was a randomized, placebo-controlled, double-blind, crossover study on the CNS and pharmacodynamic effects of CST-103 co-administered with CST-107 in subjects with neurodegenerative disorders including PD_{RBD} (N=25 enrolled) and MCI (N=13 enrolled).

Subjects were enrolled in a 2-period, 2-way crossover design following study eligibility confirmation during the screening period.

During each treatment period, subjects received oral daily doses of 80 μg CST-103 (administered as two 40 μg capsules) co-administered with 1 mg CST-107 (administered as one 1 mg capsule) or matching placebos for 14 days. Subjects were randomized to the sequence in which they received the 2 blinded treatments, (a) active (80 μg CST-103+1 mg CST-107) and (b) placebo. Each treatment period was separated by a washout period of at least 14 days.

### Diagnosis and Key Criteria for Inclusion

Subjects with PD_{RBD}: ≥40 and ≤80 years of age; diagnosed with PD as defined by the United Kingdom Parkinson Disease Brain Bank criteria, associated with REM sleep behavior disorder (RBD), diagnosed according to the International Classification of Sleep Disorders, Third Edition (ICSD-3) (documentation by polysomnography was not required) and positive response to the REM Sleep Behavior Disorder Single-Question Screen (RBD1Q); Modified Hoehn & Yahr (MHYS) ≥stage 1 and ≤stage 3 during “On” period as documented in the 3 months prior to Screening or completed at Screening; and Montreal Cognitive Assessment (MoCA) score ≥18 and ≤28

Subjects with MCI: ≥50 and ≤80 years of age; met the criteria for amnestic MCI as per the National Institute on Aging-Alzheimer's Association core clinical criteria; MoCA score ≥18 and ≤26; no dementia according to the International Classifications of Diseases (ICD)-10 and Diagnostic and Statistical Manual of Mental Disorders (DSM)-IV; a memory complaint reported by the subject or his/her partner, family member, or caregiver; a score of ≥1 standard deviation below age and educational norms in the Digit Symbol Substitution Test (DSST) during Screening; and cognitive decline not primarily caused by vascular, traumatic, or medical problems (alternative causes of cognitive decline were ruled out).

### Treatments Administered

Study treatments were:
Clenbuterol+nadolol taken orally in the morning after the first meal of the day during each Treatment Period as:
80 μg CST-103, administered as two 40 μg capsules once daily on Days 1 to 14
1 mg CST-107, administered as one 1 mg capsule once daily on Days 1 to 14
Matching placebo taken daily during each Treatment Period

### Selection of Doses in the Study

The daily 80 μg CST-103 (clenbuterol) dose chosen for this study is below the recommended maximum daily dose in Germany (100 μg) for the treatment of reactive airways diseases (Spiropent Summary of Product Characteristics). A favorable safety and tolerability profile of the 80 μg CST-103 dose was supported by preliminary data from CuraSen's signal-seeking studies and published clinical studies, including a 52-week, double-blind, randomized, placebo-controlled study of clenbuterol 80 μg twice daily (Koeberl 2018).

The neuroimaging data from CuraSen study CST103/CST107/CST109-CLIN-002 (CLIN-002) using arterial spin labeling (ASL) MRI demonstrated that doses of CST-103 80 and 160 μg produce a robust global increase in cerebral perfusion, with a particularly robust increase of up to 15% in the thalamus and up to 28% in the hippocampus, which are both areas of the brain relevant to neurodegenerative diseases (Bartsch 2015, Leh 2016). It was hypothesized that by improving cerebral perfusion, particularly in areas of the brain that are relevant for symptoms that are commonly found in PD and MCI, the administration of a β-AR agonist would have a positive effect on clinically relevant symptoms such as memory and cognition. In particular for cognition, preliminary data from Curasen study CST101/CST103/CST109-CLIN-003 (CLIN-003) suggest that a daily dose of 80 μg of CST-103 improves cognitive performance in healthy subjects.

CST-107 was included in the present study to attenuate the peripheral effects commonly caused by β₂-AR agonists, which include increased heart rate, palpitations, tremors, decreases in potassium, and increases in blood glucose. Previous CuraSen studies exploring low doses of CST-107 co-administered with 80 μg of CST-103 suggested that a 1 mg dose could attenuate these peripheral effects. This is in marked contrast to the usual initial dose for hypertension, which is 40 mg CST-107 tablets once daily. The usual maintenance dose is 40 or 80 mg administered once daily and doses up to 240 or 320 mg administered once daily may be needed.

In the treatment of reactive airways disease, the use of a β-AR blocker is not normally recommended along with CST-103 in that the therapeutic effect of the agonist can be antagonized and would risk triggering a severe bronchospasm in subjects with asthma. Since this study did not include subjects with reactive airways disease and CST-103 was not being used for its bronchodilatory properties, this contraindication was not applicable to this study.

### Prior and Concomitant Therapy

Subjects who required routine medication to manage and treat concurrent conditions (e.g., hyperlipidemia, diabetes, hypertension) must have been on stable doses 3 months prior to Screening. Any concomitant therapy taken from the time the subject signed the informed consent form through the final visit was recorded on the eCRF. The medication name, dosage, date, and indication for use were recorded. The Medical Monitor or designee should be notified in advance of (or as soon as possible after) any instances in which prohibited therapies were administered.

Prohibited concomitant medications included the following:
Paracetamol >2 g per day
PD medication changes within 3 months prior to Screening (applicable to RBD+PD, DLB, and PDD subjects)
Vitamin E >400 IU daily
Aspirin >300 mg daily
Cannabis and products containing tetrahydrocannabinol (THC) and/or cannabidiol (CBD)
Melatonin or hypnotics, such as zolpidem or zopiclone, within 48 hours prior to a study visit involving cognitive testing
Levodopa >1.5 g daily
Changes in clonazepam and monoamine oxidase inhibitor (MAOI) dosing within 4 weeks before Screening
Use of St. John's Wort or Ginkgo Biloba within 48 hours prior to study enrollment (defined as the Lead-in Period)
Use of benzodiazepine (expect clonazepam) was prohibited from Screening throughout the study.
Use of gabapentinoids, phenylephrine, and pseudoephedrine was prohibited from Screening throughout the study.
Prior treatment with any β-AR agonists or β-AR blockers (included oral medications, eye drops with adrenergic agents such as timolol or atenolol, intravenous, or inhaled), or any medications that impact adrenergic signaling within the last month prior to Screening excluded a subject from study enrollment. Use of β-AR agonists or β-AR blockers was not allowed during the study.
Subjects who had prior treatment with any investigational drug ≤90 days prior to dosing (Day 1), or ≤5 half-lives of the drug (whichever was longer) were excluded.
Subjects were not to receive the influenza vaccine within 2 weeks of the Screening visit through to the Follow-Up Visit.
Opioid use was prohibited within 1 month prior to Screening and during the study.

### Facial Expression Recognition Task (FERT)

The FERT assesses the recognition of facial emotions. Faces with 6 different basic emotions (happiness, fear, anger, disgust, sadness, and surprise) are briefly displayed on the screen and participants are required to indicate the expression of the face via a button-press. Different intensity levels of each emotion are presented, which increases the ambiguity of the facial expression and the sensitivity of the task. Early change in measures of emotional bias in depressed subjects treated with antidepressants has been positively correlated with the improvement in subjects' symptoms of depression across a full 6 to 8 weeks of treatment (Tranter 2009), suggesting that early changes detected through the use of the FERT may be predictive of antidepressant response.

### CANTAB Assessments

The CANTAB is a standardized and automated administration of cognitive testing via touch tablet, which included the following assessments:
The Reaction Time (RTI) task is a processing and psychomotor speed task. It begins with a simple stage with only one target and can be increased to 5 targets to increase demand. Once a yellow circle flashes on screen, subjects must select that circle as fast as possible. The dependent measures are thinking time and movement time in milliseconds (usually ranging between 200 and 2000 msec), which are decomposed as the time between onset of the stimulus and leaving the holding button (thinking time or initiation time) and the time between leaving the holding button and pressing the target button (movement time). The total reaction time is the sum of thinking time and movement time.
The immediate and delayed Verbal Recognition Memory (VRM) recall and recognition tests measure the ability to encode and subsequently retrieve verbal information. Eighteen words are presented and subjects are subsequently asked to recall them; this is repeated 2 times. Dependent measures are immediate recall expressed as the average number of words correctly recalled over 3 trials (range 0 to 18 words) and delayed recall as the number of words freely recalled 45 minutes after their first presentation (range 0 to 18 words) and delayed recognition (range 0 to 36 words; 0 to 18 target words correctly recognized and 0 to 18 non-target words correctly rejected in a forced choice paradigm). With these parameters a higher number indicates better performance.
The Adaptive Tracking Task (ATT) measures visuomotor coordination and vigilance. In this test, a small circle (target) continuously moves across the screen in a semi randomized fashion to minimize the subject's ability to predict the trajectory of the target. The subject is instructed to use his/her finger on the touch screen to move a small dot so that it is consistently within the center of the moving target on the screen. During the test, the speed of the circle is adjusted in response to the subject's ability to keep the dot in the circle, ensuring that the test is adapted to the individual subject. The main dependent measure is “difficulty multiplier mean,” which is a number that indicates how well the subject can cope with increasing the difficulty level (usual range 0 to 10). With this parameter a higher number indicates better performance.
The Paired Associates Learning (PAL) task is a measure of visuo-spatial episodic memory that relies on the functional integrity of the hippocampus. The task becomes gradually more difficult, benchmarking a subject's memory capacity. Subjects have to remember the location of an abstract pattern in a specific location. The dependent measure is the total number of errors adjusted for the difficulty level achieved (range 0 to 120). With this parameter a lower number indicates better performance. The total number of errors adjusted for the difficulty level achieved is seen as the main outcome variable of PAL and is considered as a measure of nonverbal episodic memory.
The Rapid Visual Information Processing (RVP) task is a sensitive measure of sustained attention, outputting measures of response accuracy, target sensitivity and reaction times. For the RVP task single digits appear in a pseudo-random order at a rate of 100 digits per minute in box in the center of the screen. Subjects must detect a series of 3-digit target sequences (3-5-7; 2-4-6; 4-6-8) and respond by touching the button at the bottom of the screen when they see the final number of the sequence. Nine target sequences appear every minute.
The Stop Signal Task (SST) measures response inhibition (impulse control). The subject must respond to an arrow stimulus, by selecting one of two options, depending on the direction in which the arrow points. If an audio tone is present, the subject must withhold making that response (inhibition).

### Neuromelanin MRI Scan

Neuromelanin-sensitive MRI (NM-MRI) was measured at baseline to evaluate locus coeruleus integrity (e.g., volume, signal intensity contrast-to-noise ratio [CNR]). Neuromelanin is a dark insoluble complex that is synthesized as an oxidative byproduct of dopamine and noradrenaline in regions of high catecholamine activity such as the substantia nigra and LC (Wakamatsu 2015). It sequesters potentially toxic organic chemical, exogenous and endogenous metals such as iron. When bound to metals such as iron and copper, neuromelanin is highly paramagnetic, leading to T1-shortening and hyperintense signal on T1-weighted turbo spin-echo MRI sequences. As a result, neuromelanin can be detected by non-invasive MRI methodology.

### Duration of Treatment

Duration of treatment was from Day 1 to Day 14 during each treatment period, with a 14-day washout between periods.

### Results

### Efficacy

The clearest treatment effects with clenbuterol+nadolol was observed in the subgroup of participants with PD_{RBD} (N=23-25). Tendencies were also observed in the subgroup of participants with MCI, however these are typically not statistically significant, presumably due to the relatively small sample size (N=10-12). Except where indicated, the efficacy observations below are summarized for participants with PD_{RBD}.

### Effects on CANTAB

Statistically significant increases in the number of words accurately recalled were observed with participants with PD_{RBD} while receiving clenbuterol+nadolol compared with placebo. These effects were evident (a) immediately after the words were presented, and (b) 45 minutes after the words were presented.

### Effects on FERT

Participants with PD_{RBD} were statistically significantly more accurate at identifying happy faces while one clenbuterol+nadolol compared with placebo. In a related manner, participants with MCI were generally able to identify happy faces more quickly while on clenbuterol+nadolol compared with placebo. However, the latter observation did not attain statistical significance, presumably due to the relatively small sample size (N=10-12).

The observed effects of clenbuterol+nadolol on recognition of happy faces, as measured by accuracy (%) and reaction time (msec) in participants with PD_{RBD} or MCI are consistent with an antidepressant-like effect.

### Safety

Coadministration of clenbuterol with low-dose nadolol was well tolerated in this study. No safety signals were observed and there were no clinically significant findings on laboratory evaluations, ECGs, or vital signs.

### Adverse Events

Adverse events (AEs) were approximately balanced between the active and placebo treatment periods (Table 1) except for tremor, which was observed only in subjects with PD_{RBD} (N=6, 25%), and deemed to be related to study drug in 4 (16/7%) Tachycardia was observed in the treatment period during which subjects received clenbuterol+nadolol in 1 subject in each of the PD_{RBD} and MCI groups, and in no subjects during the placebo dosing period.

TABLE 1
Treatment-Emergent Adverse Events Reported for 2
or More Subjects on Clenbuterol/Nadolol Overall

| | RBD + PD Subjects, | | | |
|---|---|---|---|---|
| | n (%) | | MCI Subjects, n (%) | |
| | | Clenbuterol/ | | Clenbuterol/ |
| System Organ Class | Placebo | Nadolol | Placebo | Nadolol |
| Preferred Term | (N = 25) | (N = 24) | (N = 12) | (N = 13) |
| Nervous system | | | | |
| disorders | | | | |
| Headache | 3 (12.0) | 5 (20.8) | 0 | 2 (15.4) |
| Tremor | 0 | 6 (25.0) | 0 | 0 |
| Parkinson's disease | 0 | 1 (4.2) | 0 | 0 |
| Psychiatric disorders | | | | |
| Nervousness | 0 | 1 (4.2) | 0 | 2 (15.4) |
| Anxiety | 0 | 0 | 0 | 2 (15.4) |
| Skin and | | | | |
| subcutaneous | | | | |
| disorders | | | | |
| Erythema | 2 (8.0) | 4 (16.7) | 0 | 0 |
| Skin irritation | 0 | 3 (12.5) | 0 | 0 |
| Gastrointestinal | | | | |
| disorders | | | | |
| Diarrhoea | 1 (4.0) | 1 (4.2) | 1 (8.3) | 1 (7.7) |
| Nausea | 0 | 2 (8.3) | 0 | 0 |
| Investigations | | | | |
| Blood creatine | 0 | 1 (4.2) | 0 | 1 (7.7) |
| phosphokinase | | | | |
| increased | | | | |
| Cardiac disorders | | | | |
| Tachycardia | 0 | 1 (4.2) | 0 | 1 (7.7) |

A treatment-emergent adverse event was defined as any adverse event (AE) where onset occurred following the first dose up to the study completion date. Adverse events were coded using MedDRA version 24.0. Adverse events were summarized according to the treatment received, based on the period in which the AE occurred.
Source: Table 14.3.1.2

### Vital Signs and ECG

Modest increases in heart rate were evident in participants while on active drug compared with placebo and there were no clinically significant findings ECG.

### Safety Laboratory Measures

There were no clinically significant findings on laboratory evaluations including lab parameters that are known to be affected by β₂-AR agonists (e.g., potassium and glucose).

### Conclusions.

14-day treatment with clenbuterol+nadolol was associated with improved performance in CANTAB tasks, including statistically significant increases compared with placebo in the numbers of words recalled immediately and after a 45-minute delay. These effects were most marked in participants with PD_{RBD}, however, similar tendencies were noted in subjects with MCI.

14-day treatment with clenbuterol+nadolol was associated with improved recognition of happy faces in the FERT task, as measured by increased accuracy and decreased response times. These effects may be viewed as an improvement in mood, or social cognition, and were most marked in participants with PD_{RBD}.

There were no clinically significant findings on laboratory evaluations including lab parameters that are known to be affected by β₂-AR agonists (e.g., potassium and glucose).

### REFERENCE LIST

Bartsch T, Wulff P. The hippocampus in aging and disease: from plasticity to vulnerability. Neuroscience. 2015 Nov. 19; 309:1-16.
Bissonnette E Y, Befus A D. Anti-inflammatory effect of beta 2-agonists: inhibition of TNF-alpha release from human mast cells. J Allergy Clin Immunol. 1997; 100(6 Pt 1):825-831. Bjelland I, Dahl A A, Haug T T, Neckelmann D. The validity of the Hospital Anxiety and Depression Scale. An updated literature review, J Psychosom Res. 2002 February; 52(2):69-77.
de Rover M, Pironti V A, McCabe J A, et al. Hippocampal dysfunction in subjects with mild cognitive impairment: a functional neuroimaging study of a visuospatial paired associates learning task. Neuropsychologia. 2011 June; 49(7):2060-2070.
Emre M, Aarsland D, Brown R, et al. Clinical diagnostic criteria for dementia associated with Parkinson's disease. Mov Disord. 2007; 22(12):1689-1707.
Fahn S, Elton R L. UPDRS Development Committee. The Unified Parkinson's Disease Rating Scale. In: Fahn S, Marsden C D, Calne D B, Goldstein M, editors. Recent Developments in Parkinson's Disease. 2nd ed Macmillan Healthcare Information; Florham Park, NJ: 1987. pp. 153-163, 293-304.
Faizi M, Bader P L, Saw N, et al. Thy1-hAPP(Lond/Swe+) mouse model of Alzheimer's disease displays broad behavioral deficits in sensorimotor, cognitive and social function. Brain Behav. 2012 March; 2(2):142-154.
Gaertner B, Wagner M, Luck T, et al. Normative data for the Digit Symbol Substitution Test in a population-based sample aged 65-79 years: results from the German Health Interview and Examination Survey for Adults (DEGS1). Clin Neuropsychol. 2018; 32(supl):114-132. Giladi N, Shabtai H, Simon E S, Biran S, Tal J, Korczyn A D. Construction of freezing of gait questionnaire for subjects with Parkinsonism. Parkinsonism Relat Disord. 2000 Jul. 1; 6(3):165-170.
Harrison J E, Buxton P, Husain M, Wise R. Short test of semantic and phonological fluency: Normal performance, validity and test retest reliability. Br J Clin Psychol. 2000; 39:181-119.
Hart E P, Alvarez-Jimenez R, Davidse E, et al. A computerized Test Battery to study pharmacodynamic effects on the central nervous system of cholinergic drugs in early phase drug development. J Vis Exp. 2019 Feb. 11; (144).
Heneka M T, Galea E, Gavriluyk V, et al. Noradrenergic depletion potentiates beta-amyloid-induced cortical inflammation: implications for Alzheimer's disease. J Neurosci. 2002; 22:2434-2442.
Houx P J, Jolles J. Age-related decline of psychomotor speed: effects of age, brain health, sex, and education. Percept Mot Skills. 1993 February; 76(1):195-211.
Kjaergaard M, Arfwedson Wang C E, Waterloo K, Jorde R. A study of the psychometric properties of the Beck Depression Inventory-II, the Montgomery and Åsberg Depression Rating Scale, and the Hospital Anxiety and Depression Scale in a sample from a healthy population. Scand J Psychol. 2014 February; 55(1):83-89.
Kempsford R, Allen A, Kelly K, et al. A repeat-dose thorough QT study of inhaled fluticasone furoate/vilanterol combination in healthy subjects. Br J Clin Pharmacol. 2014; 77(3):466-479.
Koeberl D D, Case L E, Smith E C, et al. Correction of biochemical abnormalities and improved muscle function in a phase I/II clinical trial of clenbuterol in Pompe disease. Mol Ther. 2018 Sep. 5; 26(9):2304-2314.
Löfdahl C G, Sigvaldasson A, Skoogh B E, Svedmyr N. Broxaterol, a new beta 2-adrenoceptor agonist compared to salbutamol in asthmatics, oral and inhalation treatment. Respiration, 1989; 55 Suppl 2:15-9. doi: 10.1159/000195765.
Lötvall J, Ankerst J. Long duration of airway but not systemic effects of inhaled formoterol in asthmatic subjects. Respir Med. 2008 March; 102(3):449-56. doi: 10.1016/j.rmed.2007.10.003. Epub 2007 Nov. 19. PMID: 18023335.
Lee D R, McKeith I, Mosimann U, Ghosh-Nodial A, Grayson L, Wilson B, Thomas A J. The dementia cognitive fluctuation scale, a new psychometric test for clinicians to identify cognitive fluctuations in people with dementia. Am J Geriatr Psychiatry. 2014 September; 22(9):926-35. doi: 10.1016/j.jagp.2013.01.072. Epub 2013 Dec. 12. PMID: 24332982.
Leh S E, Kilin A M, Schroeder C, et al. Volumetric and shape analysis of the thalamus and striatum in amnestic mild cognitive impairment. Alzheimers Dis. 2016; 49(1):237-249.
McKeith I G, Boeve B F, Dickson D W, et al. Diagnosis and management of dementia with Lewy bodies: Fourth consensus report of the DLB Consortium. Neurology. 2017; 89(1):88-100.
Meltzer J A, Constable R T. Activation of human hippocampal formation reflects success in both encoding and cued recall of paired associates. Neuroimage. 2005 Jan. 15; 24(2):384-397.
Mittal S, Bjørnevik K, Im D S, et al. β2-Adrenoreceptor is a regulator of the α-synuclein gene driving risk of Parkinson's disease. Science. 2017 Sep. 1; 357(6354):891-898.
Nasreddine Z R, Phillips N A, Bedirian V, et al. The Montreal Cognitive Assessment, MoCA: a brief screening tool for mild cognitive impairment. J Am Geriatr Soc. 2005; 53(4):695-699.
Parkinson J K, Murray E A, Mishkin M. A selective mnemonic role for the hippocampus in monkeys: memory for the location of objects. J Neurosci. 1988 November; 8(11):4159-4167.
Postuma R B, Arnulf I, Hogl B, et al. A single-question screen for rapid eye movement sleep behavior disorder: a multicenter validation study. Mov Disord. 2012 June; 27(7):913-916.
Riedel W J, Blokland A. Declarative memory. Handb Exp Pharmacol. 2015; 228:215-336. Spiropent (clenbuterol) 0.02 mg tablets [Summary of Product Characteristics]. Ingelheim am Rhein, Germany: Boehringer Ingelheim; March 2014.
Stylianou M, Murphy N, Peraza L R, et al. Quantitative electroencephalography as a marker of cognitive fluctuations in dementia with Lewy bodies and an aid to differential diagnosis. Clin Neurophysiol. 2018; 129:1209-1220.
Tranter R, Bell D, Gutting P, et al. The effect of serotonergic and noradrenergic antidepressants on face emotion processing in depressed subjects. J Affect Disord. 2009; 118:87-93.
Ventolin package insert, GSK Pharmaceuticals Inc., August 2021 https://www.accessdata.fda.gov/drugsatfda_docs/label/2021/020983s041lbl.pdf
Wakamatsu K, Tabuchi K, Ojika M, et al. Norepinephrine and its metabolites are involved in the synthesis of neuromelanin derived from the locus coeruleus. J Neurochem. 2015; 135(4):768-776.
Walker M P, Ayre G A, Cummings J L, et al. Quantifying fluctuation in dementia with Lewy bodies, Alzheimer's disease, and vascular dementia. Neurology. 2000; 54:1616-1625.
Yamaguchi T, Soma T, Takaku Y, et al. Salbutamol modulates the balance of Th1 and Th2 cytokines by mononuclear cells from allergic asthmatics. Int Arch Allergy Immunol. 2010; 152 Suppl 1:32-40.
Yesavage J A, Brink T L, Rose T L, et al. Development and validation of a geriatric depression screening scale: a preliminary report. J Psychiatr Res. 1982-1983; 17(1):37-49.

### Example 6. Safety, Tolerability and Effects on Cerebral Blood Flow after Single Doses of the β₂-Adrenoceptor Agonist, Clenbuterol, in Healthy Volunteers and Subjects with Mild Cognitive Impairment and Parkinson's Disease (Dose-Ranging Clenbuterol with and without Low-Dose Nadolol)

### Abstract

BACKGROUND: Cerebral hypometabolism, possibly caused by depletion in noradrenergic signalling, is an early event occurring years prior to a diagnosis of multiple neurodegenerative diseases including Alzheimer's (AD) and Parkinson's Disease (PD). Preclinical data with β-adrenoceptor (β-AR) agonists demonstrate an increase in cerebrocortical glucose metabolism and could therefore provide an alternative therapeutic option for AD[1].

METHODS: This study evaluated the safety and effects on cerebral activity of the β₂-AR clenbuterol in subjects with Mild Cognitive Impairment (MCI) or PD. Cerebral blood flow (CBF), which is tightly coupled to glucose metabolism, was measured by arterial spin labeling magnetic resonance imaging (ASL MRI). Administration of a low dose of nadolol, a non-selective β-AR antagonist with minimal brain penetration, was also evaluated on the cardiovascular, metabolic and CNS effects of clenbuterol as a secondary endpoint.

KEY RESULTS: Significant increases in CBF were seen in multiple brain regions, including those known to be involved in cognition and alertness such as the hippocampus and thalamus after administration of 80 μg clenbuterol, with and without pre-administration of 1 mg nadolol. Clenbuterol was overall safe and well tolerated in all subjects. The known side effects of β₂-agonists were observed in mild intensity, including increased heart rate, tremor and palpitations. These effects were mostly eliminated by the pre-administration of nadolol.

CONCLUSION: Clenbuterol's effects on CBF in MCI and PD subjects were evident both in the absence and presence of nadolol, suggesting that they are centrally mediated. Pre-treatment with a low dose of nadolol mitigated the peripheral effects of the β₂-AR clenbuterol, thereby confirming meaningful β₂-AR antagonism in the periphery without interruption of the central effects of clenbuterol on CBF.

### Introduction

Early stage deficiency in the noradrenergic system, originating largely from neurons in the locus coeruleus (LC), is demonstrated to be a common pathogenic mechanism in multiple neurodegenerative disorders[2]. In addition to the well-known peripheral cardiovascular effects of the noradrenergic system, noradrenaline (also known as norepinephrine) has a well-established central function on cognition, neuroinflammation and metabolism by eliciting its effects on α and β adrenergic receptors (α-ARs and β-ARs)[3]. In subjects with Alzheimer's disease (AD) or Parkinson's disease (PD), the LC is amongst the earliest sites of deposition of hyperphosphorylated tau[4] and α-synuclein, respectively[5]. Multiple pharmacoepidemiologic studies identify an association between exposure to β₂-AR agonists and a reduction in risk of progression to PD[6-10]. In nonclinical studies confirm a role of the LC in cognition. Specifically, LC lesioning in transgenic mice that express familial mutations of amyloid precursor protein linked to AD increased deposition of β-amyloid plaques [11]. In 6-month-old transgenic Tg344-19 AD rats, targeted lesioning of LC projections to the prefrontal cortex using dopamine-β-hydroxylase IgG-saporin worsened performance in the Barnes maze in measures of spatial and working memory[12]. Similarly, in a mouse model of paraquat and maneb-induced PD, LC lesioning was associated with memory deficits [13].

Cerebral blood flow (CBF) and its associated cerebral perfusion is essential for brain health. Observations across 1,171 subjects from the by the Alzheimer's Disease Neuroimaging Initiative (ADNI) database show that, across the spectrum of healthy controls, early MCI, late MCI and late-onset AD, vascular changes, including decreases in CBF as measured by arterial spin labelling MRI (ASL MRI), are the earliest to arise, and are evident before changes in b amyloid deposition, cognitive decline, tau and phosphorylated-tau in cerebrospinal fluid, or structural changes are detected [14]. In agreement with this, CBF, measured using arterial spin labelling MRI (ASL MRI), and cerebral vasoreactivity to CO2 were evaluated over 3 years in a longitudinal study that followed a cohort of older healthy adults and found to decrease in subjects in whom early signs of cognitive instability were present [15]. In a second longitudinal study, CBF measured in 2 ASL MRI scans 2 years apart, appears to follow a continuum of decrease over the evaluation period, with decreases in CBF in the hippocampus and precuneus of normal elderly controls (average age 73 years), and more extensive decreases in the hippocampus, middle temporal lobe, ventral striatum, prefrontal cortex, and cerebellum of MCI and AD subjects [16]. Regional hypoperfusion is also reported in PD subjects with and without dementia [17,18].

A depletion in noradrenergic signalling may lead to a state of regional hypometabolism in key areas including the thalamus, hippocampus and amygdala[6]. The noradrenergic system, and β₂-ARs in particular, could therefore be a promising therapeutic target for a variety of neurodegenerative disorders including AD and PD [19].

Both preclinical and clinical data suggest a potential role for β₂-AR agonists in the treatment of neurodegenerative disorders [6,20]. However, despite decades of clinical data on β-AR agonists, most studies have concentrated on the pulmonary and cardiovascular effects of this drug class and have provided minimal data on CNS effects.

This study evaluated the safety and effects of the β₂-AR agonist, clenbuterol, on cerebral blood flow (CBF), which is believed to be tightly coupled to glucose metabolism, in subjects with mild cognitive impairment (MCI) and PD using arterial spin labelled (ASL) perfusion magnetic resonance imaging (MRI) [21]. Clenbuterol is a high-affinity, full agonist at the β₂-AR with the ability to cross the blood-brain barrier.

In addition, since β₂-AR agonists are known to induce undesirable peripheral effects including increases in heart rate, tremor, palpitations, hyperglycemia and hypokalemia, selective inhibition of β₂-ARs in the periphery using a peripherally restricted P-antagonist was investigated using limited dose-ranging with nadolol to explore doses that selectively inhibit the peripheral but not the CNS effects of clenbuterol. Based on its low measured lipophilicity (as determined by octanol/water partition coefficient), nadolol has a low potential to cross the blood-brain barrier and therefore low drug concentration in the brain[22].

### Methods.

This clinical trial was conducted in accordance with standards of current Good Clinical Practice (GCP), as defined by the International Council for Harmonisation (ICH). Ethical approval from the Ethics Committee “Ethische Commissie onderzoek UZ/KU Leuven” was obtained. Written informed consent was obtained from all participants. All data were collected at the Center for Clinical Pharmacology and the Department of Nuclear Medicine of the University of Leuven, Belgium.

### Participants

To be eligible for inclusion in the study, healthy volunteers were required to be male, between 40-55 years or age, with a body weight of at least 50 kg and BMI 18-30 kg/mm², with no ongoing medication, or clinically significant condition.

For the MCI subjects had to meet the criteria for MCI as per the National Institute on Aging-Alzheimer's Association core clinical criteria. Subjects who already received a formal diagnosis of dementia according to the International Classifications of Diseases (ICD)-10 and Diagnostic and Statistical Manual of Mental Disorders (DSM)-IV were excluded from the study. A Clinical Dementia Rating scale score of 0.5 with a memory box score of ≥0.5 had to be attained at the first screening visit.

For the PD subjects, a Hoehn & Yahr stage ≤3 and a Unified Parkinson's Disease Rating scale (UPDRS) motor score of ≥10 and ≤30 had to obtained by a neurologist in the 3 months prior to screening. Subjects who were being treated with monoamine oxidase type B inhibitors, dopamine agonists or catechol-O-methyltransferase inhibitors were excluded from participation.

The cognitive decline or neurological symptoms were not to be caused primarily by vascular, traumatic or alternative medical problems. This was assessed by performing a medical anamnesis, physical and neurological exam. Additionally, structural brain abnormalities were excluded by conducting a brain MRI scan during a second screening visit.

### Study Design

Open-label clenbuterol was administered orally in the absence or presence of pre-administered or co-administered oral nadolol to 4 cohorts of healthy volunteers and 1 cohort of subjects with MCI or PD. Other cohorts were also enrolled in this study in which other drugs (with or without clenbuterol) were administered which are not discussed in this report.

In the 4 cohorts of healthy volunteers, study drug was administered as follows:

All subjects received single oral dose of 20 μg clenbuterol on Day 1, and a single oral dose of 40 μg clenbuterol on Day 8, with ASL MRI scans before and 3 hours after dosing on both days. A single oral dose of 5 mg nadolol was administered approximately 5 hours after clenbuterol.

All subjects received a single oral dose of 80 μg clenbuterol on Day 1, with ASL MRI scans before, and 3 hours 1 day and 2 days after clenbuterol administration to evaluate the longevity of the ASL MRI signal

All subjects received once-daily repeat oral doses of 80 μg clenbuterol for 1 week, with ASL MRI scans before, and 3 hours 1 day and 2 days after clenbuterol administration to evaluate the effects on ASL MRI and the safety and tolerability of repeat administration of clenbuterol in the absence or presence of nadolol. In this cohort, clenbuterol was administered as monotherapy in 4 subjects, and co-administered with 1 mg nadolol on all dosing days in the remaining subjects. ASL MRI scans were conducted before and 3 hours after clenbuterol administration on Day 1 and Day 7.

All subjects received a single oral dose of 160 μg clenbuterol on each of Day 2 and Day 9. A single oral dose of 5 mg nadolol was administered on the evening before and again on the morning of the second clenbuterol dose (approx. 2.5 hours prior to clenbuterol). ASL MRI scans were conducted before and 3 hours after clenbuterol administration on Day 2 and Day 9.

One cohort of subjects (MCI and PD) was enrolled in this study in which all subjects received a single oral dose of 80 μg clenbuterol on each of Day 2 and Day 9. In this cohort, clenbuterol was administered as monotherapy in 4 subjects, and following prior administration of 1 mg nadolol 2.5 hours earlier in the remaining 4 subjects. ASL MRI scans were conducted before and 3 hours after clenbuterol administration on Day 2 and Day 9.

Additional assessments of the effects of treatment included [¹⁸F]-fluordeoxyglucose positron emission tomography and blood oxygenation level dependent (BOLD MRI) imaging were also undertaken in one or more of the above cohorts. However, these findings will be reported elsewhere [23].

The 80 μg clenbuterol dose is supported by published clinical studies, including a 52-week clinical trial of clenbuterol 80 μg twice daily. [24]. Allocation subject ID and treatment regimens were assigned based on the order of screening. The 1 mg nadolol dose is below the recommended dosage range (40 to 240 mg/day) and has a well-established clinical safety profile. Prior studies conducted by CuraSen determined that this low dose of nadolol was sufficient to inhibit peripheral β₂-AR agonist mediated effects e.g., on heart rate.

### Imaging

All scans were conducted using pseudo continuous ASL MRI. Only subjects with no contraindications to being in a strong magnetic field were evaluated by MRI. Participants were positioned comfortably and supine in the head coil to minimize any motion during the scan with proper back support, and support under the knees for comfort and to reduce motion in the scan, and the head was centered in the head coil and velcro straps or foam padding were used to reduce motion.

Subjects underwent single-delay 3D pseudocontinuous ASL (pCASL) and 3D T1-weighted imaging at baseline, followed by a repeated pCASL scan post administration of clenbuterol (with or without pre-administration of nadolol). Imaging was performed on a GE Signa PET-MR 3T system.

For accurate quantitative measurements of brain structures a high-resolution T1-weighted, 3D volumetric sequences (e.g., MP-RAGE, IR-FSPGR) in the sagittal plane. Scan parameters of the pCASL sequence were: straight axial/transversal slices, labeling duration=1.8s, post-labeling delay=2.0 s, 15-20 cm slab thickness, pCASL average labeling gradient 1 mT/m, pCASL slice selective labelling gradient 10 mT/m, pASL TI1 (QUIPSS II Saturation Time) 800 ms, pASL labeling slab thickness 15-20 cm, 70 volumes, spatial resolution 3 mm in-plane/5 mm through-plane/gap 1 mm, 3D RARE stack-of-spiral or 3D GRASE 4-15 ms readouts, turbo-factor of 8 to 12; echo train of up to 300 ms, 2D EPI Single shot, minimum echo time, and duration 5 min.

Prior to analysis of ASL MRI data, each subject's structural MRI image underwent grey and white matter segmentation and was co-registered to a standard reference space (MNI152) [9]. The MNI152 template brain image and associated atlas (CIC atlas) was nonlinearly warped to the subject's MR image to enable automated definition of regions of interest (ROIs).[25] Multiple ROIs were evaluated including whole brain, cortex, subcortical, thalamus, amygdala, hippocampus, and cerebellum which were chosen due to their relevance for cognition or alertness, and the periaqueductal grey for its relevance to motor control. Grey matter (GM) masking was applied for reporting of CBF in the GM of these ROIs.

Regional mean CBF values were extracted for each ROI and subsequently, the CBF for each region was compared before and after administration of clenbuterol.

Baseline/Predose versus Postdose change (%) for each subject and each region was calculated as 100*(Postdose_CBF−Baseline_CBF)/(Baseline_CBF). For each region, the cohort summary of CBF change (=mean relative displacement, MRD) was described by the group mean and standard deviation of the individual CBF changes.

### Safety

Safety and tolerability were evaluated by questioning for adverse events (AEs), triplicate 12-lead ECGs, laboratory safety tests on urine and blood with multiple chemistry panels including potassium, blood glucose levels, vital sign measurements, and physical examinations at regular time-points during the study.

### Pharmacokinetics

Blood samples were collected at multiple time points for analysis of plasma concentrations of clenbuterol and nadolol using a research-grade liquid chromatography tandem mass spectrometry method. Pharmacokinetic (PK) parameters were computed from the individual plasma drug concentrations using a non-compartmental approach.

### Results

### Subjects

A total of 32 were enrolled to receive clenbuterol with or without nadolol between July 2019 and February 2021, including 25 healthy men aged 40-55 years, and 8 subjects (4 men and 4 women) between the ages of 58 and 71 years (mean [SD]=60.9 [4.3] years) with neurodegenerative disorders (7 with MCI, 1 with PD).

### Imaging

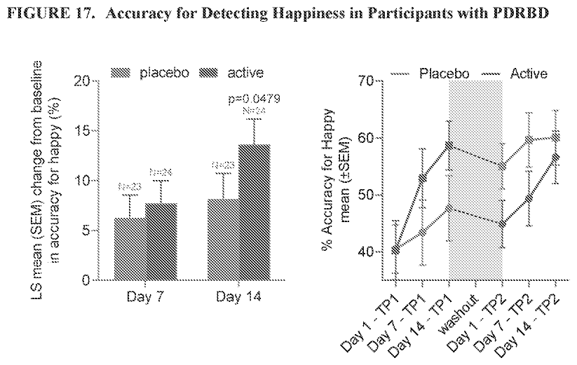

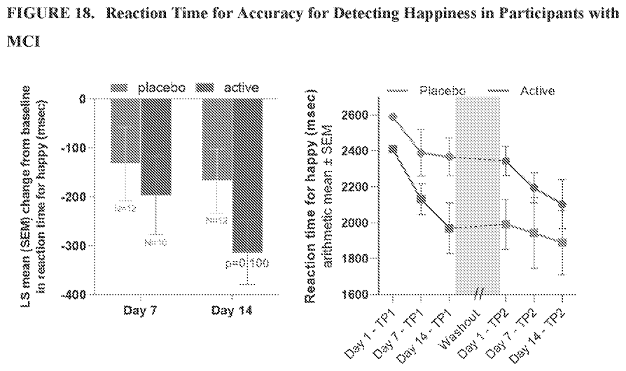

Global increases in brain cerebral blood flow (CBF) were observed by ASL MRI following administration of single doses of clenbuterol to healthy volunteers (FIG. 17). With the possible exception of the cerebellum, these effects were generally widespread across whole brain, including limbic regions involved in cognition, alertness and salience (hippocampus, thalamus and amygdala), and cortical regions including the motor cortex (precentral gyrus). Specifically, in the absence of nadolol, a dose-dependent increase in CBF was observed in healthy volunteers following administration of 20, 40, 80 and 160 μg clenbuterol with observed mean (±SEM) percent relative displacements (MRDs) from baseline in the hippocampus of −1.7±1.3%, 8.9±4.2%, 10.1±7.35%, 18.0±2.6%, 22.2±4.0%, 28.5±4.1%, respectively (FIG. 18A). In keeping with the long plasma halflife of clenbuterol, increases in CBF from baseline are evident at 3 hours after a single dose of 80 μg clenbuterol, and decreased but still evident at 24 hours later (when the plasma concentration of clenbuterol was approximately equivalent to a dose of 50 μg), but returned to baseline at 48 hours post dose (when the plasma concentration of clenbuterol was approximately equivalent to a dose of 30 μg, FIG. 18A).

Although somewhat, attenuated, increases in CBF were observed with clenbuterol in healthy volunteers even in the presence of low doses of nadolol. For example, among subjects who received 160 μg clenbuterol on Day 2, then 5 mg nadolol on Day 9 followed approximately 2.5 hours later by re-administration of 160 μg clenbuterol (FIG. 18B), widespread increases in CBF were noted following both administrations of agonist (mean SEM MRDs of 28.5±4.1% in the absence of nadolol, 14.0±6.5% in the presence of nadolol), but not following administration of 5 mg nadolol (0.49±1.64% in hippocampus).

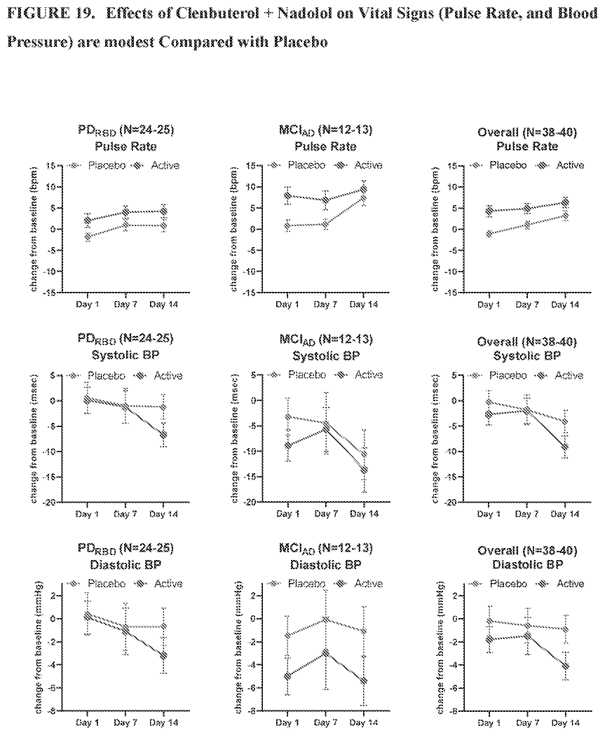

Among subjects with neurodegenerative disease enrolled in this study, the observed mean (±SEM) CBFs observed at baseline were similar across all ROIs to that seen for healthy volunteers 40-55 years of age (in whole brain 43.2±2.8 vs 41.7±1.5 mL/100 g/min, and in hippocampus 42.5±2.0 vs 44.0±1.2 mL/100 g/min for subjects [N=8] vs healthy volunteers [N=25]). As with the approximately age-matched healthy volunteers, clenbuterol monotherapy mediated widespread increases in CBF across whole brain, limbic regions involved in cognition, alertness and salience (hippocampus, thalamus and amygdala), and cortical regions including the motor cortex (precentral gyrus) which persisted in the presence of low dose nadolol. Specifically, observed mean (±SEM) MRDs following administration of clenbuterol in the absence and presence of 1 mg nadolol on Day 9 were 4.8±12.6% and 5.9±8.6% in whole brain and 14.7±10.6% vs 12.5±5.7% in in hippocampus (N=4 per treatment group, FIG. 19). Importantly, reproducible increases in CBF from baseline were observed among the 4 subjects who received 80 μg clenbuterol without nadolol on both Day 2 and Day 9 (MRD in the hippocampus=17.55±6.37% and 14.72±10.56% on Day 2 and Day 9, respectively), and the observed CBF at the end of the 7-day washout period between repeat doses of clenbuterol was very similar to predose (baseline) levels across most ROIs.

### Safety and Tolerability

Across all subjects enrolled, treatment-emergent adverse events (TEAEs) were all mild-to-moderate, and transient and were more considered to be related to clenbuterol in 12/25 healthy volunteers and 6/8 subjects with neurodegenerative disease. Common TEAEs of headache, tremor, palpitations, and tachycardia, consistent with the mechanism of action of β₂-AR agonism, were reported more frequently with clenbuterol when administered in the absence of nadolol. No TEAEs were considered to be related to nadolol.

Increases in supine heart rate were observed following administration of clenbuterol to healthy volunteers and subjects with MCI or PD. In the absence of nadolol, these effects generally increased over the dose range administered (20-160 μg), and increased over the first 3 hours after clenbuterol administration, with mean (SD) increase in heart rate from baseline of 7.5, 7.5, 9.2-12.38 and 30.5 bpm at 3 hours following 20, 40, 80 and 160 μg clenbuterol to healthy volunteers. Co-administration or pre-administration of nadolol lessened this peripheral effect of clenbuterol, e.g. pre-administration of 5 mg nadolol resulted in a decrease in heart rate from baseline of 2.75 bpm at 3 hours following administration of 160 μg clenbuterol. Similar effects were noted in subjects with MCI or PD in whom an increase in heart rate of 10.67-14.65 bpm was observed following 80 μg clenbuterol, which was effectively blocked by pre-administration of 1 mg nadolol.

There were no clinically meaningful effects of clenbuterol when administered in the absence or presence or nadolol on supine, sitting or standing blood pressure (data not shown).

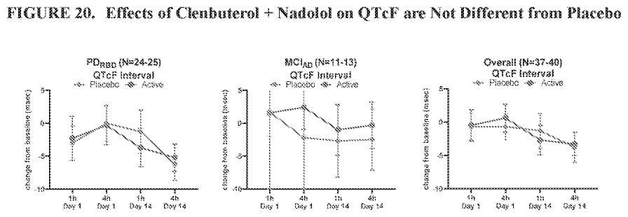

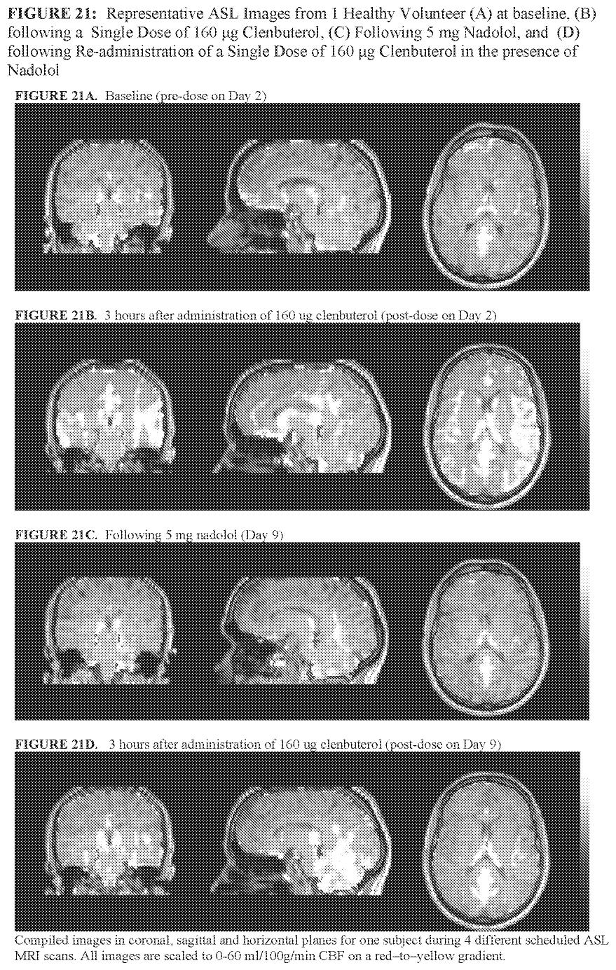

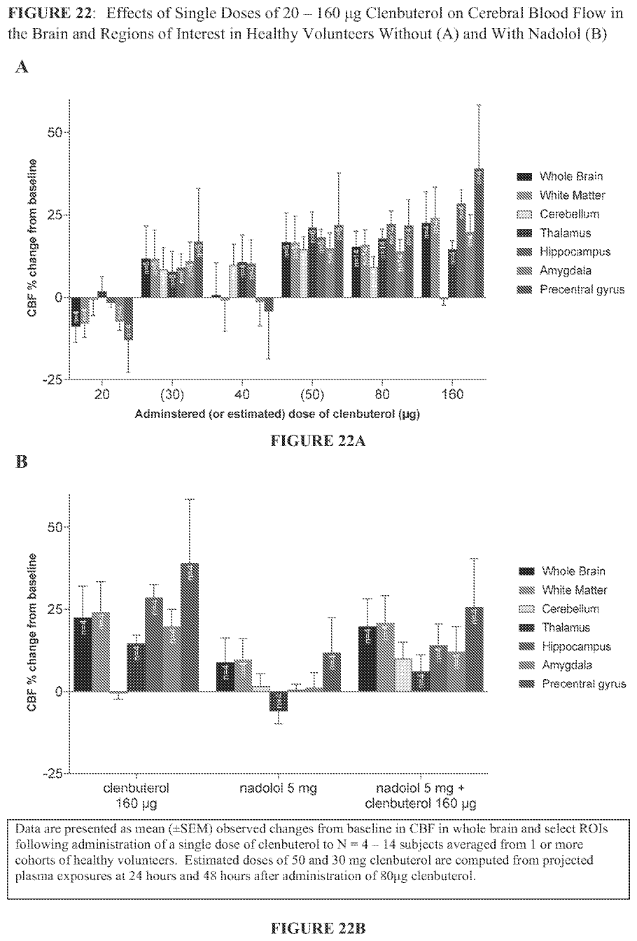

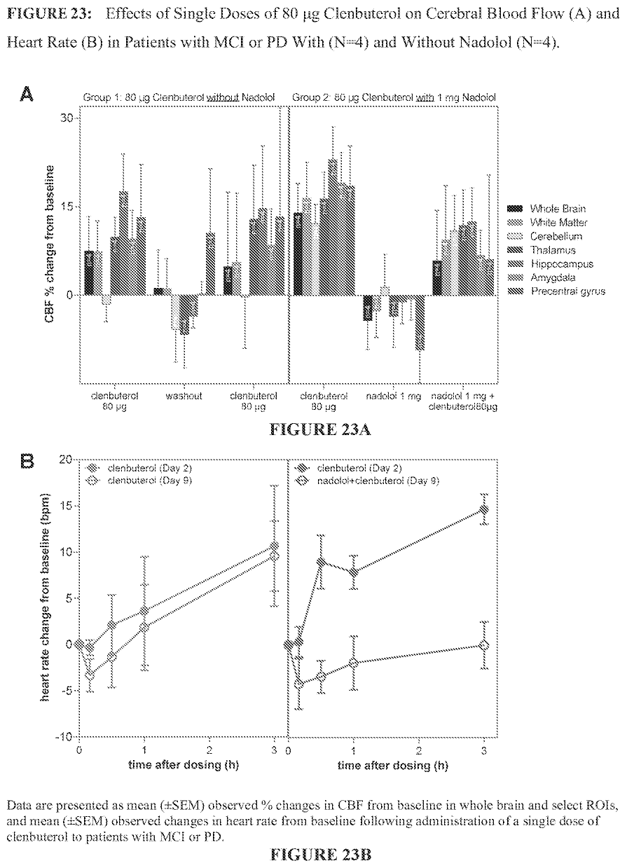

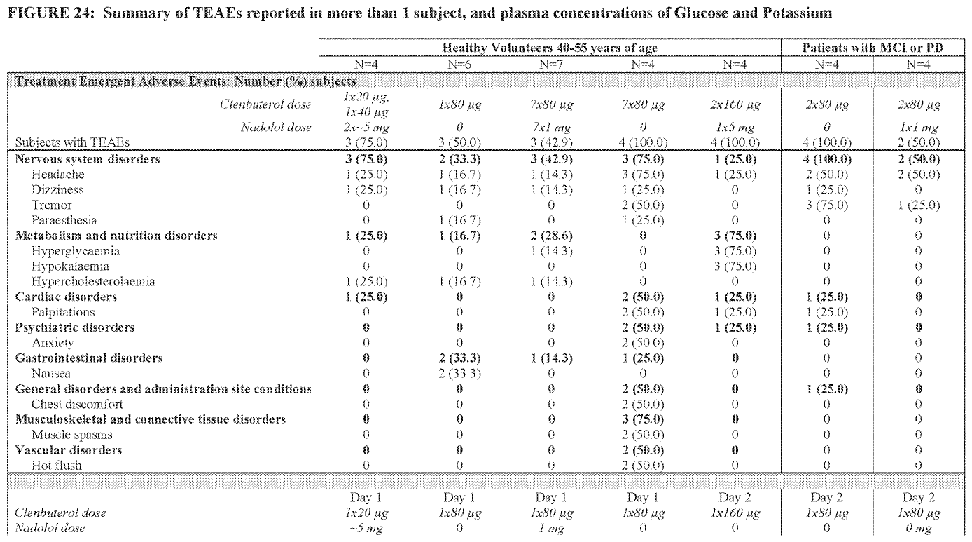

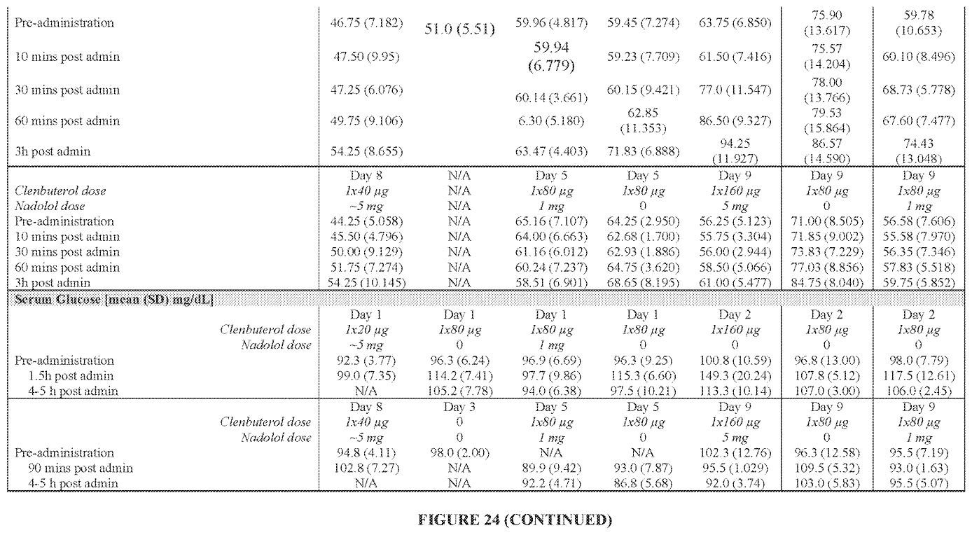

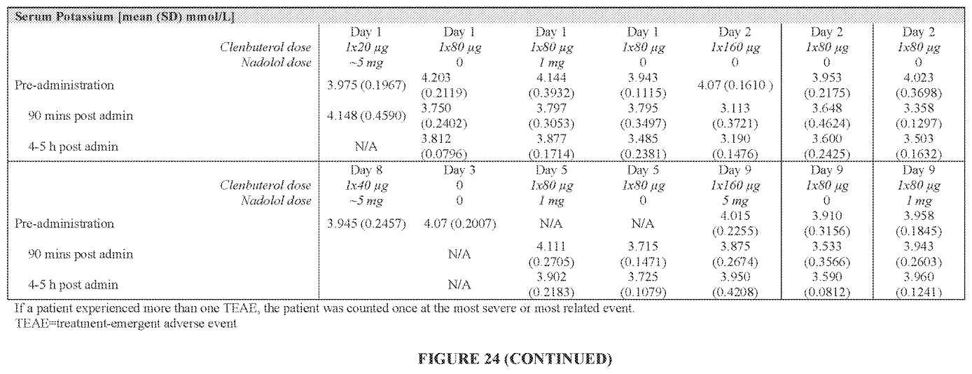

An overview of treatment-emergent adverse events (TEAEs), effects on heart rate and key laboratory measures is provided in FIG. 20.

### Pharmacokinetics

The observed pharmacokinetic (PK) properties of clenbuterol and nadolol was consistent with previously reported observations. Specifically, in healthy volunteers, the observed maximal plasma concentrations of the R-enantiomer of clenbuterol were attained at approximately 3-5 hours after administration of 20 μg-160 μg clenbuterol, with approximately dose-proportional increases in exposure (mean observed Cₘₐₓ=0.025, 0.055, 0.075-0.115, and 0.207 ng/mL following oral doses of 20, 40, 80 and 160 μg clenbuterol, respectively). Owing to slow plasma drug clearance, the plasma half-life of clenbuterol could only be determined in only some of the subjects and was highly variable (t_{1/2}=12-31 hours). Under this preliminary assessment, there was no effect of nadolol on the PK properties of clenbuterol. In addition, the observed PK was similar between healthy volunteers and subjects with MCI or PD.

Similarly, the observed PK properties of nadolol were similar to previous reports, with a maximum plasma concentration observed approximately 1-4 hours following a dose of 1-5 mg nadolol, and approximately dose-proportional increases in exposure (mean observed Cₘₐₓ=1-1.5, 2.8-4.0, and 4.39 ng/mL following oral doses of 1, 4 and 5 mg nadolol, respectively). As with clenbuterol, sparse sampling and slow plasma drug clearance resulted in plasma half-life estimates for nadolol in only a few subjects (t_{1/2}=7-13 hours).

### Discussion

This was an open-label pharmacodynamic study to evaluate the safety, tolerability and pharmacodynamic effects of the β₂-AR agonist, clenbuterol, on cerebral blood flow in healthy volunteers and subjects with MCI or PD.

Clenbuterol administration resulted in significant increases in CBF in the regions of interest, including hippocampus, thalamus and amygdala which are associated with cognition, attention, alertness and emotional salience and of interest for treatment of cognitive impairment in neurodegenerative diseases such as AD and PD. In addition, increases in CBF were observed in the precentral gyrus, also known as the motor cortex, and potentially of interest for treatment of diseases such as amyotrophic lateral sclerosis (ALS) or motor effects in PD. Clenbuterol's effects on CBF were evident both in the absence and presence of the peripherally restricted non-selective β-AR antagonist nadolol, suggesting that the increased perfusion was at least partially centrally mediated.

Increases in CBF are expected under conditions of increased neuronal activity. This phenomenon of neurovascular coupling arises from integrated responses from multiple cell types, many of which are known to express β₂ receptors, including neurons, cerebral blood vessels, microglia, oligodendrocytes and astrocytes. The observed increases in CBF may therefore suggest that the deficiency in the noradrenergic system that arises early in neurodegenerative disease progression may be at least partially restored by direct β₂ receptor activation with clenbuterol.

The most common clinical observations with clenbuterol of increases in heart rate, headache, tremor, palpitations, anxiety, decreases in potassium and increases in blood glucose, are consistent with β₂-agonist drug class [26-28], which has been in clinical use for over 50 years. These effects were observed more frequently when clenbuterol was administered in the absence of nadolol to healthy volunteers and subjects with MCI or PD in whom they were transient and mostly mild in intensity. Hypokalemia and hyperglycemia were temporally associated with clenbuterol dosing, with maximum effects observed between 90 minutes and 5 hours after the administration of clenbuterol but returning to normal values within a few hours after, and were inhibited by nadolol.

These safety findings in the absence of nadolol suggest that oral doses of 80 μg and 160 μg clenbuterol attained exposures in the periphery at which the β₂-AR was activated. Conversely, in the presence of nadolol, the peripheral effects on heart rate, tremor, palpitations potassium, and glucose were substantially inhibited while the effects to increase CBF, though attenuated, were still evident, suggesting that the effect on CBF is at least partially mediated through CNS actions of clenbuterol. That nadolol would have a predominantly peripheral action is expected from its octanol/water partition coefficient (ref #8) and very low uptake into the brain observed in mice [29], and supported by measures taken in this study to identify and administer the lowest dose of nadolol needed to inhibit β₂-AR effects in the periphery while preserving the CNS. Specifically, doses of 1 and 5 mg nadolol are respectively 40- and 8-fold lower than the typical starting dose of nadolol for treatment of hypertension (40 mg to 320 mg per day, per Corgard label). The pharmacokinetic results in this study confirm the fairly long half-life of clenbuterol which might be advantageous in terms of low treatment adherence in subjects with Alzheimer's or Parkinson's disease but consequently requires a longer time to reach steady state concentrations and a longer time for washout after discontinuation of the treatment.

The safety and pharmacodynamic observations in this study were generated in small numbers of participants and need to be confirmed. Nonetheless, the findings suggest that it is possible to selectively stimulate β₂-ARs in the CNS with clenbuterol to increase CBF while limiting the peripheral effects using nadolol, and warrant further clinical research with the 2 drugs in combination.

### REFERENCES

[1] Gutierrez I L, Russo C D, Novellino F, Caso J R, Garcia-Bueno B, Leza J C and Madrigal J L M 2022 Noradrenaline in Alzheimer's Disease: A New Potential Therapeutic Target Int J Mol Sci 23 6143
[2] Marien M R, Colpaert F C and Rosenquist A C 2004 Noradrenergic mechanisms in neurodegenerative diseases: a theory Brain Res Rev 45 38-78
[3] Gannon M, Che P, Chen Y, Jiao K, Roberson E D and Wang Q 2015 Noradrenergic dysfunction in Alzheimer's disease Front Neurosci-switz 9 220
[4] Braak H, Thal D R, Ghebremedhin E and Tredici K D 2011 Stages of the Pathologic Process in Alzheimer Disease: Age Categories From 1 to 100 Years J Neuropathology Exp Neurology 70 960-9
[5] Braak H, Tredici K D, Rub U, Vos R A I de, Steur E N H J and Braak E 2003 Staging of brain pathology related to sporadic Parkinson's disease Neurobiol Aging 24 197-211
[6] Mittal S, Bjornevik K, Im D S, Flierl A, Dong X, Locascio J J, Abo K M, Long E, Jin M, Xu B, Xiang Y K, Rochet J-C, Engeland A, Rizzu P, Heutink P, Bartels T, Selkoe D J, Caldarone B J, Glicksman M A, Khurana V, Schile B, Park D S, Riise T and Scherzer C R 2017 β2-Adrenoreceptor is a regulator of the α-synuclein gene driving risk of Parkinson's disease Science 357 891-8
[7] Gronich N, Abernethy D R, Auriel E, Lavi I, Rennert G and Saliba W 2018 β2-adrenoceptor agonists and antagonists and risk of Parkinson's disease Movement Disord 33 1465-71
[8] Nielsen S S, Gross A, Camacho-Soto A, Willis A W and Racette B A 2018 β2-adrenoreceptor medications and risk of Parkinson disease Ann Neurol 84 683-93
[9] Hopfner F, Wod M, Höglinger G U, Blaabjerg M, Rösler T W, Kuhlenbiumer G, Christensen K, Deuschl G and Pottegard A 2019 Use of β2-adrenoreceptor agonist and antagonist drugs and risk of Parkinson disease Neurology 93 e135-42
[10] Cepeda M S, Kern D M, Seabrook G R and Lovestone S 2019 Comprehensive Real-World Assessment of Marketed Medications to Guide Parkinson's Drug Discovery Clin Drug Invest 39 1067-75
[11] Kalinin S, Gavrilyuk V, Polak P E, Vasser R, Zhao J, Heneka M T and Feinstein D L 2007 Noradrenaline deficiency in brain increases β-amyloid plaque burden in an animal model of Alzheimer's disease Neurobiol Aging 28 1206-14
[12] Kelly S C, McKay E C, Beck J S, Collier T J, Dorrance A M and Counts S E 2019 Locus Coeruleus Degeneration Induces Forebrain Vascular Pathology in a Transgenic Rat Model of Alzheimer's Disease J Alzheimer's Dis 70 371-88
[13] Hou L, Sun F, Sun W, Zhang L and Wang Q 2019 Lesion of the Locus Coeruleus Damages Learning and Memory Performance in Paraquat and Maneb-induced Mouse Parkinson's Disease Model Neuroscience 419 129-40
[14] Iturria-Medina Y, Sotero R C, Toussaint P J, Mateos-Perez J M, Evans A C, Weiner M W, Aisen P, Petersen R, Jack C R, Jagust W, Trojanowki J Q, Toga A W, Beckett L, Green R C, Saykin A J, Morris J, Shaw L M, Khachaturian Z, Sorensen G, Kuller L, Raichle M, Paul S, Davies P, Fillit H, Hefti F, Holtzman D, Mesulam M M, Potter W, Snyder P, Schwartz A, Montine T, Thomas R G, Donohue M, Walter S, Gessert D, Sather T, Jiminez G, Harvey D, Bernstein M, Fox N, Thompson P, Schuff N, Borowski B, Gunter J, Senjem M, Vemuri P, Jones D, Kantarci K, Ward C, Koeppe R A, Foster N, Reiman E M, Chen K, Mathis C, Landau S, Cairns N J, Householder E, Taylor-Reinwald L, Lee V, Korecka M, Figurski M, Crawford K, Neu S, Foroud T M, Potkin S, Shen L, Faber K, Kim S, Nho K, Thal L, Buckholtz N, Albert M, Frank R, Hsiao J, Kaye J, Quinn J, Lind B, Carter R, Dolen S, Schneider L S, Pawluczyk S, Beccera M, Teodoro L, Spann B M, Brewer J, Vanderswag H, Fleisher A, Heidebrink J L, Lord J L, Mason S S, Albers C S, Knopman D, Johnson K, Doody R S, Villanueva-Meyer J, Chowdhury M, Rountree S, Dang M, et al 2016 Early role of vascular dysregulation on late-onset Alzheimer's disease based on multifactorial data-driven analysis Nat Commun 7 11934
[15] Thiel M van der, Rodriguez C, Ville D V D, Giannakopoulos P and Haller S 2019 Regional Cerebral Perfusion and Cerebrovascular Reactivity in Elderly Controls With Subtle Cognitive Deficits Front Aging Neurosci 11 19
[16] Camargo A, Wang Z and Initiative A D N 2021 Longitudinal Cerebral Blood Flow Changes in Normal Aging and the Alzheimer's Disease Continuum Identified by Arterial Spin Labeling MRI J Alzheimer's Dis 81 1727-35
[17] Heron C J L, Wright S L, Melzer T R, Myall D J, MacAskill M R, Livingston L, Keenan R J, Watts R, Dalrymple-Alford J C and Anderson T J 2014 Comparing Cerebral Perfusion in Alzheimer's Disease and Parkinson's Disease Dementia: An ASL-MRI Study J Cereb Blood Flow Metabolism 34 964-70
[18] Kamagata K, Motoi Y, Hori M, Suzuki M, Nakanishi A, Shimoji K, Kyougoku S, Kuwatsuru R, Sasai K, Abe O, Mizuno Y, Aoki S and Hattori N 2011 Posterior hypoperfusion in parkinson's disease With and without dementia measured with arterial spin labeling MRI J Magn Reson Imaging 33 803-7
[19] Peterson A C and Li C R 2018 Noradrenergic Dysfunction in Alzheimer's and Parkinson's Diseases—An Overview of Imaging Studies Front Aging Neurosci 10 127
[20] Heneka M T, Galea E, Gavriluyk V, Dumitrescu-Ozimek L, Daeschner J, O'Banion M K, Weinberg G, Klockgether T and Feinstein D L 2002 Noradrenergic Depletion Potentiates β-Amyloid-Induced Cortical Inflammation: Implications for Alzheimer's Disease J Neurosci 22 2434-42
[21] Wolk D A and Detre J A 2012 Arterial spin labeling MRI Curr Opin Neurol 25 421-8
[22] Poirier L and Tobe S W 2014 Contemporary Use of P-Blockers: Clinical Relevance of Subclassification Can J Cardiol 30 S9-15
[23] Bishop C, Rizzo G, Lodeweyckx T, Hoon J de, Laere K van, Koole M, Vandenberghe W, Rabiner E, Martin R, A A F and Vargas G 2022 Disentangling apparent discordance between ASL-MRI and [18F]-FDG PET following a single dose of the β2-agonist clenbuterol. International Society for Magnetic Resonance in Medicine
[24] Koeberl D D, Case L E, Smith E C, Walters C, Han S, Li Y, Chen W, Hornik C P, Huffman K M, Kraus W E, Thurberg B L, Corcoran D L, Bali D, Bursac N and Kishnani P S 2018 Correction of Biochemical Abnormalities and Improved Muscle Function in a Phase I/II Clinical Trial of Clenbuterol in Pompe Disease Mol Ther 26 2304-14
[25] Tziortzi A C, Searle G E, Tzimopoulou S, Salinas C, Beaver J D, Jenkinson M, Laruelle M, Rabiner E A and Gunn R N 2011 Imaging dopamine receptors in humans with [11C]-(+)-PHNO: Dissection of D3 signal and anatomy Neuroimage 54 264-77
[26] Kempsford R, Allen A, Kelly K, Saggu P and Crim C 2014 Thorough QT study of inhaled fluticasone furoate and vilanterol Brit J Clin Pharmaco 77 466-79
[27] Lötvall J and Ankerst J 2008 Long duration of airway but not systemic effects of inhaled formoterol in asthmatic subjects Resp Med 102 449-56
[28] Löfdahl C, Sigvaldasson A, Skoogh B and Svedmyr N 1989 Broxaterol, a New β₂-Adrenoceptor Agonist Compared to Salbutamol in Asthmatics, Oral and Inhalation Treatment Respiration Suppl 2 15-9
[29] Laurens C, Abot A, Delarue A and Knauf C 2019 Central Effects of Beta-Blockers May Be Due to Nitric Oxide and Hydrogen Peroxide Release Independently of Their Ability to Cross the Blood-Brain Barrier Front Neurosci-switz 13 33

### Aspects and Embodiments of the Disclosure

In one aspect, the disclosure provides a method that includes: administering to said subject a β-agent and a peripherally acting β-blocker (PABRA), wherein the peripherally acting β-blocker (PABRA) is administered in a dose of about 15 mg or less. The method can further include subjecting a subject to brain imaging to determine cognitive function and/or to identify whether said subject is in need of or desiring improvement of cognitive function and/or treatment of a neurodegenerative disease, and/or identifying a particular type of neurodegenerative disease based on a spatial pattern of the brain imaging result.

In another aspect, the disclosure provides a method including: administering to said subject a β-agent and a peripherally acting β-blocker (PABRA) to improve cognition and/or treat a neurodegenerative disease in said subject, wherein the peripherally acting β-blocker (PABRA) is administered in a dose of about 15 mg or less. The method can further include subjecting a subject to brain imaging to determine cognitive function to identify whether said subject is in need of or desiring improvement of cognitive function and/or treatment of a neurodegenerative disease, identifying a particular type of neurodegenerative disease based on a spatial pattern of the brain imaging result, and/or subsequently re-subjecting said subject to brain imaging to determine any improvement in cognitive function and/or treatment of said neurodegenerative disease.

In yet another aspect, the disclosure provides a method including: subjecting a subject to brain imaging to determine cognitive function in said subject; identifying a particular type of neurodegenerative disease based on a spatial pattern of the brain imaging result; administering to said subject a β-agent and a peripherally acting β-blocker (PABRA), wherein the peripherally acting β-blocker (PABRA) is administered in a dose of about 15 mg or less; and subsequently re-subjecting said subject to brain imaging to determine any improvement in cognitive function.

In still another aspect, the disclosure provides a method including: administering to said subject a β-agent and a peripherally acting β-blocker (PABRA), wherein the peripherally acting β-blocker (PABRA) is administered in a dose of about 15 mg or less. The method can further include subjecting a subject to brain imaging to determine cognitive function and/or to identify whether said subject is in need of or desiring improvement of cognitive function and/or treatment of a neurodegenerative disease, and/or identifying a particular type of neurodegenerative disease based on a spatial pattern of the brain imaging result.

In another aspect, the disclosure provides a method including: administering to said subject a β-agent and a peripherally acting β-blocker (PABRA) to improve cognition and/or treat a neurodegenerative disease in said subject, wherein the peripherally acting β-blocker (PABRA) is administered in a dose of about 15 mg or less. The method can further include subjecting a subject to brain imaging to determine cognitive function and/or to identify whether said subject is in need of or desiring improvement of cognitive function and/or treatment of a neurodegenerative disease, identifying a particular type of neurodegenerative disease based on a spatial pattern of the brain imaging result; and/or subsequently re-subjecting said subject to brain imaging to determine any improvement in cognitive function and/or treatment of said neurodegenerative disease.

In another aspect, the disclosure provides a method including: subjecting a subject to brain imaging to determine cognitive function in said subject; identifying a particular type of neurodegenerative disease based on a spatial pattern of the brain imaging result; administering to said subject a β-agent and a peripherally acting β-blocker (PABRA), wherein the peripherally acting β-blocker (PABRA) is administered in a dose of about 15 mg or less; and subsequently re-subjecting said subject to brain imaging to determine any improvement in cognitive function.

In another aspect, the disclosure provides a method including: administering to said subject clenbuterol and a peripherally acting β-blocker (PABRA), wherein the peripherally acting β-blocker (PABRA) is administered in a dose of about 15 mg or less. The method can further include subjecting a subject to brain imaging to determine cognitive function and/or to identify whether said subject is in need of or desiring improvement of cognitive function and/or treatment of a neurodegenerative disease, and/or identifying a particular type of neurodegenerative disease based on a spatial pattern of the brain imaging result.

In another aspect, the disclosure provides a method including: administering to said subject clenbuterol and a peripherally acting β-blocker (PABRA) to improve cognition and/or treat a neurodegenerative disease in said subject, wherein the peripherally acting β-blocker (PABRA) is administered in a dose of about 15 mg or less. The method can further include subjecting a subject to brain imaging to determine cognitive function and/or to identify whether said subject is in need of or desiring improvement of cognitive function and/or treatment of a neurodegenerative disease, identifying a particular type of neurodegenerative disease based on a spatial pattern of the brain imaging result, and subsequently re-subjecting said subject to brain imaging to determine any improvement in cognitive function and/or treatment of said neurodegenerative disease.

In another aspect, the disclosure provides a method including: subjecting a subject to brain imaging to determine cognitive function in said subject; identifying a particular type of neurodegenerative disease based on a spatial pattern of the brain imaging result; administering to said subject clenbuterol and a peripherally acting β-blocker (PABRA), wherein the peripherally acting β-blocker (PABRA) is administered in a dose of about 15 mg or less; and subsequently re-subjecting said subject to brain imaging to determine any improvement in cognitive function.

In another aspect, the disclosure provides a method including: administering to said subject tulobuterol and a peripherally acting β-blocker (PABRA), wherein the peripherally acting β-blocker (PABRA) is administered in a dose of about 15 mg or less. The method can further include subjecting a subject to brain imaging to determine cognitive function and/or to identify whether said subject is in need of or desiring improvement of cognitive function and/or treatment of a neurodegenerative disease, and/or identifying a particular type of neurodegenerative disease based on a spatial pattern of the brain imaging result; and subsequently administering to said subject tulobuterol and a peripherally acting β-blocker (PABRA).

In another aspect, the disclosure provides a method including: administering to said subject tulobuterol and a peripherally acting β-blocker (PABRA) to improve cognition and/or treat a neurodegenerative disease in said subject, wherein the peripherally acting β-blocker (PABRA) is administered in a dose of about 15 mg or less. The method can further include subjecting a subject to brain imaging to determine cognitive function and/or to identify whether said subject is in need of or desiring improvement of cognitive function and/or treatment of a neurodegenerative disease, identifying a particular type of neurodegenerative disease based on a spatial pattern of the brain imaging result and/or subsequently re-subjecting said subject to brain imaging to determine any improvement in cognitive function and/or treatment of said neurodegenerative disease.

In another aspect, the disclosure provides a method including: subjecting a subject to brain imaging to determine cognitive function in said subject; identifying a particular type of neurodegenerative disease based on a spatial pattern of the brain imaging result; administering to said subject tulobuterol and a peripherally acting β-blocker (PABRA), wherein the peripherally acting β-blocker (PABRA) is administered in a dose of about 15 mg or less; and subsequently re-subjecting said subject to brain imaging to determine any improvement in cognitive function.

In another aspect, the disclosure provides a method including treating a subject identified as having diminished cognitive function and/or being in need of or desiring improvement of cognitive function and/or treatment of a neurodegenerative disease by administering the subject a pharmaceutical composition including a β-AR agonist (such as a β-agent), β₁-AR agonist, a β₂-AR agonist, a peripherally acting β-blocker (PABRA), or any combination thereof, wherein the peripherally acting β-blocker (PABRA) is administered in a dose of about 15 mg or less. In some embodiments, the method further includes assessing effectiveness of the treatment, the treatment can be assessed by subjecting the subject to a test to assess improved cognitive function or amelioration of the neurodegenerative disease. In some embodiments, the method further includes adjusting administration of the pharmaceutical composition by adjusting dosage of the pharmaceutical composition and/or timing of administration of the pharmaceutical composition.

In one aspect, the disclosure provides a method that includes: administering to said subject a β-AR agonist (such as a β-agent) and a peripherally acting β-blocker (PABRA), wherein the peripherally acting β-blocker (PABRA) is administered in a sub-therapeutic dose. The method can further include subjecting a subject to brain imaging to determine cognitive function and/or to identify whether said subject is in need of or desiring improvement of cognitive function and/or treatment of a neurodegenerative disease, and/or identifying a particular type of neurodegenerative disease based on a spatial pattern of the brain imaging result.

In another aspect, the disclosure provides a method including: administering to said subject a β-AR agonist (such as a β-agent) and a peripherally acting β-blocker (PABRA) to improve cognition and/or treat a neurodegenerative disease in said subject, wherein the peripherally acting β-blocker (PABRA) is administered in a sub-therapeutic dose. The method can further include subjecting a subject to brain imaging to determine cognitive function to identify whether said subject is in need of or desiring improvement of cognitive function and/or treatment of a neurodegenerative disease, identifying a particular type of neurodegenerative disease based on a spatial pattern of the brain imaging result, and/or subsequently re-subjecting said subject to brain imaging to determine any improvement in cognitive function and/or treatment of said neurodegenerative disease.

In yet another aspect, the disclosure provides a method including: subjecting a subject to brain imaging to determine cognitive function in said subject; identifying a particular type of neurodegenerative disease based on a spatial pattern of the brain imaging result; administering to said subject a β-AR agonist (such as a β-agent) and a peripherally acting β-blocker (PABRA), wherein the peripherally acting β-blocker (PABRA) is administered in a sub-therapeutic dose; and subsequently re-subjecting said subject to brain imaging to determine any improvement in cognitive function.

In still another aspect, the disclosure provides a method including: administering to said subject a β-AR agonist (such as a β-agent) and a peripherally acting β-blocker (PABRA), wherein the peripherally acting β-blocker (PABRA) is administered in a sub-therapeutic dose. The method can further include subjecting a subject to brain imaging to determine cognitive function and/or to identify whether said subject is in need of or desiring improvement of cognitive function and/or treatment of a neurodegenerative disease, and/or identifying a particular type of neurodegenerative disease based on a spatial pattern of the brain imaging result.

In another aspect, the disclosure provides a method including: administering to said subject a β-AR agonist (such as a β-agent) and a peripherally acting β-blocker (PABRA) to improve cognition and/or treat a neurodegenerative disease in said subject, wherein the peripherally acting β-blocker (PABRA) is administered in a sub-therapeutic dose. The method can further include subjecting a subject to brain imaging to determine cognitive function and/or to identify whether said subject is in need of or desiring improvement of cognitive function and/or treatment of a neurodegenerative disease, identifying a particular type of neurodegenerative disease based on a spatial pattern of the brain imaging result; and/or subsequently re-subjecting said subject to brain imaging to determine any improvement in cognitive function and/or treatment of said neurodegenerative disease.

In another aspect, the disclosure provides a method including: subjecting a subject to brain imaging to determine cognitive function in said subject; identifying a particular type of neurodegenerative disease based on a spatial pattern of the brain imaging result; administering to said subject a β-AR agonist (such as a β-agent) and a peripherally acting β-blocker (PABRA), wherein the peripherally acting β-blocker (PABRA) is administered in a sub-therapeutic dose; and subsequently re-subjecting said subject to brain imaging to determine any improvement in cognitive function.

In another aspect, the disclosure provides a method including: administering to said subject clenbuterol and a peripherally acting β-blocker (PABRA), wherein the peripherally acting β-blocker (PABRA) is administered in a sub-therapeutic dose. The method can further include subjecting a subject to brain imaging to determine cognitive function and/or to identify whether said subject is in need of or desiring improvement of cognitive function and/or treatment of a neurodegenerative disease, and/or identifying a particular type of neurodegenerative disease based on a spatial pattern of the brain imaging result.

In another aspect, the disclosure provides a method including: administering to said subject clenbuterol and a peripherally acting β-blocker (PABRA) to improve cognition and/or treat a neurodegenerative disease in said subject, wherein the peripherally acting β-blocker (PABRA) is administered in a sub-therapeutic dose. The method can further include subjecting a subject to brain imaging to determine cognitive function and/or to identify whether said subject is in need of or desiring improvement of cognitive function and/or treatment of a neurodegenerative disease, identifying a particular type of neurodegenerative disease based on a spatial pattern of the brain imaging result, and subsequently re-subjecting said subject to brain imaging to determine any improvement in cognitive function and/or treatment of said neurodegenerative disease.

In another aspect, the disclosure provides a method including: subjecting a subject to brain imaging to determine cognitive function in said subject; identifying a particular type of neurodegenerative disease based on a spatial pattern of the brain imaging result; administering to said subject clenbuterol and a peripherally acting β-blocker (PABRA), wherein the peripherally acting β-blocker (PABRA) is administered a sub-therapeutic dose; and subsequently re-subjecting said subject to brain imaging to determine any improvement in cognitive function.

In another aspect, the disclosure provides a method including: administering to said subject tulobuterol and a peripherally acting β-blocker (PABRA), wherein the peripherally acting β-blocker (PABRA) is administered in a sub-therapeutic dose. The method can further include subjecting a subject to brain imaging to determine cognitive function and/or to identify whether said subject is in need of or desiring improvement of cognitive function and/or treatment of a neurodegenerative disease, and/or identifying a particular type of neurodegenerative disease based on a spatial pattern of the brain imaging result; and subsequently administering to said subject tulobuterol and a peripherally acting β-blocker (PABRA).

In another aspect, the disclosure provides a method including: administering to said subject tulobuterol and a peripherally acting β-blocker (PABRA) to improve cognition and/or treat a neurodegenerative disease in said subject, wherein the peripherally acting β-blocker (PABRA) is administered in a sub-therapeutic dose. The method can further include subjecting a subject to brain imaging to determine cognitive function and/or to identify whether said subject is in need of or desiring improvement of cognitive function and/or treatment of a neurodegenerative disease, identifying a particular type of neurodegenerative disease based on a spatial pattern of the brain imaging result and/or subsequently re-subjecting said subject to brain imaging to determine any improvement in cognitive function and/or treatment of said neurodegenerative disease.

In another aspect, the disclosure provides a method including: subjecting a subject to brain imaging to determine cognitive function in said subject; identifying a particular type of neurodegenerative disease based on a spatial pattern of the brain imaging result; administering to said subject tulobuterol and a peripherally acting β-blocker (PABRA), wherein the peripherally acting β-blocker (PABRA) is administered in a sub-therapeutic dose; and subsequently re-subjecting said subject to brain imaging to determine any improvement in cognitive function.

In another aspect, the disclosure provides a method including treating a subject identified as having diminished cognitive function and/or being in need of or desiring improvement of cognitive function and/or treatment of a neurodegenerative disease by administering the subject a pharmaceutical composition including a β-agent, a β₁-AR agonist, a β₂-AR agonist, a peripherally acting β-blocker (PABRA), or any combination thereof, wherein the peripherally acting β-blocker (PABRA) is administered in a sub-therapeutic dose. In some embodiments, the method further includes assessing effectiveness of the treatment, the treatment can be assessed by subjecting the subject to a test to assess improved cognitive function or amelioration of the neurodegenerative disease. In some embodiments, the method further includes adjusting administration of the pharmaceutical composition by adjusting dosage of the pharmaceutical composition and/or timing of administration of the pharmaceutical composition.

In embodiments of any aspect or embodiment of the disclosure described herein, the brain imaging is fluorodeoxyglucose positron emission tomography (FDG-PET) scan, magnetic resonance imaging-arterial spin labeling (MRI-ASL), or magnetic resonance imaging-blood oxygenation level dependent computerized tomography (MRI-BOLD).

In embodiments of any aspect or embodiment of the disclosure described herein, said β-agent is administered at a dose of from about 0.01 to 100 mg.

In embodiments of any aspect or embodiment of the disclosure described herein, said β-agent is administered at a dose of from about 30 to 160 μg.

In embodiments of any aspect or embodiment of the disclosure described herein, said β-agent is administered at a dose of from about 30 to 160 μg, 50 to 160 μg, 80 to 160 μg, 100 to 160 μg, 120 to 160 μg, 140 to 160 μg, 30 to 140 μg, 50 to 140 μg, 80 to 140 μg, 100 to 140 μg, 120 to 140 μg, 30 to 120 μg, 50 to 120 μg, 80 to 120 μg, 100 to 120 μg, 30 to 100 μg, 50 to 100 μg, 80 to 100 μg, 30 to 80 μg, 50 to 80 μg, 30 to 50 μg, 30 μg, 40 μg, 50 μg, 60 μg, 70 μg, 80 μg, 90 μg, 100 μg, 110 μg, 120 μg, 130 μg, 140 μg, 150 μg, or 160 μg.

In embodiments of any aspect or embodiment of the disclosure described herein, said β-agent is administered at a dose of from about 0.5-20 mg; or 1-10 mg; or 2-8 mg; or about 1 mg; or about 2 mg; or about 3 mg; or about 4 mg; or about 5 mg; or about 6 mg; or about 7 mg; or about 8 mg; or about 10 mg.

In embodiments of any aspect or embodiment of the disclosure described herein, the above-mentioned dose is a total daily dose of β-agent agonist and is administered daily for a period of weeks or more.

In embodiments of any aspect or embodiment of the disclosure described herein, the above-mentioned dose is a total weekly dose of β-agent and is administered weekly for a period of weeks or more.

In embodiments of any aspect or embodiment of the disclosure described herein, said β-agent is Compound 03-5, or an optically pure stereoisomer, pharmaceutically acceptable salt, solvate, or prodrug thereof.

In embodiments of any aspect or embodiment of the disclosure described herein, said peripherally acting β-blocker (PABRA) is nadolol.

In embodiments of any aspect or embodiment of the disclosure described herein, nadolol is a mixture of four diastereomers.

In embodiments of any aspect or embodiment of the disclosure described herein, the nadolol administered is a specific enantiomerically pure isomer.

In embodiments of any aspect or embodiment of the disclosure described herein, said peripherally acting β-blocker (PABRA) is administered at a dose of from about 0.1 mg to 15 mg.

In embodiments of any aspect or embodiment of the disclosure described herein, said peripherally acting β-blocker (PABRA) is administered at a dose of from about 0.1 to 15 mg, 0.1-10 mg, 0.1 to 1 mg, 0.1 to 5 mg, 1 to 15 mg, 1 to 10 mg, 1 to 5 mg, 5 to 10 mg, 10 mg or less, 7 mg or less, 5 mg or less, 1 mg or less, 0.1 mg, 0.5 mg, 1 mg, 2 mg, 3 mg, 4 mg, 5 mg, 6 mg, 7 mg, 8 mg, 9 mg, or 10 mg.

In embodiments of any aspect or embodiment of the disclosure described herein, the above-mentioned dose is a total daily dose of said peripherally acting β-blocker (PABRA) and is administered daily for a period of weeks or more.

In embodiments of any aspect or embodiment of the disclosure described herein, said β-agent, β₁-AR agonist, β₂-AR agonist and/or peripherally acting β-blocker (PABRA) are each administered orally.

In embodiments of any aspect or embodiment of the disclosure described herein, said β-agent and peripherally acting β-blocker (PABRA) are each administered orally and both agents are present in a tablet.

In embodiments of any aspect or embodiment of the disclosure described herein, nadolol is provided in an amount from about 0.1 to 15 mg, 0.1 to 10 mg, 0.1 to 1 mg, 0.1 to 5 mg, 1 to 15 mg, 1 to 10 mg, 1 to 5 mg, 5 to 10 mg, 10 mg or less, 7 mg or less, 5 mg or less, 1 mg or less, 0.1 mg, 0.5 mg, 1 mg, 2 mg, 3 mg, 4 mg, 5 mg, 6 mg, 7 mg, 8 mg, 9 mg, or 10 mg.

In embodiments of any aspect or embodiment of the disclosure described herein, said neurodegenerative disease is one or more selected from MCI, aMCI, Vascular Dementia, Mixed Dementia, FTD (fronto-temporal dementia; Pick's disease), HD (Huntington disease), Rett Syndrome, PSP (progressive supranuclear palsy), CBD (corticobasal degeneration), SCA (spinocerebellar ataxia), MSA (Multiple system atrophy), SDS (Shy-Drager syndrome), olivopontocerebellar atrophy, TBI (traumatic brain injury), CTE (chronic traumatic encephalopathy), stroke, WKS (Wernicke-Korsakoff syndrome; alcoholic dementia & thiamine deficiency), normal pressure hydrocephalus, hypersomnia/narcolepsy, ASD (autistic spectrum disorders), FXS (fragile X syndrome), TSC (tuberous sclerosis complex), prion-related diseases (CJD etc.), depressive disorders, DLB (dementia with Lewy bodies), PD (Parkinson's disease), PDD (PD dementia), ADHD (attention deficit hyperactivity disorder), and Down Syndrome.

In embodiments of any aspect or embodiment of the disclosure described herein, said neurodegenerative disease is one or more selected from MCI, aMCI, Vascular Dementia, Mixed Dementia, FTD (fronto-temporal dementia; Pick's disease), HD (Huntington disease), Rett Syndrome, PSP (progressive supranuclear palsy), CBD (corticobasal degeneration), SCA (spinocerebellar ataxia), MSA (Multiple system atrophy), SDS (Shy-Drager syndrome), olivopontocerebellar atrophy, TBI (traumatic brain injury), CTE (chronic traumatic encephalopathy), stroke, WKS (Wernicke-Korsakoff syndrome; alcoholic dementia & thiamine deficiency), normal pressure hydrocephalus, hypersomnia/narcolepsy, ASD (autistic spectrum disorders), FXS (fragile X syndrome), TSC (tuberous sclerosis complex), prion-related diseases (CJD etc.), depressive disorders, DLB (dementia with Lewy bodies), PD (Parkinson's disease), PDD (PD dementia), and ADHD (attention deficit hyperactivity disorder).

In embodiments of any aspect or embodiment of the disclosure described herein, said subject does not have Alzheimer's disease.

In embodiments of any aspect or embodiment of the disclosure described herein, said subject does not have Down Syndrome.

In embodiments of any aspect or embodiment of the disclosure described herein, said subject does not have Parkinson's disease.

In embodiments of any aspect or embodiment of the disclosure described herein, said subject does not have dementia with Lewy bodies.

In embodiments of any aspect or embodiment of the disclosure described herein, a pharmaceutical tablet is provided, comprising: a β-agent in an amount from about 30 to 160 μg, and a peripherally acting β-blocker (PABRA) in an amount from about 15 mg or less.

In embodiments of any aspect or embodiment of the disclosure described herein, the β-agent is in an amount from about 0.01 to 100 mg.

In embodiments of any aspect or embodiment of the disclosure described herein, the β-agent is in an amount from about 0.5-50 mg; or 1-25 mg; or 1-10 mg; or 10-20 mg; or 25-50 mg; or mg; or 2-8 mg; or about 1 mg; or about 2 mg; or about 3 mg; or about 4 mg, or about 5 mg; or about 6 mg; or about 7 mg; or about 8 mg; or about 10 mg; or abut 15 mg; or about 20 mg; or about 25 mg; or about 30 mg; or about 40 mg; or about 50 mg.

In embodiments of any aspect or embodiment of the disclosure described herein, said β-agent is administered at a dose of from about 30 to 160 μg, 50 to 160 μg, 80 to 160 μg, 100 to 160 μg, 120 to 160 μg, 140 to 160 μg, 30 to 140 μg, 50 to 140 μg, 80 to 140 μg, 100 to 140 μg, 120 to 140 μg, 30 to 120 μg, 50 to 120 μg, 80 to 120 μg, 100 to 120 μg, 30 to 100 μg, 50 to 100 μg, 80 to 100 μg, 30 to 80 μg, 50 to 80 μg, 30 to 50 μg, 30 μg, 40 μg, 50 μg, 60 μg, 70 μg, 80 μg, 90 μg, 100 μg, 110 μg, 120 μg, 130 μg, 140 μg, 150 μg, or 160 μg.

In embodiments of any aspect or embodiment of the disclosure described herein, the above-mentioned dose of β-agent is a total daily dose and is administered daily for a period of weeks or more.

In embodiments of any aspect or embodiment of the disclosure described herein, the above-mentioned dose of β-agent is a weekly and is administered weekly for a period of two weeks or more.

In embodiments of any aspect or embodiment of the disclosure described herein, the peripherally acting β-blocker (PABRA) is provided in an amount from about 0.1 to 15 mg, 0.1 to 10 mg, 0.1 to 1 mg, 0.1 to 5 mg, 1 to 15 mg, 1 to 10 mg, 1 to 5 mg, 5 to 10 mg, 10 mg or less, 7 mg or less, 5 mg or less, 1 mg or less, 0.1 mg, 0.5 mg, 1 mg, 2 mg, 3 mg, 4 mg, 5 mg, 6 mg, 7 mg, 8 mg, 9 mg, or 10 mg.

In embodiments of any aspect or embodiment of the disclosure described herein, the peripherally acting β-blocker (PABRA) is provided in an amount from about 0.1 to 15 mg.

In embodiments of any aspect or embodiment of the disclosure described herein, the peripherally acting β-blocker (PABRA) is provided in an amount from about 5 to 10 mg.

In embodiments of any aspect or embodiment of the disclosure described herein, the above-mentioned dose of the peripherally acting β-blocker (PABRA) is a total daily dose and is administered daily for a period of weeks or more.

In embodiments of any aspect or embodiment of the disclosure described herein, the above-mentioned dose of the peripherally acting β-blocker (PABRA) is a weekly dose and is administered weekly for a period of weeks or more.

In embodiments of any aspect or embodiment of the disclosure described herein, the peripherally acting β-blocker (PABRA) is nadolol.

In embodiments of any aspect or embodiment of the disclosure described herein, nadolol is a mixture of four diastereomers.

In embodiments of any aspect or embodiment of the disclosure described herein, the nadolol administered is a specific enantiomerically pure isomer.

In embodiments of any aspect or embodiment of the disclosure described herein, a joint formulation is provided, comprising: a β-agent in an amount from about 30 to 160 μg, and a peripherally acting β-blocker (PABRA) in an amount from 15 mg or less.

In embodiments of any aspect or embodiment of the disclosure described herein, the β-agent is in an amount from about 50 to 160 μg.

In embodiments of any aspect or embodiment of the disclosure described herein, the β-agent is in an amount from about 80 to 160 μg.

In embodiments of any aspect or embodiment of the disclosure described herein, the β-agent is in an amount from about 0.5 to 20 mg.

In embodiments of any aspect or embodiment of the disclosure described herein, the β-agent is in an amount from about 2 to 8 mg.

In embodiments of any aspect or embodiment of the disclosure described herein, the above-mentioned dose of β-agent is a total daily dose and is administered daily for a period of weeks or more.

In embodiments of any aspect or embodiment of the disclosure described herein, the above-mentioned dose of β-agent is a weekly dose and is administered weekly for a period of two weeks or more.

In embodiments of any aspect or embodiment of the disclosure described herein, the peripherally acting β-blocker (PABRA) is in an amount from about 0.1 to 15 mg.

n embodiments of any aspect or embodiment of the disclosure described herein, the peripherally acting β-blocker (PABRA) is in an amount from about 5 to 10 mg.

In embodiments of any aspect or embodiment of the disclosure described herein, the above-mentioned dose of the peripherally acting β-blocker (PABRA) is a total daily dose and is administered daily for a period of weeks or more.

In embodiments of any aspect or embodiment of the disclosure described herein, the peripherally acting β-blocker (PABRA) is nadolol.

In embodiments of any aspect or embodiment of the disclosure described herein, nadolol is a mixture of four diastereomers.

In embodiments of any aspect or embodiment of the disclosure described herein, the nadolol administered is a specific enantiomerically pure isomer.

In embodiments of any aspect or embodiment of the disclosure described herein, a single formulation is provided, comprising: a β-agent in an amount from about 30 to 160 μg, and a peripherally acting β-blocker (PABRA) in an amount from 15 mg or less.

n embodiments of any aspect or embodiment of the disclosure described herein, the β-agent is in an amount from about 0.01 to 100 mg.

In embodiments of any aspect or embodiment of the disclosure described herein, the β-agent is in an amount from about 0.5-50 mg; or 1-25 mg; or 1-10 mg; or 10-20 mg; or 25-50 mg; or mg; or 2-8 mg; or about 1 mg; or about 2 mg; or about 3 mg; or about 4 mg, or about 5 mg; or about 6 mg; or about 7 mg; or about 8 mg; or about 10 mg; or abut 15 mg; or about 20 mg; or about 25 mg; or about 30 mg; or about 40 mg; or about 50 mg.

In embodiments of any aspect or embodiment of the disclosure described herein, the above-mentioned dose of β-agent is a total daily dose and is administered daily for a period of weeks or more.

In embodiments of any aspect or embodiment of the disclosure described herein, the peripherally acting β-blocker (PABRA) is in an amount from about 0.1 to 15 mg.

In embodiments of any aspect or embodiment of the disclosure described herein, the peripherally acting β-blocker (PABRA) is in an amount from about 5 to 10 mg.

In embodiments of any aspect or embodiment of the disclosure described herein, the above-mentioned dose of the peripherally acting β-blocker (PABRA) is a total daily dose and is administered daily for a period of weeks or more.

In embodiments of any aspect or embodiment of the disclosure described herein, the peripherally acting β-blocker (PABRA) is nadolol.

In embodiments of any aspect or embodiment of the disclosure described herein, nadolol is a mixture of four diastereomers.

In embodiments of any aspect or embodiment of the disclosure described herein, the nadolol administered is a specific enantiomerically pure isomer.

While the disclosure has been particularly shown and described with reference to specific embodiments (some of which are preferred embodiments), it should be understood by those having skill in the art that various changes in form and detail may be made therein without departing from the spirit and scope of the present disclosure as disclosed herein.

All references referred to in the present disclosure are hereby incorporated by reference in their entirety. Various embodiments of the present disclosure may be characterized by the potential claims listed in the paragraphs following this paragraph (and before the actual claims provided at the end of this application). These potential claims form a part of the written description of this application. Accordingly, subject matter of the following potential claims may be presented as actual claims in later proceedings involving this application or any application claiming priority based on this application. Inclusion of such potential claims should not be construed to mean that the actual claims do not cover the subject matter of the potential claims. Thus, a decision to not present these potential claims in later proceedings should not be construed as a donation of the subject matter to the public.

The embodiments of the disclosure described above are intended to be merely exemplary; numerous variations and modifications will be apparent to those skilled in the art. All such variations and modifications are intended to be within the scope of the present disclosure as defined in any appended claims.

1. A method comprising:
administering to a subject a β-agent and a peripherally acting β-blocker (PABRA), wherein the peripherally acting β-blocker (PABRA) is administered in a sub-therapeutic dose, wherein the subject has, or has been identified as having one or more conditions selected from the group consisting of major depressive disorder, treatment resistant depression (MDD/TRD), late age depression, anhedonia, post-traumatic stress disorder (PTSD), schizophrenia, cognitive/emotional impairment associated with schizophrenia, seasonal affective disorder (SAD).

2. (canceled)

3. The method of claim 1, further comprising:
subjecting said subject to brain imaging to determine cognitive function and/or to identify whether said subject is in need of or desiring improvement of cognitive function and/or treatment of a neurodegenerative disease.

4. The method of claim 3, further comprising:
identifying a particular type of neurodegenerative disease based on a spatial pattern of the brain imaging result.

5. The method of claim 3, further comprising:
subsequently re-subjecting said subject to brain imaging to determine any improvement in cognitive function and/or treatment of said neurodegenerative disease.

6. The method of claim 3, wherein the brain imaging is fluorodeoxyglucose positron emission tomography (FDG-PET) scan, magnetic resonance imaging-arterial spin labeling (MRI-ASL), or magnetic resonance imaging-blood oxygenation level dependent computerized tomography (MRI-BOLD).

7. The method of claim 1, wherein said β-agent is administered at a dose of from about 30 to 160 μg.

8-11. (canceled)

12. The method of claim 1, wherein the peripherally acting β-blocker (PABRA) is administered in a dose of about 0.1 to 30 mg.

13-14. (canceled)

15. The method of claim 1, wherein the peripherally acting β-blocker (PABRA) is one or more selected from the group consisting of nadolol, atenolol, sotalol and labetalol.

16. The method of claim 1, wherein the peripherally acting β-blocker (PABRA) is nadolol.

17-19. (canceled)

20. The method of claim 1, wherein the β-agent and peripherally acting β-blocker (PABRA) are each administered orally.

21. The method of claim 1, wherein the β-agent is administered at a dose of from about 30 to 160 μg.

22-26. (canceled)

27. The method of claim 1, wherein the peripherally acting β-blocker (PABRA) is administered in a dose of about 0.1 to 15 mg.

28-30. (canceled)

31. The method of claim 3, wherein the neurodegenerative disease is one or more selected from the group consisting of MCI, aMCI, Vascular Dementia, Mixed Dementia, FTD (fronto-temporal dementia; Pick's disease), HD (Huntington disease), Rett Syndrome, PSP (progressive supranuclear palsy), CBD (corticobasal degeneration), SCA (spinocerebellar ataxia), MSA (Multiple system atrophy), SDS (Shy-Drager syndrome), olivopontocerebellar atrophy, TBI (traumatic brain injury), CTE (chronic traumatic encephalopathy), stroke, WKS (Wernicke-Korsakoff syndrome; alcoholic dementia & thiamine deficiency), normal pressure hydrocephalus, hypersomnia/narcolepsy, ASD (autistic spectrum disorders), FXS (fragile X syndrome), TSC (tuberous sclerosis complex), prion-related diseases (CJD etc.), depressive disorders, DLB (dementia with Lewy bodies), PD (Parkinson's disease), PDD (PD dementia), ADHD (attention deficit hyperactivity disorder), and Down Syndrome.

32-46. (canceled)

47. A method, comprising:
subjecting a subject to a test to determine cognitive function and/or to identify whether said subject is in need of or desiring improvement of cognitive function and/or treatment of a neurodegenerative disease;
identifying a particular type of neurodegenerative disease based on a spatial pattern of the test result;
and subsequently administering to said subject a pharmaceutical composition comprising a β-agent, wherein the peripherally acting β-blocker (PABRA) is administered in a dose of about 15 mg or less.

48-54. (canceled)

55. The method of claim 47, wherein the subject has, or has been identified as having one or more conditions selected from the group consisting of major depressive disorder, treatment resistant depression (MDD/TRD), late age depression, anhedonia, post-traumatic stress disorder (PTSD), schizophrenia, cognitive/emotional impairment associated with schizophrenia, seasonal affective disorder (SAD).

56-63. (canceled)

64. The method of claim 47, wherein the test is brain imaging.

65. The method of claim 47, wherein the test is fluorodeoxyglucose positron emission tomography (FDG-PET) scan, magnetic resonance imaging-arterial spin labeling (MRI-ASL), or magnetic resonance imaging-blood oxygenation level dependent computerized tomography (MRI-BOLD).

66. The method of claim 1, wherein the β-agent is administered at a dose of from about 30 to 160 μg.

67-71. (canceled)

72. The method of claim 1, wherein the peripherally acting β-blocker (PABRA), if present, is one or more selected from the group consisting of nadolol, atenolol, sotalol and labetalol.

73. The method of claim 1, wherein the peripherally acting β-blocker (PABRA) is nadolol.

74. (canceled)

75. The method of claim 1, wherein the peripherally acting β-blocker (PABRA) is administered in a dose of about 0.1 to 30 mg.

76-85. (canceled)

86. A pharmaceutical tablet or capsule, comprising:
a therapeutically effective amount of a β-agent, and
a peripherally acting β-blocker (PABRA) in an amount from 15 mg or less.

87-89. (canceled)

90. A pharmaceutical tablet or capsule, comprising:
a β-agent in an amount from about 0.5-20 mg; or 1-10 mg; or 2-8 mg; or about 1 mg; or about 2 mg; or about 3 mg; or about 4 mg; or about 5 mg; or about 6 mg; or about 7 mg; or about 8 mg; or about 10 mg, and
a peripherally acting β-blocker (PABRA) in an amount from 15 mg or less.

91-113. (canceled)

114. The method or composition of claim 1, wherein the dose of the PABRA is 90% or less; or 85% or less; or 80% or less; or 75% or less; or 70% or less; or 65% or less; or 60% or less; or 55% or less; or 50% or less; or 45% or less; or 40% or less; or 35% or less; or 30% or less; or 25% or less; or 20% or less; or 15% or less; or 10% or less; or 5% or less; or 4% or less; or 3% or less; or 2.5% or less; or 2% or less; or 1.5% or less; or 1% or less; or 0.5% or less as compared to a dose that the agent is effective for, or approved for treating a specific disease indication.

115. (canceled)

116. The method or composition of claim 1, wherein the total daily dose of the β-agent is from about 1 to 300 μg, 5 to 200 μg, 10 to 180 μg, 10 to 40 g, 20 to 50 μg, 40 to 80 g, 50 to 100 μg, 100 to 200 μg, 30 to 160 μg, 50 to 160 μg, 80 to 160 μg, 100 to 160 μg, 120 to 160 μg, 140 to 160 μg, 150 to 170 μg, 30 to 140 μg, 50 to 140 μg, 80 to 140 μg, 100 to 140 μg, 120 to 140 μg, 30 to 120 μg, 50 to 120 μg, 80 to 120 μg, 100 to 120 μg, 30 to 100 μg, 50 to 100 μg, 80 to 100 μg, 30 to 80 μg, 50 to 80 μg, 30 to 50 μg, about 10 μg, about 20 μg, about 25 μg, about 30 μg, about 40 μg, about 50 μg, about 60 μg, about 70 μg, about 80 μg, about 90 μg, about 100 μg, about 110 μg, about 120 μg, about 125 μg, about 130 μg, about 140 μg, about 150 μg, or about 160 μg, about 170 μg, about 175 μg, about 180 μg, about 190 μg, about or 200 μg.

117. The method or composition of claim 1, wherein the β-agent is administered at a dose from 0.5-20 mg; or 1-10 mg; or 2-8 mg; or 1-15 mg; or 3-12 mg; or about 1 mg; or about 2 mg; or about 3 mg; or about 4 mg; or about 5 mg; or about 6 mg; or about 7 mg; or about 8 mg; or about 10 mg; or about 11 mg; or about 12 mg; or about 13 mg; or about 15 mg.

118. The method or composition of claim 1, wherein the β-agent is a compound with a structure of Formula (I), Formula (I″), Formula (II), Formula (III), Formula (I′), Formula (II′), Formula (III′), Formula (IV′), Formula (V′), Formula (VI′), Formula (VII′), Formula (VIII′), Formula (IX′), Formula (X′), Formula (XI′), Formula (XII′), Formula (XIII′), Formula (XIV′), Formula (XV′), Formula (XVI′), Formula (XVII′), Formula (XVIII′), Formula (XIX′), Formula (XX′), Formula (XXI′), Formula (XXII′), Formula (XXIII′), Formula (XXIV′), or Formula (XXV′); or an optically pure stereoisomer, pharmaceutically acceptable salt, solvate, or prodrug thereof.

119-143. (canceled)

144. The method or composition of claim 1, wherein the β-agent is a compound having the structure:

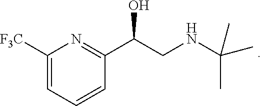 [SMILES: CC(C)(C)NC[C@H](O)c1cccc(C(F)(F)F)n1]

145. The method or composition of claim 1, wherein the β-agent is a compound having the structure:

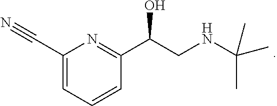 [SMILES: CC(C)(C)NC[C@H](O)c1cccc(C#N)n1]

146. The method or composition of claim 1, wherein the β-agent is a compound having the structure: [chemical structure]
